(19) United States
(12) Patent Application Publication
(10) Pub. No.: US 2026/0242790 A1
(43) Pub. Date: Aug. 20, 2026

(54) OPTIMIZED ANTISENSE SEQUENCES THAT ENHANCE TARGETED RNA EDITING BY ADAR PROTEINS

(71) Applicant: The Board of Trustees of the Leland Stanford Junior University, Stanford, CA (US)

(72) Inventors: Jin Billy Li, Stanford, CA (US); Inga Jarmoskaite, Stanford, CA (US); Rohini Datta, Stanford, CA (US); Paul Vogel, Stanford, CA (US)

(21) Appl. No.: 19/165,432

(22) PCT Filed: Mar. 15, 2024
(86) PCT No.: PCT/US2024/020192

§ 371 Date: Sep. 15, 2025

Related U.S. Application Data
(60) Provisional application No. 63/452,772, filed on Mar. 17, 2023.

(51) Int. Cl. C12N 15/113

(52) U.S. Cl. CPC C12N 15/113; C12N 15/1136; C12N 15/1137; C12Y 102/01003; C12Y 306/05002; C12N 2310/11; C12N 2310/321; C12N 2310/322; C12N 2310/351

(57) ABSTRACT

Provided here in is an oligonucleotide for adenosine to inosine (A-to-I) editing of a target RNA sequence, wherein the oligonucleotide comprises a general core sequence: 3′-N₋₁₀ N₋₉ N₋₈ N₋₇ N₋₆ N₋₅ N₋₄ N₋₃ N₋₂ N₋₁ N₀ N₊₁ N₊₂ N₊₃ N₊₄ N₊₅ N₊₆ N₊₇ N₊₈ N₊₉-5′; wherein (3′-N₋₁ N₀ N₊₁-5′) is a central base triplet (CBT) with the central nucleotide (N₀) directly opposite the target adenosine (A) to be edited, when the oligonucleotide is hybridized to the target RNA sequence, and wherein the target RNA sequence has a core sequence of: 5′-n₋₁₀ n₋₉ n₋₈ n₋₇ n₋₆ n₋₅ n₋₄ n₋₃ n₋₂ n₋₁ A n₊₁ n₊₂ n₊₃ n₊₄ n₊₅ n₊₆ n₊₇ n₊₈ n₊₉-3′; and wherein (i) there is a mismatch at N₀, when the oligonucleotide is hybridized to the target RNA sequence; (ii) there is one or more nucleotide mismatches outside of the CBT that is/are not complementary to the corresponding position in the target RNA sequence; and (iii) the one or more nucleotide mismatch(es) outside of the CBT is/are as defined by generalizable rules 1 to 8.

### PRIORITY STATEMENT

This application claims priority to U.S. Provisional Patent Application No. 63/452,772, filed Mar. 17, 2023, the entire contents of which are incorporated herein by reference for all purposes.

### SEQUENCE LISTING

The text of the computer readable sequence listing filed herewith, titled “STDU2_41750_601”, created Mar. 14, 2024, having a file size of 1,169,503 bytes, is hereby incorporated by reference in its entirety.

### FIELD

The present invention relates to the field of medicine, in particular to the field of site-directed A-to-I RNA editing, whereby a target RNA sequence is targeted by an antisense oligonucleotide (ASO). The present disclosure relates to novel guide RNAs for use in site-directed RNA editing. In particular, provided herein are guide RNAs (gRNAs) effective for site directed RNA editing, and generalized rules for engineering highly effective guide RNAs. The scope of the invention further encompasses methods of using the gRNAs disclosed herein for use in site-directed RNA editing, and for the treatment of various diseases.

### BACKGROUND

Site-Directed RNA Editing (SDRE) describes the alteration of an RNA sequence by introducing or removing nucleotides from an RNA or by changing the character of a nucleobase by deamination. RNA editing enzymes are known in the art. As described in Zinshteyn and Nishikura (2009), the first RNA editing process discovered in mammals was the deamination of cytidine (C) to uridine (U) by apolipoprotein B mRNA editing enzyme, catalytic polypeptide (APOBEC). To date, the two most useful and most studied types of RNA editing are cytidine (C) to uridine (U) (“C-to-U”) and adenosine (A) to inosine (I) (“A-to-I”) conversions. Notably, the most relevant for therapeutic purposes and the most prevalent type of RNA editing in higher eukaryotes is the “A-to-I”′ conversion.

“A-to-I” conversion is catalysed by the adenosine deaminases acting on RNA (ADARs) family. “A-to-I” editing was first identified in Xenopus eggs (Bass and Weintraub, 1987; Rebagliati and Melton, 1987). During this enzymatically catalyzed reaction adenosine is changed via a hydrated intermediate to inosine. While guanosine can form three hydrogen bonds to the complementary base cytidine, inosine can form only two hydrogen bonds to cytidine. The translational machinery reads inosine as a guanosine. Therefore, ADARs have the effect of introducing a functional adenosine to guanosine mutation on the RNA level. The ability of ADARs to alter the sequence of RNAs has also been used to artificially target RNAs in vitro and in vivo for RNA editing. Potentially this approach may be used to repair genetic defects and alter genetic information at the RNA level.

Over the years, three vertebrate ADAR genes have been identified, which give rise to several ADAR proteins that result from alternative promoters or represent splice variants (Wulff and Nishikura, 2010). These proteins are expressed across various types of human tissues and can alter splicing patterns, amino acid sequences, double-stranded RNA (dsRNA) structures and the binding affinity between RNA and RNA-binding proteins (Tomaselli et al., 2014; Zinshteyn and Nishikura, 2009). Of the three known ADAR genes, hADAR1 and hADAR2 are expressed in most tissues and encode active deaminases. Human ADAR3 (hADAR3) has been described to only be expressed in the central nervous system and reportedly has no deaminase activity. While all ADARs are multidomain proteins, comprising two or three dsRNA-binding domains (dsRBDs) and a catalytic domain, ADAR1 proteins additionally comprise one or more Z-form DNA/RNA binding domains, while splice variant ADAR2R and ADAR3 comprise an R domain (Zinshteyn and Nishikura, 2009; Wulff and Nishikura, 2010). Accordingly, in some embodiments, the ADAR is hADAR1, hADAR2 or hADAR3. “A-to-I” RNA editing systems employing endogenous adenosine deaminase enzymes have been extensively studied, i.e., the use of exogenous oligonucleotides to specifically recruit endogenous adenosine deaminases to a specific target site of a target RNA thereby providing an improved system for targeted RNA editing.

For instance, oligonucleotide constructs for site-directed RNA editing are described in patent applications WO 2016/097212 and WO 2017/010556, which utilize endogenous cellular pathways, i.e., endogenous ADAR, to edit endogenous RNA.

Antisense oligonucleotides (ASOs) are generally short single-stranded synthetic RNA or DNA molecules, which use Watson-Crick base pairing to bind sequence specifically to the target RNA. ASOs can work through many mechanisms depending, in part, on the region in the RNA sequence that is targeted and ASO design/chemical properties.

Oligonucleotide-guided RNA editing by endogenous ADAR proteins has recently emerged as a powerful platform for therapeutic transcriptome engineering. Hence, ASO-based therapies have been gaining more traction over the past years for the treatment of different medical conditions and diseases, and in specific genetic disorders. Endogenous ADAR proteins can be directed to edit mRNA targets of interest by providing short guide RNAs (gRNAs or ASOs). However, the optimal design of ADAR gRNAs remains to be determined. Moreover, general rules for designing effective oligonucleotide guides that direct ADAR proteins to desired mRNA sites are yet to be defined. Accordingly, there remains a need in the art for optimized gRNA or ASO sequences for the editing of disease-relevant targets, and novel methods for optimizing oligonucleotides for ADAR-mediated RNA editing that are broadly applicable to a variety of disease targets.

### SUMMARY

Generally provided herein are guide RNA or antisense oligonucleotide (ASO) sequences for adenosine to inosine (A-to-I) editing of a target RNA sequence. In some aspects, provided herein generalizable rules. In some aspects, provided herein are compositions comprising a guide RNA sequence or oligonucleotide as provided herein. Also provided herein are methods for creating de-novo A-to-G beneficial protein variants and methods of treating or preventing a disease or condition associated with a point mutation or a wild-type protein. In some aspects, provided herein are oligonucleotide and compositions for use in a method of treating or preventing a disease or condition associated with a point mutation or a wild-type protein.

Therefore, the problem solved by the invention lies in the provision of improved gRNAs or antisense oligonucleotides for A-to-I editing and ‘generalizable rules’ for such oligonucleotides. The present invention specifically solves the problem by providing various, target-specific, synthetic oligonucleotides that create at least two mismatches when hybridized to the target RNA sequence, i.e., a nucleotide mismatch directly opposite the target adenosine (A) to be edited in the target RNA, and one or more nucleotide mismatches 5′ or 3′ to target adenosine (A) that is/are not complementary to the corresponding nucleotide position in the target RNA sequence. Overall, the oligonucleotides provided herein show several differences and associated advantages over those disclosed in the prior art. Above all, the inventive character of the inventions lies in the identification of sets of ‘generalizable rules’ applicable to oligonucleotides depending on their target. To date, no prior art has been identified that teaches or suggests ‘generalizable rules’ that can be used and applied to oligonucleotides to increase A-to-I editing of a target RNA.

The solution to the problem addressed by the instant application is achieved by the embodiments described herein and defined by the appended claims.

In some embodiments, the guide RNA sequence comprises a recruitment domain and an antisense sequence that is sufficiently complementary to the target sequence to promote ADAR mediated RNA editing. In some embodiments, guide RNA sequence conforms to one or more generalizable rules provided herein. In some aspects, provided herein are guide RNAs with increased site-directed RNA editing efficiency of various target sites in disease-relevant transcripts. In some aspects, provided herein are methods for site-directed RNA editing and treatment of disease.

### BRIEF DESCRIPTION OF THE DRAWINGS

The figures shown in the following are merely illustrative and shall describe the present invention in a further way. The figures shall not be construed to limit the present invention thereto.

FIG. 1 is a schematic ADAR-mediated site-directed A-to-I RNA editing.

FIG. 2 is a schematic showing an exemplary method for high-throughput screening of gRNA variants.

FIG. 3A and FIG. 3B are schematics showing 8 generalizable rules for optimizing gRNA sequences.

FIG. 4 is a schematic showing the numbering of bases in target RNA and gRNA.

### DETAILED DESCRIPTION

### 1. Definitions

To facilitate an understanding of the present technology, a number of terms and phrases are defined below. Additional definitions are set forth throughout the detailed description.

Unless otherwise defined herein, scientific and technical terms used in connection with the present disclosure shall have the meanings that are commonly understood by those of ordinary skill in the art. For example, any nomenclature used in connection with, and techniques of, cell and tissue culture, biochemistry, molecular biology, immunology, microbiology, genetics and protein and nucleic acid chemistry and hybridization described herein are those that are well known and commonly used in the art. The meaning and scope of the terms should be clear; in the event, however, of any latent ambiguity, definitions provided herein take precedent over any dictionary or extrinsic definition. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element, e.g., a plurality of elements.

The terms “about” and “approximately” may be understood to permit standard variation as would be understood by those of ordinary skill in the art.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “amino acid” refers to natural amino acids, unnatural amino acids, and amino acid analogs, all in their D and L stereoisomers, unless otherwise indicated, if their structures allow such stereoisomeric forms.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%, 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

The terms “target transcript” and “target sequence” are used interchangeably herein. The target transcript will comprise an RNA sequence, e.g., a messenger RNA, comprising an adenosine at a target site, also referred to herein as the “target adenosine” or “target A”. In a primary implementation, the target A comprises an undesirable mutation in the RNA that imparts deleterious properties, for example, a mutation that introduces a premature stop codon, a mutation that induces an amino acid substitution that disrupts normal protein function, a mutation that disrupts normal RNA splicing, etc. In alternative implementations, the target A is edited to change the wild-type RNA sequence in a way that imparts new properties to the resulting protein, for example, improving or changing the function of the protein. It is an objective of the invention, and ADAR-mediated RNA editing in general, to correct mutations or edit target A's by harnessing RNA editing enzymes, in a primary embodiment, ADAR enzymes, for example, endogenous ADAR enzymes, to achieve an A-to-I substitution in the target transcript, functionally achieving an adenosine to guanosine or “A-to-G” transition.

The term “adenosine deaminases acting on RNA” or “ADARs” are used herein to refer to a class of enzymes that naturally catalyze A-to-I editing of sites within target transcripts. The I nucleotide is read as G by the ribosome during translation; consequently, A-to-I editing effectively introduces A-to-G substitutions in protein-coding RNA. ADARs can play important roles in the regulation of protein function, RNA splicing, immunity, and RNA interference. Isoforms of ADAR include ADAR1 p110, ADAR1 p150, ADAR2, and the catalytically inactive ADAR3; for example, human ADAR1 p110, p150, or ADAR2. Engineered forms of ADAR are employed in some therapeutic strategies.

As used herein, a “nucleic acid” or a “nucleic acid sequence” refers to a polymer or oligomer of pyrimidine and/or purine bases, preferably cytosine I, thymine (T), and uracil (U), and adenine (A) and guanine (G), respectively. The term is intended to include any DNA molecules (e.g., cDNA or genomic DNA) and any RNA molecules (e.g., mRNA) and analogues of DNA or RNA generated using nucleotide analogues. The present technology contemplates any deoxyribonucleotide (DNA), ribonucleotide (RNA), or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506, incorporated herein by reference), locked nucleic acid (LNA; see Wahlestedt et al., Proc. Natl. Acad. Sci. U.S.A., 97:5633-5638 (2000), incorporated herein by reference), cyclohexenyl nucleic acids (see, Wang, J. Am. Chem. Soc., 122:8595-8602 (2000), incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (i.e., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof. Each component of the DNA or RNA structure can be modified and be categorized by modification of (1) the internucleoside linkage, (2) the deoxyribose/ribose, and/or (3) the nucleobase.

The term “oligonucleotide” or “oligonucleotides” as used herein are defined as it is generally understood by the skilled person as a molecule including two or more covalently linked nucleosides (e.g., short nucleic acid polymer(s)). They can comprise DNA and/or RNA. The oligonucleotides provided herein have a backbone comprising deoxyribonucleotides and/or ribonucleotides. The term “oligonucleotide” may be used interchangeably with the term “antisense oligonucleotide (ASO)”. This means that, as used herein the term “antisense oligonucleotide” or “ASO” refers to a short strand of nucleotide analogue that hybridizes with a (partially) complementary mRNA in a sequence-specific manner.

The terms “hairpin”, “hairpin loop”, “stem loop”, and “stem loop secondary structure” are used interchangeably herein to refer to a structure formed in a single stranded oligonucleotide when sequences within the single strand which are complementary when read in opposite directions base pair to form a region whose conformation resembles a hairpin or loop.

As used herein, the term “wobble base pair” is a pairing between two nucleotides that does not follow Watson-Crick base pair rules. The four main wobble base pairs are guanine-uracil (G-U), hypoxanthine-uracil (I-U), hypoxanthine-adenine (I-A), and hypoxanthine-cytosine (I-C).

As used herein, the term “chemical modification” preferably refers to a chemical modification selected from backbone modifications, sugar modifications or base modifications, including abasic sites, A “chemically modified oligonucleotide” in the context of the present invention may refer to a nucleic acid comprising at least one chemically modified nucleotide.

The term “modified” nucleobase” and “modified base” may be used interchangeably with the term “nucleobase”. Nucleobases may be modified or unmodified. Hence, in some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In one embodiment, the modified nucleobase is capable of increasing hydrogen bonding, base pair stacking interactions and/or stabilizing a nucleic acid complex. In another embodiment, the modified nucleobase (e.g., Benner's base) is capable of mimicking the N3 protonated cytosine base. In some embodiments, a modified nucleobase is substituted A. T, C, G, or U, or a substituted tautomer of A, T, C, G, or U. In some embodiments, a modified nucleobase in the context of oligonucleotides refer to a nucleobase that is not A, T, C, G or U. Modifications include but are not limited to nonstandard nucleobases 5-methyl-2′-deoxycytidine (m5C), pseudouridine (pU), dihydrouridine, inosine (I), and 7-methylguanosine. Other modifications may include nucleobase replacement by (N) heterocycles (e.g., nebularine) or aromatic rings that stack well in the RNA duplex, such as, e.g., a Benner's base Z (and/or analogues) or 8-oxo-adenosine (8-oxo-A). As used herein, the term “Benner's base Z” refers to the pyrimidine analogue 6-amino-5-nitro-3-(1′-B-D-2′-deoxyribofuranosyl)-2 (1H)-pyridone (dZ). In one embodiment, a modification includes the introduction of nucleobase analogues or simple heterocycles that boost editing. As used herein, and as commonly understood by the skilled person in the art, the expression “derivative thereof” refers to a derivative of a (modified) nucleobase, nucleoside or nucleotide. For example, a derivative may be a corresponding nucleobase, nucleoside or nucleotide that has been chemically derived from said nucleobase, nucleoside or nucleotide. For instance, a derivative of deoxycytidine may include fluoro-modified deoxycytidine, 5-methyl-2′-deoxycytidine (m5C), or ribocytidine.

The term “FANA” or “FANA-modified” refers to 2′-fluoroarabinoside modified nucleobases and/or oligonucleotides comprising such nucleobases. For example, the expression “FANA-cytidine” refers to a cytidine that comprises a 2′-fluoro-beta-D-arabinonucleic acid sugar modification. Within the context of this invention, the expression “a derivate thereof” refers to a corresponding nucleotide(s) or oligonucleotide(s) that has been chemically derived from said nucleotide or oligonucleotide(s).

As used herein, the term “internucleoside linkage” refers to a linkage between adjacent nucleosides. “Internucleoside linkage” and “linkage” may be used interchangeably. Linkages may be continuous or consecutive. Linkages may be discontinuous or interrupted. As used herein, the term “discontinuous” or “interrupted” means that there are not more than, e.g., 4, 5, 6, 7 or more consecutive internucleoside linkage modifications of the same modification. In some embodiments, the naturally occurring PO linkages are replaced by modified internucleoside linkages. Hence, in some embodiments, the linkage is a non-natural internucleoside linkage. In some embodiments, internucleoside linkage(s) include, but are not limited to phosphorothioate (PS), 3′-methylenephosphonate, 5′-methylenephosphonate, 3′-phosphoroamidate, 2′-5′-phosphodiester, and phosphoryl guanidine (PN) linkages. In another embodiment, the internucleoside linkage modification is a 3′-3′ or 5′-5′ phosphate ester bonds (3′-P-3′ and 5′-P-5′). The internucleoside linkage may be stereopure or stereorandom. Thus, within a particular oligonucleotide, internucleoside linkages may comprise stereopure and stereorandom linkages. In one embodiment, the natural 3′-5′ phosphodiester linkage is replaced by modified internucleoside linkages. In some embodiments, the naturally occurring one or more PO linkages are replaced by modified internucleoside linkages in order to introduce one or more PS linkages or non-phosphorus derived internucleoside linkages.

The term “linker,” as used herein, refers to a bond (e.g., covalent bond), chemical group, or a molecule linking two molecules or moieties, e.g., two domains of a fusion protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20-30, 40-50, 50-60, 60-70, 70-80, 80-90, 90-100, 100-150, or 150-200 amino acids in length. Longer or shorter linkers are also contemplated herein. In some embodiments, the linker is 3 amino acids.

The term “guide RNA” (gRNA) or “guide oligonucleotide”, as used herein refers to a nucleic acid designed to be substantially complementary to the “target sequence”. The guide RNA or guide (antisense) oligonucleotide can comprise endogenous and/or exogenous sequences. Guides can be used in vitro and in vivo. The terms “target RNA sequence”, “target nucleic acid”, “target sequence”, and “target site” are used interchangeably herein to refer to a polynucleotide (nucleic acid, gene, chromosome, genome, etc.) to which a guide RNA sequence is designed to have complementarity. The terms “antisense domain” and “antisense sequence” are used interchangeably herein. The antisense domain (i.e., antisense sequence) of the gRNA (or oligonucleotide of the invention) binds to the target RNA. As used herein, the term “target RNA” typically refers to an RNA, which is subject to the editing reaction, and “targeted” by the respective antisense oligonucleotides

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Notably, the invention is not limited to correcting mutations, as it may instead be useful to change a wild-type sequence into a mutated sequence by applying an oligonucleotide of the invention.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA. A “variant of a sequence” or a “sequence “variant,” as used herein comprises, in various embodiments, a sequence having at least 60%, at least 65%, at least 70%, at least 75%, at least 80%, at least 85%, at least 90%, at least 95%, or at least 99% to an enumerated sequence.

A “vector” or “expression vector” is a replicon, such as plasmid, phage, virus, or cosmid, to which another DNA segment, e.g., an “insert” may be attached or incorporated so as to bring about the replication of the attached segment in a cell. For example, the “insert” may be a construct as described herein. For example, the “insert” may be a construct comprising a target sequence and a guide RNA sequence as described herein.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified”, “mutant”, or “polymorphic” refers to a gene or gene product that displays modifications in sequence and or functional properties (e.g., altered characteristics) when compared to the wild-type gene or gene product. It is noted that naturally occurring mutants can be isolated; these are identified by the fact that they have altered characteristics when compared to the wild-type gene or gene product.

As used herein, the terms “disease” or “disorder” are used interchangeably to refer to a condition in a subject. In certain embodiments, the condition is a disease or disorder associated with one of the genes selected form the list consisting of: SNCA, SERPINA1, GFER, ALDH2, TP53, SCN9A, HBB, KRAS, MeCP2, F5, ABCA4, STAT3, STAT1, IFNAR1, CTNNB1, TMEM173, and/or an RNA encoding the respective polypeptide. In one embodiment, the condition is a disease or disorder associated with one of the following mutations: SNCA A53T, SERPINA1 E342K, GFER R194H, ALDH2 E504K, TP53 R175H, SCN9A W897X, HBB W38X, KRAS (G12D, G12S), MeCP2 R106Q, F5 R534Q, ABCA4 G1961E, STAT3 Y705C, STAT1 Y701C, IFNAR1 Y466C, CTNNB1 T41A, TMEM173 (N154S and R284G).

As used herein, the term “effective amount” in the context of administering a therapy to a subject refers to the amount of a therapy which has a prophylactic and/or therapeutic effect(s).

As used herein, the terms “prevent”, “preventing” and “prevention” in the context of the present invention and the administration of a therapy (ies) to a subject refers to the inhibition of the development or onset of a disease or a symptom thereof. In one embodiment, it relates to the administration of the compound to a patient who is known to have an increased risk of developing a certain condition, disorder, or disease.

As used herein, the terms “treat”, “treatment”, and “treating” refer in the context of the present invention to the administration of the compound to a patient, which has already developed signs and/or symptoms of a certain condition, disorder, or disease. Beneficial or desired clinical results include, but are not limited to, alleviation of symptoms; diminishment of the extent of a condition, disorder, or disease stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

The terms “subject” or “patient” are used interchangeably and relate to an animal (e.g., mammals) that may need administration of the compound of the invention in the field of human or veterinary medicine. In specific embodiments, the subject is a human subject. The subject may be administered the oligonucleotide of the invention for beneficial editing. The subject may be administered the oligonucleotide of the invention for compensatory editing.

As used herein, the term “pharmaceutically acceptable” means approved by a regulatory agency. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. The formulation should suit the mode of administration.

### 2. Guide RNAs, Recruitment Domain, and Target Transcripts

### 2.1 Guide RNAS

The scope of the invention encompasses various Guide RNAs (“gRNAs”) or antisense oligonucleotides (ASOs). Oligonucleotides may only consist of a gRNA. These gRNAs comprise a specificity domain comprising a single-stranded antisense sequence that is sufficiently complementary to a selected region of the target transcript to facilitate ADAR-mediated A-to-I editing of a target adenosine (“target A”) in the target region of the transcript. The terms “specificity sequence”, “antisense specificity sequence”, “antisense domain” and “antisense sequence” are used interchangeably herein.

Site-directed RNA editing finds use to reverse the disease phenotypes caused by point mutations without the safety concerns which are associated with genome engineering and requiring only administration of the gRNA as a therapeutic. Site-directed RNA editing may also be used to insert a desired mutation into a given sequence. Alternatively, the gRNA can be expressed from a plasmid, e.g., with adeno-associated virus (AAV) delivery.

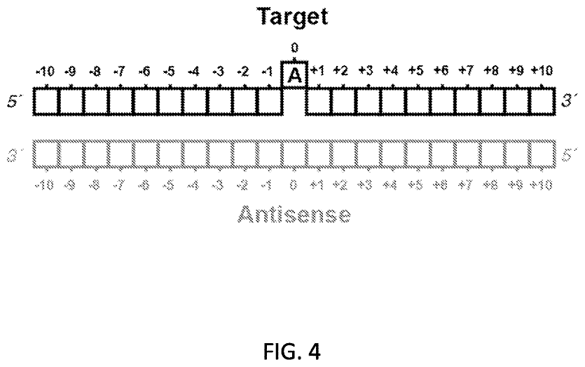

The specificity sequence may be described as a 3′ to 5′ (antisense) oligonucleotide or polynucleotide sequence. The specificity sequence and target region will be described with reference to the target “A” (adenosine to be edited). The target A is described as the “zero position” or “n₀”. Target region base positions upstream of the target A are numbered −1, −2, −3, etc. (n₋₁, n₋₂, n₋₃, etc.), by their distance upstream the target A (5′ to the target A). Target region base positions downstream of the target A are numbered +1, +2, +3, etc. (n₊₁, n₊₂, n₊₃, etc.), by their distance downstream of the target A (3′ to the target A). The antisense specificity or oligonucleotide sequence is likewise described. That is, the specificity sequence site directly opposite the target A to be edited is referred to as the zero position (N₀). The upstream (3′ to the zero position) positions are marked −1, −2, −3, etc. (N₋₁, N₋₂, N₋₃, etc.), while the downstream (5′ to the zero position) positions are numbered +1, +2, +3 (N₊₁, N₊₂, N₊₃, etc.) (see, e.g., FIG. 4).

The specificity sequence (i.e., gRNA or oligonucleotide) and target sequence (i.e., target RNA) need not exhibit complete complementarity, provided that there is sufficient complementarity to cause some hybridization. Suitable gRNA: RNA binding conditions include physiological conditions normally present in a cell. The gRNA specificity sequence will typically comprise a cytosine (C) at the zero position (N₀). In some embodiments, a mismatch (i.e., not comprising a uracil (U) or thymine (T)) will be present at the zero position. The remainder of the gRNA specificity sequence may be perfectly complementary to the targeted area, or may comprise one or more mismatches, wobble base-pairs or bulges. In some embodiments, the gRNA or oligonucleotide sequence comprises at least one, at least two, at least three, at least four, at least five, at least six, or more than six mismatches when hybridized to the target sequence.

Provided herein is an oligonucleotide for adenosine to inosine (A-to-I editing) editing of a target RNA sequence, wherein the oligonucleotide comprises a general core sequence: 3′-N₋₁₀ N₋₉ N₋₈ N₋₇ N₋₆ N₋₅ N₋₄ N₋₃ N₋₂ N₋₁ N₀ N₊₁ N₊₂ N₊₃ N₊₄ N₊₅ N₊₆ N₊₇ N₊₈ N₊₉-5′; wherein (3′-N₋₁ N₀ N₊₁-5′) is a central base triplet (CBT) with the central nucleotide (N₀) directly opposite the target adenosine (A) to be edited, when the oligonucleotide is hybridized to the target RNA sequence, and wherein the target RNA sequence has a core sequence of: 5′-n₋₁₀ n₋₉ n₋₈ n₋₇ n₋₆ n₋₅ n₋₄ n₋₃ n₋₂ n₋₁ A n₊₁ n₊₂ n₊₃ n₊₄ n₊₅ n₊₆ n₊₇ n₊₈ n₊₉-3′; and wherein (i) there is a mismatch at No, when the oligonucleotide is hybridized to the target RNA sequence; (ii) there is one or more nucleotide mismatches outside of the CBT that is/are not complementary to the corresponding position in the target RNA sequence; and (iii) the one or more nucleotide mismatch(es) outside of the CBT is/are as defined by:
a) a third generalizable rule (Rule 3), wherein the oligonucleotide contains a C or U at N₋₇, and wherein the target sequence contains a C residue at n₋₇, flanked by a 5′ G or C and a 3′ A or U;
b) a first generalizable rule (Rule 1), wherein the oligonucleotide contains a Cor U at N₋₉, and wherein the target sequence contains a C residue at n₋₉, flanked by a 5′ C or G and 3′ A or U;
c) a second generalizable rule (Rule 2), wherein the oligonucleotide contains a Cor U at N₋₈, and wherein the target sequence contains a C at n₋₈, flanked by a 5′ C or G and 3′ A or U;
d) a fourth generalizable rule (Rule 4), wherein the oligonucleotide contains a Cor U at N₋₆, and wherein the target sequence contains a C residue at n₋₆, flanked by a 5′ G or C and a 3′ A or U;
e) a fifth generalizable rule (Rule 5), wherein the oligonucleotide contains a U at N₊₂, and wherein the target sequence contains a G residue at n₊₂;
f) a sixth generalizable rule (Rule 6), wherein the oligonucleotide contains a Cor U at N₊₂, and wherein the target sequence contains a U at n₊₂;
g) a seventh generalizable rule (Rule 7), wherein the oligonucleotide contains an A at N₊₄, and wherein the target sequence contains a C residue at n₊₄, flanked by a 5′ G and 3′ C; and/or
h) an eighth generalizable rule (Rule 8), wherein the oligonucleotide contains a G at N₊₅, and wherein the target sequence contains a U residue at n₊₅, flanked by a 5′ C and 3′ U.

These generalized rules may be associated with a particular target gene. In one embodiment, under section a) above, the target sequence is SERPINA1 E342K (Rule 3). In one embodiment, under section b), the target sequence is SNCA A53T or STAT3 Y705 (Rule 1). In one embodiment, under section c) above, the target sequence is ALDH2 E504K, MeCP2 R106Q, or IFNAR1 Y466 (Rule 2). In one embodiment, under section d) above, the target sequence is SCN9A W897X (Rule 4). In one embodiment, under section e) above, the target sequence is SERPINA1 E342K, KRAS G12D, F5 R534Q, ABCA4 G1961E, IFNAR1 Y466, TMEM173 N154, or TMEM173 R284 (Rule 5). In one embodiment, under section f) above, the target sequence is GFER R194H, TP53 R175H, or KRAS G12S (Rule 6). In one embodiment, under section g) above, the target sequence is TP53 R175H (Rule 7). In one embodiment, under section h) above, the target sequence is IFNAR1 Y466 (Rule 8).

Notably, in some instances a particular oligonucleotide specific to a particular target may fall within one or more of the general rules. In one embodiment, an oligonucleotide comprises two nucleotide mismatches outside of the CBT that are not complementary to the corresponding position in the target nucleic sequence when the oligonucleotides is hybridized to the target nucleic acid sequence, wherein a) a first mismatch is defined according to Rule 3 and a second mismatch is defined according to Rule 5, optionally wherein the target sequence is SERPINA1 E342K; b) a first mismatch is defined according to Rule 6 and a second mismatch is defined according to Rule 7, optionally wherein the target sequence is TP R175H; c) a first mismatch is defined according to Rule 5 and a second mismatch is defined according to Rule 8 optionally wherein the target sequence is IFNAR1 Y466; or d) a first mismatch is defined according to Rule 2 and a second mismatch is defined according to Rule 8, optionally wherein the target sequence is IFNAR1 Y466.

In some embodiments, provided herein are methods for harnessing endogenous ADARs for the correction of mutations known to cause various disease states. Exemplary disease states and mutations are provided in Example 2, Example 3, Example 4, Example 5, Example 6, Example 7, Example 8, Example 9, and Tables 1-3.

In some aspects, provided herein are guide RNAs (ASOs). In some embodiments, provided herein is an ASO comprising the 3′-to-5′ sequence X1-X2-X3-X4-X5-X6-X7-X8-X9-X10-C, wherein X1 (−10 relative to the editing site) is C or G, forming a Watson-Crick base-pair with G or C in the target sequence, respectively; X2 (−9) is C or U, forming a mismatch with C in the target sequence; X3 (−8) is A or U, forming a Watson-Crick base-pair with U or A in the target sequence, respectively; and wherein the ASO facilitates ADAR-mediated A-to-G base replacement of a 5′-3′ target sequence comprising X1-X2-X3 -X4-X5-X6-X7-X8-X9-X10-A*, wherein X1 is Cor G, X2 is C, X3 is A or U, and A* is the target base. In one embodiment, an oligonucleotide comprises the following 3′-to-5′ core sequence: 3′-N₋₁₀ N₋₉ N₋₈ N₋₇ N₋₆ N₋₅ N₋₄ N₋₃ N₋₂ N₋₁ C-5′, wherein C is the cytosine opposite the target adenosine in the target nucleic acid sequence; wherein N₋₁₀ is C or G, forming a Watson-Crick base-pair with G or C in the target sequence; N₋₉ is C or U, forming a mismatch with C in the target sequence; and N₋₈ is A or U, forming a Watson-Crick base-pair with U or A in the target sequence.

In some embodiments, provided herein is an ASO comprising the 3′-to-5′ sequence X1-X2-X3-X4-X5-X6-X7-X8-X9-C, wherein X1 (−9 relative to the editing site) is C or G, forming a Watson-Crick base-pair with G or C in the target sequence, respectively; X2 (−8) is C or U, forming a mismatch with C in the target sequence; X3 (−7) is A or U, forming a Watson-Crick base-pair with U or A in the target sequence, respectively; and wherein the ASO facilitates ADAR-mediated A-to-G base replacement of a 5′-3′ target sequence comprising X1-X2-X3-X4-X5-X6-X7-X8-X9-A*, wherein X1 is C or G, X2 is C, X3 is A or U, and A* is the target base. In one embodiment, an oligonucleotide comprises the following 3′-to-5′ core sequence: 3′-N₋₉ N₋₈ N₋₇ N₋₆ N₋₅ N₋₄ N₋₃ N₋₂ N₋₁ C-5′, wherein C is the cytosine opposite the target adenosine in the target nucleic acid sequence; wherein N₋₉ is C or G, forming a Watson-Crick base-pair with G or C in the target sequence; N₋₈ is C or U, forming a mismatch with C in the target sequence; and N-7 is A or U, forming a Watson-Crick base-pair with U or A in the target sequence.

In some embodiments, provided herein is an ASO comprising the 3′-to-5′ sequence X1-X2-X3-X4-X5-X6-X7-X8-C, wherein X1 (−8 relative to the editing site) is C or G, forming a Watson-Crick base-pair with G or C in the target sequence, respectively; X2 (−7) is C or U, forming a mismatch with C in the target sequence; X3 (−6) is A or U, forming a Watson-Crick base-pair with U or A in the target sequence, respectively; and wherein the ASO facilitates ADAR-mediated A-to-G base replacement of a 5′-3′ target sequence comprising X1-X2-X3-X4-X5-X6-X7-X8-A*, wherein X1 is C or G, X2 is C, X3 is A or U, and A* is the target base. In one embodiment, an oligonucleotide comprises the following 3′-to-5′ core sequence: 3′-N₋₈ N₋₇ N₋₆ N₋₅ N₋₄ N₋₃ N₋₂ N₋₁ C-5′, wherein C is the cytosine opposite the target adenosine in the target nucleic acid sequence; wherein N-8 is C or G, forming a Watson-Crick base-pair with G or C in the target sequence; N₋₇ is C or U, forming a mismatch with C in the target sequence; and N-6 is A or U, forming a Watson-Crick base-pair with U or A in the target sequence.

In some embodiments, provided herein is an ASO comprising the 3′-to-5′ sequence X1-X2-X3-X4-X5-X6-X7-C, wherein X1 (−7 relative to the editing site) is C or G, forming a Watson-Crick base-pair with G or C in the target sequence, respectively; X2 (−6) is C or U, forming a mismatch with C in the target sequence; X3 (−5) is A or U, forming a Watson-Crick base-pair with U or A in the target sequence, respectively; and wherein the ASO facilitates ADAR-mediated A-to-G base replacement of a 5′-3′ target sequence comprising X1-X2-X3-X4-X5-X6-X7-A*, wherein X1 is C or G, X2 is C, X3 is A or U, and A* is the target base. In one embodiment, an oligonucleotide comprises the following 3′-to-5′ core sequence: 3′-N₋₇ N₋₆ N-5 N-4 N₋₃ N₋₂ N₋₁ C-5′, wherein C is the cytosine opposite the target adenosine in the target nucleic acid sequence; wherein N₋₇ is C or G, forming a Watson-Crick base-pair with G or C in the target sequence; N-6 is C or U, forming a mismatch with C in the target sequence; and N₋₅ is A or U, forming a Watson-Crick base-pair with U or A in the target sequence.

In some embodiments, provided herein is an ASO comprising the 3′-to-5′ sequence C-X1-X2-X3, wherein X2 (+2 relative to the editing site) is U, forming a wobble base-pair with G in the target sequence, and X1 and X3 are complementary to the corresponding positions in the target sequence; and wherein the ASO facilitates ADAR-mediated A-to-G base replacement of a 5′-3′ target sequence comprising A *-X1-X2-X3, wherein X2 is G, X1 and X3 are A, C, G or U, and A* is the target base. In one embodiment, an oligonucleotide comprises the following 3′-to-5′ core sequence: 3′-C N₊₁ N₊₂ N₊₃-5′, wherein C is the cytosine opposite the target adenosine in the target nucleic acid sequence; wherein N42 is U, forming a wobble base-pair with G in the target sequence, and N₊₁ and N₊₃ are complementary to the corresponding positions in the target sequence.

In some embodiments, provided herein is an ASO comprising the 3′-to-5′ sequence C-X1-X2-X3, wherein X2 (+2 relative to the editing site) is C or U, forming a mismatch with U in the target sequence, and X1 and X3 are complementary to the corresponding positions in the target sequence; and wherein the ASO facilitates ADAR-mediated A-to-G base replacement of a 5′-3′ target sequence comprising A *-X1-X2-X3, wherein X2 is U, X1 and X3 are A, C, G or U, and A* is the target base. In one embodiment, an oligonucleotide comprises the following 3′-to-5′ core sequence: 3′-C N₊₁ N₊₂ N₊₃-5′, wherein C is the cytosine opposite the target adenosine in the target nucleic acid sequence; wherein N₊₂ is C or U, forming a mismatch with U in the target sequence, and N₊₁ and N₊₃ are complementary to the corresponding positions in the target sequence.

In some embodiments, provided herein is an ASO comprising the 3′-to-5′ sequence C-X1-X2-X3-X4-X5, wherein X3 (+3 relative to the editing site) is C, forming a Watson-Crick base-pair with G in the target sequence, X4 is A, forming a mismatch with C in the target sequence, and X5 is G, forming a Watson-Crick base-pair with C in the target sequence; and wherein the ASO facilitates ADAR-mediated A-to-G base replacement of a 5′-3′ target sequence comprising A *-X1-X2-X3-X4-X5, wherein X3 is G, X4 is C, X5 is C, and A* is the target base. In one embodiment, an oligonucleotide comprises the following 3′-to-5′ core sequence: 3′-C N₊₁ N₊₂ N₊₃ N₊₄ N₊₅-5′, wherein C is the cytosine opposite the target adenosine in the target nucleic acid sequence; wherein N₊₃ is C, forming a Watson-Crick base-pair with G in the target sequence, N₊₄ is A, forming a mismatch with C in the target sequence, and N₊₅ is G, forming a Watson-Crick base-pair with C in the target sequence.

In some embodiments, provided herein is an ASO comprising the 3′-to-5′ sequence C-X1-X2-X3-X4-X5-X6, wherein X4 (+4 relative to the editing site) is G, forming a Watson-Crick base-pair with C in the target sequence, X5 is G, forming a wobble base-pair with U in the target sequence, and X6 is A, forming a Watson-Crick base-pair with U in the target sequence; and wherein the ASO facilitates ADAR-mediated A-to-G base replacement of a 5′-3′ target sequence comprising A *-X1-X2-X3-X4-X5-X6, wherein X4 is C, X5 is U, X6 is U, and A* is the target base. In one embodiment, an oligonucleotide comprises the following 3′-to-5′ core sequence: 3′-C N₊₁ N₊₂ N₊₃ N₊₄ N₊₅ N₊₆-5′, wherein C is the cytosine opposite the target adenosine in the target nucleic acid sequence; wherein N₊₄ is G, forming a Watson-Crick base-pair with C in the target sequence, N₊₅ is G, forming a wobble base-pair with U in the target sequence, and N₊₆ is A, forming a Watson-Crick base-pair with U in the target sequence.

According to the invention, there is a mismatch between the gRNA or oligonucleotide and the target RNA at the “zero position” (N₀/n₀). In some cases, there may be a further mismatch within the central base triple, i.e., immediately 3′ and/or 5′ to the adenosine to be edited. Hence, in some embodiments, there is a mismatch at the N₋₁ position (n₋₁ in the target RNA) and/or N₊₁ position (n₊₁ in the target RNA). This means, in one embodiment, there is a mismatch at position N₋₁ and/or at position N₊₁, when the oligonucleotide is hybridized to the target RNA sequence. In one embodiment, the oligonucleotide contains a G at N₋₁, and wherein there is a mismatch at N₋₁ when the oligonucleotide is hybridized to the target RNA sequence. In a preferred embodiment, the mismatch is a G/A mismatch. In one embodiment, the target RNA sequence is SNCA A53T. In one embodiment, the target RNA sequence is ABCA4 G1961E. In one embodiment, the target RNA sequence is KRAS G12D.

The invention relates to various gRNAs or oligonucleotides for A-to-I editing of a target RNA sequence. Accordingly, the invention provides various gRNAs or oligonucleotides that comprise one of the sequences selected from a) SEQ ID NOs: 1 to 926; or SEQ ID NOs: 927 to 1165; or a sequence with at least 80% sequence identity to any of said sequences.

In one embodiment, the oligonucleotide has at least 85%, at least 90%, at least 95%, at least 99%, or 100% sequence identity to any of SEQ ID NOs: 223 to 291; SEQ ID NOs: 1-222; SEQ ID NOs: 292 to 339; SEQ ID NOs: 340 to 399; SEQ ID NOs: 400 to 518; SEQ ID NOs: 519 to 593; SEQ ID NOs: 594 to 634; SEQ ID NOs: 635 to 694; SEQ ID NOs: 695 to 728; SEQ ID NOs: 729 to 753; SEQ ID NOs: 754 to 796; SEQ ID NOs: 797 to 926; SEQ ID NOs: 927-966; SEQ ID NOs: 967 to 1005; SEQ ID NOs: 1006 to 1029; SEQ ID NOs: 1030 to 1070; SEQ ID NOs: 1071 to 1134; and SEQ ID NOs: 1135 to 1165.

Exemplary target genes and antisense specificity sequences described herein are disclosed below:

SNCA A53T mutation. In one implementation, the scope of the invention encompasses guide RNAs comprising antisense specificity sequences for correction of the SNCA A53T mutation. For example, in some embodiments the target transcript encodes SNCA, carrying the A53T mutation associated with Parkinson's disease. In some embodiments, the target transcript comprises a target RNA region comprising the following sequence, with the mutated target adenosine underlined:

(SEQ ID NO: 1168)
5′GUGUUCUCUAUGUAGGCUCCAAAACCAAGGAGGGAGUGGUGCAUGGUGUGACAACAGU
GGCUGAGAAGACCAAAGAGCAAGUGACAAAUGUUGGAGGAGCA 3′.

In various embodiments, the guide RNA comprises a sequence having at least 80%, at least 85%, at least 90%, at least 95%, or at least 99% sequence identity to any of SEQ ID NO. 1-222 from Table 3. In other implementations, the scope of the invention comprises a method of correcting the SNCA A53T mutation in a cell by contacting the cell with a guide RNA comprising a sequence having at least 80%, at least 85%, at least 90%, at least 95%, or at least 99% sequence identity to any of SEQ ID NO. 1-222 from Table 3. In one implementation, the scope of the invention encompasses a method of preventing or treating Parkinson's Disease associated with the SNCA A53T mutation in a subject by administering to the subject a guide RNA comprising a sequence having at least 80%, at least 85%, at least 90%, at least 95%, or at least 99% sequence identity to any of SEQ ID NO. 1-222 from Table 3.

SERPINA1 E342K Mutation. In one implementation, the scope of the invention encompasses guide RNAs comprising antisense specificity sequences for correction of the SERPINA1 E342K mutation. For example, in some embodiments the target transcript encodes SERPINA 1 carrying the E342K mutation associated with deficiency in the circulating serine protease inhibitor α(1)-Antitrypsin (α(1)AT). α(1)AT deficiency is a major contributor to pathologies such as pulmonary emphysema and liver disease in persons of European ancestry. In some embodiments, the target transcript comprises a target RNA region comprising the following sequence, with the mutated target adenosine underlined:

(SEQ ID NO: 1169)
5′ CACCCCUGAAGCUCUCCAAGGCCGUGCAUAAGGCUGUGCUGACCAU
CGACAAGAAAGGGACUGAAGCUGCUGGGGCCAUGUUUUUAGAGGCCAUA
CCCAUG 3′.

In various embodiments, the guide RNA comprises a sequence having at least 80%, at least 85%, at least 90%, at least 95%, or at least 99% sequence identity to any of SEQ ID NO. 223-291 from Table 3. In other implementations, the scope of the invention comprises a method of correcting the SERPINA A1 E342K mutation in a cell by contacting the cell with a guide RNA comprising a sequence having at least 80%, at least 85%, at least 90%, at least 95%, or at least 99% sequence identity to any of SEQ ID NO. 223-291 from Table 3. In one implementation, the scope of the invention encompasses a method of preventing or treating a (1) AT deficiency or an associate pathology associated with the SERPINA A1 E342K mutation in a subject by administering to the subject a guide RNA comprising a sequence having at least 80%, at least 85%, at least 90%, at least 95%, or at least 99% sequence identity to any of SEQ ID NO. 223-291 from Table 3.

GFER R194H Mutation. In one implementation, the scope of the invention encompasses guide RNAs comprising antisense specificity sequences for correction of the GFER R194H mutation. For example, in some embodiments the target transcript encodes GFER carrying the R194H mutation associated with defects in mitochondrial protein import, resulting in mitochondrial disorders and associated pathologies such as progressive myopathy, partial combined respiratory-chain deficiency, and congenital cataract. In some embodiments, the target transcript comprises a target RNA region comprising the following sequence, with the mutated target adenosine underlined:

(SEQ ID NO: 1170)
5′ GAACCGCAAGCUGGGCAAGCCUGACUUCGACUGCUCAAAAGUGGAU
GAGCACUGGCGCGACGGCUGGAAGGAUGGCUCCUGUGACUAGAGGGUGG
UCAGCC 3′.

In various embodiments, the guide RNA comprises a sequence having at least 80%, at least 85%, at least 90%, at least 95%, or at least 99% sequence identity to any of SEQ ID NO. 292-339 from Table 3. In other implementations, the scope of the invention comprises a method of correcting the GFER R194H mutation in a cell by contacting the cell with a guide RNA comprising a sequence having at least 80%, at least 85%, at least 90%, at least 95%, or at least 99% sequence identity to any of SEQ ID NO. 292-339 from Table 3. In one implementation, the scope of the invention encompasses a method of preventing or treating diseases and conditions associated with the GFER R194H mutation in a subject by administering to the subject a guide RNA comprising a sequence having at least 80%, at least 85%, at least 90%, at least 95%, or at least 99% sequence identity to any of SEQ ID NO. 292-339 from Table 3.

ALDH2 E504K Mutation. In one implementation, the scope of the invention encompasses guide RNAs comprising antisense specificity sequences for correction of the ALDH2 E504K mutation. For example, in some embodiments the target transcript encodes ALDH2 carrying the E504K mutation associated with deficiencies in aldehyde metabolism and associated conditions such as alcohol flushing, increased risk of cancer, neurodegenerative, and cardiac disease attributed to inefficient aldehyde metabolism. In some embodiments, the target transcript comprises a target RNA region comprising the following sequence, with the mutated target adenosine underlined:

(SEQ ID NO: 1171)
5′ UGUCGGGGAGUGGCCGGGAGUUGGGCGAGUACGGGCUGCAGGCAUA
CACUAAAGUGAAAACUGUCACAGUCAAAGUGCCUCAGAAGAACUCAUAA
GAAUCA 3′.

In various embodiments, the guide RNA comprises a sequence having at least 80%, at least 85%, at least 90%, at least 95%, or at least 99% sequence identity to any of SEQ ID NO. 340-399 from Table 3. In other implementations, the scope of the invention comprises a method of correcting the ALDH2 E504K mutation in a cell by contacting the cell with a guide RNA comprising a sequence having at least 80%, at least 85%, at least 90%, at least 95%, or at least 99% sequence identity to any of SEQ ID NO. 340-399 from Table 3. In one implementation, the scope of the invention encompasses a method of preventing or treating aldehyde metabolism deficits and associated conditions associated with the ALDH2 E504K mutation in a subject by administering to the subject a guide RNA comprising a sequence having at least 80%, at least 85%, at least 90%, at least 95%, or at least 99% sequence identity to any of SEQ ID NO. 340-399 from Table 3.

TP53 R175H Mutation. In one implementation, the scope of the invention encompasses guide RNAs comprising antisense specificity sequences for correction of the TP53 R175H mutation. For example, in some embodiments the target transcript encodes TP53 carrying the oncogenic R175H mutation associated with increased risk and severity of multiple cancers. In some embodiments, the target transcript comprises a target RNA region comprising the following sequence, with the mutated target adenosine underlined:

(SEQ ID NO: 1172)
5′ CGCCAUGGCCAUCUACAAGCAGUCACAGCACAUGACGGAGGUUGUG
AGGCACUGCCCCCACCAUGAGCGCUGCUCAGAUAGCGAUGGUCUGGCCC
CUCCUC 3′.

In various embodiments, the guide RNA comprises a sequence having at least 80%, at least 85%, at least 90%, at least 95%, or at least 99% sequence identity to any of SEQ ID NO. 400-518 from Table 3. In other implementations, the scope of the invention comprises a method of correcting the TP53 R175H mutation in a cell by contacting the cell with a guide RNA comprising a sequence having at least 80%, at least 85%, at least 90%, at least 95%, or at least 99% sequence identity to any of SEQ ID NO. 400-518 from Table 3. In one implementation, the scope of the invention encompasses a method of preventing or treating cancer or cancer risk associated with the TP53 R175H mutation in a subject by administering to the subject a guide RNA comprising a sequence having at least 80%, at least 85%, at least 90%, at least 95%, or at least 99% sequence identity to any of SEQ ID NO. 400-518 from Table 3.

SCN9A W897X Mutation. In one implementation, the scope of the invention encompasses guide RNAs comprising antisense specificity sequences for correction of the SCN9A W897X stop mutation. For example, in some embodiments the target transcript encodes SCN9A carrying the W897X mutation associated with defects in the alpha subunit of a sodium channel NaV1.7, and associated pathologies such as channelopathy-associated congenital insensitivity to pain. In some embodiments, the target transcript comprises a target RNA region comprising the following sequence, with the mutated target adenosine underlined: 5′CUACAAAGAAUGUGUCUGCAAGAUCAAUGAUGACUGUACGCUCCCACGGUA GCACAUGAACGACUUCUUCCACUCCUUCCUGAUUGUGUUCCGCGUGCUGU 3′ (SEQ ID NO: 1173).

In various embodiments, the guide RNA comprises a sequence having at least 80%, at least 85%, at least 90%, at least 95%, or at least 99% sequence identity to any of SEQ ID NO. 519-593 from Table 3. In other implementations, the scope of the invention comprises a method of correcting the SCN9A W897X mutation in a cell by contacting the cell with a guide RNA comprising a sequence having at least 80%, at least 85%, at least 90%, at least 95%, or at least 99% sequence identity to any of SEQ ID NO. 519-593 from Table 3. In one implementation, the scope of the invention encompasses a method of preventing or treating sodium channel NaV1.7 defects and associated pathologies associated with the SCN9A W897X mutation in a subject by administering to the subject a guide RNA comprising a sequence having at least 80%, at least 85%, at least 90%, at least 95%, or at least 99% sequence identity to any of SEQ ID NO. 519-593 from Table 3.

HBB W38X Mutation. In one implementation, the scope of the invention encompasses guide RNAs comprising antisense specificity sequences for correction of the HBB W38X stop mutation. For example, in some embodiments the target transcript encodes HBB carrying the W38X mutation associated with hemoglobin subunit beta defects that are associated with beta thalassemia. In some embodiments, the target transcript comprises a target RNA region comprising the following sequence, with the mutated target adenosine underlined:

(SEQ ID NO: 1174)
5′ GGAUGAAGUUGGUGGUGAGGCCCUGGGCAGGCUGCUGGUGGUCUAC
CCUUAGACCCAGAGGUUCUUUGAGUCCUUUGGGGAUCUGUCCACUCCUG
AUGCUG 3′.

In various embodiments, the guide RNA comprises a sequence having at least 80%, at least 85%, at least 90%, at least 95%, or at least 99% sequence identity to any of SEQ ID NO. 594-634 from Table 3. In other implementations, the scope of the invention comprises a method of correcting the HBB W38X mutation in a cell by contacting the cell with a guide RNA comprising a sequence having at least 80%, at least 85%, at least 90%, at least 95%, or at least 99% sequence identity to any of SEQ ID NO. 594-634 from Table 3. In one implementation, the scope of the invention encompasses a method of preventing or treating hemoglobin subunit beta defects or associated conditions such as beta thalassemia associated with the HBB W38X mutation in a subject by administering to the subject a guide RNA comprising a sequence having at least 80%, at least 85%, at least 90%, at least 95%, or at least 99% sequence identity to any of SEQ ID NO. 594-634 from Table 3.

KRAS G12D Mutation. In one implementation, the scope of the invention encompasses guide RNAs comprising antisense specificity sequences for correction of the KRAS G12D mutation. For example, in some embodiments the target transcript encodes KRAS carrying the oncogenic G12D mutation associated with increased cancer risk and severity, for example in pancreatic cancer and colorectal cancer. In some embodiments, the target transcript comprises a target RNA region comprising the following sequence, with the mutated target adenosine underlined:

(SEQ ID NO: 1175)
5′GAGAGGCCUGCUGAAAAUGACUGAAUAUAAACUUGUGGUAGUUGGAG
CUGAUGGCGUAGGCAAGAGUGCCUUGACGAUACAGCUAAUUCAGAAUC
AUUUUG 3′.

In various embodiments, the guide RNA comprises a sequence having at least 80%, at least 85%, at least 90%, at least 95%, or at least 99% sequence identity to any of SEQ ID NO. 635-694 from Table 3. In other implementations, the scope of the invention comprises a method of correcting the KRAS G12D mutation in a cell by contacting the cell with a guide RNA comprising a sequence having at least 80%, at least 85%, at least 90%, at least 95%, or at least 99% sequence identity to any of SEQ ID NO. 635-694 from Table 3. In one implementation, the scope of the invention encompasses a method of preventing or treating cancer or cancer risk associated with the KRAS G12D mutation in a subject by administering to the subject a guide RNA comprising a sequence having at least 80%, at least 85%, at least 90%, at least 95%, or at least 99% sequence identity to any of SEQ ID NO. 635-694 from Table 3.

KRAS G12S Mutation. In one implementation, the scope of the invention encompasses guide RNAs comprising antisense specificity sequences for correction of the KRAS G12S mutation. For example, in some embodiments the target transcript encodes KRAS carrying the oncogenic G12S mutation associated with increased cancer risk and severity, for example in colon cancer and lung cancer. In some embodiments, the target transcript comprises a target RNA region comprising the following sequence, with the mutated target adenosine underlined:

(SEQ ID NO: 1176)
5′AGAGAGGCCUGCUGAAAAUGACUGAAUAUAAACUUGUGGUAGUUGGAGCUAGUGGCGU
AGGCAAGAGUGCCUUGACGAUACAGCUAAUUCAGAAUCAUUUU 3′.

In various embodiments, the guide RNA comprises a sequence having at least 80%, at least 85%, at least 90%, at least 95%, or at least 99% sequence identity to any of SEQ ID NO. 695-728 from Table 3. In other implementations, the scope of the invention comprises a method of correcting the KRAS G12S mutation in a cell by contacting the cell with a guide RNA comprising a sequence having at least 80%, at least 85%, at least 90%, at least 95%, or at least 99% sequence identity to any of SEQ ID NO. 695-694 from Table 3. In one implementation, the scope of the invention encompasses a method of preventing or treating cancer or cancer risk associated with the KRAS G12S mutation in a subject by administering to the subject a guide RNA comprising a sequence having at least 80%, at least 85%, at least 90%, at least 95%, or at least 99% sequence identity to any of SEQ ID NO. 695-694 from Table 3.

MeCP2 R106Q Mutation. In one implementation, the scope of the invention encompasses guide RNAs comprising antisense specificity sequences for correction of the MeCP2 R106Q mutation. For example, in some embodiments the target transcript encodes MeCP2 carrying the R106Q mutation associated with Rett Syndrome. In some embodiments, the target transcript comprises a target RNA region comprising the following sequence, with the mutated target adenosine underlined:

(SEQ ID NO: 1177)
5′ UGACCGGGGACCCAUGUAUGAUGACCCCACCCUGCCUGAAGGCUGG
ACACAGAAGCUUAAGCAAAGGAAAUCUGGCCGCUCUGCUGGGAAGUAUG
AUGUGU 3′.

In various embodiments, the guide RNA comprises a sequence having at least 80%, at least 85%, at least 90%, at least 95%, or at least 99% sequence identity to any of SEQ ID NO. 729-753 from Table 3. In other implementations, the scope of the invention comprises a method of correcting the MeCP2 R106Q mutation in a cell by contacting the cell with a guide RNA comprising a sequence having at least 80%, at least 85%, at least 90%, at least 95%, or at least 99% sequence identity to any of SEQ ID NO. 729-753 from Table 3. In one implementation, the scope of the invention encompasses a method of preventing or treating Rett Syndrome associated with the MeCP2 R106Q mutation in a subject by administering to the subject a guide RNA comprising a sequence having at least 80%, at least 85%, at least 90%, at least 95%, or at least 99% sequence identity to any of SEQ ID NO. 729-753 from Table 3.

F5 R534Q Mutation. In one implementation, the scope of the invention encompasses guide RNAs comprising antisense specificity sequences for correction of the F5 R534Q mutation. For example, in some embodiments the target transcript encodes F5 carrying the R534Q mutation associated with Factor V defects and associated conditions such as venous thrombosis and thrombophilia. In some embodiments, the target transcript comprises a target RNA region comprising the following sequence, with the mutated target adenosine underlined:

(SEQ ID NO: 1178)
5′ UGGGCUAAUAGGACUACUUCUAAUCUGUAAGAGCAGAUCCCUGGAC
AGGCAAGGAAUACAGAGGGCAGCAGACAUCGAACAGCAGGCUGUGUUUG
CUGUGU 3′.

In various embodiments, the guide RNA comprises a sequence having at least 80%, at least 85%, at least 90%, at least 95%, or at least 99% sequence identity to any of SEQ ID NO. 754-796 from Table 3. In other implementations, the scope of the invention comprises a method of correcting the F5 R534Q mutation in a cell by contacting the cell with a guide RNA comprising a sequence having at least 80%, at least 85%, at least 90%, at least 95%, or at least 99% sequence identity to any of SEQ ID NO. 754-796 from Table 3. In one implementation, the scope of the invention encompasses a method of preventing or treating Factor V defects and associated conditions such as venous thrombosis and thrombophilia associated with the F5 R534Q mutation in a subject by administering to the subject a guide RNA comprising a sequence having at least 80%, at least 85%, at least 90%, at least 95%, or at least 99% sequence identity to any of SEQ ID NO. 754-796 from Table 3.

ABCA4 G1961E Mutation. In one implementation, the scope of the invention encompasses guide RNAs comprising antisense specificity sequences for correction of the ABCA4 G1961E mutation. For example, in some embodiments the target transcript encodes ABCA4 carrying the G1961E mutation associated with retinal disease and maculopathies such as Stargardt disease. In some embodiments, the target transcript comprises a target RNA region comprising the following sequence, with the mutated target adenosine underlined:

(SEQ ID NO: 1179)
5′ CAAGAUUUAUCCAGGCACCUCCAGCCCAGCAGUGGACAGGCUGUGU
GUCGAAGUUCGCCCUGGAGAGUGCUUUGGCCUCCUGGGAGUGAAUGGUG
CCGGCA 3′.

In various embodiments, the guide RNA comprises a sequence having at least 80%, at least 85%, at least 90%, at least 95%, or at least 99% sequence identity to any of SEQ ID NO. 797-926 from Table 3. In other implementations, the scope of the invention comprises a method of correcting the ABCA4 G1961E mutation in a cell by contacting the cell with a guide RNA comprising a sequence having at least 80%, at least 85%, at least 90%, at least 95%, or at least 99% sequence identity to any of SEQ ID NO. 797-926 from Table 3. In one implementation, the scope of the invention encompasses a method of preventing or treating retinal disease and maculopathies such as Stargardt disease associated with the ABCA4 G1961E mutation in a subject by administering to the subject a guide RNA comprising a sequence having at least 80%, at least 85%, at least 90%, at least 95%, or at least 99% sequence identity to any of SEQ ID NO. 797-926 from Table 3.

STAT3 Y705C Variant. In one implementation, the scope of the invention encompasses guide RNAs comprising antisense specificity sequences for creation of the STAT3 Y705C variant. For example, in some embodiments the target transcript encodes wild-type STAT3 containing the Y705 phosphorylation site associated with dysregulated STAT3 signaling in cancer. Creating the Y705C variant inhibits STAT3 phosphorylation and prevents overactivation of STAT3 associated with cancer. In some embodiments, the target transcript comprises a target RNA region comprising the following sequence, with the target adenosine underlined:

(SEQ ID NO: 1180)
5′ GCCAGAGAGCCAGGAGCAUCCUGAAGCUGACCCAGGUAGCGCUGCCCC
AUACCUGAAGACCAAGUUUAUCUGUGUGACACCAACGACCUGCAGCAAUAC
CA 3′.

In various embodiments, the guide RNA comprises a sequence having at least 80%, at least 85%, at least 90%, at least 95%, or at least 99% sequence identity to any of SEQ ID NO. 927-966 from Table 3. In other implementations, the scope of the invention comprises a method of creating the STAT3 Y705C variant in a cell by contacting the cell with a guide RNA comprising a sequence having at least 80%, at least 85%, at least 90%, at least 95%, or at least 99% sequence identity to any of SEQ ID NO. 927-966 from Table 3. In one implementation, the scope of the invention encompasses a method of preventing or treating dysregulated STAT3 signaling and associated conditions associated with STAT3 Y705 phosphorylation in a subject by administering to the subject a guide RNA comprising a sequence having at least 80%, at least 85%, at least 90%, at least 95%, or at least 99% sequence identity to any of SEQ ID NO. 927-966 from Table 3.

STAT1 Y701C Variant. In one implementation, the scope of the invention encompasses guide RNAs comprising antisense specificity sequences for creation of the STAT1 Y701C variant. For example, in some embodiments the target transcript encodes wild-type STAT1 containing the Y701phosphorylation site associated with interferon signaling. Creating the Y701C variant inhibits STAT1 phosphorylation and consequently suppresses immune signaling associated with inflammatory and immune diseases. In some embodiments, the target transcript comprises a target RNA region comprising the following sequence, with the target adenosine underlined:

(SEQ ID NO: 1181)
5′ AAAGGAAGCACCAGAGCCAAUGGAACUUGAUGGCCCUAAAGGAACUGG
AUAUAUCAAGACUGAGUUGAUUUCUGUGUCUGAAGUUCACCCUUCUAGACU
UC 3′.

In various embodiments, the guide RNA comprises a sequence having at least 80%, at least 85%, at least 90%, at least 95%, or at least 99% sequence identity to any of SEQ ID NO. 967-1005 from Table 3. In other implementations, the scope of the invention comprises a method of creating the STAT1 Y701C variant in a cell by contacting the cell with a guide RNA comprising a sequence having at least 80%, at least 85%, at least 90%, at least 95%, or at least 99% sequence identity to any of SEQ ID NO. 967-1005 from Table 3. In one implementation, the scope of the invention encompasses a method of suppressing immune signaling and associated immune and inflammatory diseases associated with STAT1 Y701 phosphorylation in a subject by administering to the subject a guide RNA comprising a sequence having at least 80%, at least 85%, at least 90%, at least 95%, or at least 99% sequence identity to any of SEQ ID NO. 967-1005 from Table 3.

IFNAR1 Y466C Variant. In one implementation, the scope of the invention encompasses guide RNAs comprising antisense specificity sequences for creation of the IFNAR1 Y466C variant. For example, in some embodiments the target transcript encodes wild-type IFNAR1 containing the Y466 phosphorylation site associated with interferon signaling. Creating the Y466C variant inhibits IFNAR 1 phosphorylation and consequently suppresses dysregulated interferon signaling in immune and inflammatory diseases. In some embodiments, the target transcript comprises a target RNA region comprising the following sequence, with the target adenosine underlined:

(SEQ ID NO: 1182)
5′UCUCCCGUUUGUCAUUUAUGCUGCGAAAGUCUUCUUGAGAUGCAUCAAUUAUGUCUUC
UUUCCAUCACUUAAACCUUCUUCCAGUAUAGAUGAGUAUUUCU 3′.

In various embodiments, the guide RNA comprises a sequence having at least 80%, at least 85%, at least 90%, at least 95%, or at least 99% sequence identity to any of SEQ ID NO. 1006-1029 from Table 3. In other implementations, the scope of the invention comprises a method of creating the IFNAR1 Y466C variant in a cell by contacting the cell with a guide RNA comprising a sequence having at least 80%, at least 85%, at least 90%, at least 95%, or at least 99% sequence identity to any of SEQ ID NO. 1006-1029 from Table 3. In one implementation, the scope of the invention encompasses a method of preventing or treating dysregulated interferon signaling and associated conditions associated with IFNAR1 Y466 phosphorylation in a subject by administering to the subject a guide RNA comprising a sequence having at least 80%, at least 85%, at least 90%, at least 95%, or at least 99% sequence identity to any of SEQ ID NO. 1006-1029 from Table 3.

CTNNB1 T41A Variant. In one implementation, the scope of the invention encompasses guide RNAs comprising antisense specificity sequences for creation of the CTNNB1 T41A variant. For example, in some embodiments the target transcript encodes wild-type CTNNB 1 containing the T41 phosphorylation site associated with WNT/beta-catenin signaling. Creating the T41A variant inhibits beta-catenin phosphorylation and activates the WNT/beta-catenin pathway, inducing tissue regeneration, including liver regeneration after acute or chronic injury. In some embodiments, the target transcript comprises a target RNA region comprising the following sequence, with the target adenosine underlined:

(SEQ ID NO: 1183)
5′ ACUGGCAGCAACAGUCUUACCUGGACUCUGGAAUCCAUUCUGGUGCC
ACUACCACAGCUCCUUCUCUGAGUGGUAAAGGCAAUCCUGAGGAAGAGG
AUGUG 3′.

In various embodiments, the guide RNA comprises a sequence having at least 80%, at least 85%, at least 90%, at least 95%, or at least 99% sequence identity to any of SEQ ID NO. 1030-1070 from Table 3. In other implementations, the scope of the invention comprises a method of creating the CTNNB1 T41A variant in a cell by contacting the cell with a guide RNA comprising a sequence having at least 80%, at least 85%, at least 90%, at least 95%, or at least 99% sequence identity to any of SEQ ID NO. 1030-1070 from Table 3. In one implementation, the scope of the invention encompasses a method of inducing WNT/beta-catenin signaling and associated tissue regeneration such as liver regeneration associated with CTNNB1 T41 phosphorylation in a subject by administering to the subject a guide RNA comprising a sequence having at least 80%, at least 85%, at least 90%, at least 95%, or at least 99% sequence identity to any of SEQ ID NO. 1030-1070 from Table 3.

TMEM173 N154S Variant. In one implementation, the scope of the invention encompasses guide RNAs comprising antisense specificity sequences for creation of the TMEM173 N154S variant. For example, in some embodiments the target transcript encodes wild-type TMEM173 containing the N154 residue. TMEM173 gene encodes the protein STING (stimulator of interferon genes), a key player in innate immunity. Creating the N154S variant results in constitutive activation of STING and upregulation of interferon signaling, which may be beneficial in cancer immunotherapy. In some embodiments, the target transcript comprises a target RNA region comprising the following sequence, with the target adenosine underlined:

(SEQ ID NO: 1184)
5′ GGGCCUGGCCCCAGCUGAGAUCUCUGCAGUGUGUGAAAAAGGGAAUU
UCAACGUGGCCCAUGGGCUGGCAUGGUCAUAUUACAUCGGAUAUCUGCG
GCUGA 3′.

In various embodiments, the guide RNA comprises a sequence having at least 80%, at least 85%, at least 90%, at least 95%, or at least 99% sequence identity to any of SEQ ID NO. 1071-1134 from Table 3. In other implementations, the scope of the invention comprises a method of creating the TMEM173 N154S variant in a cell by contacting the cell with a guide RNA comprising a sequence having at least 80%, at least 85%, at least 90%, at least 95%, or at least 99% sequence identity to any of SEQ ID NO. 1071-1134 from Table 3. In one implementation, the scope of the invention encompasses a method of transient activation of STING and associated immune responses in cancer immunotherapy setting by administering to the subject a guide RNA comprising a sequence having at least 80%, at least 85%, at least 90%, at least 95%, or at least 99% sequence identity to any of SEQ ID NO. 1071-1134 from Table 3.

TMEM173 R284G Variant. In one implementation, the scope of the invention encompasses guide RNAs comprising antisense specificity sequences for creation of the TMEM173 R284G variant. For example, in some embodiments the target transcript encodes wild-type TMEM173 containing the R284 residue. Creating the TMEM173 R284G variant results in constitutive activation of STING and upregulation of interferon signaling, which may be beneficial in cancer immunotherapy. In some embodiments, the target transcript comprises a target RNA region comprising the following sequence, with the target adenosine underlined:

(SEQ ID NO: 1185)
5′ CUUUGUUUGCCAUGUCACAAUACAGUCAAGCUGGCUUUAGCCGGGAG
GAUAGGCUUGAGCAGGCCAAACUCUUCUGCCGGACACUUGAGGACAUCC
UGGCA 3′.

In various embodiments, the guide RNA comprises a sequence having at least 80%, at least 85%, at least 90%, at least 95%, or at least 99% sequence identity to any of SEQ ID NO. 1135-1165 from Table 3. In other implementations, the scope of the invention comprises a method of creating the TMEM173 R284G variant in a cell by contacting the cell with a guide RNA comprising a sequence having at least 80%, at least 85%, at least 90%, at least 95%, or at least 99% sequence identity to any of SEQ ID NO. 1135-1165 from Table 3. In one implementation, the scope of the invention encompasses a method of transient activation of STING and associated immune responses in cancer immunotherapy setting in a subject by administering to the subject a guide RNA comprising a sequence having at least 80%, at least 85%, at least 90%, at least 95%, or at least 99% sequence identity to any of SEQ ID NO. 1135-1165 from Table 3.

Hence, in one embodiment, the intended target RNA sequence codes for one of the following proteins selected from a list consisting of: a) SERPINA1 carrying the E342K mutation, and wherein the oligonucleotide comprises one of the sequences selected from a group consisting of SEQ ID NOs: 223 to 291; b) SNCA carrying the A53T mutation, and wherein the oligonucleotide comprises one of the sequences selected from a group consisting of SEQ ID NOs: 1 to 222; c) GFER carrying the R194H mutation, and wherein the oligonucleotide comprises one of the sequences selected from a group consisting of SEQ ID NOs: 292 to 339; d) ALDH2 carrying the E504K mutation, and wherein the oligonucleotide comprises one of the sequences selected from a group consisting of SEQ ID NOs: 340 to 399; e) TP53 carrying the oncogenic R175H mutation, and wherein the oligonucleotide comprises one of the sequences selected from a group consisting of SEQ ID NOs: 400 to 518; f) SCN9A carrying the W897X mutation, and wherein the oligonucleotide comprises one of the sequences selected from a group consisting of SEQ ID NOs: 519 to 593; g) HBB carrying the W38X mutation, and wherein the oligonucleotide comprises one of the sequences selected from a group consisting of SEQ ID NOs: 594 to 634; h) KRAS carrying the oncogenic G12D mutation, and wherein the oligonucleotide comprises one of the sequences selected from a group consisting of SEQ ID NOs: 635 to 694; i) KRAS carrying the oncogenic G12S mutation, and wherein the oligonucleotide comprises one of the sequences selected from a group consisting of SEQ ID NOs: 695 to 728; j) MeCP2 carrying the R106Q mutation, and wherein the oligonucleotide comprises one of the sequences selected from a group consisting of SEQ ID NOs: 729 to 753; k) F5 carrying the R534Q mutation, and wherein the oligonucleotide comprises one of the sequences selected from a group consisting of SEQ ID NOs: 754 to 796; and 1) ABCA4 carrying the G1961E mutation, and wherein the oligonucleotide comprises one of the sequences selected from a group consisting of SEQ ID NOs: 797 to 926.

Oligonucleotides of the invention may fall under one or more of the generalizable rules depending on the target gene. For instance, in case of SERPINA1, an oligonucleotide may be partially complementary to the SERPINA1 target RNA and comprise a sequence that falls under Rule 3 and/or Rule 5. In one embodiment, the oligonucleotide is partially complementary to SEQ ID NO: 1169 and comprises one of the following sequences selected from: SEQ ID NOs: 242, 243, 248, 252-275, 277-279, 281-285, and 287-291.

In other cases, the target is SNCA A53T. In case of the SNCA A53T target, an oligonucleotide may be partially complementary to the SNCA A53T target RNA and comprise a sequence that falls under Rule 1. In one embodiment, the oligonucleotide is partially complementary to SEQ ID NO: 1168, and comprises one of the following sequences selected from: SEQ ID NOs: 2, 4, 6, 9-14, 16-18, 20, 22, 23, 24, 26, 27, 30, 35-37, 39, 42, 43, 45, 47-50, 52-56, 58, 59, 61, 63, 66, 68, 74, 78, 82, 83, 86, 87, 90, 93, 97, 98, 101, 102, 105, 106, 112, 115, 116, 118, 122, 126-128, 137-140, 143, 146, 147, 154, 155, 159, 161-164, 169, 170, 174, 182, 185, 189, 190, 198-200, 203, 208, and 209.

In some cases, the target is GFER R194H. In case of the GFER R194H target, an oligonucleotide may be partially complementary to the GFER R194H target RNA and comprise a sequence that falls under Rule 6. In one embodiment, the oligonucleotide is partially complementary to SEQ ID NO: 1170 and comprises one of the following sequences selected from: SEQ ID NOs: 292, 298, 300, 305, 311-313, 319, 325, 327-330, 332, 333, 335, and 338.

In some cases, the target is ALDH2 E504K. In case of the ALDH2 E504K target, an oligonucleotide may be partially complementary to the ALDH2 E504K target RNA and comprise a sequence that falls under Rule 2. In one embodiment, the oligonucleotide is partially complementary to SEQ ID NO: 1171 and comprises one of the following sequences selected from: SEQ ID NOs: 347, 350, 358, 360, 361, 363, 364, 366, 368, 369, 371, 375, and 381.

In other cases, the target is TP53 R175H. In case of the TP53 R175H target, an oligonucleotide may be partially complementary to the TP53 R175H target RNA and comprise a sequence that falls under Rules 6 and7 or Rule 7. In one embodiment, the oligonucleotide is partially complementary to SEQ ID NO: 1172 and comprises one of the following sequences selected from: a) SEQ ID NOs: 402, 407, 415-417, 421, 425, 428, 431, 432, 440, 443, 447, 449, 450, 453, 467, 468, 476, 480, 482, 489, 490, 492, 495, 496, 497, 499, 502, 503, 505, and 513; or b) SEQ ID NOs: 405, 408, 409, 412-414, 418-420, 422-424, 426-427, 429, 430, 433-437, 439, 441, 442, 444-446, 448, 451, 452, 454-466, 469-472, 474, 478, 479, 481, 483-486, 493, 498, 500, 504, 506, 508, 509, 511, 512, and 514-518.

In some cases, the target is SCN9A W897X. In case of the SCN9A W897X target, an oligonucleotide may be partially complementary to the SCN9A W897X target RNA and comprise a sequence that falls under Rule 4. In one embodiment, the oligonucleotide is partially complementary to SEQ ID NO: 1173 and comprises one of the following sequences selected from: SEQ ID NOs: 521, 523- 530, 532, 534, 539-542, 544, 545, 547-549, 551-554, 556, 558-561, 564, 566, 567, 570, 571, 573-580, 582, 583, 585-587, and 590-593.

In some cases the target is HBB W38X. In one embodiment, the oligonucleotide is partially complementary to SEQ ID NO: 1174 and comprises one of the following sequences selected from: SEQ ID NOs: 594-633.

In other cases, the target is KRAS G12D. In case of the KRAS G12D target, an oligonucleotide may be partially complementary to the KRAS G12D target RNA and comprise a sequence that falls under Rule 5. In one embodiment, the oligonucleotide is partially complementary to SEQ ID NO: 1175 and comprises one of the following sequences selected from: SEQ ID NOs: 635, 638, 642, 643, 645, 646, 651, 658, 659, 664, 667, 673, 674, 683, 685, 686, and 693.

In other cases, the target is KRAS G12S. In case of the KRAS G12S target, an oligonucleotide may be partially complementary to the KRAS G12S target RNA and comprise a sequence that falls under Rule 6. In one embodiment, the oligonucleotide is partially complementary to SEQ ID NO: 1176 and comprises one of the following sequences selected from: SEQ ID NOs: 697, 699-702, 704-706, 708, 709, 711, 714, 716, 719, 721-723, 726, and 727.

In other cases, the target is MeCP2 R106Q. In case of the MeCP2 R106Q target, an oligonucleotide may be partially complementary to the MeCP2 R106Q target RNA and comprise a sequence that falls under Rule 2. In one embodiment, the oligonucleotide is partially complementary to SEQ ID NO: 1177 and comprises one of the following sequences selected from: SEQ ID NOs: 732, 735, 738, 739, 744, 745, 748, and 753.

In other cases, the target is F5 R534Q. In case of the F5 R534Q target, an oligonucleotide may be partially complementary to the F5 R534Q target RNA and comprise a sequence that falls under Rule 5. In one embodiment, the oligonucleotide is partially complementary to SEQ ID NO: 1178 and comprises one of the following sequences selected from: SEQ ID NOs: 760, 762, 768, 772, 784, and 787.

In other cases, the target is ABCA4 G1961E. In case of the ABCA4 G1961E target, an oligonucleotide may be partially complementary to the ABCA4 G1961E target RNA and comprise a sequence that falls under Rule 5. In one embodiment, the oligonucleotide is partially complementary to SEQ ID NO: 1179 and comprises one of the following sequences selected from: SEQ ID NOs: 811, 832, 846, 889, and 907.

Oligonucleotides provided herein may also be used to introduce the gain-of-function (GOF) mutations in a target RNA sequence. In some embodiments, the target RNA sequence codes for one of the following proteins selected from a list consisting of: a) STAT3 containing the Y705 phosphorylation site, and wherein the oligonucleotide comprises one of the sequences selected from a group consisting of SEQ ID NOs: 927-966; b) STAT1 containing the Y701phosphorylation site, and wherein the oligonucleotide comprises one of the sequences selected from a group consisting of SEQ ID NO: 967-1005; c) IFNAR1 containing the Y466 phosphorylation site, and wherein the oligonucleotide comprises one of the sequences selected from a group consisting of SEQ ID NOs: 1006-1029; d) CTNNB1 containing the T41 phosphorylation site, and wherein the oligonucleotide comprises one of the sequences selected from a group consisting of SEQ ID NOs: 1030-1070; e) TMEM173 containing the N154 residue, and wherein the oligonucleotide comprises one of the sequences selected from a group consisting of SEQ ID NOs: 1071-1134; and f) TMEM173 containing the R284 residue, and wherein the oligonucleotide comprises one of the sequences selected from a group consisting of SEQ ID NOs: 1135-1165.

In one instance an oligonucleotides provided herein may be used to introduce the gain-of-function (GOF) mutations in a target RNA sequence encoding the STAT3 Y705C variant. In one embodiment, the oligonucleotide is partially complementary to SEQ ID NO: 1180 and comprises one of the following sequences selected from: SEQ ID NOs: 936, 939, and 947.

In one instance an oligonucleotides provided herein may be used to introduce the gain-of-function (GOF) mutations in a target RNA sequence encoding STAT1 Y701C. In one embodiment, the oligonucleotide is partially complementary to SEQ ID NO: 1181 and comprises one of the following sequences selected from: SEQ ID NOs: 967-1005.

In one instance an oligonucleotides provided herein may be used to introduce the gain-of-function (GOF) mutations in a target RNA sequence encoding IFNAR1 Y466C. In one embodiment, the oligonucleotide is partially complementary to SEQ ID NO: 1182 and comprises one of the following sequences selected from: SEQ ID NOs: 1006-1020, 1022, 1024, 1026, and 1029.

In one instance an oligonucleotides provided herein may be used to introduce the gain-of-function (GOF) mutations in a target RNA sequence encoding CTNNB1 T41A. In one embodiment, the oligonucleotide is partially complementary to SEQ ID NO: 1183 and comprises one of the following sequences selected from: SEQ ID NOs: 1030-1070.

In one instance an oligonucleotides provided herein may be used to introduce the gain-of-function (GOF) mutations in a target RNA sequence encoding TMEM173 N154S. In one embodiment, the oligonucleotide is partially complementary to SEQ ID NO: 1184 and comprises one of the following sequences selected from: SEQ ID NOs: 1074, 1079, 1087, 1094, 1096, 1107, 1112, 1120, 1125, 1128, and 1133.

In one instance an oligonucleotides provided herein may be used to introduce the gain-of-function (GOF) mutations in a target RNA sequence encoding TMEM173 R284G. In one embodiment, the oligonucleotide is partially complementary to SEQ ID NO: 1185 and comprises one of the following sequences selected from: SEQ ID NOs: 1148 and 1156.

Editing enhancements or efficacy of each oligonucleotide tested was calculated relative the prototype design, in which the gRNA or oligonucleotide is fully complementary to the target sequence except for a mismatch opposite the target A to be edited. In one embodiment, the oligonucleotide has an editing enhancement that is at least 1.1-fold higher than that of a control. In one embodiment, the editing enhancement is 1.1-3 fold higher. In one embodiment, the oligonucleotide has an editing efficacy of at least 20%, at least 30%, at least 40%, at least 50%, at least 60%, or at least 70%, wherein the percentage is defined as % of A converted to I at the target site. In one embodiment, the oligonucleotide has an editing efficacy of between 20% and 100%. In one embodiment, the control is an oligonucleotide fully complementary to the target sequence except for a mismatch at No opposite the target A.

The length of the specificity sequence may vary, for example, in some embodiments, the antisense domain comprises 10-150 nucleotides. In some embodiments, the antisense domain comprises 10-125 nucleotides. In some embodiments, the antisense domain comprises 10-110 nucleotides. In some embodiments, the antisense domain comprises 15-101 nucleotides. In some embodiments, the antisense domain comprises 10-50 nucleotides. In one embodiment, the oligonucleotide has a length of 15-101nt, preferably wherein the oligonucleotide has a length of 18-45 nt. For example, in some embodiments the antisense domain comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In some embodiments, the antisense domain comprises more than 50 nucleotides. In some embodiments, the antisense domain comprises 10-30 nucleotides. In some embodiments, the antisense domain comprises 15-25 nucleotides. In some embodiments, the antisense domain comprises 18-20 nucleotides. In some embodiments, the antisense domain comprises 18 nucleotides. In some embodiments, the antisense domain comprises 19 nucleotides. In some embodiments, the antisense domain comprises 20 nucleotides.

In some embodiments, the specificity sequence may be symmetrical or nearly symmetrical around the zero position (N₀), i.e., wherein there are equal or nearly equal numbers of bases flanking the zero position. In one embodiment, the oligonucleotide comprises between 4-50 nucleotides on either the 3′ or 5′ side of the target adenosine to be edited. In one embodiment, an oligonucleotide comprises 7-10 nucleotides upstream of the zero position and 7-10 nucleotides downstream of the zero position. Alternatively, the oligonucleotide may be asymmetrical. This means that there are different amounts of nucleotides 3′ and 5′ of the zero position. In some embodiments, the oligonucleotide is asymmetric. In some embodiments, there are at least 6 nucleotides (nt) at the 5′ terminus (i.e., 5′ to the zero position) and at least 10 nt at the 3′ terminus (i.e., 3′ to the zero position). In one embodiment, there are at least 6 nucleotides (nt) at the 5′ terminus. In one embodiment, there are at least 10 nt at the 3′ terminus.

In some embodiments, the target sequence comprises one or more mutations of which correction using a guide RNA is desired. In some embodiments, a guide RNA is used to insert one or more mutations into a target sequence.

### 2.2 Recruitment Domain

The guide RNA sequence or oligonucleotide may further include a recruitment domain. The recruitment domain (also referred to herein as the ADAR-recruiting element), facilitates the interaction between the RNA editing enzyme, e.g., ADAR1, and the target transcript. The recruitment domain is configured to bind (i.e., recruit) one or more ADAR proteins to the guide RNA. Hence, in some embodiments, the guide RNA sequence or oligonucleotide comprises a recruitment domain. In some embodiments, the recruitment domain will comprise complementary sequences that form a stem loop, which achieves enzyme recruitment by binding the double-stranded RNA binding domains of ADAR or another RNA enzyme. For example, the recruitment domain may be configured to recruit an ADAR1, an ADAR2 protein or an engineered ADAR or other RNA-editing enzyme. The recruitment domain may comprise any suitable number of nucleotides. For example, the recruitment domain may comprise 15-100 nucleotides. In some embodiments, the recruitment domain comprises about 15, about 20, about 25, about 30, about 35, about 40, about 45, about 50, about 55, about 60, about 65, about 70, about 75, about 80, about 85, about 90, about 95, or about 100 nucleotides. In some embodiments, the recruitment domain is part of a construct that possesses a stem-loop secondary structure. In some embodiments, the recruitment domain forms a part of a stem loop structure. In some embodiments, the loop portion of the stem loop structure consists of 5 nucleotides (i.e., a pentaloop).

In some embodiments, the guide RNA possesses a stem loop secondary structure. The terms “hairpin”, “hairpin loop”, “stem loop”, and/or “loop” are used interchangeably herein to refer to a structure formed in a single stranded oligonucleotide when sequences within the single strand which are complementary when read in opposite directions base pair to form a region whose conformation resembles a hairpin. The recruitment domain may be 3′ or 5′ to the antisense specificity domain. In one embodiment, the recruitment domain is 5′ to the antisense specificity domain.

In some embodiments, the recruitment domain is based upon the sequence of an endogenous (i.e., naturally occurring) ADAR target. The recruitment domain may possess one or more modifications compared to the endogenous ADAR target, which may enhance ADAR recruitment or interactions. For example, the recruitment domain may be based upon the sequence of the GRIA2 R/G site, an endogenous target for ADAR2.

In some embodiments, the recruitment domain comprises the sequence: GGUGUCGAGAAGAGGAGAACAAUAUGCCAAAUGUUGUUCUCGUCUCCUCGACACC (SEQ ID NO: 1166). In some embodiments, the recruitment domain comprises a sequence having at least 80% sequence identity (e.g., at least 80%, at least 85%, at least 90%, at least 91%, at least 92%, at least 93%, at least 94%, at least 95%, at least 96%, at least 97%, at least 98%, at least 99%, or 100% identity) to SEQ ID NO: 1166. In some embodiments, the recruitment domain comprises a sequence having at least 80% sequence identity (e.g., at least 80%, at least 85%, at least 90%, at least 91%, at least 92%, at least 93%, at least 94%, at least 95%, at least 96%, at least 97%, at least 98%, at least 99%, or 100% identity) to SEQ ID NO: 1166, and the pentaloop structure of the recruitment domain comprises GCCAA (SEQ ID NO: 1167). Additional suitable recruitment domains and/or pentaloop sequences for the recruitment domain may be used, including those described in WO2022087272A1, the entire contents of which are incorporated herein by reference for all purposes.

Oligonucleotides containing a stem-loop structured recruitment domain have previously been described (WO 2020/001793) and used successfully to harness ADARs with chemically modified oligonucleotides. However, they are comparably large and—without being bound by any theory—the inventors believe that a more intelligent design of the ASO can form a substrate duplex that is also very well and quickly recognized by endogenous ADAR so that the large recruitment motifs can be omitted. Hence, in one embodiment, a guide RNA provided herein lacks a recruitment domain. In some embodiments, an oligonucleotide does not comprise a recruitment domain. In some embodiments, an oligonucleotide does contain a stem loop secondary structures. In some embodiments, the length of the antisense domain depends on whether the guide RNA additionally comprises a recruitment domain. For example, guide RNA sequences lacking a recruitment domain may contain antisense domains of greater length compared to guide RNA sequences containing both a recruitment domain and an antisense domain.

### 2.3 Nucleobase Modifications

The guide RNAs of the invention are delivered as antisense oligonucleotides to the target cells. In such implementations, the gRNA may comprise one or more modified nucleobases or internucleoside linkage modifications. While not intending to be bound by any particular theory of operation, it is believed that nucleobase and backbone linkage modifications of said oligonucleotides are useful in stabilizing the oligonucleotide, improving on-target binding affinity and editing efficacy and/or persistence of the oligonucleotides of the invention in vivo.

That oligonucleotides can be modified to improve their properties has been shown in WO2022/253810 and WO2023/099494, which are herewith incorporated by reference in their entirety. Hence, in one embodiment, an oligonucleotide of the invention is chemically modified. In one embodiment, an oligonucleotide of the invention comprises one or more chemical modifications.

Any nucleotide modification known in the art may be employed, for example 2′-O-methylation (e.g., 2′-O-Methyl and 2′-O-methoxyethyl), phosphorothioate modifications, locked nucleic acid (LNA) modifications. Exemplary modifications include 2′-O-methyl(2′-OMe), phosphorothioate (PS), 2′-O-methyl thioPACE (MSP), 2′-O-methyl-PACE (MP), 2′-fluoro (2′-F), constrained ethyl (S-cEt), and the use of phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, and other modifications known in the art.

In some embodiments, the oligonucleotides comprise a modification at the 2′-position of the sugar residue. In some embodiments, the oligonucleotides comprise a mixture of different modifications at the 2′-position of the sugar residue. In some embodiments, the oligonucleotides of the invention comprise a mixture of different modifications at the 2′-position of the sugar residue, and no more than 6 nucleotides with the same 2′-modification. Also, avoiding uniform blocks of more than 6 nucleotides with the same 2′-modification prevented a strong loss of editing activity with natural ADARs.

In some embodiments, 20-70% of nucleotides are 2′-F-modified. In a preferred embodiment, 35-65% of nucleotides are 2′-F-modified. In one embodiment, 20-60% of nucleotides are 2′-O-methyl(2′-OMe)-modified. In a preferred embodiment, 25-55% of nucleotides are 2′-OMe-modified. In one embodiment, 20-70% of nucleotides are 2′-F-modified, preferably wherein 35-65% of nucleotides are 2′-F-modified; and/or wherein 20-60% of nucleotides are 2′-O-methyl(2′-OMe)-modified, preferably wherein 25-55% of nucleotides are 2′-OMe-modified.

In one embodiment, at least 10% of nucleotides of the oligonucleotide are 2′-F-modified and at least 10% of nucleotides of the oligonucleotide arc 2′-O-alkyl-modified, wherein no more than 6 consecutive nucleotides have the same 2′-modification.

In some embodiments, no more than 4, 5, or 6 consecutive nucleotides are 2′-F-modified. In some embodiments, no more than 4, 5, or 6 consecutive nucleotides are 2′-O-alkyl-modified. In some embodiments, (i) no more than 4, 5, or 6 consecutive nucleotides are 2′-F-modified; and/or (ii) no more than 4, 5, or 6 consecutive nucleotides are 2′-O-alkyl-modified.

In one embodiment, the internucleoside linkage modification is selected from the group consisting of phosphorothioate (PS), 3′-methylenephosphonate, 5′-methylenephosphonate, 3′-phosphoroamidate, 2′-5′phosphodiester, and phosphoryl guanidine (PN). In a preferred embodiment, the internucleoside linkage is PS.

The oligonucleotides may benefit from having a base level of internucleoside linkage modifications. For example, the oligonucleotides may have at least 15% of their internucleoside modified to achieve good RNA editing. Hence, in one embodiment, the internucleoside linkage modification content is at least 15%. In one embodiment, the internucleoside linkage modification content is at least 20%. In one embodiment, the internucleoside linkage modification content is at least 30%. In one embodiment, no more than 95%, 90%, 85%, 80%, 70%, 60%, 50%, 40%, 30% or 20% of the internucleoside linkages are internucleoside linkage modifications. In one embodiment, 15-90% of the internucleoside linkages are internucleoside linkage modifications. In one embodiment, 30-90% of the internucleoside linkages are internucleoside linkage modifications. In a preferred embodiment, 40-80%, most preferably 45-60%, of the internucleoside linkages are internucleoside linkage modifications.

Without being bound by any theory, it is submitted that-contrary to the antisense oligonucleotides of the prior art-high levels of stereopure linkages may neither be useful nor necessary to provide efficient RNA editing. Hence, in one embodiment, the chemically modified oligonucleotide does not comprise a stereopure phosphorothioate (PS) linkage modification.

WO2018/041973 relates to chemically modified single-stranded RNA-editing oligonucleotides for the deamination of a target adenosine by an ADAR enzyme whereby the central base triplet (CBT) of three sequential nucleotides comprises a sugar modification and/or a base modification. It was found that deoxyribose at all three positions of the CBT is well tolerated and provides substantial stabilization against nuclease digestion. In one embodiment, at least two of the three nucleotides of the CBT are chemically modified at the 2′ position of the sugar moiety, are deoxyribonucleosides, or a combination thereof.

According to the invention, the oligonucleotides comprise a central base triplet (CBT) of 3 nucleotides (5′-N₊₁ N₀ N₋₁-3′) with the central nucleotide (N₀) directly opposite to the target adenosine (A) in the target RNA. In some embodiment, N₊₁ is 2′-F, 2′-FANA (2′-deoxy-2′-fluoro-D-arabinonucleic acid), DNA, or 2′-O-methyl. In some embodiment, No is 2′-FANA or DNA. In some embodiment, N₋₁ is 2′-FANA, DNA, or 2′-O-methyl.

In one embodiment, the N₋₂ nucleotide carries a 2′-O-alkyl-modification; and wherein the N₋₃ nucleotide carries a 2′-fluoro (2′-F)-modification. In one preferred embodiment, the N₋₂ nucleotide carries a 2′-O-methyl-modification.

In one embodiment, the oligonucleotide does not contain a stem loop secondary structure.

The oligonucleotides may comprise a moiety, which enhances cellular uptake of the oligonucleotide, e.g., N-acetylgalactosamine (GalNAc). Hence, in some embodiments, the oligonucleotide comprises a moiety or is conjugated to a moiety that enhances cellular uptake of the oligonucleotide. Preferably, the moiety enhancing cellular uptake is a triantennary N-acetyl galactosamine (GalNAc₃).

### 3. Generalizable Rules

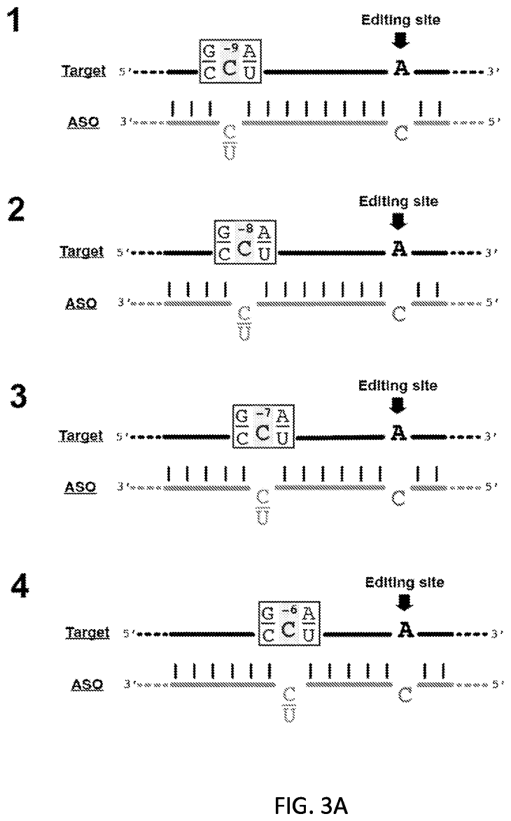

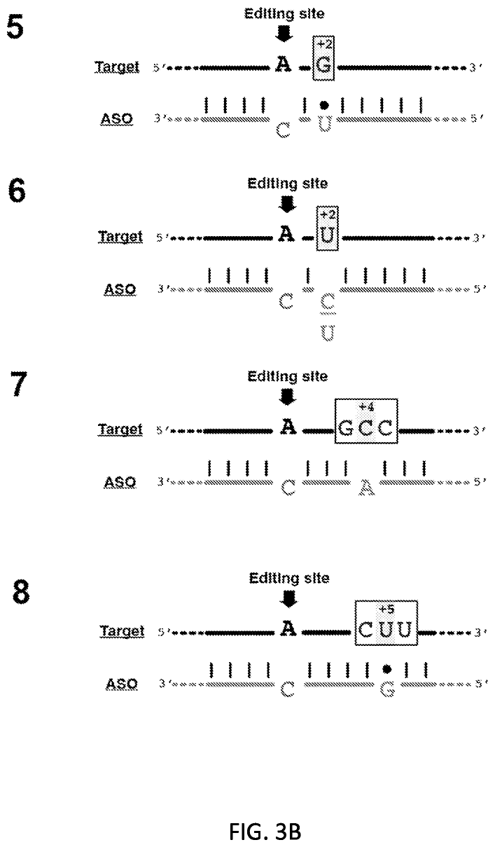

In some aspects, provided herein are methods for designing guide RNAs (antisense oligonucleotides) with optimized editing efficiency for a variety of target RNA sequences. These methods are referred to herein as “generalizable rules”. The scope of the invention encompasses guide RNAs comprising selected features of the generalizable rule, as described below. The scope of the invention further encompasses the use of such guide RNAs embodying a selected generalizable rule to achieve A-to-I editing of amenable target transcripts, for example, in a therapeutic application or protein engineering context. The different ‘generalizable rules’ are depicted and summarized in FIG. 3.

A first generalizable rule identified herein is that if the target sequence contains a C residue at position-9 relative to the editing site, flanked by a 5′ C or G and 3′ A or U, then a mismatched base in the ASO across-9C (i.e., C or U) increases editing. A schematic version of this rule is shown in FIG. 3A, item 1. The ASO facilitates ADAR-mediated A-to-G base replacement of a 5′-3′ target sequence comprising X1-X2-X3-X4-X5-X6-X7-X8-X9-X10-A*, wherein X1 is C or G, X2 is C, X3 is A or U, and A* is the target base.

A second generalizable rule identified herein is that if the target sequence contains a C at position-8 relative to the editing site, flanked by a 5′ C or G and 3′ A or U, then a mismatched base in the ASO across-8C (i.e., C or U) increases editing. A schematic version of this rule is shown in FIG. 3A, item 2. The ASO facilitates ADAR-mediated A-to-G base replacement of a 5′-3′ target sequence comprising X1-X2-X3-X4-X5-X6-X7-X8-X9-A*, wherein X1 is C or G, X2 is C, X3 is A or U, and A* is the target base.

A third generalizable rule identified herein is that if the target sequence contains a C residue at position-7 relative to the editing site, flanked by a 5′ G or C and a 3′ A or U, then a mismatched base in the ASO across-7C (i.e., C or U) increases editing. A schematic version of this rule is shown in FIG. 3A, item 3. The ASO facilitates ADAR-mediated A-to-G base replacement of a 5′-3′ target sequence comprising X1-X2-X3-X4-X5-X6-X7-X8-A*, wherein X1 is C or G, X2 is C, X3 is A or U, and A* is the target base.

A fourth generalizable rule identified herein is that if the target sequence contains a C residue at position-6 relative to the editing site, flanked by a 5′ G or C and a 3′ A or U, then a mismatched base in the ASO across-6C (i.e., C or U) increases editing. A schematic version of this rule is shown in FIG. 3A, item 4. The ASO facilitates ADAR-mediated A-to-G base replacement of a 5′-3′ target sequence comprising X1-X2-X3-X4-X5-X6-X7-A*, wherein X1 is C or G, X2 is C, X3 is A or U, and A* is the target base.

A fifth generalizable rule identified herein is that if the target sequence contains a G residue at position +2 relative to the editing site, then a U in the ASO across +2G (presumably forming a G·U wobble base-pair) increases editing. A schematic version of this rule is shown in FIG. 3B, item 5. The ASO facilitates ADAR-mediated A-to-G base replacement of a 5′-3′ target sequence comprising A *-X1-X2-X3, wherein X2 is G, X1 and X3 are A, C, G or U, and A* is the target base.

A sixth generalizable rule identified herein is that if the target sequence contains a U at position +2 relative to the editing site, then a mismatched base in the ASO across from +2 (i.e., C or U) increases editing. A schematic version of this rule is shown in FIG. 3B, item 6. The ASO facilitates ADAR-mediated A-to-G base replacement of a 5′-3′ target sequence comprising A *-X1-X2-X3, wherein X2 is U, X1 and X3 are A, C, G or U, and A* is the target base.

A seventh generalizable rule identified herein is that if the target sequence contains a C residue at position +4 relative to the editing site, flanked by a 5′ G and 3′ C, then an A mismatch in the ASO across +4C increases editing. A schematic version of this rule is shown in FIG. 3B, item 7. The ASO facilitates ADAR-mediated A-to-G base replacement of a 5′-3′ target sequence comprising A *-X1-X2-X3-X4-X5, wherein X3 is G, X4 is C, X5 is C, and A* is the target base.

An eighth generalizable rule identified herein is that if the target sequence contains a U residue at position +5 relative to the editing site, flanked by a 5′ C and 3′ U, then a G in the ASO across +5U (presumably forming a G·U wobble base-pair) increases editing. A schematic version of this rule is shown in FIG. 3B, item 8. The ASO facilitates ADAR-mediated A-to-G base replacement of a 5′-3′ target sequence comprising A *-X1-X2-X3-X4-X5-X6, wherein X4 is C, X5 is U, X6 is U, and A* is the target base.

### 5. Editing Methods and Therapeutic Use

Site-directed A-to-I RNA editing facilitated by the guide RNAs of the inventions presents the opportunity to temporarily manipulate essential biological processes, such as cell signaling or inflammation, whose permanent alteration would otherwise have serious consequences, and allows for the precise regulation of the biological outcome due to the tunability of introducing a change in RNA (e.g., potentially from 0% to 100%). Inosine is biochemically interpreted as guanosine by the cellular machinery, as such A-to-I editing formally introduces A-to-G point mutations in RNA, which offers the opportunity to manipulate or restore genetic information. Site-directed editing may take place in vitro, in vivo or ex vivo.

Generally described herein are methods for site-directed RNA editing. According to the invention, provided herein is a method for site-directed A-to-I editing of a target RNA. The methods of the invention may be applied to any cell. Suitable cells include eukaryotic cells including but not limited to yeast cells, higher plant cells, animal cells, insect cells, and mammalian cells. Non-limiting examples of eukaryotic cells include simian, bovine, porcine, murine, rat, avian, reptilian, and human cells.

Provided herein is a method for site-directed A-to-I editing of a target RNA, comprising providing to a cell or subject an oligonucleotide according to the invention or a composition according to the invention. In some embodiments, provided herein are methods for site-directed RNA editing. In some embodiments, the methods comprise selecting a gRNA, including selecting a gRNA by a method/platform as described herein, and providing a construct comprising the guide RNA to a cell or a subject. In some embodiments, the methods comprise selecting a gRNA known to have increased editing efficiency based upon conformity to one or more generalizable rules described herein and providing the gRNA to a cell or a subject. In some embodiments, the guide RNA is a gRNA as described herein. In some embodiments, the construct may additionally comprise a targeting domain to direct the construct to a given cell or tissue.

In a primary implementation, the scope of the invention encompasses a method of achieving site-directed A-to-I RNA editing in a target transcript of a cell by introducing to the cell a selected guide RNA or oligonucleotide. In other implementations, the scope of the invention encompasses a method of correcting a point mutation in a target transcript in a cell by introducing to the cell a selected guide RNA that facilitates A-to-I RNA editing at the target position. In another implementation, the scope of the invention encompasses a method of treating a selected disease or condition in a subject by administering to the subject a therapeutically effective amount of a selected guide RNA that facilitates A-to-I RNA editing in a target transcript, wherein such A-to-I RNA editing achieves a therapeutic outcome, for example, increasing the abundance or activity of a functional protein, reducing the abundance of a pathological protein, alleviating the symptoms of the disease or condition, preventing the onset of the disease or condition, slowing or halting the progression of the disease or condition, or curing the condition.

Also provided herein is a method for creating a de-novo A-to-G beneficial protein variant, wherein the beneficial protein variant is derived from a wild-type protein selected from the group consisting of: a) STAT3 containing the Y705 phosphorylation site; b) STAT1 containing the Y701phosphorylation site; c) IFNAR1 containing the Y466 phosphorylation site; d) CTNNB1 containing the T41 phosphorylation site; and e) TMEM173 containing the N154 or R284 residue; wherein the method comprises contacting a cell with an oligonucleotide of the invention. In one embodiment, the beneficial protein variant is derived from the wild-type protein STAT3 containing the Y705 phosphorylation. In one embodiment, the beneficial protein variant is derived from the wild-type protein STAT1 containing the Y701phosphorylation site. In one embodiment, the beneficial protein variant is derived from the wild-type protein IFNAR1 containing the Y466 phosphorylation site. In one embodiment, the beneficial protein variant is derived from the wild-type protein CTNNB1 containing the T41 phosphorylation site. In one embodiment, the beneficial protein variant is derived from the wild-type protein TMEM173 containing the N154. In one embodiment, the beneficial protein variant is derived from the wild-type protein TMEM173 containing the R284 residue.

The present disclosure also describes gRNA or oligonucleotide compositions. Provided herein are compositions comprising the gRNA or oligonucleotides (or construct comprising the same) of the invention. In some embodiments, the compositions are pharmaceutical compositions. As used herein, pharmaceutical composition means a mixture of substances suitable for administering to an individual. For example, a pharmaceutical composition may comprise one or more active pharmaceutical agents, such as an oligonucleotide, and a sterile aqueous solution. In one embodiment, the composition contains one or more oligonucleotides of the invention. In one embodiment, provided herein is a pharmaceutical composition or a pharmaceutically acceptable salt thereof comprising an antisense oligonucleotide of the invention or a vector of the invention. In some embodiments, the guide RNA, oligonucleotide, or construct comprising the same is formulated into a composition for delivery to the cell or subject.

Also described herein are methods for treating or preventing diseases or conditions associated with point mutations or wild-type proteins. In one aspect provided herein is a method of treating or preventing a disease or condition associated with a point mutation or a wild-type protein in a subject, wherein the method comprises administering to the subject a therapeutically effective amount of an oligonucleotide or a composition of the invention.

Provided herein are also oligonucleotides of the invention or compositions of the invention for use in therapy.

In another aspect provided herein is an oligonucleotide or composition for use in a method of treating or preventing a disease or condition associated with a point mutation in a subject. In one embodiment, the disease or condition is selected from the group consisting of: a (1) AT (AAT) deficiency, Parkinson's disease, Myopathy, ALDH2 deficiency, Cancer, Congenital insensitivity to pain, Beta-Thalassemia, Thrombophilia, Rett Syndrome and Stargardt disease. In one embodiment, the disease or condition is a (1) AT (AAT) deficiency. In one embodiment, the disease or condition is Parkinson's disease. In one embodiment, the disease or condition is Myopathy. In one embodiment, the disease or condition is ALDH2 deficiency. In one embodiment, the disease or condition is Cancer. In one embodiment, the disease or condition is Congenital insensitivity to pain. In one embodiment, the disease or condition is Beta-Thalassemia. In one embodiment, the disease or condition is Thrombophilia. In one embodiment, the disease or condition is Rett Syndrome. In one embodiment, the disease or condition is Stargardt disease. In one embodiment, the oligonucleotide or composition is for use in a method of treating or preventing a disease or condition associated with a point mutation in a protein, wherein the point mutation is selected from the group consisting of: SERPINA1 carrying the E342K mutation; SNCA carrying the A53T mutation; GFER carrying the R194H mutation; ALDH2 carrying the E504K mutation; TP53 carrying the oncogenic R175H mutation; SCN9A carrying the W897X mutation; HBB carrying the W38X mutation; KRAS carrying the oncogenic G12D mutation; KRAS carrying the oncogenic G12S mutation; MeCP2 carrying the R106Q mutation; F5 carrying the R534Q mutation; and ABCA4 carrying the G1961E mutation.

In some embodiments, the guide RNA or oligonucleotide comprises an antisense sequence, or variant thereof, selected from SEQ ID NO. 1-1165 of Table 3. In one embodiment, the disease or condition is Parkinson's Disease, the target transcript comprises the SNCA A53T mutation, and the guide sequence comprises a sequence, or variant thereof, selected from any of SEQ ID NO. 1-222. In one embodiment, the disease or condition is a (1) AT deficiency or an associated pathology, the target transcript comprises SERPINA1 carrying the E342K mutation, and the guide sequence comprises a sequence, or variant thereof, selected from any of SEQ ID NO. 223-291. In one embodiment, the disease or condition is associated with the GFER R194H mutation, the target transcript comprises GFER carrying the R194H mutation, and the guide sequence comprises a sequence, or variant thereof, selected from any of SEQ ID NO. 292-339. In one embodiment, the disease or condition is deficiency in aldehyde metabolism, the target transcript comprises ALDH2 carrying the E504K mutation, and the guide sequence comprises a sequence, or variant thereof, selected from any of SEQ ID NO. 340-399. In one embodiment, the disease or condition is cancer or risk of cancer, the target transcript comprises TP53 carrying the R175H mutation, and the guide sequence comprises a sequence, or variant thereof, selected from any of SEQ ID NO. 400-518. In one embodiment, the disease or condition is associated with defects in the alpha subunit of a sodium channel NaV1.7, the target transcript comprises SCN9A carrying the W897X mutation, and the guide sequence comprises a sequence, or variant thereof, selected from any of SEQ ID NO. 519-593. In one embodiment, the disease or condition is beta thalassemia or a condition associated with hemoglobin subunit beta defects, the target transcript comprises HBB carrying the W38X mutation, and the guide sequence comprises a sequence, or variant thereof, selected from any of SEQ ID NO. 594-634. In one embodiment, the disease or condition is cancer or risk of cancer, the target transcript comprises KRAS carrying the G12D mutation, and the guide sequence comprises a sequence, or variant thereof, selected from any of SEQ ID NO. 635-694. In one embodiment, the disease or condition is cancer or risk of cancer, the target transcript comprises KRAS carrying the G12S mutation, and the guide sequence comprises a sequence, or variant thereof, selected from any of SEQ ID NO. 695-728. In one embodiment, the disease or condition is Rett Syndrome, the target transcript comprises MeCP2 carrying the R106Q mutation, and the guide sequence comprises a sequence, or variant thereof, selected from any of SEQ ID NO. 729-753. In one embodiment, the disease or condition is associated with Factor V defects, the target transcript comprises F5 carrying the R534Q mutation, and the guide sequence comprises a sequence, or variant thereof, selected from any of SEQ ID NO. 754-796. In one embodiment, the disease or condition is retinal disease, a maculopathy, or Stargardt disease, the target transcript comprises ABCA4 carrying the G1961E mutation, and the guide sequence comprises a sequence, or variant thereof, selected from any of SEQ ID NO. 797-926.

In some instances, the subject is not in need of treatment involving the targeting of a transcript that comprises a mutation. Rather the oligonucleotides are being used for “beneficial editing” of a target nucleic acid. That is, the subject receives the oligonucleotide or composition to edit a target nucleic acid derived from a wild-type allele (not a mutated allele) in order to modulate the function of the wild-type protein in a useful way. In this way, new protein variants of the wild-type protein can be created. For example, beneficial editing may include sites, such as STAT1 Y701, that are not causes for genetic diseases but rather represent wild-type protein sites. These sites may be changed (no underlying G-to-A mutation) to alter the function of the wild-type protein. In one embodiment, the site-directed RNA editing creates a beneficial protein variant and the target sequence comprises a wild-type sequence. In one embodiment, the target transcript comprises wild-type STAT3, the guide sequence comprises a sequence, or variant thereof, selected from any of SEQ ID NO. 927-966, and the beneficial protein variant comprises STAT3 Y705C. In one embodiment, the target transcript comprises wild-type STAT1, the guide sequence comprises a sequence, or variant thereof, selected from any of SEQ ID NO. 967-1005, and the beneficial protein variant comprises STAT1 Y701C. In one embodiment, the target transcript comprises wild-type IFNAR1, the guide sequence comprises a sequence, or variant thereof, selected from any of SEQ ID NO. 1006-1029, and the beneficial protein variant comprises IFNAR1 Y466C. In one embodiment, the target transcript comprises wild-type CTNNB1, the guide sequence comprises a sequence, or variant thereof, selected from any of SEQ ID NO. 1030-1070, and the beneficial protein variant comprises CTNNB1 T41A. In one embodiment, the target transcript comprises wild-type TMEM173, the guide sequence comprises a sequence, or variant thereof, selected from any of SEQ ID NO. 1071-1134, and the beneficial protein variant comprises TMEM173 N154S. In one embodiment, the target transcript comprises wild-type TMEM173, the guide sequence comprises a sequence, or variant thereof, selected from any of SEQ ID NO. 1135-1165, and the beneficial protein variant comprises TMEM173 R284G.

In a primary implementation, the guide RNAs or oligonucleotides of the invention are introduced to the target cell or administered to a subject in the form of an oligonucleotide comprising a selected guide RNA of the invention. In alternative implementations, the guide RNAs or oligonucleotides of the invention are introduced to the cell or administered to the subject as an expression vector that produces the guide RNA or oligonucleotides in vivo. In a primary implementation, the guide RNAs or oligonucleotides achieve RNA editing at the target site by the action of endogenous ADARs or RNA editing enzymes. In alternative implementations, the guide RNAs or oligonucleotides achieve RNA editing at the target site by the action of introduced, for example, co-administered, ADARs or RNA editing enzymes, such as engineered forms of ADAR.

Oligonucleotides may be administered in a pharmaceutical composition and one or more additional elements, including carriers, excipients, drug delivery and targeting compositions, etc. In one embodiment, a composition comprises an oligonucleotide of the invention in an admixture with a pharmaceutically acceptable carrier. In some embodiments, the pharmaceutically acceptable carrier can simply be a saline solution. This can be isotonic or hypotonic.

In some embodiments, a pharmaceutical composition comprises one or more other therapies in addition to an oligonucleotide of the invention.

In certain embodiments, the compositions of the invention further include diluents of various buffer content (e.g., Tris-HCl, acetate, phosphate), pH, and ionic strength, and additives such as detergents and solubilizing agents (e.g., Tween 80, Polysorbate 80), anti-oxidants (e.g., ascorbic acid, sodium metabisulfite), preservatives (e.g., Thimersol, benzyl alcohol), and bulking substances (e.g., lactose, mannitol). In some embodiments, the material is incorporated into particulate preparations of polymeric compounds such as polylactic acid, polyglycolic acid, etc. or into liposomes. In some embodiments, hyaluronic acid is used. Such compositions may influence the physical state, stability, rate of in vivo release, and/or rate of in vivo clearance of the present ASOs and derivatives. In some embodiments, the compositions are prepared in liquid form, or are in dried powder, such as lyophilized form.

In certain embodiments, the pharmaceutical compositions described herein additionally comprise one or more salts, e.g., sodium chloride, calcium chloride, sodium phosphate, monosodium glutamate, and aluminium salts (e.g., aluminium hydroxide, aluminium phosphate, alum (potassium aluminium sulfate), or a mixture of such aluminium salts). In other embodiments, the pharmaceutical compositions described herein do not comprise salts.

The pharmaceutical compositions described herein can be included in a container, pack, or dispenser together with instructions for administration.

In the case of genetically introduced guide RNAs, any suitable vector may be used, for example, viral vectors (e.g., lentiviral vectors, retroviral vectors, adenovirus vectors, adeno-associated virus vectors, alphavirus vectors, etc.) and non-viral vectors (e.g., plasmids, cosmids, phages, etc.).

The compositions may be used in methods of treating and/or preventing a genetic disorder, condition, or disease. In a specific embodiment, the pharmaceutical compositions are suitable for veterinary and/or human administration. In some embodiments, an oligonucleotide or composition containing an oligonucleotide described herein is administered to a human. In some embodiments, the human subject to be administered an oligonucleotide or composition containing an oligonucleotide is any individual at risk of developing a disease or disorder associated with one of the targets disclosed herein.

The invention generally describes the use of an oligonucleotide and/or composition comprising the same in the medical setting. The present invention describes antisense oligonucleotide and/or composition for use in the treatment or prevention of a genetic disorder, condition, or disease as well as methods for treating or preventing a genetic disorder, condition, or disease.

The oligonucleotide or composition comprising the same may be used in the treatment and/or prevention of a medical condition. In one aspect provided herein is the use of an oligonucleotide of the invention and/or a composition comprising the same in the treatment or prevention of a genetic disorder, condition, or disease. In one embodiment, the genetic disorder, condition, or disease is associated with a G-to-A mutation.

Also provided herein is an oligonucleotide or a composition of the invention for use in therapy.

Further provided herein is an oligonucleotide or a composition of the invention for use in a method of treating or preventing a disease or condition associated with a point mutation in a subject. In one embodiment, the disease or condition is selected from the group consisting of: a (1) AT (AAT) deficiency, Parkinson's disease, Myopathy, ALDH2 deficiency, Cancer, Congenital insensitivity to pain, Beta-Thalassemia, Thrombophilia, Rett Syndrome and Stargardt disease.

Also provided herein is an oligonucleotide or composition of the invention for use in a method of treating or preventing a disease or condition associated with a point mutation in a protein, wherein the point mutation is selected from the group consisting of: a) SERPINA1 carrying the E342K mutation; b) SNCA carrying the A53T mutation; c) GFER carrying the R194H mutation; d) ALDH2 carrying the E504K mutation; e) TP53 carrying the oncogenic R175H mutation; f) SCN9A carrying the W897X mutation; g) HBB carrying the W38X mutation; h) KRAS carrying the oncogenic G12D mutation; i) KRAS carrying the oncogenic G12S mutation; j) MeCP2 carrying the R106Q mutation; k) F5 carrying the R534Q mutation; and 1) ABCA4 carrying the G1961E mutation.

Also provided is an oligonucleotide or a composition of the invention for use in a method of treating or preventing a disease or condition associated with wild-type proteins in a subject. That is, the oligonucleotides or compositions of the invention can be used to mediate an A-to-G substitution in a wild-type protein. In one embodiment, the wild-type protein is selected from the group consisting of: a) STAT3 containing the Y705 phosphorylation site; b) STAT1 containing the Y701phosphorylation site; c) IFNAR1 containing the Y466 phosphorylation site; d) CTNNB1 containing the T41 phosphorylation site; and e) TMEM173 containing the N154 or R²⁸⁴ residue.

The oligonucleotide of the invention or the (pharmaceutical) composition may be administered, for example, orally in any orally acceptable dosage form including, but not limited to, capsules, tablets, aqueous suspensions, or solutions, or parenterally. In some embodiments, formulations suitable for parenteral administration comprise sterile aqueous preparations of at least one embodiment of the present disclosure, which are approximately isotonic with the blood of the intended recipient. The amount of oligonucleotide or composition to be administered, the dosage and the dosing regimen can vary from cell type to cell type, the disease to be treated, the target population, the mode of administration (e.g., systemic versus local), the severity of disease and the acceptable level of side activity. In some embodiments, the amount of oligonucleotides administered in a pharmaceutical composition is dependent on the subject being treated, the subject's weight, the manner of administration, and/or disease to be treated.

All references, including publications, patent applications, and patents, cited herein are hereby incorporated by reference to the same extent as if each reference were individually and specifically indicated to be incorporated by reference and were set forth in its entirety herein.

Preferred embodiments are described herein, including the best mode known to the inventors for carrying out the methods described herein. Variations of those preferred embodiments may become apparent to those of ordinary skill in the art upon reading the foregoing description. The inventors expect skilled artisans to employ such variations as appropriate, and the inventors intend for the methods to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### EXAMPLES

The examples shown in the following are merely illustrative and shall describe the present invention in a further way. These examples shall not be construed to limit the present invention thereto.

### Example 1

Adenosine deaminases acting on RNA, or ADAR proteins, ubiquitously expressed in human cells, bind double-stranded RNA and edit adenosine to inosine, which is biochemically interpreted as guanosine. Antisense oligonucleotides or guide RNAs can be used to recruit endogenous ADARs for site-directed RNA editing. Targeting of several endogenous transcripts with these guide RNAs yielded RNA editing efficiencies of up to 80-90% in various cell types. Site-directed editing is highly specific and maintains the natural editing homeostasis (Merkle et al, 2019). Importantly, this new technology holds several advantages over alternative genome engineering techniques like CRISPR by (1) harnessing endogenous ADAR proteins, which are already abundantly expressed throughout human tissues, thus eliminating the need of Cas9 delivery and associated toxicity; (2) editing patients' RNA, which, unlike DNA editing, allows for reversible and therefore safer manipulation; (3) exhibiting very high specificity. Accordingly, this approach offers a precise and efficient platform for transcriptome engineering, including the repair of pathogenic G-to-A mutations, without the significant safety risks associated with other genome engineering approaches.

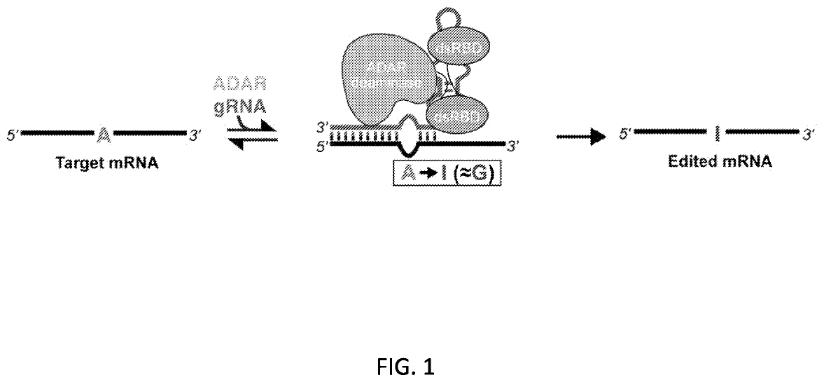

ADAR-recruiting gRNAs contain two functional domains; the first, antisense domain binds to the target RNA, while the second, ADAR-recruiting domain, binds and recruits the endogenous ADAR protein. A schematic of an ADAR-recruiting gRNA is shown in FIG. 1. gRNAs can be chemically modified to improve the target affinity, in vivo stability, and cellular uptake (Levin, 2019; Merkle et al, 2019). However, the efficiency of site-directed ADAR editing continues to lag behind the up-to 100% editing efficiencies seen in some natural ADAR substrates (Nishikura, 2016). Therefore, what is needed are high-throughput screens to optimize ADAR gRNA designs, along with effective gRNAs targeting a variety of disease phenotypes.

Deviations from perfect double-stranded RNA (dsRNA) may enhance the editing efficiency and specificity. Without wishing to be bound by theory, it is possible that deviations from perfect dsRNA may enhance editing efficiency and/or specificity by defining the productive ADAR binding registers along the RNA duplex and creating unfavorable contexts for ‘off-target’ A residues.

Guides can be designed at least in part based on published prototypes that gave the highest editing for other targets (Merkle et al, 2019) and those described in WO2022087272A1, the entire contents of which are incorporated herein by reference for all purposes. High-throughput screening to optimize guide RNAs can also be performed, which will further inform the guide design. The gene editing level can be assessed, such as by sequencing, and the transcriptome-wide off-target editing can be determined, such as by RNA-Seq.

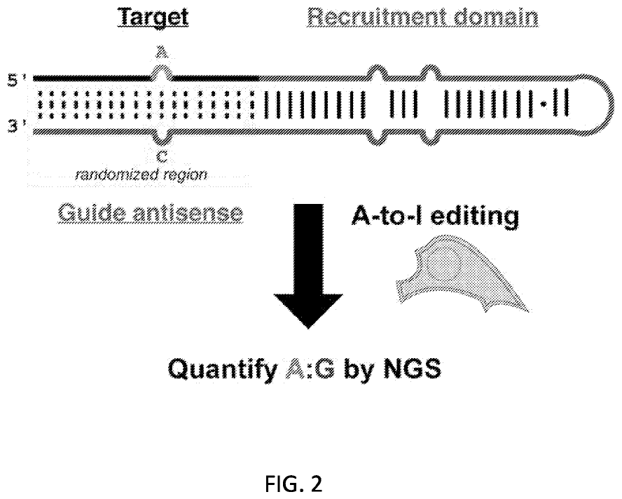

To achieve the highest possible efficiency and specificity of guide RNAs, a workflow that screens a library of thousands of gRNA sequences for their ability to edit G-to-A point mutations in various disease states can be employed. An exemplary workflow is shown in FIG. 2.

A next-generation sequencing approach can be used, including both unbiased investigation of randomized guide sequences and rationally designed gRNA variants. To enable simultaneous assessment of the guide sequence and associated editing level in a high-throughput fashion, a hybrid construct can be used, in which the target region from a desired sequence is covalently fused to the guide RNA. The resulting hairpin structures simulate the duplex formed between the target RNA and a trans-acting guide RNA, while enabling preservation of the information on the editing level and the guide sequence in the same sequencing construct.

The starting point for library design is based on a prototype comprising a short (18-20 nt) antisense region and a recruitment domain derived from an endogenous ADAR substrate (R/G motif from GluR2 mRNA, or ‘GluR/G’; SEQ ID NO: 1166).

Given the apparent importance of unpaired residues (located in bulges and loops) for high editing efficiency, the antisense sequence can first be randomized. Hand-mixed bases can be used during library synthesis to ensure comprehensive coverage of single, double, and triple mismatches, while still enabling decent coverage of higher-order mutants. Designed variants containing all possible single-nucleotide insertions and deletions in the antisense strand, as well as structural perturbations observed in natural targets, can also be used.

The recruitment domain variants can include high-efficiency natural ADAR substrates, as well as designed structures, including double recruitment domains, Z-RNA forming motifs (targeting ADAR1's Z-RNA binding domain) and other perturbations. Recruitment domain length, sequence, stability, and distance from the editing site can be systematically varied. In some embodiments, the recruitment domain can be omitted altogether by extending the length of the antisense region. Hairpin variants consisting entirely of the desired target sequence, and its complement can also be used.

The hairpin library, comprising all variants discussed above, can be introduced into a reporter plasmid and delivered into cells expressing or inducibly expressing ADAR proteins. For example, the hairpin library can be introduced into a reporter plasmid and delivered into FlpIn TRex 293 cells expressing endogenous ADARs or inducibly expressing individual ADAR proteins (ADAR1 or ADAR2). Upon extraction of total RNA, the variants can be reverse transcribed using a primer targeting a constant flanking region. The reverse transcription primer can also include a randomized unique molecular identifier (UMI) sequence, to account for PCR and sequencing artifacts in subsequent sequencing analysis (Buenrostro et al, 2014; Kivioja et al, 2012). Sequencing can be performed using the Illumina HiSeq platform.

The most promising sequence and structural features identified in the screen can subsequently be implemented in trans, i.e., by delivering plasmid- or AAV-expressed guides to cells expressing the mutated target sequence affiliated with a given disease state. The extent of editing can be assessed by Sanger sequencing and amplicon deep sequencing.

Using methods such as those describe above, antisense sequences were designed and randomized to introduce varying numbers of mismatches with the target sequence. Antisense sequences for 18 disease-relevant target sequences were tested. High-throughput screening revealed a number of generalizable rules (e.g., applicable to a variety of disease targets) for determining which ASO will possess increased editing efficiency. Schematics for these generalizable rules are shown in FIG. 3A and FIG. 3B, and descriptions of each generalizable rule along with exemplary sequences for each are in the following examples. A summary of screening results is shown in Table 1. As indicated in the table, gRNA libraries targeting pathogenic G-to-A point mutations affiliated with AAT deficiency, Parkinson's disease, Myopathy, ALDH2-deficiency, Cancer, Congenital insensitivity to pain, Beta-Thalassemia, Cancer, Thrombophilia, Rett syndrome, and Stargardt disease were investigated. Moreover, gRNA libraries introducing beneficial de novo A-to-G variants were also investigated that could manipulate cellular signaling or induce innate immune signaling. Accordingly, the methods described herein and the generalized rules for developing ASOs with high editing efficiency are broadly applicable to a variety of disease states and are applicable for introducing beneficial mutations in addition to correcting pathogenic mutations.

TABLE 1
Summary of screening results for gRNA libraries targeting pathogenic G-to-A point mutations

| | | | | | Editing | |
|---|---|---|---|---|---|---|
| | | | | Number of | enhancement by | Number of |
| | | | | gRNAs | gRNAs listed in | optimized gRNAs |
| | Disease | Gene | Mutation | screened¹ | Table 3 (≥n-fold)² | listed in Table 3 |
| 1 | AAT deficiency | SERPINA1 | E342K | 15346 | 2 | 69 |
| 2 | Parkinson's disease | SNCA | A53T | 14024 | 3 | 222 |
| 3 | Myopathy | GFER | R194H | 5818 | 1.3 | 48 |
| 4 | ALDH2 deficiency | ALDH2 | E504K | 2808 | 1.3 | 60 |
| 5 | Cancer | TP53 | R175H | 17794 | 1.5 | 119 |
| 6 | Congenital insensitivity | SCN9A | W897X | 11005 | 1.3 | 75 |
| | to pain | | | | | |
| 7 | Beta-Thalassemia | HBB | W38X | 5754 | 1.1 | 41 |
| 8 | Cancer | KRAS | G12D | 6015 | 3 | 60 |
| 9 | Cancer | KRAS | G12S | 8449 | 1.5 | 34 |
| 10 | Thrombophilia | F5 | R534Q | 6721 | 1.3 | 43 |
| 11 | Rett Syndrome | MeCP2 | R106Q | 6545 | 1.2 | 25 |
| 12 | Stargardt disease | ABCA4 | G1961E | 6798 | 3 | 130 |

¹Variants meeting the following criteria: 1) represented by ≥10 cDNAs (barcodes); 2) for libraries with replicate measurements (in bold), only reproducible variants are included (relative standard deviation ≤0.3).
²Enhancements are calculated relative the prototype design, in which the gRNA is fully complementary to the target sequence except for an A-C mismatch opposite the target A.

TABLE 2
Summary of screening results for gRNA libraries creating beneficial de-novo A-to-G variants

| | | | | | Editing | |
|---|---|---|---|---|---|---|
| | | | | Number of | enhancement by | Number of |
| | | | Beneficial | gRNAs | gRNAs in Table 3 | optimized gRNAs |
| | Purpose | Gene | variant | screened | (≥n-fold) | listed in Table 3 |
| 1 | Manipulate cellular signaling | STAT1 | Y701C | 7786 | 1.3 | 39 |
| 2 | Manipulate cellular signaling | STAT3 | Y705C | 8386 | 1.2 | 40 |
| 3 | Manipulate cellular signaling | IFNAR1 | Y466C | 3072 | 1.3 | 24 |
| 4 | Induce innate immune signaling | TMEM173 | R284G | 7565 | 1.5 | 31 |
| 5 | Induce innate immune signaling | TMEM173 | N154S | 5683 | 2 | 64 |
| 6 | Induce tissue regeneration | CTNNB1 | T41A | 9068 | 1.3 | 41 |

### Example 2

### Generalizable Rule 1

A first generalizable rule identified herein is that if the target sequence contains a C residue at position-9 relative to the editing site, flanked by a 5′ C or G and 3′ A or U, then a mismatched base in the ASO across-9C (i.e., C or U) increases editing. A schematic version of this rule is shown in FIG. 3A, item 1. The ASO facilitates ADAR-mediated A-to-G base replacement of a 5′-3′ target sequence comprising X1-X2-X3-X4-X5-X6-X7-X8-X9-X10-A*, wherein X1 is C or G, X2 is C, X3 is A or U, and A* is the target base.

For example, in some embodiments the target RNA encodes SNCA, carrying the A53T mutation associated with Parkinson's disease. In some embodiments, the target RNA comprises the following sequence, with the mutated target adenosine underlined:

5′GUGUUCUCUAUGUAGGCUCCAAAACCAAGGAGGGAGUGGUGCAUGGUGU GACAACAGUGGCUGAGAAGACCAAAGAGCAAGUGACAAAUGUUGGAGGAGCA 3′ (SEQ ID NO: 1168). In some embodiments, the corresponding complementary sequence (with C opposite from the target A) is as follows:

(SEQ ID NO: 1186)
3′CACAAGAGAUACAUCCGAGGUUUUGGUUCCUCCCUCACCACGUACCA
CACCGUUGUCACCGACUCUUCUGGUUUCUCGUUCACUGUUUACAACCUC
CUCGU 5′.

The following exemplary ASOs, containing a C-C or C-U mismatch at position-9 (in square brackets and bold) relative to the editing site will enhance editing of the target adenosine:

(SEQ ID NO: 1187)
3′CACAAGAGAUACAUCCGAGGUUUUGGUUCCUCCCUCACCAC[C]U
ACCACACCGUUGUCACCGACUCUUCUGGUUUCUCGUUCACUGUUUACAA
CCUCCUCGU 5′.
(SEQ ID NO: 1188)
3′CACAAGAGAUACAUCCGAGGUUUUGGUUCCUCCCUCACCAC[U]UAC
CACACCGUUGUCACCGACUCUUCUGGUUUCUCGUUCACUGUUUACAACC
UCCUCGU 5′.
(SEQ ID NO: 1189)
3′ CAC[C]UACCACACCGUUGUCACCGACUCUUCUGG 5′.
(SEQ ID NO: 1190)
3′ CAC[U]UACCACACCGUUGUCACCGACUCUUCUGG 5′.

The C-C or C-U mismatch at position-9 can be used within ASO's of any length and symmetry. For example, in some embodiments, the C-C or C-U mismatch at position-9 can be used in ASOs having a length of 15-101 nt, for example, in some implementations comprising between 4-50 nucleotides on either the 3′ or 5′ side of the target.

In some embodiments, the C-C or C-U mismatch at position-9 may be combined with other features, such as a G-G mismatch at position −1 relative to the editing site (in square brackets and bold):

(SEQ ID NO: 1191)
3′CACAAGAGAUACAUCCGAGGUUUUGGUUCCUCCCUCACCAC[C]UAC
CACA[G]CGUUGUCACCGACUCUUCUGGUUUCUCGUUCACUGUUUACAA
CCUCCUCGU 5′.
(SEQ ID NO: 1192)
3′CACAAGAGAUACAUCCGAGGUUUUGGUUCCUCCCUCACCAC[U]UAC
CACA[G]CGUUGUCACCGACUCUUCUGGUUUCUCGUUCACUGUUUACAA
CCUCCUCGU 5′.
(SEQ ID NO: 1193)
3′ CAC[C]UACCACA[G]CGUUGUCACCGACUCUUCUGG 5′.
(SEQ ID NO: 1194)
3′ CAC[U]UACCACA[G]CGUUGUCACCGACUCUUCUGG 5′.

In some embodiments, the ASO comprises a recruitment domain. An exemplary recruitment domain (SEQ ID NO: 1166) is shown in italics in the exemplary ASOs below:

(SEQ ID NO: 1195)
3′CAC[C]UACCACACCGUUGUCAGGUGUCGAGAAGAGGAGAACAAUAU
GCCAAAUGUUGUUCUCGUCUCCUCGACACC 5′.
(SEQ ID NO: 1196)
3′CAC[U]UACCACACCGUUGUCAGGUGUCGAGAAGAGGAGAACAAUAU
GCCAAAUGUUGUUCUCGUCUCCUCGACACC 5′.

As another example, in some embodiments, the target RNA encodes PDE6A, carrying the V685M mutation associated with Retinitis pigmentosa. In some embodiments, the target RNA comprises the following sequence, with the mutated target adenosine underlined:

5′CCACAGACCUCGCCCUGUAUUUCAAGAAGAGGACGAUGUUCCAAAAGAUC AUGGAUCAGUCUAAGACAUAUGAGAGUGAACAGGAGUGGACACAGUACAUG 3′ (SEQ ID NO: 1197). In some embodiments, the corresponding complementary sequence (with C opposite from the target A) is as follows:

(SEQ ID NO: 1198)
3′GGUGUCUGGAGCGGGACAUAAAGUUCUUCUCCUGCUACAAGGUUUUC
UAGCACCUAGUCAGAUUCUGUAUACUCUCACUUGUCCUCACCUGUGUCA
UGUAC 5′.

The following exemplary ASOs, containing a C-C or C-U mismatch at position-9 (in square brackets and bold) relative to the editing site will enhance editing of the target adenosine:

(SEQ ID NO: 1199)
3′GGUGUCUGGAGCGGGACAUAAAGUUCUUCUCCUGCUACAAG[C]UUU
UCUAGCACCUAGUCAGAUUCUGUAUACUCUCACUUGUCCUCACCUGUGU
CAUGUA 5′.
(SEQ ID NO: 1200)
3′GGUGUCUGGAGCGGGACAUAAAGUUCUUCUCCUGCUACAAG[U]UUU
UCUAGCACCUAGUCAGAUUCUGUAUACUCUCACUUGUCCUCACCUGUGU
CAUGUA 5′.
(SEQ ID NO: 1201)
3′ AAG[C]UUUUCUAGCACCUAGUCAGAUUCUGUAUA 5′.
(SEQ ID NO: 1202)
3′ AAG[U]UUUUCUAGCACCUAGUCAGAUUCUGUAUA 5′.

Furthermore, the C-C or C-U mismatch at position-9 may be combined with other features, such as a G-U wobble base-pair at position +2 relative to the editing site (Rule 5, in square brackets and bold):

(SEQ ID NO: 1203)
3′GGUGUCUGGAGCGGGACAUAAAGUUCUUCUCCUGCUACAAG[C]UUU
UCUAGCA[U]CUAGUCAGAUUCUGUAUACUCUCACUUGUCCUCACCUGU
GUCAUGUA 5′.
(SEQ ID NO: 1204)
3′GGUGUCUGGAGCGGGACAUAAAGUUCUUCUCCUGCUACAAG[U]UUU
UCUAGCA[U]CUAGUCAGAUUCUGUAUACUCUCACUUGUCCUCACCUGU
GUCAUGUA 5′.
(SEQ ID NO: 1205)
3′ AAG[C]UUUUCUAGCA[U]CUAGUCAGAUUCUGUAUA 5′.
(SEQ ID NO: 1206)
3′ AAG[U]UUUUCUAGCA[U]CUAGUCAGAUUCUGUAUA 5′.

In some embodiments, the ASO includes a recruitment domain. An exemplary recruitment domain (SEQ ID NO: 1166) is shown in italics in the following sequences:

(SEQ ID NO: 1207)
3′AAG[C]UUUUCUAGCA[U]CUAGUGGUGUCGAGAAGAGGAGAACAAU
AUGCCAAAUGUUGUUCUCGUCUCCUCGACACC 5′.
(SEQ ID NO: 1208)
3′AAG[U]UUUUCUAGCA[U]CUAGUGGUGUCGAGAAGAGGAGAACAAU
AUGCCAAAUGUUGUUCUCGUCUCCUCGACACC 5′.

### Example 3

### Generalizable Rule 2

A second generalizable rule identified herein is that if the target sequence contains a C at position-8 relative to the editing site, flanked by a 5′ C or G and 3′ A or U, then a mismatched base in the ASO across-8C (i.e., C or U) increases editing. A schematic version of this rule is shown in FIG. 3A, item 2. The ASO facilitates ADAR-mediated A-to-G base replacement of a 5′-3′ target sequence comprising X1-X2-X3-X4-X5-X6-X7-X8-X9-A*, wherein X1 is C or G, X2 is C, X3 is A or U, and A* is the target base.

For example, in some embodiments the target RNA encodes ALDH2, carrying the E504K mutation associated with ALDH2 deficiency/alcohol flush. In some embodiments, the target RNA comprises a sequence as follows, with the mutated target adenosine underlined:

5′ UGUCGGGGAGUGGCCGGGAGUUGGGCGAGUACGGGCUGCAGGCAUACACU AAAGUGAAAACUGUCACAGUCAAAGUGCCUCAGAAGAACUCAUAAGAAUCA 3′ (SEQ ID NO: 1171). In some embodiments, the corresponding complementary sequence (with C opposite from the target A) is as follows:

(SEQ ID NO: 1209)
3′ ACAGCCCCUCACCGGCCCUCAACCCGCUCAUGCCCGACGUCCGUAU
GUGACUUCACUUUUGACAGUGUCAGUUUCACGGAGUCUUCUUGAGUAUU
CUUAGU 5′.

The following exemplary ASOs, containing a C-C mismatch at position-8 (in square brackets and bold) relative to the editing site will enhance editing of the target adenosine:

a)
(SEQ ID NO: 1210)
3′ACAGCCCCUCACCGGCCCUCAACCCGCUCAUGCCCGACGUCC[
C]UAUGUGACUUCACUUUUGACAGUGUCAGUUUCACGGAGUCUUC
UUGAGUAUUCUUAGU 5′
and
b)
(SEQ ID NO: 1211)
3′ GUCC[C]UAUGUGACUUCACUUUUGACAGUGUCAG 5′.

The C-C mismatch at position-8 can be used within ASO's of any length and symmetry. For example, in some embodiments ASOS have a length of 15-101 nt, for example in some implementations comprising between 4-50 nucleotides on either the 3′ or 5′ side of the target.

In some embodiments, the ASO further comprises a recruitment domain. An exemplary recruitment domain (SEQ ID NO: 1166) is shown in italics:

(SEQ ID NO: 1212)
3′GUCC[C]UAUGUGACUUCACUUGGUGUCGAGAAGAGGAGAACA
AUAUGCCAAAUGUUGUUCUCGUCUCCUCGACACC 5′.

As another example, in some embodiments, the target RNA encodes MeCP2, carrying the R106Q mutation associated with Rett syndrome. In some embodiments, the target RNA comprises a sequence as follows, with the mutated target adenosine underlined:

5′ UGACCGGGGACCCAUGUAUGAUGACCCCACCCUGCCUGAAGGCUGGACAC AGAAGCUUAAGCAAAGGAAAUCUGGCCGCUCUGCUGGGAAGUAUGAUGUGU 3′ (SEQ ID NO: 1177). In some embodiments, the corresponding complementary sequence (with C opposite from the target A) is:

(SEQ ID NO: 1213)
3′ ACUGGCCCCUGGGUACAUACUACUGGGGUGGGACGGACUUCC
GACCUGUGCCUUCGAAUUCGUUUCCUUUAGACCGGCGAGACGACC
CUUCAUACUACACA 5′.

The following exemplary ASOs, containing a C-C or C-U mismatch at position-8 (in square brackets and bold) relative to the editing site will enhance editing of the target adenosine:

(SEQ ID NO: 1214)
3′ACUGGCCCCUGGGUACAUACUACUGGGGUGGGACGGACUUCC[
C]ACCUGUGCCUUCGAAUUCGUUUCCUUUAGACCGGCGAGACGAC
CCUUCAUACUACACA 5′.
(SEQ ID NO: 1215)
3′ACUGGCCCCUGGGUACAUACUACUGGGGUGGGACGGACUUCC[
U]ACCUGUGCCUUCGAAUUCGUUUCCUUUAGACCGGCGAGACGAC
CCUUCAUACUACACA 5′.
(SEQ ID NO: 1216)
3′ UUCC[C]ACCUGUGCCUUCGAAUUCGUUUCCUUUA 5′.
(SEQ ID NO: 1217)
3′ UUCC[U]ACCUGUGCCUUCGAAUUCGUUUCCUUUA 5′

The C-C or C-U mismatch at position-8 can be used within ASO's of any length and symmetry. In some embodiments ASOs have a length of 15-101 nt, for example, in some implementations comprising between 4-50 nucleotides on either the 3′ or 5′ side of the target.

In some embodiments, the ASO further comprises a recruitment domain. An exemplary recruitment domain (SEQ ID NO: 1166) is shown in italics:

(SEQ ID NO: 1218)
3′UUCC[C]ACCUGUGCCUUCGAAGGUGUCGAGAAGAGGAGAACA
AUAUGCCAAAUGUUGUUCUCGUCUCCUCGACACC 5′.
(SEQ ID NO: 1219)
3′UUCC[U]ACCUGUGCCUUCGAAGGUGUCGAGAAGAGGAGAACA
AUAUGCCAAAUGUUGUUCUCGUCUCCUCGACACC 5′.

As another example, in some embodiments the target RNA encodes IFNAR1. In some embodiments, introducing a de-novo mutation in the wild-type sequence of IFNAR1 is conducted using an ASO. In some embodiments, the target RNA features a phosphorylation site at Y466 and an ASO is employed to eliminate the phosphorylation site. In some embodiments the target RNA comprises the following sequence, with the target adenosine underlined:

5′UCUCCCGUUUGUCAUUUAUGCUGCGAAAGUCUUCUUGAGAUGCAUCAAUU AUGUCUUCUUUCCAUCACUUAAACCUUCUUCCAGUAUAGAUGAGUAUUUCU 3′ (SEQ ID NO: 1182). In some embodiments, the corresponding complementary sequence (with C opposite from the target A) is as follows:

(SEQ ID NO: 1220)
3′AGAGGGCAAACAGUAAAUACGACGCUUUCAGAAGAACUCUACG
UAGUUAACACAGAAGAAAGGUAGUGAAUUUGGAAGAAGGUCAUAU
CUACUCAUAAAGA 5′.

The following exemplary ASOs, containing a C-C or C-U mismatch at position-8 (in square brackets and bold) relative to the editing site will enhance editing of the target adenosine:

(SEQ ID NO: 1221)
3′AGAGGGCAAACAGUAAAUACGACGCUUUCAGAAGAACUCUAC[
C]UAGUUAACACAGAAGAAAGGUAGUGAAUUUGGAAGAAGGUCAU
AUCUACUCAUAAAGA 5′.
(SEQ ID NO: 1222)
3′AGAGGGCAAACAGUAAAUACGACGCUUUCAGAAGAACUCUAC[
U]UAGUUAACACAGAAGAAAGGUAGUGAAUUUGGAAGAAGGUCAU
AUCUACUCAUAAAGA 5′.
(SEQ ID NO: 1223)
3′ CUAC[C]UAGUUAACACAGAAGAAAGGUAGUGAAU 5′.
(SEQ ID NO: 1224)
3′ CUAC[U]UAGUUAACACAGAAGAAAGGUAGUGAAU 5′.

The C-C or C-U mismatch at position-8 can be used within ASO's of any length and symmetry. In some embodiments ASOs have a length of 15-101 nt, for example in some implementations comprising between 4-50 nucleotides on either the 3′ or 5′ side of the target.

The mismatch at position-8 may be combined with other features, such as a G-U wobble base-pair at position +2 relative to the editing site (Rule 5, in square brackets and bold). Exemplary ASOs containing a mismatch at position-8 in combination with a G-U wobble base pair at position +2 are:

(SEQ ID NO: 1225)
3′AGAGGGCAAACAGUAAAUACGACGCUUUCAGAAGAACUCUAC[
C]UAGUUAACA[U]AGAAGAAAGGUAGUGAAUUUGGAAGAAGGUC
AUAUCUACUCAUAAAGA 5′,
(SEQ ID NO: 1226)
3′AGAGGGCAAACAGUAAAUACGACGCUUUCAGAAGAACUCUAC[
U]UAGUUAACA[U]AGAAGAAAGGUAGUGAAUUUGGAAGAAGGUC
AUAUCUACUCAUAAAGA 5′,
(SEQ ID NO: 1227)
3′ CUAC[C]UAGUUAACA[U]AGAAGAAAGGUAGUGAAU 5′,
(SEQ ID NO: 1228)
3′ CUAC[U]UAGUUAACA[U]AGAAGAAAGGUAGUGAAU 5′.

In some embodiments, ASOS further include a recruitment domain. An exemplary recruitment domain (SEQ ID NO: 1166) is shown in italic:

(SEQ ID NO: 1229)
3′CUAC[C]UAGUUAACACAGAAGGGUGUCGAGAAGAGGAGAACA
AUAUGCCAAAUGUUGUUCUCGUCUCCUCGACACC 5′.
(SEQ ID NO: 1230)
3′CUAC[U]UAGUUAACACAGAAGGGUGUCGAGAAGAGGAGAACA
AUAUGCCAAAUGUUGUUCUCGUCUCCUCGACACC 5′.

### Example 4

### Generalizable Rule 3

A third generalizable rule identified herein is that if the target sequence contains a C residue at position-7 relative to the editing site, flanked by a 5′ G or C and a 3′ A or U, then a mismatched base in the ASO across-7C (i.e., C or U) increases editing. A schematic version of this rule is shown in FIG. 3A, item 3. The ASO facilitates ADAR-mediated A-to-G base replacement of a 5′-3′ target sequence comprising X1-X2-X3-X4-X5-X6-X7-X8-A*, wherein X1 is C or G, X2 is C, X3 is A or U, and A* is the target base.

For example, in some embodiments the target RNA encodes SERPINA1, carrying the E342K mutation associated with AAT deficiency. In some embodiments, the target RNA comprises a sequence as follows, with the mutated target adenosine underlined:

5′ CACCCCUGAAGCUCUCCAAGGCCGUGCAUAAGGCUGUGCUGACCAUCGAC AAGAAAGGGACUGAAGCUGCUGGGGCCAUGUUUUUAGAGGCCAUACCCAUG 3′ (SEQ ID NO: 1169). In some embodiments, the corresponding complementary sequence (with C opposite from the target A) is:

(SEQ ID NO: 1031)
3′ GUGGGGACUUCGAGAGGUUCCGGCACGUAUUCCGACACGACU
GGUAGCUGCUCUUUCCCUGACUUCGACGACCCCGGUACAAAAAUC
UCCGGUAUGGGUAC 5′.

The following exemplary ASOs, containing a C-C or C-U mismatch at position-7 (in square brackets and bold) relative to the editing site will enhance editing of the target adenosine:

(SEQ ID NO: 1232)
3′GUGGGGACUUCGAGAGGUUCCGGCACGUAUUCCGACACGACUG
[C]UAGCUGCUCUUUCCCUGACUUCGACGACCCCGGUACAAAAAU
CUCCGGUAUGGGUAC 5′,
(SEQ ID NO: 1233)
3′GUGGGGACUUCGAGAGGUUCCGGCACGUAUUCCGACACGACUG
[U]UAGCUGCUCUUUCCCUGACUUCGACGACCCCGGUACAAAAAU
CUCCGGUAUGGGUAC 5′,
(SEQ ID NO: 1234)
3′ GACUG[C]UAGCUGCUCUUUCCCUGACUUCGACGA 5′,
(SEQ ID NO: 1235)
3′ GACUG[U]UAGCUGCUCUUUCCCUGACUUCGACGA 5′.

The C-C or C-U mismatch at position-7 can be used within ASOs of any length and symmetry. In some embodiments, ASOs have a length of 15-101 nt, for example, in some implementations comprising between 4-50 nucleotides on either the 3′ or 5′ side of the target.

The C-C or C-U mismatch at position-7 may be combined with other features, such as a G-U wobble base-pair at position +2 relative to the editing site (Rule 5, in square brackets and bold). Exemplary ASOs containing a mismatch at position-7 in combination with a G-U wobble base pair at position +2 are:

(SEQ ID NO: 1236)
3′GUGGGGACUUCGAGAGGUUCCGGCACGUAUUCCGACACGACUG
[C]UAGCUGCU[U]UUUCCCUGACUUCGACGACCCCGGUACAAAA
AUCUCCGGUAUGGGUAC 5′,
(SEQ ID NO: 1237)
3′GUGGGGACUUCGAGAGGUUCCGGCACGUAUUCCGACACGACUG
[U]UAGCUGCU[U]UUUCCCUGACUUCGACGACCCCGGUACAAAA
AUCUCCGGUAUGGGUAC 5′,
(SEQ ID NO: 1238)
3′ GACUG[C]UAGCUGCU[U]UUUCCCUGACUUCGACGA 5′,
(SEQ ID NO: 1239)
3′ GACUG[U]UAGCUGCU[U]UUUCCCUGACUUCGACGA 5′.

In some embodiments, the ASO further includes a recruitment domain. An exemplary recruitment domain (SEQ ID NO: 1166) is shown in italic:

(SEQ ID NO: 1240)
3′GACUG[C]UAGCUGCUCUUUCCGGUGUCGAGAAGA
GGAGAACAAUAUGCCAAAUGUUGUUCUCGUCUCCUCG
ACACC 5′.
(SEQ ID NO: 1241)
3′GACUG[U]UAGCUGCUCUUUCCGGUGUCGAGAAGAGG
AGAACAAUAUGCCAAAUGUUGUUCUCGUCUCCUCGACAC
C 5′.

### Example 5

### Generalizable Rule 4

A fourth generalizable rule identified herein is that if the target sequence contains a C residue at position-6 relative to the editing site, flanked by a 5′ G or C and a 3′ A or U, then a mismatched base in the ASO across-6C (i.e., C or U) increases editing. A schematic version of this rule is shown in FIG. 3A, item 4. The ASO facilitates ADAR-mediated A-to-G base replacement of a 5′-3′ target sequence comprising X1-X2-X3-X4-X5-X6-X7-A*, wherein X1 is C or G, X2 is C, X3 is A or U, and A* is the target base.

For example, in some embodiments the target RNA encodes SCN9A, carrying the W897X mutation associated with congenital insensitivity to pain. In some embodiments, the target RNA comprises the following sequence, with the mutated target adenosine underlined:

5′CUACAAAGAAUGUGUCUGCAAGAUCAAUGAUGACUGUACGCUCCCACGGU A GCACAUGAACGACUUCUUCCACUCCUUCCUGAUUGUGUUCCGCGUGCUGU 3′ (SEQ ID NO: 1173). In some embodiments, the corresponding complementary sequence (with C opposite from the target A) is:

(SEQ ID NO: 1242)
3′GAUGUUUCUUACACAGACGUUCUAGUUACUACUGACAUGCGAG
GGUGCCACCGUGUACUUGCUGAAGAAGGUGAGGAAGGACUAACAC
AAGGCGCACGACA 5′,

The following exemplary ASOs, containing a C-C or C-U mismatch at position-6 (in square brackets and bold) relative to the editing site, will enhance editing of the target adenosine:

(SEQ ID NO: 1243)
3′GAUGUUUCUUACACAGACGUUCUAGUUACUACUGACAUGCGAG
G[C]UGCCACCGUGUACUUGCUGAAGAAGGUGAGGAAGGACUAAC
ACAAGGCGCACGACA 5′,
(SEQ ID NO: 1244)
3′GAUGUUUCUUACACAGACGUUCUAGUUACUACUGACAUGCGAG
G[U]UGCCACCGUGUACUUGCUGAAGAAGGUGAGGAAGGACUAAC
ACAAGGCGCACGACA 5′,
(SEQ ID NO: 1245)
3′ GCGAGG[C]UGCCACCGUGUACUUGCUGAAGAAGG 5′,
(SEQ ID NO: 1246)
3′ GCGAGG[U]UGCCACCGUGUACUUGCUGAAGAAGG 5′.

The C-C or C-U mismatch at position-6 can be used within ASOs of any length and symmetry. For example, in some embodiments ASOs have a length of 15-101 nt, for example, in some implementations comprising between 4-50 nucleotides on either the 3′ or 5′ side of the target.

In some embodiments, the ASO further comprises a recruitment domain. An exemplary recruitment domain (SEQ ID NO: 1166) is shown in italic:

(SEQ ID NO: 1247)
3′GCGAGG[C]UGCCACCGUGUACGGUGUCGAGAAGAGGAGAACA
AUAUGCCAAAUGUUGUUCUCGUCUCCUCGACACC 5′.
(SEQ ID NO: 1248)
3′GCGAGG[U]UGCCACCGUGUACGGUGUCGAGAAGAGGAGAACA
AUAUGCCAAAUGUUGUUCUCGUCUCCUCGACACC 5′.

### Example 6

### Generalizable Rule 5

A fifth generalizable rule identified herein is that if the target sequence contains a G residue at position +2 relative to the editing site, then a U in the ASO across +2G (presumably forming a G·U wobble base-pair) increases editing. A schematic version of this rule is shown in FIG. 3B, item 5. The ASO facilitates ADAR-mediated A-to-G base replacement of a 5′-3′ target sequence comprising A *-X1-X2-X3, wherein X2 is G, X1 and X3 are A, C, G or U, and A* is the target base.

For example, in some embodiments, the target RNA encodes SERPINA1, carrying the E342K mutation associated with AAT deficiency. In some embodiments, the target RNA comprises the following sequence, with the mutated target adenosine underlined:

5′ CACCCCUGAAGCUCUCCAAGGCCGUGCAUAAGGCUGUGCUGACCAUCGAC AAGAAAGGGACUGAAGCUGCUGGGGCCAUGUUUUUAGAGGCCAUACCCAUG 3′ (SEQ ID NO: 1169). In some embodiments, the corresponding complementary sequence (with C opposite from the target A) is:

(SEQ ID NO: 1231)
3′ GUGGGGACUUCGAGAGGUUCCGGCACGUAUUCCGACACGACU
GGUAGCUGCUCUUUCCCUGACUUCGACGACCCCGGUACAAAAAUC
UCCGGUAUGGGUAC 5′.

The following exemplary ASOs, containing a G-U wobble base-pair at position +2 will enhance editing of the target adenosine:

3′

(SEQ ID NO: 1249)
GUGGGGACUUCGAGAGGUUCCGGCACGUAUUCCGACACGACUGGU
AGCUGCU[U]UUUCCCUGACUUCGACGACCCCGGUACAAAAAUCU
CCGGUAUGGGUAC 5′.
(SEQ ID NO: 1250)
3′ GACUGGUAGCUGCU[U]UUUCCCUGACUUCGACGA 5′

As another example, in some embodiments, the target RNA encodes PDE6A, carrying the V685M mutation associated with Retinitis pigmentosa. In some embodiments, the target RNA comprises the following sequence, with the mutated target adenosine underlined:

5′CCACAGACCUCGCCCUGUAUUUCAAGAAGAGGACGAUGUUCCAAAAGAUC AUGGAUCAGUCUAAGACAUAUGAGAGUGAACAGGAGUGGACACAGUACAUG 3′ (SEQ ID NO: 1197). In some embodiments, the corresponding complementary sequence (with C opposite from the target A) is:

(SEQ ID NO: 1198)
3′GGUGUCUGGAGCGGGACAUAAAGUUCUUCUCCUGCUACAAGGU
UUUCUAGCACCUAGUCAGAUUCUGUAUACUCUCACUUGUCCUCAC
CUGUGUCAUGUAC 5′.

The following exemplary ASOs, containing a G-U wobble base-pair at position +2 will enhance editing of the target adenosine:

(SEQ ID NO: 1251)
3′GGUGUCUGGAGCGGGACAUAAAGUUCUUCUCCUGCUACAAGGU
UUUCUAGCA[U]CUAGUCAGAUUCUGUAUACUCUCACUUGUCCUC
ACCUGUGUCAUGUAC 5′.
(SEQ ID NO: 1252)
3′ AAGGUUUUCUAGCA[U]CUAGUCAGAUUCUGUAUA 5′

As another example, in some embodiments, the target RNA encodes KRAS, carrying the G12D mutation associated with cancer. In some embodiments, the target RNA comprises the following sequence, with the mutated target adenosine underlined:

5′GAGAGGCCUGCUGAAAAUGACUGAAUAUAAACUUGUGGUAGUUGGAGCU GAUGGCGUAGGCAAGAGUGCCUUGACGAUACAGCUAAUUCAGAAUCAUUUUG 3′ (SEQ ID NO: 1175). In some embodiments, the corresponding complementary sequence (with C opposite from the target A) is:

(SEQ ID NO: 1253)
3′CUCUCCGGACGACUUUUACUGACUUAUAUUUGAACACCAUCAA
CCUCGACCACCGCAUCCGUUCUCACGGAACUGCUAUGUCGAUUAA
GUCUUAGUAAAAC 5′.

The following exemplary ASOs containing a G-U wobble base-pair at position +2 will enhance editing of the target adenosine:

(SEQ ID NO: 1254)
3′CUCUCCGGACGACUUUUACUGACUUAUAUUUGAACACCAUCAA
CCUCGACCA[U]CGCAUCCGUUCUCACGGAACUGCUAUGUCGAUU
AAGUCUUAGUAAAAC 5′.
(SEQ ID NO: 1255)
3′ AUCAACCUCGACCA[U]CGCAUCCGUUCUCACGGA 5′

As another example, in some embodiments the target RNA encodes F5, carrying the R534Q mutation associated with thrombophilia. In some embodiments, the target RNA comprises the following sequence, with the mutated target adenosine underlined:

5′UGGGCUAAUAGGACUACUUCUAAUCUGUAAGAGCAGAUCCCUGGACAGGC AAGGAAUACAGAGGGCAGCAGACAUCGAACAGCAGGCUGUGUUUGCUGUGU 3′ (SEQ ID NO: 1178). In some embodiments, the corresponding complementary sequence (with C opposite from the target A) is:

(SEQ ID NO: 1256)
3′ ACCCGAUUAUCCUGAUGAAGAUUAGACAUUCUCGUCUAGGGA
CCUGUCCGCUCCUUAUGUCUCCCGUCGUCUGUAGCUUGUCGUCCG
ACACAAACGACACA 5′.

The following exemplary ASOs containing a G-U wobble base-pair at position +2 will enhance editing of the target adenosine:

(SEQ ID NO: 1257)
3′ ACCCGAUUAUCCUGAUGAAGAUUAGACAUUCUCGUCUAGGGA
CCUGUCCGCU[U]CUUAUGUCUCCCGUCGUCUGUAGCUUGUCGUC
CGACACAAACGACACA 5′,
(SEQ ID NO: 1258)
3′ GGGACCUGUCCGCU[U]CUUAUGUCUCCCGUCGUC 5′.

As another example, in some embodiments the target RNA encodes ABCA4, carrying the G1961E mutation associated with Stargardt disease. In some embodiments, the target RNA comprises the following sequence, with the mutated target adenosine underlined:

5′ CAAGAUUUAUCCAGGCACCUCCAGCCCAGCAGUGGACAGGCUGUGUGUCG AAGUUCGCCCUGGAGAGUGCUUUGGCCUCCUGGGAGUGAAUGGUGCCGGCA 3′ (SEQ ID NO: 1179). In some embodiments, the corresponding complementary sequence (with C opposite from the target A) is:

(SEQ ID NO: 1259)
3′GUUCUAAAUAGGUCCGUGGAGGUCGGGUCGUCACCUGUCCGAC
ACACAGCCUCAAGCGGGACCUCUCACGAAACCGGAGGACCCUCAC
UUACCACGGCCGU 5′.

The following exemplary ASOs, containing a G-U wobble base-pair at position +2 will enhance editing of the target adenosine:

(SEQ ID NO: 1260)
3′ GUUCUAAAUAGGUCCGUGGAGGUCGGGUCGUCACCUGUCCGA
CACACAGCCU[U]AAGCGGGACCUCUCACGAAACCGGAGGACCCU
CACUUACCACGGCCGU 5′,
(SEQ ID NO: 1261)
3′ CCGACACACAGCCU[U]AAGCGGGACCUCUCACGA 5′

As another example, in some embodiments the target RNA encodes IFNAR1 featuring a phosphorylation site at Y466. In some embodiments the ASO is designed to eliminate the phosphorylation site at Y466. In some embodiments, the target RNA comprises the following sequence, with the target adenosine underlined:

5′UCUCCCGUUUGUCAUUUAUGCUGCGAAAGUCUUCUUGAGAUGCAUCAAUU AUGUCUUCUUUCCAUCACUUAAACCUUCUUCCAGUAUAGAUGAGUAUUUCU 3′ (SEQ ID NO: 1182). In some embodiments, the corresponding complementary sequence (with C opposite from the target A) is:

(SEQ ID NO: 1220)
3′AGAGGGCAAACAGUAAAUACGACGCUUUCAGAAGAACUCUACG
UAGUUAACACAGAAGAAAGGUAGUGAAUUUGGAAGAAGGUCAUAU
CUACUCAUAAAGA 5′.

The following exemplary ASOs, containing a G-U wobble base-pair at position +2 will enhance editing of the target adenosine:

(SEQ ID NO: 1262)
3′AGAGGGCAAACAGUAAAUACGACGCUUUCAGAAGAACUCUACG
UAGUUAACA[U]AGAAGAAAGGUAGUGAAUUUGGAAGAAGGUCAU
AUCUACUCAUAAAGA 5′.
(SEQ ID NO: 1263)
3′ CUACGUAGUUAACA[U]AGAAGAAAGGUAGUGAAU 5′

As another example, in some embodiments the target RNA encodes TMEM173. In some embodiments an oligonucleotide is employed to introduce the gain-of-function mutation R284G in the target sequence. In some embodiments, the target RNA comprises the following sequence, with the target adenosine underlined:

5′ CUUUGUUUGCCAUGUCACAAUACAGUCAAGCUGGCUUUAGCCGGGAGGAU AGGCUUGAGCAGGCCAAACUCUUCUGCCGGACACUUGAGGACAUCCUGGCA 3′ (SEQ ID NO: 1185). In some embodiments, the corresponding complementary sequence (with C opposite from the target A) is:

(SEQ ID NO: 1264)
3′GAAACAAACGGUACAGUGUUAUGUCAGUUCGACCGAAAUCGGC
CCUCCUACCCGAACUCGUCCGGUUUGAGAAGACGGCCUGUGAACU
CCUGUAGGACCGU 5′.

The following exemplary ASOs, containing a G-U wobble base-pair at position +2 will enhance editing of the target adenosine:

(SEQ ID NO: 1265)
3′GAAACAAACGGUACAGUGUUAUGUCAGUUCGACCGAAAUCGGC
CCUCCUACC[U]GAACUCGUCCGGUUUGAGAAGACGGCCUGUGAA
CUCCUGUAGGACCGU 5′.
(SEQ ID NO: 1266)
3′ UCGGCCCUCCUACC[U]GAACUCGUCCGGUUUGAG 5′.

The G-U wobble base-pair at position +2 can be used within ASOs of any length and symmetry. In some embodiments, ASOs have a length of 15-101 nt, for example, in some implementations comprising between 4-50 nucleotides on either the 3′ or 5′ side of the target.

The G-U wobble base-pair at position +2 may be combined with other features, such as a G-G mismatch at position −1 relative to the editing site. Such an ASO is exemplified by ABCA4 G1961E (mismatches in square brackets and bold):

(SEQ ID NO: 1267)
3′GUUCUAAAUAGGUCCGUGGAGGUCGGGUCGUCACCUGUCCGAC
ACACAG[G]CU[U]AAGCGGGACCUCUCACGAAACCGGAGGACCC
UCACUUACCACGGCCGU 5′.
(SEQ ID NO: 1268)
3′ CCGACACACAG[G]CU[U]AAGCGGGACCUCUCACGA 5′.

As another example the G-U wobble base-pair at position +2 can be combined with A C-C or C-U mismatch at position-9 relative to the editing site. Such an ASO is exemplified by PDE6A V685M (Rule 1; mismatches in square brackets and bold):

(SEQ ID NO: 1203)
3′GGUGUCUGGAGCGGGACAUAAAGUUCUUCUCCUGCUACAAG[C
]UUUUCUAGCA[U]CUAGUCAGAUUCUGUAUACUCUCACUUGUCC
UCACCUGUGUCAUGUA 5′.
(SEQ ID NO: 1204)
3′GGUGUCUGGAGCGGGACAUAAAGUUCUUCUCCUGCUACAAG[U
]UUUUCUAGCA[U]CUAGUCAGAUUCUGUAUACUCUCACUUGUCC
UCACCUGUGUCAUGUA 5′.
(SEQ ID NO: 1205)
3′ AAG[C]UUUUCUAGCA[U]CUAGUCAGAUUCUGUAUA 5′.
(SEQ ID NO: 1206)
3′ AAG[U]UUUUCUAGCA[U]CUAGUCAGAUUCUGUAUA 5′.

As another example, the G-U wobble base-pair at position +2 can be combined with a C-C or C-U mismatch at position-8 relative to the editing site. Such an ASO is exemplified by IFNAR1 Y466 (Rule 2; mismatches in square brackets and bold):

(SEQ ID NO: 1225)
3′AGAGGGCAAACAGUAAAUACGACGCUUUCAGAAGAACUCUAC[
C]UAGUUAACA[U]AGAAGAAAGGUAGUGAAUUUGGAAGAAGGUC
AUAUCUACUCAUAAAGA 5′,
(SEQ ID NO: 1226)
3′AGAGGGCAAACAGUAAAUACGACGCUUUCAGAAGAACUCUAC[
U]UAGUUAACA[U]AGAAGAAAGGUAGUGAAUUUGGAAGAAGGUC
AUAUCUACUCAUAAAGA 5′,
(SEQ ID NO: 1227)
3′ CUAC[C]UAGUUAACA[U]AGAAGAAAGGUAGUGAAU 5′,
(SEQ ID NO: 1228)
3′ CUAC[U]UAGUUAACA[U]AGAAGAAAGGUAGUGAAU 5′.

As another example, the G-U wobble base-pair at position +2 can be combined with a C-C or C-U mismatch at position-7 relative to the editing site. Such an ASO is exemplified by SERPINA1 E342K (Rule 3; mismatches in square brackets and bold)

(SEQ ID NO: 1236)
3′GUGGGGACUUCGAGAGGUUCCGGCACGUAUUCCGACACGACUG
[C]UAGCUGCU[U]UUUCCCUGACUUCGACGACCCCGGUACAAAA
AUCUCCGGUAUGGGUAC 5′,
(SEQ ID NO: 1237)
3′GUGGGGACUUCGAGAGGUUCCGGCACGUAUUCCGACACGACUG
[U]UAGCUGCU[U]UUUCCCUGACUUCGACGACCCCGGUACAAAA
AUCUCCGGUAUGGGUAC 5′,
(SEQ ID NO: 1238)
3′ GACUG[C]UAGCUGCU[U]UUUCCCUGACUUCGACGA 5′,
(SEQ ID NO: 1239)
3′ GACUG[U]UAGCUGCU[U]UUUCCCUGACUUCGACGA 5′.

In some embodiments, the ASO includes a recruitment domain. An exemplary recruitment domain (SEQ ID NO: 1166) is shown in italic:

(SEQ ID NO: 1269)
3′GACUGGUAGCUGC[U]CUUUCCGGUGUCGAGAAGAGGAGAACA
AUAUGCCAAAUGUUGUUCUCGUCUCCUCGACACC 5′.

### Example 7

### Generalizable Rule 6

A sixth generalizable rule identified herein is that if the target sequence contains a U at position +2 relative to the editing site, then a mismatched base in the ASO across from +2 (i.e., C or U) increases editing. A schematic version of this rule is shown in FIG. 3B, item 6. The ASO facilitates ADAR-mediated A-to-G base replacement of a 5′-3′ target sequence comprising A *-X1-X2-X3, wherein X2 is U, X1 and X3 are A, C, G or U, and A* is the target base.

For example, in some embodiments the target RNA encodes GFER, carrying the R194H mutation associated with myopathy. In some embodiments, the target RNA comprises the following sequence, with the mutated target adenosine underlined:

5′ GAACCGCAAGCUGGGCAAGCCUGACUUCGACUGCUCAAAAGUGGAUGAGC ACUGGCGCGACGGCUGGAAGGAUGGCUCCUGUGACUAGAGGGUGGUCAGCC 3′ (SEQ ID NO: 1170). In some embodiments, the corresponding complementary sequence (with C opposite from the target A) is:

(SEQ ID NO: 1270)
3′CUUGGCGUUCGACCCGUUCGGACUGAAGCUGACGAGUUUUCAC
CUACUCGCGACCGCGCUGCCGACCUUCCUACCGAGGACACUGAUC
UCCCACCAGUCGG 5′.

The following exemplary ASOs, containing a U-C or U-U mismatch at position +2 will enhance editing of the target adenosine:

(SEQ ID NO: 1271)
3′ CUUGGCGUUCGACCCGUUCGGACUGAAGCUGACGAGUUUUCA
CCUACUCGCG[C]CCGCGCUGCCGACCUUCCUACCGAGGACACUG
AUCUCCCACCAGUCGG 5′.
(SEQ ID NO: 1272)
3′ CUUGGCGUUCGACCCGUUCGGACUGAAGCUGACGAGUUUUCA
CCUACUCGCG[U]CCGCGCUGCCGACCUUCCUACCGAGGACACUG
AUCUCCCACCAGUCGG 5′.
(SEQ ID NO: 1273)
3′ UUCACCUACUCGCG[C]CCGCGCUGCCGACCUUCC 5′,
(SEQ ID NO: 1274)
3′ UUCACCUACUCGCG[U]CCGCGCUGCCGACCUUCC 5′.

As another example, in some embodiments, the RNA encodes KRAS, carrying the G12S mutation associated with cancer. In some embodiments the target RNA comprises the following sequence, with the mutated target adenosine underlined:

(SEQ ID NO: 1176)
5′ AGAGAGGCCUGCUGAAAAUGACUGAAUAUAAACUUGUGGUAGU
UGGAGCUAGUGGCGUAGGCAAGAGUGCCUUGACGAUACAGCUAAU
UCAGAAUCAUUUU 3′.
In some embodiments, the corresponding
complementary sequence (with C opposite
from the target A) is:
(SEQ ID NO: 1275)
3′ UCUCUCCGGACGACUUUUACUGACUUAUAUUUGAACACCAUCA
ACCUCGACCACCGCAUCCGUUCUCACGGAACUGCUAUGUCGAUUA
AGUCUUAGUAAAA 5′.

The following exemplary ASOs, containing a U-C or U-U mismatch at position +2 will enhance editing of the target adenosine:

(SEQ ID NO: 1276)
3′ UCUCUCCGGACGACUUUUACUGACUUAUAUUUGAACACCAUCA
ACCUCGACC[C]CCGCAUCCGUUCUCACGGAACUGCUAUGUCGAU
UAAGUCUUAGUAAAA 5′,
(SEQ ID NO: 1277)
3′ UCUCUCCGGACGACUUUUACUGACUUAUAUUUGAACACCAUCA
ACCUCGACC[U]CCGCAUCCGUUCUCACGGAACUGCUAUGUCGAU
UAAGUCUUAGUAAAA 5′,
(SEQ ID NO: 1278)
3′ CAUCAACCUCGACC[C]CCGCAUCCGUUCUCACGG 5′,
(SEQ ID NO: 1279)
3′ CAUCAACCUCGACC[U]CCGCAUCCGUUCUCACGG 5′.

As another example, in some embodiments the target RNA encodes TP53, carrying the R175H mutation associated with cancer. In some embodiments, the target RNA comprises the following sequence, with the mutated target adenosine underlined:

5′ CGCCAUGGCCAUCUACAAGCAGUCACAGCACAUGACGGAGGUUGUGAGGC ACUGCCCCCACCAUGAGCGCUGCUCAGAUAGCGAUGGUCUGGCCCCUCCUC 3′ (SEQ ID NO: 1172). In some embodiments, the corresponding complementary sequence (with C opposite from the target A) is:

(SEQ ID NO: 1280)
3′GCGGUACCGGUAGAUGUUCGUCAGUGUCGUGUACUGCCUCCAA
CACUCCGCGACGGGGGUGGUACUCGCGACGAGUCUAUCGCUACCA
GACCGGGGAGGAG 5′.

The following exemplary ASOs, containing a U-C or U-U mismatch at position +2 will enhance editing of the target adenosine:

(SEQ ID NO: 1281)
3′ GCGGUACCGGUAGAUGUUCGUCAGUGUCGUGUACUGCCUCCA
ACACUCCGCG[C]CGGGGGUGGUACUCGCGACGAGUCUAUCGCUA
CCAGACCGGGGAGGAG 5′,
(SEQ ID NO: 1282)
3′ GCGGUACCGGUAGAUGUUCGUCAGUGUCGUGUACUGCCUCCA
ACACUCCGCG[U]CGGGGGUGGUACUCGCGACGAGUCUAUCGCUA
CCAGACCGGGGAGGAG 5′,
(SEQ ID NO: 1283)
3′ UCCAACACUCCGCG[C]CGGGGGUGGUACUCGCGA 5′,
(SEQ ID NO: 1284)
3′ UCCAACACUCCGCG[U]CGGGGGUGGUACUCGCGA 5′.

As another example, in some embodiments, the target RNA encodes DDX58. In some embodiments, an ASO is employed to introduce the gain-of-function mutation E373G into the target sequence. In some embodiments, the target RNA comprises a sequence as follows, with the target adenosine underlined:

5′

AAAGGGAACGAUUCCAUCACUAUCCAUCUUUACUUUGAUGAUAUUUGAUGAAUGCCACA ACACUAGUAAACAACACCCGUACAAUAUGAUCAUGUUUAAUU 3′ (SEQ ID NO: 1285). In some embodiments, the corresponding complementary sequence (with C opposite from the target A) is:

(SEQ ID NO: 1286)
3′UUUCCCUUGCUAAGGUAGUGAUAGGUAGAAAUGAAACUACUAU
AAACUACCUACGGUGUUGUGAUCAUUUGUUGUGGGCAUGUUAUAC
UAGUACAAAUUAA 5′.

The following exemplary ASOs, containing a U-C or U-U mismatch at position +2 will enhance editing of the target adenosine:

(SEQ ID NO: 1287)
3′UUUCCCUUGCUAAGGUAGUGAUAGGUAGAAAUGAAACUACUAU
AAACUACCU[C]CGGUGUUGUGAUCAUUUGUUGUGGGCAUGUUAU
ACUAGUACAAAUUAA 5′,
(SEQ ID NO: 1288)
3′UUUCCCUUGCUAAGGUAGUGAUAGGUAGAAAUGAAACUACUAU
AAACUACCU[U]CGGUGUUGUGAUCAUUUGUUGUGGGCAUGUUAU
ACUAGUACAAAUUAA 5′,
(SEQ ID NO: 1289)
3′ ACUAUAAACUACCU[C]CGGUGUUGUGAUCAUUUG 5′,
(SEQ ID NO: 1290)
3′ ACUAUAAACUACCU[U]CGGUGUUGUGAUCAUUUG 5′.

As another example, in some embodiments, the target RNA encodes IFIH1. In some embodiments, an ASO is employed to introduce the gain-of-function mutation E444G. In some embodiments, the target RNA comprises the following sequence, with the target adenosine underlined:

5′AGAAGAUGCUGGUGUUCAAUUGUCAGACUUUUCCCUCAUUAUCAUUGAUG AAUGUCAUCACACCAACAAAGAAGCAGUGUAUAAUAACAUCAUGAGGCAUU 3′ (SEQ ID NO: 1291). In some embodiments, the corresponding complementary sequence (with C opposite from the target A) is:

(SEQ ID NO: 1292)
3′UCUUCUACGACCACAAGUUAACAGUCUGAAAAGGGAGUAAUAG
UAACUACCUACAGUAGUGUGGUUGUUUCUUCGUCACAUAUUAUUG
UAGUACUCCGUAA 5′.

The following exemplary ASOs, containing a U-C or U-U mismatch at position +2 will enhance editing of the target adenosine:

(SEQ ID NO: 1293)
3′UCUUCUACGACCACAAGUUAACAGUCUGAAAAGGGAGUAAUAG
UAACUACCU[C]CAGUAGUGUGGUUGUUUCUUCGUCACAUAUUAU
UGUAGUACUCCGUAA 5′,
(SEQ ID NO: 1294)
3′UCUUCUACGACCACAAGUUAACAGUCUGAAAAGGGAGUAAUAG
UAACUACCU[U]CAGUAGUGUGGUUGUUUCUUCGUCACAUAUUAU
UGUAGUACUCCGUAA 5′,
(SEQ ID NO: 1295)
3′ AAUAGUAACUACCU[C]CAGUAGUGUGGUUGUUUC 5′,
(SEQ ID NO: 1296)
3′ AAUAGUAACUACCU[U]CAGUAGUGUGGUUGUUUC 5′.

In some embodiments the target RNA encodes IFIH1 and an ASO is used to introduce the gain-of-function mutation 1583V. In some embodiments, the target RNA comprises the following sequence, with the target adenosine underlined:

5′AAAUGAGUCCAAUGUCAGAUUUUGGAACUCAACCCUAUGAACAAUGGGCC AUUCAAAUGGAAAAAAAAGCUGCAAAAGAAGGAAAUCGCAAAGAACGUGUU 3′ (SEQ ID NO: 1297). In some embodiments, the corresponding complementary sequence (with C opposite from the target A) is:

(SEQ ID NO: 1298)
3′UUUACUCAGGUUACAGUCUAAAACCUUGAGUUGGGAUACUUGU
UACCCGGCAAGUUUACCUUUUUUUUCGACGUUUUCUUCCUUUAGC
GUUUCUUGCACAA 5′.

The following exemplary ASOs, containing a U-C or U-U mismatch at position +2 will enhance editing of the target adenosine:

(SEQ ID NO: 1299)
3′UUUACUCAGGUUACAGUCUAAAACCUUGAGUUGGGAUACUUGU
UACCCGGCA[C]GUUUACCUUUUUUUUCGACGUUUUCUUCCUUUA
GCGUUUCUUGCACAA 5′,
(SEQ ID NO: 1300)
3′UUUACUCAGGUUACAGUCUAAAACCUUGAGUUGGGAUACUUGU
UACCCGGCA[U]GUUUACCUUUUUUUUCGACGUUUUCUUCCUUUA
GCGUUUCUUGCACAA 5′,
(SEQ ID NO: 1301)
3′ CUUGUUACCCGGCA[C]GUUUACCUUUUUUUUCG 5′,
(SEQ ID NO: 1302)
3′ CUUGUUACCCGGCA[U]GUUUACCUUUUUUUUCG 5′.

The U-U or U-C mismatch at position +2 can be used within ASOs of any length and symmetry. In some embodiments, ASOs have a length of 15-101 nt, for example, in some implementations comprising between 4-50 nucleotides on either the 3′ or 5′ side of the target.

The U-U or U-C mismatch at position +2 may be combined with other features, such as a C-A mismatch at position +4 relative to the editing site. Such an ASO is exemplified by TP53 R175H (Rule 7, mismatches in square brackets and bold):

(SEQ ID NO: 1303)
3′ GCGGUACCGGUAGAUGUUCGUCAGUGUCGUGUACUGCCU
CCAACACUCCGCG[C]C[A]GGGGUGGUACUCGCGACGAGUC
UAUCGCUACCAGACCGGGGAGGAG 5′,
(SEQ ID NO: 1304)
3′ GCGGUACCGGUAGAUGUUCGUCAGUGUCGUGUACUGCCU
CCAACACUCCGCG[U]C[A]GGGGUGGUACUCGCGACGAGUC
UAUCGCUACCAGACCGGGGAGGAG 5′,
(SEQ ID NO: 1305)
3′ UCCAACACUCCGCG[C]C[A]GGGGUGGUACUCGCGA 5′,
(SEQ ID NO: 1306)
3′ UCCAACACUCCGCG[U]C[A]GGGGUGGUACUCGCGA 5′.

In some embodiments, the ASO further includes a recruitment domain. An exemplary recruitment domain (SEQ ID NO: 1166) is shown in italic:

(SEQ ID NO: 1307)
3′UCCAACACUCCGCG[C]CGGGGGGUGUCGAGAAGAGGAGAA
CAAUAUGCCAAAUGUUGUUCUCGUCUCCUCGACACC 5′,
(SEQ ID NO: 1308)
3′UCCAACACUCCGCG[U]CGGGGGGUGUCGAGAAGAGGAGAA
CAAUAUGCCAAAUGUUGUUCUCGUCUCCUCGACACC 5′.

### Example 8

### Generalizable Rule 7

A seventh generalizable rule identified herein is that if the target sequence contains a C residue at position +4 relative to the editing site, flanked by a 5′ G and 3′ C, then an A mismatch in the ASO across +4C increases editing. A schematic version of this rule is shown in FIG. 3B, item 7. The ASO facilitates ADAR-mediated A-to-G base replacement of a 5′-3′ target sequence comprising A *-X1-X2-X3-X4-X5, wherein X3 is G, X4 is C, X5 is C, and A* is the target base.

For example, in some embodiments the target RNA encodes TP53, carrying the R175H mutation associated with cancer. In some embodiments, the target RNA comprises the following sequence, with the mutated target adenosine underlined:

5′ CGCCAUGGCCAUCUACAAGCAGUCACAGCACAUGACGGAGGUUGUGAGGC ACUGCCCCCACCAUGAGCGCUGCUCAGAUAGCGAUGGUCUGGCCCCUCCUC 3′ (SEQ ID NO: 1172). In some embodiments, the corresponding complementary sequence (with C opposite from the target A) is:

(SEQ ID NO: 1280)
3′GCGGUACCGGUAGAUGUUCGUCAGUGUCGUGUACUGCC
UCCAACACUCCGCGACGGGGGUGGUACUCGCGACGAGUCU
AUCGCUACCAGACCGGGGAGGAG 5′.

The following exemplary ASOs, containing a C-A mismatch at position +4 will enhance editing of the target adenosine:

(SEQ ID NO: 1309)
3′GCGGUACCGGUAGAUGUUCGUCAGUGUCGUGUACUGCCUCCAA
CACUCCGCGAC[A]GGGGUGGUACUCGCGACGAGUCUAUCGCUAC
CAGACCGGGGAGGAG 5′,
(SEQ ID NO: 1310)
3′ UCCAACACUCCGCGAC[A]GGGGUGGUACUCGCGA 5′

As another example, in some embodiments, the target RNA encodes DDX58. In some embodiments, an ASO is employed to introduce the gain-of-function mutation E373G. in some embodiments, the target RNA comprises the following sequence, with the target adenosine underlined:

(SEQ ID NO: 1285)
5′AAAGGGAACGAUUCCAUCACUAUCCAUCUUUACUUUGA
UGAUAUUUGAUGAAUGCCACAACACUAGUAAACAACACCC
GUACAAUAUGAUCAUGUUUAAUU 3′.

In some embodiments, the corresponding complementary sequence (with C opposite from the target A) is:

(SEQ ID NO: 1286)
3′UUUCCCUUGCUAAGGUAGUGAUAGGUAGAAAUGAAACU
ACUAUAAACUACCUACGGUGUUGUGAUCAUUUGUUGUGGG
CAUGUUAUACUAGUACAAAUUAA 5′,

The following exemplary ASOs, containing a C-A mismatch at position +4 will enhance editing of the target adenosine:

(SEQ ID NO: 1311)
3′UUUCCCUUGCUAAGGUAGUGAUAGGUAGAAAUGAA
ACUACUAUAAACUACCUAC[A]GUGUUGUGAUCAUUUG
UUGUGGGCAUGUUAUACUAGUACAAAUUAA 5′,
(SEQ ID NO: 1312)
3′ ACUAUAAACUACCUAC[A]GUGUUGUGAUCAUUUG 5′.

The C-A mismatch at position +4 can be used within ASOs of any length and symmetry. In some embodiments, ASOs have a length of 15-101 nt, for example, in some implementations comprising between 4-50 nucleotides on either the 3′ or 5′ side of the target.

The C-A mismatch at position +4 may be combined with other features, such as a U-C or U-U mismatch at position +2 relative to the editing site. Such an ASO is exemplified by TP53 R175H (Rule 6, mismatches in square brackets and bold):

(SEQ ID NO: 1303)
3′ GCGGUACCGGUAGAUGUUCGUCAGUGUCGUGUACUGCC
UCCAACACUCCGCG[C]C[A]GGGGUGGUACUCGCGACGAG
UCUAUCGCUACCAGACCGGGGAGGAG 5′,
(SEQ ID NO: 1304)
3′ GCGGUACCGGUAGAUGUUCGUCAGUGUCGUGUACUGC
CUCCAACACUCCGCG[U]C[A]GGGGUGGUACUCGCGACG
AGUCUAUCGCUACCAGACCGGGGAGGAG 5′,
(SEQ ID NO: 1305)
3′ UCCAACACUCCGCG[C]C[A]GGGGUGGUACUCGCGA 5′,
(SEQ ID NO: 1306)
3′ UCCAACACUCCGCG[U]C[A]GGGGUGGUACUCGCGA 5′.

In some embodiments, the ASO additionally comprises a recruitment domain. An exemplary recruitment domain (SEQ ID NO: 1166) is shown in italic:

(SEQ ID NO: 1313)
3′UCCAACACUCCGCGAC[A]GGGGGUGUCGAGAAGAGGAGAA
CAAUAUGCCAAAUGUUGUUCUCGUCUCCUCGACACC 5′.

### Example 9

### Generalizable Rule 8

An eighth generalizable rule identified herein is that if the target sequence contains a U residue at position +5 relative to the editing site, flanked by a 5′ C and 3′ U, then a G in the ASO across +5U (presumably forming a U·G wobble base-pair) increases editing. A schematic version of this rule is shown in FIG. 3B, item 8. The ASO facilitates ADAR-mediated A-to-G base replacement of a 5′-3′ target sequence comprising A *-X1-X2-X3-X4-X5-X6, wherein X4 is C, X5 is U, X6 is U, and A* is the target base.

For example, in some embodiments the target RNA encodes IFNAR1. In some embodiments, an ASO is employed to eliminate a phosphorylation site at Y466. In some embodiments, the target RNA comprises the following sequence, with the target adenosine underlined:

5′

UCUCCCGUUUGUCAUUUAUGCUGCGAAAGUCUUCUUGAGAUGCAUCAAUUAUGUCUUCU UUCCAUCACUUAAACCUUCUUCCAGUAUAGAUGAGUAUUUCU 3′ (SEQ ID NO: 1182). In some embodiments, the corresponding complementary sequence (with C opposite from the target A) is:

(SEQ ID NO: 1220)
3′AGAGGGCAAACAGUAAAUACGACGCUUUCAGAAGAA
CUCUACGUAGUUAACACAGAAGAAAGGUAGUGAAUUUG
GAAGAAGGUCAUAUCUACUCAUAAAGA 5′.

The following exemplary ASOs, containing a U-G wobble base-pair at position +5 will enhance editing of the target adenosine:

(SEQ ID NO: 1314)
3′AGAGGGCAAACAGUAAAUACGACGCUUUCAGAAGAACUC
UACGUAGUUAACACAG[G]AGAAAGGUAGUGAAUUUGGAAG
AAGGUCAUAUCUACUCAUAAAGA 5′,
(SEQ ID NO: 1315)
3′ CUACGUAGUUAACACAG[G]AGAAAGGUAGUGAAU 5′.

The U-G wobble base-pair at position +5 can be used within ASOs of any length and symmetry. In some embodiments, ASOs have a length of 15-101 nt, for example, in some implementations comprising between 4-50 nucleotides on either the 3′ or 5′ side of the target.

The U-G wobble base-pair at position +5 may be combined with other features, such as a G-U wobble base-pair at position +2 relative to the editing site. Such an ASO is exemplified by IFNAR1 Y466 (Rule 5, mismatches in square brackets and bold):

(SEQ ID NO: 1316)
3′AGAGGGCAAACAGUAAAUACGACGCUUUCAGAAGAACUC
UACGUAGUUAACA[U]AG[G]AGAAAGGUAGUGAAUUUGGA
AGAAGGUCAUAUCUACUCAUAAAGA 5′,
(SEQ ID NO: 1317)
3′ CUACGUAGUUAACA[U]AG[G]AGAAAGGUAGUGAAU 5′

In some embodiments, the ASO further comprises a recruitment domain. An exemplary recruitment domain (SEQ ID NO: 1166) is shown in italic:

(SEQ ID NO: 1318)
3′CUACGUAGUUAACACAG[G]AGGGUGUCGAGAAGAGGAGAAC
AAUAUGCCAAAUGUUGUUCUCGUCUCCUCGACACC 5′.

TABLE 3
Antisense Sequences of the oligonucleotides or guide RNAs. (C = nucleotide within the
oligonucleotide that is directly opposite target adenosine (A) in the target RNA;
underlined for the first oligonucleotide of each target transcript)

| | | | | | Editing |
|---|---|---|---|---|---|
| | | | | | Level |
| | Mismatched | | | General | (%) at |
| SEQ | Base and | Antisense Sequence | Target | Rule | Position |
| NO. | Position | (3′-5′) | Transcript | Embodied | 0 |
| SEQ | −7G, −1G, | CGUGCCACAGCGCUGUUA | SNCA | | 76.2 |
| NO. 1 | +2C, +6U | | A53T | | |
| SEQ | −9C, −5A, | CCUACAACUGCGUUGUCC | SNCA | Rule 1 | 69.2 |
| NO. 2 | −2U, −1G, | | A53T | | |
| | +7C | | | | |
| SEQ | −3G, −1G, | CGUACCAGAGCGUUCUGA | SNCA | | 63.6 |
| NO. 3 | +4C, +6G | | A53T | | |
| SEQ | −9C, −1G | CACCUACCACAGCGUUGUCA | SNCA | Rule 1 | 57.7 |
| NO. 4 | | | A53T | | |
| SEQ | −9A, −2G, | CACAUACCACGCCGUUCUCA | SNCA | | 55.4 |
| NO. 5 | +4C | | A53T | | |
| SEQ | −9C, −1G, | CACCUACCACAGCGUUGUCG | SNCA | Rulc 1 | 53.6 |
| NO. 6 | +7G | | A53T | | |
| SEQ | −1G, +6A, | CACGUACCACAGCGUUGUAU | SNCA | | 52.9 |
| NO. 7 | +7U | | A53T | | |
| SEQ | −9A, −1G, | CAUACCACAGCGUUUUCG | SNCA | | 52.9 |
| NO. 8 | +4U, +7G | | A53T | | |
| SEQ | −9U, −1G | CACUUACCACAGCGUUGUCA | SNCA | Rule 1 | 46.8 |
| NO. 9 | | | A53T | | |
| SEQ | −9C, −1G, | CCUACCACAGCGUUGUCC | SNCA | Rule 1 | 46.8 |
| NO. 10 | +7C | | A53T | | |
| SEQ | −9C, −1G, | CACCUACCACAGCGUUUUCA | SNCA | Rule 1 | 46.1 |
| NO. 11 | +4U | | A53T | | |
| SEQ | −9U, −1G, | CUUACCACAGCGUAGUCA | SNCA | Rule 1 | 46 |
| NO. 12 | +3A | | A53T | | |
| SEQ | −9C, −1G, | CCUACCACAGCGUUAUCU | SNCA | Rule 1 | 45.1 |
| NO. 13 | +4A, +7U | | A53T | | |
| SEQ | −9C, −1G, | CCUACCACAGCGCUGUGG | SNCA | Rule 1 | 43.8 |
| NO. 14 | +2C, +6G, | | A53T | | |
| | +7G | | | | |
| SEQ | +3A, +5C | CACGUACCACACCGUAGCCA | SNCA | | 43.6 |
| NO. 15 | | | A53T | | |
| SEQ | −9C, −1A | CACCUACCACAACGUUGUCA | SNCA | Rule 1 | 43.5 |
| NO. 16 | | | A53T | | |
| SEQ | −9C, −1G, | CCUACCACAGCGUUGUAG | SNCA | Rule 1 | 43.5 |
| NO. 17 | +6A, +7G | | A53T | | |
| SEQ | −9C, −1G, | CCUACCACAGCGUUGUAA | SNCA | Rule 1 | 43.4 |
| NO. 18 | +6A | | A53T | | |
| SEQ | −1G, +3A, | CGUACCACAGCGUAGUGG | SNCA | | 42.9 |
| NO. 19 | +6G, +7G | | A53T | | |
| SEQ | −9C, −1G, | CCUACCACAGCGUUGUGA | SNCA | Rule 1 | 41.8 |
| NO. 20 | +6G | | A53T | | |
| SEQ | −9A, −1G, | CAUACCACAGCGUUCUCG | SNCA | | 40 |
| NO. 21 | +4C, +7G | | A53T | | |
| SEQ | −9U, −1G, | CUUACCACAGCGUUGUGA | SNCA | Rule 1 | 38.9 |
| NO. 22 | +6G | | A53T | | |
| SEQ | −9U, −1G, | CACUUACCACAGCGCUGUCA | SNCA | Rule 1 | 38.3 |
| NO. 23 | +2C | | A53T | | |
| SEQ | −9C, −1G, | CCUACCACAGCGUUGUCU | SNCA | Rule 1 | 37.9 |
| NO. 24 | +7U | | A53T | | |
| SEQ | −9A, −1G | CACAUACCACAGCGUUGUCA | SNCA | | 37.9 |
| NO. 25 | | | A53T | | |
| SEQ | −9C, −1G, | CCUACCACAGCGUUGUGU | SNCA | Rule 1 | 37.8 |
| NO. 26 | +6G, +7U | | A53T | | |
| SEQ | −9C, −1G, | CCUACCACAGCGUUGACA | SNCA | Rule 1 | 37.8 |
| NO. 27 | +5A | | A53T | | |
| SEQ | −10U, −1G, | UGUACCACAGCGUUGUAC | SNCA | | 37.5 |
| NO. 28 | +6A, +7C | | A53T | | |
| SEQ | −10U, −9C, | UCUACCACAGCGUUGUCU | SNCA | | 37.5 |
| NO. 29 | −1G, +7U | | A53T | | |
| SEQ | −9U, −1G, | CUUACCACAGCGCUGUCC | SNCA | Rule 1 | 37.5 |
| NO. 30 | +2C, +7C | | A53T | | |
| SEQ | −10U, −1G, | UGUACCACAGCGUCGUAA | SNCA | | 37.3 |
| NO. 31 | +3C, +6A | | A53T | | |
| SEQ | −5G, −3G, | CGUACGAGCGCGUUGUCA | SNCA | | 36.6 |
| NO. 32 | −2C, −1G | | A53T | | |
| SEQ | −10G, −1G, | GGUACCACAGCGUUGUUU | SNCA | | 36.4 |
| NO. 33 | +6U, +7U | | A53T | | |
| SEQ | −10G, −9C, | GCUACCACAGCGUCGUCA | SNCA | | 36 |
| NO. 34 | −1G, +3C | | A53T | | |
| SEQ | −9C, +6A | CACCUACCACACCGUUGUAA | SNCA | Rule 1 | 35.2 |
| NO. 35 | | | A53T | | |
| SEQ | −9C, −1G, | CCUACCACAGCGUUCUGA | SNCA | Rule 1 | 34.8 |
| NO. 36 | +4C, +6G | | A53T | | |
| SEQ | −9C, +4A, | CACCUACCACACCGUUAUCU | SNCA | Rule 1 | 34.6 |
| NO. 37 | +7U | | A53T | | |
| SEQ | +6A, +7U | CACGUACCACACCGUUGUAU | SNCA | | 34.5 |
| NO. 38 | | | A53T | | |
| SEQ | −9U, −1G, | CUUACCACAGCGUUGUAA | SNCA | Rule 1 | 33.6 |
| NO. 39 | +6A | | A53T | | |
| SEQ | −10A, −9U, | AUUACCACAGCGUUGUAA | SNCA | | 33.3 |
| NO. 40 | −1G, +6A | | A53T | | |
| SEQ | −10U, −8C, | UGCACCACAGCGUUGUGA | SNCA | | 33.3 |
| NO. 41 | −1G, +6G | | A53T | | |
| SEQ | −12A, −9C, | AACCUACCACACCGUUGUGA | SNCA | Rule 1 | 33 |
| NO. 42 | +6G | | A53T | | |
| SEQ | −9U, −1G, | CUUACCACAGCGUUAUCU | SNCA | Rule 1 | 32.9 |
| NO. 43 | +4A, +7U | | A53T | | |
| SEQ | −1G, +4U, | CGUACCACAGCGUUUUCC | SNCA | | 32.2 |
| NO. 44 | +7C | | A53T | | |
| SEQ | −9C, −1G, | CCUACCACAGCGGUGUAA | SNCA | Rule 1 | 32.1 |
| NO. 45 | +2G, +6A | | A53T | | |
| SEQ | −1G, +3C, | CGUACCACAGCGUCGUCG | SNCA | | 32 |
| NO. 46 | +7G | | A53T | | |
| SEQ | −9C, −1G, | CCUACCACAGCGUUAUCA | SNCA | Rule 1 | 32 |
| NO. 47 | +4A | | A53T | | |
| SEQ | −9C, −3U, | CCUACCAUAGCGUUGUCU | SNCA | Rule 1 | 31.9 |
| NO. 48 | −1G, +7U | | A53T | | |
| SEQ | −9U, −1G, | CUUACCACAGCGUUGUCG | SNCA | Rule 1 | 31.9 |
| NO. 49 | +7G | | A53T | | |
| SEQ | −9C, −4U | CACCUACCUCACCGUUGUCA | SNCA | Rule 1 | 31.9 |
| NO. 50 | | | A53T | | |
| SEQ | −1G, +7C | CACGUACCACAGCGUUGUCC | SNCA | | 31.9 |
| NO. 51 | | | A53T | | |
| SEQ | −9C, +2C, | CACCUACCACACCGCUGUGA | SNCA | Rule 1 | 31.9 |
| NO. 52 | +6G | | A53T | | |
| SEQ | −9U, −1G, | CUUACCACAGCGUUACCU | SNCA | Rule 1 | 31.8 |
| NO. 53 | +4A, +5C, | | A53T | | |
| | +7U | | | | |
| SEQ | −9U, −1G, | CUUACCACAGCGUUGUCC | SNCA | Rule 1 | 31.6 |
| NO. 54 | +7C | | A53T | | |
| SEQ | −12U, −9C, | UACCUACCACAACGUUGUCA | SNCA | Rule 1 | 31.4 |
| NO. 55 | −1A | | A53T | | |
| SEQ | −9C, +6G | CACCUACCACACCGUUGUGA | SNCA | Rule 1 | 31.3 |
| NO. 56 | | | A53T | | |
| SEQ | −10U, −1G, | UGUACCACAGCGUCGUCA | SNCA | | 31.2 |
| NO. 57 | +3C | | A53T | | |
| SEQ | −9C, −1G, | CCUACCACAGCGUUAACA | SNCA | Rule 1 | 31.2 |
| NO. 58 | +4A, +5A | | A53T | | |
| SEQ | −9C, −1G, | CCUACCACAGCGUCGCCA | SNCA | Rule 1 | 31.2 |
| NO. 59 | +3C, +5C | | A53T | | |
| SEQ | −1G, +3C, | CGUACCACAGCGUCGUCC | SNCA | | 31 |
| NO. 60 | +7C | | A53T | | |
| SEQ | −9U, −1G, | CUUACCACAGCGUUGCAA | SNCA | Rule 1 | 30.8 |
| NO. 61 | +5C, +6A | | A53T | | |
| SEQ | −10G, −1G, | GGUACCACAGCGUUGUCC | SNCA | | 30.8 |
| NO. 62 | +7C | | A53T | | |
| SEQ | −9C, −1G, | CCUACCACAGCGUCGUUU | SNCA | Rule 1 | 30.8 |
| NO. 63 | +3C, +6U, | | A53T | | |
| | +7U | | | | |
| SEQ | −10G, −9C, | GCUACCACAGCGUAGUCG | SNCA | | 30.4 |
| NO. 64 | −1G, +3A, | | | | |
| | +7G | | A53T | | |
| SEQ | −10U, −1G, | UGUACCACAGCGUUGAAA | SNCA | | 30.4 |
| NO. 65 | +5A, +6A | | A53T | | |
| SEQ | −9C, −3U, | CACCUACCAUACCGUUGUCC | SNCA | Rule 1 | 30.4 |
| NO. 66 | +7C | | A53T | | |
| SEQ | −10G, −9C, | GCUACCACAGCGUUGUCC | SNCA | | 30.3 |
| NO. 67 | −1G, +7C | | A53T | | |
| SEQ | −9C, +3A | CACCUACCACACCGUAGUCA | SNCA | Rule 1 | 30.1 |
| NO. 68 | | | A53T | | |
| SEQ | −1G, +3A, | CGUACCACAGCGUAUGCA | SNCA | | 30 |
| NO. 69 | +4U, +5G | | A53T | | |
| SEQ | −7C, −2U, | CGUCCCACUGCGUCGGCA | SNCA | | 30 |
| NO. 70 | −1G, +3C, | | A53T | | |
| | +5G | | | | |
| SEQ | −9A, −1G, | CAUACCACAGCGUUGUAA | SNCA | | 29.8 |
| NO. 71 | +6A | | A53T | | |
| SEQ | −11C, −7C, | CCCGUCCCACAACGUUGACA | SNCA | | 29.6 |
| NO. 72 | −1A, +5A | | A53T | | |
| SEQ | −8G, −5A, | CGGACAACAGCGUUGUAA | SNCA | | 29.4 |
| NO. 73 | −1G, +6A | | A53T | | |
| SEQ | −9C, −1G, | CCUACCACAGCGUAGUCA | SNCA | Rule 1 | 29.2 |
| NO. 74 | +3A | | A53T | | |
| SEQ | −9A, −1G, | CAUACCACAGCGUAGUCA | SNCA | | 29.1 |
| NO. 75 | +3A | | A53T | | |
| SEQ | −1A | CGUACCACAACGUUGUCA | SNCA | | 29 |
| NO. 76 | | | A53T | | |
| SEQ | −10U, −1G, | UGUACCACAGCGUUGUGA | SNCA | | 28.9 |
| NO. 77 | +6G | | A53T | | |
| SEQ | −9U, +3C, | CACUUACCACACCGUCGUAA | SNCA | Rule 1 | 28.9 |
| NO. 78 | +6A | | A53T | | |
| SEQ | −1G, +7U | CACGUACCACAGCGUUGUCU | SNCA | | 28.8 |
| NO. 79 | | | A53T | | |
| SEQ | −9A, −1G, | CAUACCACAGCGUUGUCU | SNCA | | 28.7 |
| NO. 80 | +7U | | A53T | | |
| SEQ | −1G, +6A | CGUACCACAGCGUUGUAA | SNCA | | 28.6 |
| NO. 81 | | | A53T | | |
| SEQ | −9U, −4C, | CUUACCCCAGCGUUUUCA | SNCA | Rule 1 | 28.6 |
| NO. 82 | −1G, +4U | | A53T | | |
| SEQ | −9C, −1G, | CCUACCACAGCGUUCUCA | SNCA | Rule 1 | 28.6 |
| NO. 83 | +4C | | A53T | | |
| SEQ | −10A, −9C, | ACUACCACAGCGUUGUGA | SNCA | | 28.6 |
| NO. 84 | −1G, +6G | | A53T | | |
| SEQ | −9A, −2G, | CAUACCACGGCGUUGUCU | SNCA | | 28.3 |
| NO. 85 | −1G, +7U | | A53T | | |
| SEQ | −9U, +3A | CACUUACCACACCGUAGUCA | SNCA | Rule 1 | 28.1 |
| NO. 86 | | | A53T | | |
| SEQ | −9C, +4U, | CACCUACCACACCGUUUUCG | SNCA | Rule 1 | 28 |
| NO. 87 | +7G | | A53T | | |
| SEQ | −10G, −9A, | GAUACCACAGCGUUUUCA | SNCA | | 28 |
| NO. 88 | −1G, +4U | | A53T | | |
| SEQ | −1G, +3A, | CGUACCACAGCGUAGGCA | SNCA | | 27.9 |
| NO. 89 | +5G | | A53T | | |
| SEQ | −9U, −1A | CACUUACCACAACGUUGUCA | SNCA | Rule 1 | 27.9 |
| NO. 90 | | | A53T | | |
| SEQ | −9A, −3U, | CACAUACCAUAGCGUUGUCA | SNCA | | 27.9 |
| NO. 91 | −1G | | A53T | | |
| SEQ | −1G, +5G | CACGUACCACAGCGUUGGCA | SNCA | | 27.9 |
| NO. 92 | | | A53T | | |
| SEQ | −9U, −1G, | CUUACCACAGCGUCGUCG | SNCA | Rule 1 | 27.7 |
| NO. 93 | +3C, +7G | | A53T | | |
| SEQ | −1G, +6A, | CGUACCACAGCGUUGUAG | SNCA | | 27.7 |
| NO. 94 | +7G | | A53T | | |
| SEQ | −10U, −1G, | UGUACCACAGCGUUGGCC | SNCA | | 27.6 |
| NO. 95 | +5G, +7C | | A53T | | |
| SEQ | +3G, +7G | CACGUACCACACCGUGGUCG | SNCA | | 27.6 |
| NO. 96 | | | A53T | | |
| SEQ | −9C | CACCUACCACACCGUUGUCA | SNCA | Rule 1 | 27.6 |
| NO. 97 | | | A53T | | |
| SEQ | −9C, +3C | CACCUACCACACCGUCGUCA | SNCA | Rule 1 | 27.5 |
| NO. 98 | | | A53T | | |
| SEQ | −1G, +3A, | CGUACCACAGCGUAGUCC | SNCA | | 27.3 |
| NO. 99 | +7C | | A53T | | |
| SEQ | −8C, −1G, | CGCACCACAGCGUUGUAU | SNCA | | 27.3 |
| NO. 100 | +6A, +7U | | A53T | | |
| SEQ | −9C, −1G, | CCUACCACAGCGUUCUUA | SNCA | Rule 1 | 27.3 |
| NO. 101 | +4C, +6U | | A53T | | |
| SEQ | −9C, −1G, | CCUACCACAGCGUUGCAA | SNCA | Rule 1 | 27.3 |
| NO. 102 | +5C, +6A | | A53T | | |
| SEQ | −10U, −1G, | UGUACCACAGCGUUGUAA | SNCA | | 27.3 |
| NO. 103 | +6A | | A53T | | |
| SEQ | −10A, −9C, | ACUACCACAGCGUUAUCC | SNCA | | 27.3 |
| NO. 104 | −1G, +4A, | | A53T | | |
| | +7C | | | | |
| SEQ | −9C, +3G | CACCUACCACACCGUGGUCA | SNCA | Rule 1 | 27.2 |
| NO. 105 | | | A53T | | |
| SEQ | −12U, −9C, | UACCUACCACACCGUAGUCA | SNCA | Rule 1 | 27.2 |
| NO. 106 | +3A | | A53T | | |
| SEQ | −1G, +3C | CACGUACCACAGCGUCGUCA | SNCA | | 27 |
| NO. 107 | | | A53T | | |
| SEQ | −10U, +3A | CAUGUACCACACCGUAGUCA | SNCA | | 27 |
| NO. 108 | | | A53T | | |
| SEQ | −10G, −1G, | GGUACCACAGCGUUGCCU | SNCA | | 27 |
| NO. 109 | +5C, +7U | | A53T | | |
| SEQ | −3U, −1G, | CGUACCAUAGCGUUGUGG | SNCA | | 26.9 |
| NO. 110 | +6G, +7G | | A53T | | |
| SEQ | −1G, +6G | CACGUACCACAGCGUUGUGA | SNCA | | 26.7 |
| NO. 111 | | | A53T | | |
| SEQ | −9C, −2G, | CCUACCACGGCGUUGUCG | SNCA | Rule 1 | 26.7 |
| NO. 112 | −1G, +7G | | A53T | | |
| SEQ | −10A, −9U, | AUUACCACAGCGUUGACA | SNCA | | 26.7 |
| NO. 113 | −1G, +5A | | A53T | | |
| SEQ | −3U, −1G, | CGUACCAUAGCGUUCUCU | SNCA | | 26.7 |
| NO. 114 | +4C, +7U | | A53T | | |
| SEQ | −9U, −5U, | CUUACUACAGCGUUGUGA | SNCA | Rule 1 | 26.7 |
| NO. 115 | −1G, +6G | | A53T | | |
| SEQ | −9C, −1G, | CCUACCACAGCGUACUCA | SNCA | Rule 1 | 26.7 |
| NO. 116 | +3A, +4C | | A53T | | |
| SEQ | −10U, −1G, | UGUACCACAGCGUUGUCC | SNCA | | 26.7 |
| NO. 117 | +7C | | A53T | | |
| SEQ | −9C, 0U | CACCUACCACACUGUUGUCA | SNCA | Rule 1 | 26.7 |
| NO. 118 | | | A53T | | |
| SEQ | −4C, −1G | CACGUACCCCAGCGUUGUCA | SNCA | | 26.7 |
| NO. 119 | | | A53T | | |
| SEQ | −9A, −1G, | CAUACCACAGCGUUGUGA | SNCA | | 26.7 |
| NO. 120 | +6G | | A53T | | |
| SEQ | −10G, −9U, | GUUACCACAGCGUUGUGA | SNCA | | 26.5 |
| NO. 121 | −1G, +6G | | A53T | | |
| SEQ | −9C, −1G, | CCUACCACAGCGUUGGCA | SNCA | Rule 1 | 26.5 |
| NO. 122 | +5G | | A53T | | |
| SEQ | −10G, −1G, | GGUACCACAGCGUUGUGA | SNCA | | 26.3 |
| NO. 123 | +6G | | A53T | | |
| SEQ | −1G, +7G | CGUACCACAGCGUUGUCG | SNCA | | 26.3 |
| NO. 124 | | | A53T | | |
| SEQ | −5U, −1G, | CGUACUACAGCGUAGUGA | SNCA | | 26.3 |
| NO. 125 | +3A, +6G | | A53T | | |
| SEQ | −12A, −9A, | AACAUACCACACCGUUGUGA | SNCA | Rule 1 | 26.3 |
| NO. 126 | +6G | | A53T | | |
| SEQ | −9U, +2G | CACUUACCACACCGGUGUCA | SNCA | Rule 1 | 26.3 |
| NO. 127 | | | A53T | | |
| SEQ | −9U, −1G, | CUUACCACAGCGUUGUGG | SNCA | Rule 1 | 26.2 |
| NO. 128 | +6G, +7G | | A53T | | |
| SEQ | −10G, −9U, | GUUACCACAGCGUUGUCC | SNCA | | 26.2 |
| NO. 129 | −1G, +7C | | A53T | | |
| SEQ | −10G, −1G, | GGUACCACAGCGUUGAAA | SNCA | | 26.2 |
| NO. 130 | +5A, +6A | | A53T | | |
| SEQ | −1G, +4U, | CGUACCACAGCGUUUUCU | SNCA | | 26.2 |
| NO. 131 | +7U | | A53T | | |
| SEQ | −1G, +3A, | CGUACCACAGCGUAGUCU | SNCA | | 26.1 |
| NO. 132 | +7U | | A53T | | |
| SEQ | −3U, −1G, | CGUACCAUAGCGUUGUCG | SNCA | | 26.1 |
| NO. 133 | +7G | | A53T | | |
| SEQ | −11G, −1G | CGCGUACCACAGCGUUGUCA | SNCA | | 25.9 |
| NO. 134 | | | A53T | | |
| SEQ | −9A, −1G, | CAUACCACAGCGUUGUCG | SNCA | | 25.9 |
| NO. 135 | +7G | | A53T | | |
| SEQ | −11C, −1G | CCCGUACCACAGCGUUGUCA | SNCA | | 25.8 |
| NO. 136 | | | A53T | | |
| SEQ | −9U, −1G, | CUUACCACAGCGUAGUCU | SNCA | Rule 1 | 25.8 |
| NO. 137 | +3A, +7U | | A53T | | |
| SEQ | −9U, −1G, | CUUACCACAGCGUUGUCU | SNCA | Rule 1 | 25.7 |
| NO. 138 | +7U | | A53T | | |
| SEQ | −9U, −3U, | CUUACCAUAGCGUUGUAA | SNCA | Rule 1 | 25.7 |
| NO. 139 | −1G, +6A | | A53T | | |
| SEQ | −9U, −1A, | CACUUACCACAACGUUCUCA | SNCA | Rule 1 | 25.7 |
| NO. 140 | +4C | | A53T | | |
| SEQ | −1G, +3C, | CGUACCACAGCGUCGUUG | SNCA | | 25.6 |
| NO. 141 | +6U, +7G | | A53T | | |
| SEQ | −1G, +3C, | CGUACCACAGCGUCGUCU | SNCA | | 25.6 |
| NO. 142 | +7U | | A53T | | |
| SEQ | −9C, +2G | CACCUACCACACCGGUGUCA | SNCA | Rule 1 | 25.5 |
| NO. 143 | | | A53T | | |
| SEQ | −12U, −10U | UAUGUACCACACCGUUGUCA | SNCA | | 25.4 |
| NO. 144 | | | A53T | | |
| SEQ | −2G, +7C | CACGUACCACGCCGUUGUCC | SNCA | | 25.2 |
| NO. 145 | | | A53T | | |
| SEQ | −9C, −1G, | CCUACCACAGCGUUGUUA | SNCA | Rule 1 | 25 |
| NO. 146 | +6U | | A53T | | |
| SEQ | −9U, −1G, | CUUACCACAGCGUUGCGA | SNCA | Rule 1 | 25 |
| NO. 147 | +5C, +6G | | A53T | | |
| SEQ | −9A, −1G, | CAUACCACAGCGUUGUGC | SNCA | | 25 |
| NO. 148 | +6G, +7C | | A53T | | |
| SEQ | −8G, −5G, | CGGACGGCAGCGUUGUCA | SNCA | | 25 |
| NO. 149 | −4G, −1G | | A53T | | |
| SEQ | −9A, −1G, | CAUACCACAGCGUCGUCC | SNCA | | 25 |
| NO. 150 | +3C, +7C | | A53T | | |
| SEQ | −1G, +4A, | CGUACCACAGCGUUAAAA | SNCA | | 25 |
| NO. 151 | +5A, +6A | | A53T | | |
| SEQ | −2G, −1G, | CGUACCACGGCGUUGUCU | SNCA | | 25 |
| NO. 152 | +7U | | A53T | | |
| SEQ | −5U, −1G, | CGUACUACAGCGUUAUCG | SNCA | | 25 |
| NO. 153 | +4A, +7G | | A53T | | |
| SEQ | −9U, −1G, | CUUACCACAGCGUUAUUA | SNCA | Rule 1 | 25 |
| NO. 154 | +4A, +6U | | A53T | | |
| SEQ | −9U, −5U, | CUUACUACAGAGUUGUAA | SNCA | Rule 1 | 25 |
| NO. 155 | −1G, 0A, | | A53T | | |
| | +6A | | | | |
| SEQ | −10G, −1G, | GGUACCACAGCGUUGUGG | SNCA | | 25 |
| NO. 156 | +6G, +7G | | A53T | | |
| SEQ | −9A, −1G, | CAUACCACAGCGUUGUUA | SNCA | | 25 |
| NO. 157 | +6U | | A53T | | |
| SEQ | −10U, −9A, | UAUACCACAGCGUUAUCA | SNCA | | 25 |
| NO. 158 | −1G, +4A | | A53T | | |
| SEQ | −9U, −3U, | CUUACCAUAGCGUAGUAA | SNCA | Rule 1 | 25 |
| NO. 159 | −1G, +3A, | | A53T | | |
| | +6A | | | | |
| SEQ | −1G, +3C, | CGUACCACAGCGUCGUUC | SNCA | | 25 |
| NO. 160 | +6U, +7C | | A53T | | |
| SEQ | −9U, −2G, | CACUUACCACGCCGUUGUAA | SNCA | Rule 1 | 25 |
| NO. 161 | +6A | | A53T | | |
| SEQ | −9U, +4C | CACUUACCACACCGUUCUCA | SNCA | Rule 1 | 25 |
| NO. 162 | | | A53T | | |
| SEQ | −9U, +4U | CACUUACCACACCGUUUUCA | SNCA | Rule 1 | 25 |
| NO. 163 | | | A53T | | |
| SEQ | −9C, −1G, | CCUACCACAGCGUUGCCA | SNCA | Rule 1 | 24.9 |
| NO. 164 | +5C | | A53T | | |
| SEQ | −1G, +6U, | CGUACCACAGCGUUGUUU | SNCA | | 24.9 |
| NO. 165 | +7U | | A53T | | |
| SEQ | −1G, +3C, | CGUACCACAGCGUCGUGA | SNCA | | 24.7 |
| NO. 166 | +6G | | A53T | | |
| SEQ | −1G, +3A, | CGUACCACAGCGUAGUGA | SNCA | | 24.7 |
| NO. 167 | +6G | | A53T | | |
| SEQ | −11C, −1G, | CCCGUACCACAGCGUUCUCA | SNCA | | 24.7 |
| NO. 168 | +4C | | A53T | | |
| SEQ | −9U, +3C | CACUUACCACACCGUCGUCA | SNCA | Rule 1 | 24.6 |
| NO. 169 | | | A53T | | |
| SEQ | −9U, +3C, | CACUUACCACACCGUCGUCG | SNCA | Rule 1 | 24.6 |
| NO. 170 | +7G | | A53T | | |
| SEQ | −10U, −1G | CAUGUACCACAGCGUUGUCA | SNCA | | 24.6 |
| NO. 171 | | | A53T | | |
| SEQ | −1G, +4A, | CGUACCACAGCGUUAUCU | SNCA | | 24.6 |
| NO. 172 | +7U | | A53T | | |
| SEQ | −1G, +6G, | CGUACCACAGCGUUGUGG | SNCA | | 24.6 |
| NO. 173 | +7G | | A53T | | |
| SEQ | −9U, +3C, | CACUUACCACACCGUCAUUA | SNCA | Rule 1 | 24.5 |
| NO. 174 | +4A, +6U | | A53T | | |
| SEQ | −9A, −1G, | CAUACCACAGCGUGGUCC | SNCA | | 24.5 |
| NO. 175 | +3G, +7C | | A53T | | |
| SEQ | −1G, +3A | CACGUACCACAGCGUAGUCA | SNCA | | 24.4 |
| NO. 176 | | | A53T | | |
| SEQ | −1G, +4C, | CGUACCACAGCGUUCUCG | SNCA | | 24.4 |
| NO. 177 | +7G | | A53T | | |
| SEQ | −3U, −1G, | CGUACCAUAGCGUUUUAA | SNCA | | 24.3 |
| NO. 178 | +4U, +6A | | A53T | | |
| SEQ | −1G, +2C, | CACGUACCACAGCGCUGUAA | SNCA | | 24.3 |
| NO. 179 | +6A | | A53T | | |
| SEQ | −10G, −1G, | GGUACCACAGCGUAGUCA | SNCA | | 24.3 |
| NO. 180 | +3A | | A53T | | |
| SEQ | −1G, +4U, | CGUACCACAGCGUUUUCG | SNCA | | 24.2 |
| NO. 181 | +7G | | A53T | | |
| SEQ | −9U, −1G, | CUUACCACAGCGUUGUUA | SNCA | Rule 1 | 24.2 |
| NO. 182 | +6U | | A53T | | |
| SEQ | −2G, −1G, | CGUACCACGGCGUUGUGA | SNCA | | 24.2 |
| NO. 183 | +6G | | A53T | | |
| SEQ | −1G, +4U, | CGUACCACAGCGUUUUAA | SNCA | | 24.2 |
| NO. 184 | +6A | | A53T | | |
| SEQ | −9U, −1G, | CUUACCACAGCGUAGUCG | SNCA | Rule 1 | 24.2 |
| NO. 185 | +3A, +7G | | A53T | | |
| SEQ | −9A, −3U, | CAUACCAUAGCGUUGUCU | SNCA | | 24.1 |
| NO. 186 | −1G, +7U | | A53T | | |
| SEQ | −9A, −1G, | CAUACCACAGCGUUGACC | SNCA | | 24.1 |
| NO. 187 | +5A, +7C | | A53T | | |
| SEQ | −1G, +4U, | CGUACCACAGCGUUUAUA | SNCA | | 24 |
| NO. 188 | +5A, +6U | | A53T | | |
| SEQ | −9A, +6A | CACAUACCACACCGUUGUAA | SNCA | Rule 1 | 23.9 |
| NO. 189 | | | A53T | | |
| SEQ | −9U, −1G, | CUUACCACAGCGUUGACA | SNCA | Rule 1 | 23.9 |
| NO. 190 | +5A | | A53T | | |
| SEQ | −10G, −9C, | GCUACCACAGCGUUGUCA | SNCA | | 23.9 |
| NO. 191 | −1G | | A53T | | |
| SEQ | −1G, +4A, | CGUACCACAGCGUUAUGA | SNCA | | 23.9 |
| NO. 192 | +6G | | A53T | | |
| SEQ | −2G, −1G, | CGUACCACGGCGUUCUCA | SNCA | | 23.8 |
| NO. 193 | +4C | | A53T | | |
| SEQ | −10G, −1G, | GGUACCACAGCGUUGUCU | SNCA | | 23.8 |
| NO. 194 | +7U | | A53T | | |
| SEQ | −10U, −9U, | UUUACCACAGCGUUCUCA | SNCA | | 23.8 |
| NO. 195 | −1G, +4C | | A53T | | |
| SEQ | −10U, −1G, | UGUACCACAGCGUUGUAG | SNCA | | 23.6 |
| NO. 196 | +6A, +7G | | A53T | | |
| SEQ | −1G, +2C, | CGUACCACAGCGCUGUCU | SNCA | | 23.6 |
| NO. 197 | +7U | | A53T | | |
| SEQ | −9U | CACUUACCACACCGUUGUCA | SNCA | Rule 1 | 23.6 |
| NO. 198 | | | A53T | | |
| SEQ | −9A, +6G | CACAUACCACACCGUUGUGA | SNCA | Rule 1 | 23.6 |
| NO. 199 | | | A53T | | |
| SEQ | −9C, +5C | CACCUACCACACCGUUGCCA | SNCA | Rule 1 | 23.6 |
| NO. 200 | | | A53T | | |
| SEQ | −10A, −9C, | ACUACCAUAGCGUUGUCA | SNCA | | 23.5 |
| NO. 201 | −3U, −1G | | A53T | | |
| SEQ | −10U, −6U, | UGUAUCACUGCGCUGUCA | SNCA | | 23.5 |
| NO. 202 | −2U, −1G, | | A53T | | |
| | +2C | | | | |
| SEQ | −9U, −1G, | CUUACCACAGCGUCAUCA | SNCA | Rule 1 | 23.5 |
| NO. 203 | +3C, +4A | | A53T | | |
| SEQ | −1G, +3A, | CGUACCACAGCGUAGUAA | SNCA | | 23.5 |
| NO. 204 | +6A | | A53T | | |
| SEQ | −10U, −1G, | UGUACCACAGCGUUGUCG | SNCA | | 23.4 |
| NO. 205 | +7G | | A53T | | |
| SEQ | −10U, −1G, | UGUACCACAGCGCUGUAA | SNCA | | 23.4 |
| NO. 206 | +2C, +6A | | A53T | | |
| SEQ | −3U, −1G, | CGUACCAUAGCGUCGUCA | SNCA | | 23.4 |
| NO. 207 | +3C | | A53T | | |
| SEQ | −9A, +7U | CACAUACCACACCGUUGUCU | SNCA | Rule 1 | 23.4 |
| NO. 208 | | | A53T | | |
| SEQ | −9C, −1G, | CCUACCACAGCGUUAUAG | SNCA | Rule 1 | 23.3 |
| NO. 209 | +4A, +6A, | | A53T | | |
| | +7G | | | | |
| SEQ | −1G, +3C, | CGUACCACAGCGUCAUUA | SNCA | | 23.3 |
| NO. 210 | +4A, +6U | | A53T | | |
| SEQ | −1G, +5A, | CGUACCACAGCGUUGAAA | SNCA | | 23.2 |
| NO. 211 | +6A | | A53T | | |
| SEQ | −10A, −1G, | AGUACCACAGCGUUGUCG | SNCA | | 23.2 |
| NO. 212 | +7G | | A53T | | |
| SEQ | −1G, +4C, | CGUACCACAGCGUUCUUA | SNCA | | 23.2 |
| NO. 213 | +6U | | A53T | | |
| SEQ | −10U, −9C, | UCUACCACAGCGUUGCCA | SNCA | | 23.2 |
| NO. 214 | −1G, +5C | | A53T | | |
| SEQ | −10U, −1G, | UGUACCACAGCGUAGUCA | SNCA | | 23.1 |
| NO. 215 | +3A | | A53T | | |
| SEQ | −8A, −1G, | CGAACCACAGCGUUCUCU | SNCA | | 23.1 |
| NO. 216 | +4C, +7U | | A53T | | |
| SEQ | −10A, −1G, | AGUACCACAGCGUCAUCA | SNCA | | 23.1 |
| NO. 217 | +3C, +4A | | A53T | | |
| SEQ | −10A, +6G, | CAAGUACCACACCGUUGUGU | SNCA | | 23.1 |
| NO. 218 | +7U | | A53T | | |
| SEQ | −3U, −1G, | CGUACCAUAGCGUAGUCC | SNCA | | 23.1 |
| NO. 219 | +3A, +7C | | A53T | | |
| SEQ | −10A, −9C, | ACUACCACAGCGUUGUCA | SNCA | | 23.1 |
| NO. 220 | −1G | | A53T | | |
| SEQ | −10U, −1G, | UGUACCACAGCGUUCUCA | SNCA | | 23.1 |
| NO. 221 | +4C | | A53T | | |
| SEQ | −1G, +3G, | CGUACCACAGCGUGGUAG | SNCA | | 23.1 |
| NO. 222 | +6A, +7G | | A53T | | |
| SEQ | −10U, +5A, | UUGGUAGCUGCUCUUACG | SERPINA1 | | 91.8 |
| NO. 223 | +7G | | E342K | | |
| SEQ | −5C, −4U, | CUGGUCUCUUUUCUUUCC | SERPINA1 | | 91.2 |
| NO. 224 | −1U, 0U | | E342K | | |
| SEQ | −9C,−6C, | CCGGCAGCUGCAUUUUCU | SERPINA1 | | 90.9 |
| NO. 225 | +1A, +2U, | | E342K | | |
| | +7U | | | | |
| SEQ | −10U, −2A, | UUGGUAGCAGCUCUUGCC | SERPINA1 | | 86.5 |
| NO. 226 | +5G | | E342K | | |
| SEQ | −7A, −3A, | CUGAUAGAUGUUCACUCC | SERPINA1 | | 86.4 |
| NO. 227 | 0U, +3A, | | E342K | | |
| | +4C | | | | |
| SEQ | −10A, +1A, | AUGGUAGCUGCACUUUGC | SERPINA1 | | 85.5 |
| NO. 228 | +6G | | E342K | | |
| SEQ | −6G, −2A, | CUGGGAGCAGCUCUUUCG | SERPINA1 | | 83.7 |
| NO. 229 | +7G | | E342K | | |
| SEQ | −10U, −9A, | UAGGUAGCUGCACUUUCC | SERPINA1 | | 82.6 |
| NO. 230 | +1A | | E342K | | |
| SEQ | −3U, +1G, | CUGGUAGUUGCGCUUCAC | SERPINA1 | | 79 |
| NO. 231 | +5C, +6A | | E342K | | |
| SEQ | −10A, −9G, | AGGGUAGCUGCUCUUUUA | SERPINA1 | | 78 |
| NO. 232 | +6U, +7A | | E342K | | |
| SEQ | −1U, +1G, | CUGGUAGCUUCGCCUUUC | SERPINA1 | | 77.4 |
| NO. 233 | +3C, +6U | | E342K | | |
| SEQ | −10A, −9G, | AGCGUAGCUGCUCUAUCC | SERPINA1 | | 76.3 |
| NO. 234 | −8C, +4A | | E342K | | |
| SEQ | −8A, −2A, | CUAGUAGCAGCUCUUACG | SERPINA1 | | 70.6 |
| NO. 235 | +5A, +7G | | E342K | | |
| SEQ | −10G, −2G, | GUGGUAGCGGCUCUUACU | SERPINA1 | | 70.3 |
| NO. 236 | +5A, +7U | | E342K | | |
| SEQ | −10G, −6G, | GUGGGAGUUACUCUUGCC | SERPINA1 | | 66.8 |
| NO. 237 | 3−U, −1A, | | E342K | | |
| | +5G | | | | |
| SEQ | −9G, −8C, | CGCGUAGCUGCUCGGACC | SERPINA1 | | 66.7 |
| NO. 238 | +3G, +4G, | | E342K | | |
| | +5A | | | | |
| SEQ | −7A, +2G, | CUGAUAGCUGCUGCUUCC | SERPINA1 | | 65.1 |
| NO. 239 | +3C | | E342K | | |
| SEQ | −6A, +2U, | CUGGAAGCUGCUUAUACC | SERPINA1 | | 61 |
| NO. 240 | +3A, +5A | | E342K | | |
| SEQ | −2C, +4A, | CUGGUAGCCGCUCUAUAC | SERPINA1 | | 60 |
| NO. 241 | +6A | | E342K | | |
| SEQ | −7U, −2G, | CUGUUAGCGUCUCUUUGU | SERPINA1 | Rule 3 | 58.8 |
| NO. 242 | −1U, +6G, | | E342K | | |
| | +7U | | | | |
| SEQ | +2U, +5G, | CUGGUAGCUGCUUUUGCA | SERPINA1 | Rule 5 | 54.3 |
| NO. 243 | +7A | | E342K | | |
| SEQ | −3G, −2C, | CUGGUAGGCGCUCGUUAC | SERPINA1 | | 53.6 |
| NO. 244 | +3G, +6A | | E342K | | |
| SEQ | −10U, −5G, | UUGGUGGCAGCUCUUACC | SERPINA1 | | 49.3 |
| NO. 245 | −2A, +5A | | E342K | | |
| SEQ | −10A, −6G, | AUGGGAACUGCUCGUUCG | SERPINA1 | | 46.4 |
| NO. 246 | −4A, +3G, | | E342K | | |
| | +7G | | | | |
| SEQ | −8U, −6A, | CUUGAAGCUGCUGAUUAC | SERPINA1 | | 44.7 |
| NO. 247 | +2G, +3A, | | E342K | | |
| | +6A | | | | |
| SEQ | −7C, +2U | CUGCUAGCUGCUUUUUCC | SERPINA1 | Rule 3, | 44.1 |
| NO. 248 | | | E342K | Rule 5 | |
| SEQ | −10U, −8U, | UUUGUAGCUGCUCUUUCG | SERPINA1 | | 43.8 |
| NO. 249 | +7G | | E342K | | |
| SEQ | −5U, −3G, | CUGGUUGGUGGGCCUUCC | SERPINA1 | | 41.3 |
| NO. 250 | 0G, +1G, | | E342K | | |
| | +3C | | | | |
| SEQ | −8U, −6G, | CUUGGAGCUGCUCCAUCC | SERPINA1 | | 40.4 |
| NO. 251 | +3C, +4A | | E342K | | |
| SEQ | −7U, +4C, | CUGUUAGCUGCUCUCUCU | SERPINA1 | Rule 3 | 40.2 |
| NO. 252 | +7U | | E342K | | |
| SEQ | −7C, +2U, | CUGCUAGCUGCUUUUUCA | SERPINA1 | Rule 3, | 38.5 |
| NO. 253 | +7A | | E342K | Rule 5 | |
| SEQ | −7U, +6A | CUGUUAGCUGCUCUUUAC | SERPINA1 | Rule 3 | 37.3 |
| NO. 254 | | | E342K | | |
| SEQ | −7C, +3C | CUGCUAGCUGCUCCUUCC | SERPINA1 | Rule 3 | 37.2 |
| NO. 255 | | | E342K | | |
| SEQ | −7C, −3U | CUGCUAGUUGCUCUUUCC | SERPINA1 | Rule 3 | 37 |
| NO. 256 | | | E342K | | |
| SEQ | −7U, +2U | CUGUUAGCUGCUUUUUCC | SERPINA1 | Rule 3, | 36.9 |
| NO. 257 | | | E342K | Rule 5 | |
| SEQ | −7U, +3C | CUGUUAGCUGCUCCUUCC | SERPINA1 | Rule 3 | 36.4 |
| NO. 258 | | | E342K | | |
| SEQ | −7C, +1C | CUGCUAGCUGCCCUUUCC | SERPINA1 | Rule 3 | 36.2 |
| NO. 259 | | | E342K | | |
| SEQ | −7C, +4G, | CUGCUAGCUGCUCUGUCU | SERPINA1 | Rule 3 | 35.9 |
| NO. 260 | +7U | | E342K | | |
| SEQ | −7C, +1C, | CUGCUAGCUGCCCUAUCC | SERPINA1 | Rule 3 | 35.5 |
| NO. 261 | +4A | | E342K | | |
| SEQ | −7C, +4C, | CUGCUAGCUGCUCUCUCU | SERPINA1 | Rule 3 | 35.4 |
| NO. 262 | +7U | | E342K | | |
| SEQ | −10A, +2U | AUGGUAGCUGCUUUUUCC | SERPINA1 | Rule 5 | 35.3 |
| NO. 263 | | | E342K | | |
| SEQ | −7C, −2A, | CUGCUAGCAGCUCUUUCU | SERPINA1 | Rule 3 | 35.1 |
| NO. 264 | +7U | | E342K | | |
| SEQ | −7C, +3A | CUGCUAGCUGCUCAUUCC | SERPINA1 | Rule 3 | 34.7 |
| NO. 265 | | | E342K | | |
| SEQ | −10G, −7C, | GUGCUAGCUGCUUUUUCC | SERPINA1 | Rule 3, | 34.7 |
| NO. 266 | +2U | | E342K | Rule 5 | |
| SEQ | −7U, +2U, | CUGUUAGCUGCUUUUUAC | SERPINA1 | Rule 3, | 34.6 |
| NO. 267 | +6A | | E342K | Rule 5 | |
| SEQ | −7U, +4C | CUGUUAGCUGCUCUCUCC | SERPINA1 | Rule 3 | 34.6 |
| NO. 268 | | | E342K | | |
| SEQ | −7C, +4C | CUGCUAGCUGCUCUCUCC | SERPINA1 | Rule 3 | 34.4 |
| NO. 269 | | | E342K | | |
| SEQ | −7C | CUGCUAGCUGCUCUUUCC | SERPINA1 | Rule 3 | 34.2 |
| NO. 270 | | | E342K | | |
| SEQ | −7C, +7U | CUGCUAGCUGCUCUUUCU | SERPINA1 | Rule 3 | 34.1 |
| NO. 271 | | | E342K | | |
| SEQ | −7C, −2A | CUGCUAGCAGCUCUUUCC | SERPINA1 | Rule 3 | 33.1 |
| NO. 272 | | | E342K | | |
| SEQ | −7C, +6A | CUGCUAGCUGCUCUUUAC | SERPINA1 | Rule 3 | 33 |
| NO. 273 | | | E342K | | |
| SEQ | −10U, −7C, | UUGCUAGCUGCUCUCUCC | SERPINA1 | Rule 3 | 32.6 |
| NO. 274 | +4C | | E342K | | |
| SEQ | −7C, +4G | CUGCUAGCUGCUCUGUCC | SERPINA1 | Rule 3 | 32.6 |
| NO. 275 | | | E342K | | |
| SEQ | −4C, +3C, | CUGGUACCUGCUCCUCCC | SERPINA1 | | 32.6 |
| NO. 276 | +5C | | E342K | | |
| SEQ | −7U, +3C, | CUGUUAGCUGCUCCCUCC | SERPINA1 | Rule 3 | 32.6 |
| NO. 277 | +4C | | E342K | | |
| SEQ | −7U, +7U | CUGUUAGCUGCUCUUUCU | SERPINA1 | Rule 3 | 32.5 |
| NO. 278 | | | E342K | | |
| SEQ | −7C, −2A, | CUGCUAGCAGCUCUCUCC | SERPINA1 | Rule 3 | 32.2 |
| NO. 279 | +4C | | E342K | | |
| SEQ | −10U, −3G, | UUGGUAGGUGCUCAUACC | SERPINA1 | | 32.1 |
| NO. 280 | +3A, +5A | | E342K | | |
| SEQ | −7C, −3U, | CUGCUAGUUGCCCUUUCC | SERPINA1 | Rule 3 | 31.8 |
| NO. 281 | +1C | | E342K | | |
| SEQ | −10A, −7C, | AUGCUAGCUGCUCUUUCU | SERPINA1 | Rule 3 | 31.8 |
| NO. 282 | +7U | | E342K | | |
| SEQ | −7C, +4C, | CUGCUAGCUGCUCUCUCG | SERPINA1 | Rule 3 | 31.6 |
| NO. 283 | +7G | | E342K | | |
| SEQ | −7C, +4G, | CUGCUAGCUGCUCUGACC | SERPINA1 | Rule 3 | 31.6 |
| NO. 284 | +5A | | E342K | | |
| SEQ | −10G, −7C, | GUGCUAGCUGCUCCUUCC | SERPINA1 | Rule 3 | 31.6 |
| NO. 285 | +3C | | E342K | | |
| SEQ | −9A, +4A, | CAGGUAGCUGCUCUAUUC | SERPINA1 | | 31.2 |
| NO. 286 | +6U | | E342K | | |
| SEQ | −7C, +5C, | CUGCUAGCUGCUCUUCGC | SERPINA1 | Rule 3 | 31 |
| NO. 287 | +6G | | E342K | | |
| SEQ | −7C, +7A | CUGCUAGCUGCUCUUUCA | SERPINA1 | Rule 3 | 31 |
| NO. 288 | | | E342K | | |
| SEQ | −7C, +4A | CUGCUAGCUGCUCUAUCC | SERPINA1 | Rule 3 | 30.9 |
| NO. 289 | | | E342K | | |
| SEQ | −7C, +5A, | CUGCUAGCUGCUCUUAUC | SERPINA1 | Rule 3 | 30.6 |
| NO. 290 | +6U | | E342K | | |
| SEQ | −7U, +4A, | CUGUUAGCUGCUCUAUCA | SERPINA1 | Rule 3 | 30.2 |
| NO. 291 | +7A | | E342K | | |
| SEQ | 0U, +2C, | CACCUACUCGUGCCAGCC | GFER | Rule 6 | 94.4 |
| NO. 292 | +4A, +7C | | R194H | | |
| SEQ | −9C, −8U, | CCUCUAGUCGCGACCGCU | GFER | | 40 |
| NO. 293 | −4G, +7U | | R194H | | |
| SEQ | −6C, +5C | CACCCACUCGCGACCCCG | GFER | | 39.3 |
| NO. 294 | | | R194H | | |
| SEQ | −4G, +6A | CACCUAGUCGCGACCGAG | GFER | | 35.5 |
| NO. 295 | | | R194H | | |
| SEQ | −3A, +6G | CACCUACACGCGACCGGG | GFER | | 33.3 |
| NO. 296 | | | R194H | | |
| SEQ | −4G, +3A, | CACCUAGUCGCGAACUUG | GFER | | 33.3 |
| NO. 297 | +5U, +6U | | R194H | | |
| SEQ | −8U, +2C | CAUCUACUCGCGCCCGCG | GFER | Rule 6 | 31.9 |
| NO. 298 | | | R194H | | |
| SEQ | −3A, +5A, | CACCUACACGCGACCACC | GFER | | 30.2 |
| NO. 299 | +7C | | R194H | | |
| SEQ | −3A, +2U, | CACCUACACGCGUCAGCG | GFER | Rule 6 | 30 |
| NO. 300 | +4A | | R194H | | |
| SEQ | −9U, +5C | CUCCUACUCGCGACCCCG | GFER | | 30 |
| NO. 301 | | | R194H | | |
| SEQ | −10G, −8G, | GAGCUACUCGCGACCGCA | GFER | | 29.8 |
| NO. 302 | +7A | | R194H | | |
| SEQ | −8G, +5A | CAGCUACUCGCGACCACG | GFER | | 29.6 |
| NO. 303 | | | R194H | | |
| SEQ | −9G, −8A, | CGACUACUCGCGAGCGCG | GFER | | 29.4 |
| NO. 304 | +3G | | R194H | | |
| SEQ | +2U, +6A | CACCUACUCGCGUCCGAG | GFER | Rule 6 | 28.9 |
| NO. 305 | | | R194H | | |
| SEQ | +4A, +7U | CACCUACUCGCGACAGCU | GFER | | 28.8 |
| NO. 306 | | | R194H | | |
| SEQ | −3C, +6G | CACCUACCCGCGACCGGG | GFER | | 28.6 |
| NO. 307 | | | R194H | | |
| SEQ | −10A, −9C, | ACCCUACUCGCGACGGCU | GFER | | 28.3 |
| NO. 308 | +4G, +7U | | R194H | | |
| SEQ | −10G, +6A | GACCUACUCGCGACCGAG | GFER | | 28.2 |
| NO. 309 | | | R194H | | |
| SEQ | −10U, −7A | UACAUACUCGCGACCGCG | GFER | | 27.6 |
| NO. 310 | | | R194H | | |
| SEQ | −9C, +2C, | CCCCUACUCGCGCCCGAG | GFER | Rule 6 | 27.5 |
| NO. 311 | +6A | | R194H | | |
| SEQ | −9G, −8U, | CGUCUACUCGCGUCCGCG | GFER | Rule 6 | 27.4 |
| NO. 312 | +2U | | R194H | | |
| SEQ | −10U, −6C, | UACCCACUCGCGCCCGCG | GFER | Rule 6 | 27 |
| NO. 313 | +2C | | R194H | | |
| SEQ | −4G, +4G, | CACCUAGUCGCGACGGCU | GFER | | 26.9 |
| NO. 314 | +7U | | R194H | | |
| SEQ | −10U, +6A | UACCUACUCGCGACCGAG | GFER | | 26.8 |
| NO. 315 | | | R194H | | |
| SEQ | −10U, +4G, | UACCUACUCGCGACGGAG | GFER | | 26.7 |
| NO. 316 | +6A | | R194H | | |
| SEQ | −9C, −8A, | CCACUACACGCGACCGCG | GFER | | 26.7 |
| NO. 317 | −3A | | R194H | | |
| SEQ | −6C, +7U | CACCCACUCGCGACCGCU | GFER | | 26.6 |
| NO. 318 | | | R194H | | |
| SEQ | −6C, +2C | CACCCACUCGCGCCCGCG | GFER | Rule 6 | 26.5 |
| NO. 319 | | | R194H | | |
| SEQ | −10A, −9G, | AGCCUACUCGCGACCGAG | GFER | | 26.3 |
| NO. 320 | +6A | | R194H | | |
| SEQ | −9C, +4A | CCCCUACUCGCGACAGCG | GFER | | 26.3 |
| NO. 321 | | | R194H | | |
| SEQ | −10G, +5A | GACCUACUCGCGACCACG | GFER | | 26.2 |
| NO. 322 | | | R194H | | |
| SEQ | −7A, +4A, | CACAUACUCGCGACAUCG | GFER | | 26.2 |
| NO. 323 | +5U | | R194H | | |
| SEQ | −10G, −7U | GACUUACUCGCGACCGCG | GFER | | 26.1 |
| NO. 324 | | | R194H | | |
| SEQ | −4G, +2U, | CACCUAGUCGCGUCCGAG | GFER | Rule 6 | 26.1 |
| NO. 325 | +6A | | R194H | | |
| SEQ | +4U, +6G | CACCUACUCGCGACUGGG | GFER | | 25.9 |
| NO. 326 | | | R194H | | |
| SEQ | +2C, +6G | CACCUACUCGCGCCCGGG | GFER | Rule 6 | 25.8 |
| NO. 327 | | | R194H | | |
| SEQ | +2C | CACCUACUCGCGCCCGCG | GFER | Rule 6 | 25.6 |
| NO. 328 | | | R194H | | |
| SEQ | +2C, +6A | CACCUACUCGCGCCCGAG | GFER | Rule 6 | 25.5 |
| NO. 329 | | | R194H | | |
| SEQ | +2C, +7U | CACCUACUCGCGCCCGCU | GFER | Rule 6 | 25.5 |
| NO. 330 | | | R194H | | |
| SEQ | −10G, −7A, | GACAUACUCGCGACCGCA | GFER | | 25.4 |
| NO. 331 | +7A | | R194H | | |
| SEQ | −7U, +2U | CACUUACUCGCGUCCGCG | GFER | Rule 6 | 25.3 |
| NO. 332 | | | R194H | | |
| SEQ | −9G, −7A, | CGCAUACUCGCGCCCGGG | GFER | Rule 6 | 25.3 |
| NO. 333 | +2C, +6G | | R194H | | |
| SEQ | −9G, −7A | CGCAUACUCGCGACCGCG | GFER | | 25.2 |
| NO. 334 | | | R194H | | |
| SEQ | −6A, +2C, | CACCAACUCGCGCCUGCG | GFER | Rule 6 | 25 |
| NO. 335 | +4U | | R194H | | |
| SEQ | −4G, +5U | CACCUAGUCGCGACCUCG | GFER | | 25 |
| NO. 336 | | | R194H | | |
| SEQ | −10A, −8A, | AAACUACUCGCGACCGCA | GFER | | 25 |
| NO. 337 | +7A | | R194H | | |
| SEQ | −8U, +2C, | CAUCUACUCGCGCCCGCC | GFER | Rule 6 | 25 |
| NO. 338 | +7C | | R194H | | |
| SEQ | +6A, +7U | CACCUACUCGCGACCGAU | GFER | | 24.9 |
| NO. 339 | | | R194H | | |
| SEQ | −1G, +7A | GUCCGUAUGUGGCUUCACUA | ALDH2 | | 100 |
| NO. 340 | | | E504K | | |
| SEQ | +4G, +5A | GUCCGUAUGUGACUUCGAUU | ALDH2 | | 100 |
| NO. 341 | | | E504K | | |
| SEQ | −11G, −7C, | GGCCGCCUGUGACUUCACUU | ALDH2 | | 100 |
| NO. 342 | −6C | | E504K | | |
| SEQ | −8C, −1G | GUCCCUAUGUGGCUUCACUU | ALDH2 | Rule 2 | 95.7 |
| NO. 343 | | | E504K | | |
| SEQ | −11A, +4U | GACCGUAUGUGACUUCUCUU | ALDH2 | | 93.1 |
| NO. 344 | | | E504K | | |
| SEQ | −11A, +1C, | GACCGUAUGUGACCUCACUC | ALDH2 | | 92.9 |
| NO. 345 | +7C | | E504K | | |
| SEQ | −9U, −7G | GUCUGGAUGUGACUUCACUU | ALDH2 | | 92.3 |
| NO. 346 | | | E504K | | |
| SEQ | −12U, −8U | UUCCUUAUGUGACUUCACUU | ALDH2 | Rule 2 | 92.3 |
| NO. 347 | | | E504K | | |
| SEQ | −8A, +6C, | GUCCAUAUGUGACUUCACCG | ALDH2 | | 91.7 |
| NO. 348 | +7G | | E504K | | |
| SEQ | −2A, +4U | GUCCGUAUGUAACUUCUCUU | ALDH2 | | 90.9 |
| NO. 349 | | | E504K | | |
| SEQ | −8U, +7G | GUCCUUAUGUGACUUCACUG | ALDH2 | Rule 2 | 90 |
| NO. 350 | | | E504K | | |
| SEQ | −3C, +4C, | GUCCGUAUGCGACUUCCUUG | ALDH2 | | 90 |
| NO. 351 | +5U, +7G | | E504K | | |
| SEQ | −8A, +6G | GUCCAUAUGUGACUUCACGU | ALDH2 | | 88.2 |
| NO. 352 | | | E504K | | |
| SEQ | −3C, +4G, | GUCCGUAUGCGACUUCGCUA | ALDH2 | | 88.2 |
| NO. 353 | +7A | | E504K | | |
| SEQ | +6A, +7C | GUCCGUAUGUGACUUCACAC | ALDH2 | | 86.7 |
| NO. 354 | | | E504K | | |
| SEQ | −6U, −3C | GUCCGUUUGCGACUUCACUU | ALDH2 | | 86.4 |
| NO. 355 | | | E504K | | |
| SEQ | −9U, −7A | GUCUGAAUGUGACUUCACUU | ALDH2 | | 85.7 |
| NO. 356 | | | E504K | | |
| SEQ | −1G, +2G | GUCCGUAUGUGGCUGCACUU | ALDH2 | | 83.3 |
| NO. 357 | | | E504K | | |
| SEQ | −8C, +3G | GUCCCUAUGUGACUUGACUU | ALDH2 | Rule 2 | 83.3 |
| NO. 358 | | | E504K | | |
| SEQ | −9U, −8C | GUCUCUAUGUGACUUCACUU | ALDH2 | | 83.3 |
| NO. 359 | | | E504K | | |
| SEQ | −8U, +3A | GUCCUUAUGUGACUUAACUU | ALDH2 | Rule 2 | 82.3 |
| NO. 360 | | | E504K | | |
| SEQ | −8C, +5U | GUCCCUAUGUGACUUCAUUU | ALDH2 | Rule 2 | 82.1 |
| NO. 361 | | | E504K | | |
| SEQ | −12U, −10G | UUGCGUAUGUGACUUCACUU | ALDH2 | | 81.8 |
| NO. 362 | | | E504K | | |
| SEQ | −8U, −4C, | GUCCUUAUCUGACUUCGCUU | ALDH2 | Rule 2 | 81.8 |
| NO. 363 | +4G | | E504K | | |
| SEQ | −8C, +7A | GUCCCUAUGUGACUUCACUA | ALDH2 | Rule 2 | 81.8 |
| NO. 364 | | | E504K | | |
| SEQ | −1G, +5U | GUCCGUAUGUGGCUUCAUUU | ALDH2 | | 81.8 |
| NO. 365 | | | E504K | | |
| SEQ | −8U, +6G | GUCCUUAUGUGACUUCACGU | ALDH2 | Rule 2 | 81 |
| NO. 366 | | | E504K | | |
| SEQ | −6U, −5A, | GUCCGUUAGUGACUUCAGGC | ALDH2 | | 80 |
| NO. 367 | +5G, +6G, | | E504K | | |
| | +7C | | | | |
| SEQ | −8U, +2C, | GUCCUUAUGUGACUCCACUC | ALDH2 | Rule 2 | 80 |
| NO. 368 | +7C | | E504K | | |
| SEQ | −12A, −8C | AUCCCUAUGUGACUUCACUU | ALDH2 | Rule 2 | 80 |
| NO. 369 | | | E504K | | |
| SEQ | −12A, −9A | AUCAGUAUGUGACUUCACUU | ALDH2 | | 80 |
| NO. 370 | | | E504K | | |
| SEQ | −12A, −8C, | AUCCCUAUGUGACUUCAGUU | ALDH2 | Rule 2 | 79.3 |
| NO. 371 | +5G | | E504K | | |
| SEQ | −3A, +2C | GUCCGUAUGAGACUCCACUU | ALDH2 | | 79.2 |
| NO. 372 | | | E504K | | |
| SEQ | −12A, −9A, | AUCAGUAUGUGACUUCAAUU | ALDH2 | | 79 |
| NO. 373 | +5A | | E504K | | |
| SEQ | −9U, −8A, | GUCUAUAUGUGACUUCACGU | ALDH2 | | 79 |
| NO. 374 | +6G | | E504K | | |
| SEQ | −8C, +7C | GUCCCUAUGUGACUUCACUC | ALDH2 | Rule 2 | 78.6 |
| NO. 375 | | | E504K | | |
| SEQ | −11C, −7A | GCCCGAAUGUGACUUCACUU | ALDH2 | | 77.4 |
| NO. 376 | | | E504K | | |
| SEQ | −11C, −9U, | GCCUGUAUGUAACUUCACUU | ALDH2 | | 76.9 |
| NO. 377 | −2A | | E504K | | |
| SEQ | −11G, −3C | GGCCGUAUGCGACUUCACUU | ALDH2 | | 76.9 |
| NO. 378 | | | E504K | | |
| SEQ | −12U, −9A | UUCAGUAUGUGACUUCACUU | ALDH2 | | 76.9 |
| NO. 379 | | | E504K | | |
| SEQ | −7C, −2A, | GUCCGCAUGUAACUUCACUC | ALDH2 | | 76.5 |
| NO. 380 | +7C | | E504K | | |
| SEQ | −8C, +4C | GUCCCUAUGUGACUUCCCUU | ALDH2 | Rule 2 | 75 |
| NO. 381 | | | E504K | | |
| SEQ | +6C, +7A | GUCCGUAUGUGACUUCACCA | ALDH2 | | 75 |
| NO. 382 | | | E504K | | |
| SEQ | −7A, +2A | GUCCGAAUGUGACUACACUU | ALDH2 | | 75 |
| NO. 383 | | | E504K | | |
| SEQ | −11G, −8A | GGCCAUAUGUGACUUCACUU | ALDH2 | | 74.4 |
| NO. 384 | | | E504K | | |
| SEQ | −12C, −9A | CUCAGUAUGUGACUUCACUU | ALDH2 | | 73.7 |
| NO. 385 | | | E504K | | |
| SEQ | −8U, −7G | GUCCUGAUGUGACUUCACUU | ALDH2 | | 73.7 |
| NO. 386 | | | E504K | | |
| SEQ | −8A, +4C | GUCCAUAUGUGACUUCCCUU | ALDH2 | | 73.5 |
| NO. 387 | | | E504K | | |
| SEQ | −11G, −2A, | GGCCGUAUGUAACUUCCCUU | ALDH2 | | 73.3 |
| NO. 388 | +4C | | E504K | | |
| SEQ | −9U, −3C | GUCUGUAUGCGACUUCACUU | ALDH2 | | 73.3 |
| NO. 389 | | | E504K | | |
| SEQ | −12C, −5A | CUCCGUAAGUGACUUCACUU | ALDH2 | | 73.3 |
| NO. 390 | | | E504K | | |
| SEQ | −8A, +7C | GUCCAUAUGUGACUUCACUC | ALDH2 | | 72.7 |
| NO. 391 | | | E504K | | |
| SEQ | −6U, +6G | GUCCGUUUGUGACUUCACGU | ALDH2 | | 72.7 |
| NO. 392 | | | E504K | | |
| SEQ | −11C, −10U, | GCUCGUCUGUGACUUGACUU | ALDH2 | | 72.7 |
| NO. 393 | −6C, +3G | | E504K | | |
| SEQ | −12C, −11C, | CCUCGUAUGUGACUUCACUU | ALDH2 | | 72.7 |
| NO. 394 | −10U | | E504K | | |
| SEQ | −6C, −3C | GUCCGUCUGCGACUUCACUU | ALDH2 | | 71.9 |
| NO. 395 | | | E504K | | |
| SEQ | −12C, −9U | CUCUGUAUGUGACUUCACUU | ALDH2 | | 71.4 |
| NO. 396 | | | E504K | | |
| SEQ | −6C, +3U | GUCCGUCUGUGACUUUACUU | ALDH2 | | 71.4 |
| NO. 397 | | | E504K | | |
| SEQ | −9U, +3G, | GUCUGUAUGUGACUUGACCU | ALDH2 | | 71.4 |
| NO. 398 | +6C | | E504K | | |
| SEQ | −12A, +4C, | AUCCGUAUGUGACUUCCCGU | ALDH2 | | 71.4 |
| NO. 399 | +6G | | E504K | | |
| SEQ | −5G, +3U, | CAACAGUCCGCGAUAGGG | TP53 | | 89.2 |
| NO. 400 | +4A | | R175H | | |
| SEQ | −8U, −7U, | CAUUACUCCGCGGCGAGC | TP53 | | 83.5 |
| NO. 401 | +2G, +5A, | | R175H | | |
| | +7C | | | | |
| SEQ | −9G, +2U, | CGACACUCCGCGUCAGGA | TP53 | Rule 6, | 80.5 |
| NO. 402 | +4A, +7A | | R175H | Rule 7 | |
| SEQ | 0U, +1U, | CAACACUCCGUUAAAGGC | TP53 | | 78.8 |
| NO. 403 | +3A, +4A, | | R175H | | |
| | +7C | | | | |
| SEQ | −9U, −7U, | CUAUACUCCGUGAGAGGG | TP53 | | 72.9 |
| NO. 404 | 0U, +3G, | | R175H | | |
| | +4A | | | | |
| SEQ | −8C, −7G, | CACGUAACCGCGACAGGG | TP53 | Rule 7 | 47.1 |
| NO. 405 | −6U, −5A, | | R175H | | |
| | −4A, +4A | | | | |
| SEQ | −9G, −6G, | CGACGGUCCGCGACGGGG | TP53 | | 43.4 |
| NO. 406 | −5G | | R175H | | |
| SEQ | +2C, +4A | CAACACUCCGCGCCAGGG | TP53 | Rule 6, | 37.3 |
| NO. 407 | | | R175H | Rule 7 | |
| SEQ | −10A, −9C, | ACACACUCCGCGACAGGG | TP53 | Rule 7 | 34.3 |
| NO. 408 | +4A | | R175H | | |
| SEQ | +4A, +6A | CAACACUCCGCGACAGAG | TP53 | Rule 7 | 33.9 |
| NO. 409 | | | R175H | | |
| SEQ | −6C, +3G, | CAACCCUCCGCGAGAGCG | TP53 | | 33.3 |
| NO. 410 | +4A, +6C | | R175H | | |
| SEQ | −2A, −1A, | CAACACUCAAGCACAAGA | TP53 | | 33.3 |
| NO. 411 | 0G, +1C, | | R175H | | |
| | +4A, +5A, | | | | |
| | +7A | | | | |
| SEQ | +4A, +6A, | CAACACUCCGCGACAGAU | TP53 | Rule 7 | 33.1 |
| NO. 412 | +7U | | R175H | | |
| SEQ | +4A, +7U | CAACACUCCGCGACAGGU | TP53 | Rule 7 | 32.9 |
| NO. 413 | | | R175H | | |
| SEQ | −4C, +4A, | CAACACCCCGCGACAGGC | TP53 | Rule 7 | 32.4 |
| NO. 414 | +7C | | R175H | | |
| SEQ | +2U, +4A, | CAACACUCCGCGUCAGGU | TP53 | Rule 6, | 32.2 |
| NO. 415 | +7U | | R175H | Rule 7 | |
| SEQ | +2C, +4A, | CAACACUCCGCGCCAGAG | TP53 | Rule 6, | 32 |
| NO. 416 | +6A | | R175H | Rule 7 | |
| SEQ | −8G, +2U, | CAGCACUCCGCGUCAGGG | TP53 | Rule 6, | 31.8 |
| NO. 417 | +4A | | R175H | Rule 7 | |
| SEQ | −5U, −4C, | CAACAUCCCGCGACAGCG | TP53 | Rule 7 | 31.8 |
| NO. 418 | +4A, +6C | | R175H | | |
| SEQ | −4C, +4A, | CAACACCCCGCGACAGAG | TP53 | Rule 7 | 31.6 |
| NO. 419 | +6A | | R175H | | |
| SEQ | +2G, +4A, | CAACACUCCGCGGCAGCG | TP53 | Rule 7 | 31.6 |
| NO. 420 | +6C | | R175H | | |
| SEQ | +2C, +4A, | CAACACUCCGCGCCAGGC | TP53 | Rule 6, | 31.4 |
| NO. 421 | +7C | | R175H | Rule 7 | |
| SEQ | +2G, +4A, | CAACACUCCGCGGCAGUA | TP53 | Rule 7 | 31.2 |
| NO. 422 | +6U, +7A | | R175H | | |
| SEQ | −9G, +4A, | CGACACUCCGCGACAGAA | TP53 | Rule 7 | 31.2 |
| NO. 423 | +6A, +7A | | R175H | | |
| SEQ | +4A, +6U, | CAACACUCCGCGACAGUU | TP53 | Rule 7 | 30.9 |
| NO. 424 | +7U | | R175H | | |
| SEQ | −9G, +2C, | CGACACUCCGCGCCAGGG | TP53 | Rule 6, | 30.9 |
| NO. 425 | +4A | | R175H | Rule 7 | |
| SEQ | +4A, +6U | CAACACUCCGCGACAGUG | TP53 | Rule 7 | 30.9 |
| NO. 426 | | | R175H | | |
| SEQ | +4A | CAACACUCCGCGACAGGG | TP53 | Rule 7 | 30.7 |
| NO. 427 | | | R175H | | |
| SEQ | −10U, +2C, | UAACACUCCGCGCCAGAG | TP53 | Rule 6, | 30.6 |
| NO. 428 | +4A, +6A | | R175H | Rule 7 | |
| SEQ | −7G, +4A, | CAAGACUCCGCGACAGGC | TP53 | Rule 7 | 30.3 |
| NO. 429 | +7C | | R175H | | |
| SEQ | −3U, +4A, | CAACACUUCGCGACAGUG | TP53 | Rule 7 | 30 |
| NO. 430 | +6U | | R175H | | |
| SEQ | +2C, +4A, | CAACACUCCGCGCCAGUG | TP53 | Rule 6, | 30 |
| NO. 431 | +6U | | R175H | Rule 7 | |
| SEQ | −10U, −4C, | UAACACCCCGCGCCAGGG | TP53 | Rule 6, | 30 |
| NO. 432 | +2C, +4A | | R175H | Rule 7 | |
| SEQ | −7G, +4A, | CAAGACUCCGCGACAGAG | TP53 | Rule 7 | 30 |
| NO. 433 | +6A | | R175H | | |
| SEQ | +2G, +4A, | CAACACUCCGCGGCAGAG | TP53 | Rule 7 | 29.8 |
| NO. 434 | +6A | | R175H | | |
| SEQ | +4A, +6A, | CAACACUCCGCGACAGAA | TP53 | Rule 7 | 29.7 |
| NO. 435 | +7A | | R175H | | |
| SEQ | −6C, −5A, | CAACCAUCCGCGACAGCG | TP53 | Rule 7 | 29.6 |
| NO. 436 | +4A, +6C | | R175H | | |
| SEQ | −10U, +4A, | UAACACUCCGCGACAGGU | TP53 | Rule 7 | 29.4 |
| NO. 437 | +7U | | R175H | | |
| SEQ NO. 438 | | CAACACUCCGCGACGGGG | TP53 | | 29.4 |
| | | | R175H | | |
| SEQ | +4A, +7C | CAACACUCCGCGACAGGC | TP53 | Rule 7 | 29.3 |
| NO. 439 | | | R175H | | |
| SEQ | +2C, +4A, | CAACACUCCGCGCCAGGU | TP53 | Rule 6, | 29.3 |
| NO. 440 | +7U | | R175H | Rule 7 | |
| SEQ | −5G, +4A, | CAACAGUCCGCGACAGUA | TP53 | Rule 7 | 29.2 |
| NO. 441 | +6U, +7A | | R175H | | |
| SEQ | +4A, +6C | CAACACUCCGCGACAGCG | TP53 | Rule 7 | 29 |
| NO. 442 | | | R175H | | |
| SEQ | +2U, +4A, | CAACACUCCGCGUCAGAG | TP53 | Rule 6, | 28.9 |
| NO. 443 | +6A | | R175H | Rule 7 | |
| SEQ | +4A, +6C, | CAACACUCCGCGACAGCC | TP53 | Rule 7 | 28.8 |
| NO. 444 | +7C | | R175H | | |
| SEQ | −10U, −4C, | UAACACCCCGCGACAGCA | TP53 | Rule 7 | 28.6 |
| NO. 445 | +4A, +6C, | | R175H | | |
| | +7A | | | | |
| SEQ | −7G, −4C, | CAAGACCCCGCGACAGGG | TP53 | Rule 7 | 28.6 |
| NO. 446 | +4A | | R175H | | |
| SEQ | −10U, +2C, | UAACACUCCGCGCCAGGG | TP53 | Rule 6, | 28.6 |
| NO. 447 | +4A | | R175H | Rule 7 | |
| SEQ | −10U, +4A, | UAACACUCCGCGACAGUG | TP53 | Rule 7 | 28.4 |
| NO. 448 | +6U | | R175H | | |
| SEQ | +2C, +4A, | CAACACUCCGCGCCAGCG | TP53 | Rule 6, | 28.4 |
| NO. 449 | +6C | | R175H | Rule 7 | |
| SEQ | −10G, +2C, | GAACACUCCGCGCCAGGG | TP53 | Rule 6, | 28.4 |
| NO. 450 | +4A | | R175H | Rule 7 | |
| SEQ | +4A, +6C, | CAACACUCCGCGACAGCU | TP53 | Rule 7 | 28.3 |
| NO. 451 | +7U | | R175H | | |
| SEQ | −4C, +4A, | CAACACCCCGCGACAGCG | TP53 | Rule 7 | 28.3 |
| NO. 452 | +6C | | R175H | | |
| SEQ | −7G, +2C, | CAAGACUCCGCGCCAGGG | TP53 | Rule 6, | 28.1 |
| NO. 453 | +4A | | R175H | Rule 7 | |
| SEQ | +2G, +4A, | CAACACUCCGCGGCAGUG | TP53 | Rule 7 | 28.1 |
| NO. 454 | +6U | | R175H | | |
| SEQ | −7U, −4C, | CAAUACCCCGCGACAGAG | TP53 | Rule 7 | 27.9 |
| NO. 455 | +4A, +6A | | R175H | | |
| SEQ | −4C, +4A, | CAACACCCCGCGACAGGU | TP53 | Rule 7 | 27.5 |
| NO. 456 | +7U | | R175H | | |
| SEQ | −7G, +4A | CAAGACUCCGCGACAGGG | TP53 | Rule 7 | 27.5 |
| NO. 457 | | | R175H | | |
| SEQ | +4A, +7A | CAACACUCCGCGACAGGA | TP53 | Rule 7 | 27.4 |
| NO. 458 | | | R175H | | |
| SEQ | −5U, −4C, | CAACAUCCCGCGACAGAG | TP53 | Rule 7 | 27.3 |
| NO. 459 | +4A, +6A | | R175H | | |
| SEQ | −6U, −4C, | CAACUCCCAGCGACAGGG | TP53 | Rule 7 | 27.3 |
| NO. 460 | −2A, +4A | | R175H | | |
| SEQ | −10U, −4C, | UAACACCCCGCGACAGAG | TP53 | Rule 7 | 27.3 |
| NO. 461 | +4A, +6A | | R175H | | |
| SEQ | −5U, +2G, | CAACAUUCCGCGGCAGGU | TP53 | Rule 7 | 27.3 |
| NO. 462 | +4A, +7U | | R175H | | |
| SEQ | −10U, +4A, | UAACACUCCGCGACAGCG | TP53 | Rule 7 | 27 |
| NO. 463 | +6C | | R175H | | |
| SEQ | −7G, +2G, | CAAGACUCCGCGGCAGGG | TP53 | Rule 7 | 26.7 |
| NO. 464 | +4A | | R175H | | |
| SEQ | −7G, +4A, | CAAGACUCCGCGACAGUG | TP53 | Rule 7 | 26.7 |
| NO. 465 | +6U | | R175H | | |
| SEQ | −7U, +4A | CAAUACUCCGCGACAGGG | TP53 | Rule 7 | 26.7 |
| NO. 466 | | | R175H | | |
| SEQ | −4C, +2C, | CAACACCCCGCGCCAGGG | TP53 | Rule 6, | 26.5 |
| NO. 467 | +4A | | R175H | Rule 7 | |
| SEQ | −9G, −5G, | CGACAGUCCGCGCCAGGC | TP53 | Rule 6, | 26.3 |
| NO. 468 | +2C, +4A, | | R175H | Rule 7 | |
| | +7C | | | | |
| SEQ | −9G, −8G, | CGGCACUCCGCGACAGGU | TP53 | Rule 7 | 26.3 |
| NO. 469 | +4A, +7U | | R175H | | |
| SEQ | −9C, +4A, | CCACACUCCGCGACAGAG | TP53 | Rule 7 | 26.3 |
| NO. 470 | +6A | | R175H | | |
| SEQ | −4C, +4A | CAACACCCCGCGACAGGG | TP53 | Rule 7 | 26.2 |
| NO. 471 | | | R175H | | |
| SEQ | +2G, +4A, | CAACACUCCGCGGCAGGU | TP53 | Rule 7 | 26 |
| NO. 472 | +7U | | R175H | | |
| SEQ | −7U, +4A, | CAAUACUCCGCGACAAGG | TP53 | | 25.9 |
| NO. 473 | +5A | | R175H | | |
| SEQ | +4A, +6U, | CAACACUCCGCGACAGUA | TP53 | Rule 7 | 25.8 |
| NO. 474 | +7A | | R175H | | |
| SEQ | −4C, +4A, | CAACACCCCGCGACAAGA | TP53 | | 25.8 |
| NO. 475 | +5A, +7A | | R175H | | |
| SEQ | +2U, +5A, | CAACACUCCGCGUCGAGU | TP53 | Rule 6 | 25.6 |
| NO. 476 | +7U | | R175H | | |
| SEQ | −7G, +4A, | CAAGACUCCGCGACAAGG | TP53 | | 25.6 |
| NO. 477 | +5A | | R175H | | |
| SEQ | −6G, +4A, | CAACGCUCCGCGACAGUG | TP53 | Rule 7 | 25.6 |
| NO. 478 | +6U | | R175H | | |
| SEQ | −6C, −5A, | CAACCAUCCGCGACAGGG | TP53 | Rule 7 | 25.4 |
| NO. 479 | +4A | | R175H | | |
| SEQ | +2U, +4A | CAACACUCCGCGUCAGGG | TP53 | Rule 6, | 25.3 |
| NO. 480 | | | R175H | Rule 7 | |
| SEQ | −9C, +2G, | CCACACUCCGCGGCAGGG | TP53 | Rule 7 | 25.2 |
| NO. 481 | +4A | | R175H | | |
| SEQ | +2C, +4A, | CAACACUCCGCGCCAGGA | TP53 | Rule 6, | 25.2 |
| NO. 482 | +7A | | R175H | Rule 7 | |
| SEQ | −3U, +4A | CAACACUUCGCGACAGGG | TP53 | Rule 7 | 25.1 |
| NO. 483 | | | R175H | | |
| SEQ | −10A, −8U, | AAUCACACCGCGACAGGG | TP53 | Rule 7 | 25 |
| NO. 484 | −4A, +4A | | R175H | | |
| SEQ | −10A, −5A, | AAACAAUCCGCGACAGGA | TP53 | Rule 7 | 25 |
| NO. 485 | +4A, +7A | | R175H | | |
| SEQ | −9C, −4C, | CCACACCCCGCGACAGGG | TP53 | Rule 7 | 25 |
| NO. 486 | +4A | | R175H | | |
| SEQ | −10A, −4A, | AAACACACCGCGCCAGGG | TP53 | Rule 6, | 25 |
| NO. 487 | +2C, +4A | | R175H | Rule 7 | |
| SEQ | +4A, +5A, | CAACACUCCGCGACAAAG | TP53 | | 25 |
| NO. 488 | +6A | | R175H | | |
| SEQ | −9G, +2U, | CGACACUCCGCGUCAGGC | TP53 | Rule 6, | 25 |
| NO. 489 | +4A, +7C | | R175H | Rule 7 | |
| SEQ | +2C, +4A, | CAACACUCCGCGCCAGAU | TP53 | Rule 6, | 25 |
| NO. 490 | +6A, +7U | | R175H | Rule 7 | |
| SEQ | +4A, +5A | CAACACUCCGCGACAAGG | TP53 | | 24.9 |
| NO. 491 | | | R175H | | |
| SEQ | −9C, +2C, | CCACACUCCGCGCCAGGG | TP53 | Rule 6, | 24.9 |
| NO. 492 | +4A | | R175H | Rule 7 | |
| SEQ | +2G, +4A | CAACACUCCGCGGCAGGG | TP53 | Rule 7 | 24.9 |
| NO. 493 | | | R175H | | |
| SEQ | +4A, +5U, | CAACACUCCGCGACAUGU | TP53 | | 24.8 |
| NO. 494 | +7U | | R175H | | |
| SEQ | +2C, +4A, | CAACACUCCGCGCCAAGG | TP53 | Rule 6 | 24.6 |
| NO. 495 | +5A | | R175H | | |
| SEQ | −10U, −9U, | UUACACUCCGCGCCAGGA | TP53 | Rule 6, | 24.5 |
| NO. 496 | +2C, +4A, | | R175H | Rule 7 | |
| | +7A | | | | |
| SEQ | −10U, +2C, | UAACACUCCGCGCCAAGG | TP53 | Rule 6 | 24.5 |
| NO. 497 | +4A, +5A | | R175H | | |
| SEQ | −10U, +4A | UAACACUCCGCGACAGGG | TP53 | Rule 7 | 24.4 |
| NO. 498 | | | R175H | | |
| SEQ | −10A, +2C, | AAACACUCCGCGCCAGGG | TP53 | Rule 6, | 24.4 |
| NO. 499 | +4A | | R175H | Rule 7 | |
| SEQ | −9C, −8U, | CCUCACUCCGCGACAGGG | TP53 | Rule 7 | 24.4 |
| NO. 500 | +4A | | R175H | | |
| SEQ | +4A, +5U | CAACACUCCGCGACAUGG | TP53 | | 24.4 |
| NO. 501 | | | R175H | | |
| SEQ | −7U, +2U, | CAAUACUCCGCGUCAGGG | TP53 | Rule 6, | 24.3 |
| NO. 502 | +4A | | R175H | Rule 7 | |
| SEQ | +2U, +4A, | CAACACUCCGCGUCAGCG | TP53 | Rule 6, | 24.3 |
| NO. 503 | +6C | | R175H | Rule 7 | |
| SEQ | −5G, +4A, | CAACAGUCCGCGACAGAG | TP53 | Rule 7 | 24.3 |
| NO. 504 | +6A | | R175H | | |
| SEQ | −8G, −5U, | CAGCAUUCCGCGCCAGGG | TP53 | Rule 6, | 24.2 |
| NO. 505 | +2C, +4A | | R175H | Rule 7 | |
| SEQ | −3U, +4A, | CAACACUUCGCGACAGAG | TP53 | Rule 7 | 24.2 |
| NO. 506 | +6A | | R175H | | |
| SEQ | −5G, +4A, | CAACAGUCCGCGACAUGG | TP53 | | 24.2 |
| NO. 507 | +5U | | R175H | | |
| SEQ | −5A, +4A, | CAACAAUCCGCGACAGCC | TP53 | Rule 7 | 24.1 |
| NO. 508 | +6C, +7C | | R175H | | |
| SEQ | −9C, +4A | CCACACUCCGCGACAGGG | TP53 | Rule 7 | 24.1 |
| NO. 509 | | | R175H | | |
| SEQ | +4A, +5A, | CAACACUCCGCGACAAGA | TP53 | | 24 |
| NO. 510 | +7A | | R175H | | |
| SEQ | −4A, +4A, | CAACACACCGCGACAGCG | TP53 | Rule 7 | 24 |
| NO. 511 | +6C | | R175H | | |
| SEQ | −10G, +4A, | GAACACUCCGCGACAGGA | TP53 | Rule 7 | 23.9 |
| NO. 512 | +7A | | R175H | | |
| SEQ | +2C, +5A | CAACACUCCGCGCCGAGG | TP53 | Rule 6 | 23.7 |
| NO. 513 | | | R175H | | |
| SEQ | −3U, +4A, | CAACACUUCGCGACAGGU | TP53 | Rule 7 | 23.6 |
| NO. 514 | +7U | | R175H | | |
| SEQ | −9C, −5U, | CCACAUUCCGCGACAGGG | TP53 | Rule 7 | 23.6 |
| NO. 515 | +4A | | R175H | | |
| SEQ | −10A, +4A | AAACACUCCGCGACAGGG | TP53 | Rule 7 | 23.6 |
| NO. 516 | | | R175H | | |
| SEQ | −10G, −9C, | GCACACUCCGCGACAGGU | TP53 | Rule 7 | 23.5 |
| NO. 517 | +4A, +7U | | R175H | | |
| SEQ | −9G, −4A, | CGACACACCGCGACAGGG | TP53 | Rule 7 | 23.5 |
| NO. 518 | +4A | | R175H | | |
| SEQ | −3A, −1G, | GAGGGUGACGCCAUGUAC | SCN9A | | 100 |
| NO. 519 | +2A | | W897X | | |
| SEQ | −10A, −4C, | AAGGGUCCCACCGCGUAC | SCN9A | | 91.7 |
| NO. 520 | +3C | | W897X | | |
| SEQ | −9G, −6C, | GGGGCUGCCACCGUGUGC | SCN9A | Rule 4 | 90.9 |
| NO. 521 | +6G | | W897X | | |
| SEQ | −10A, −8A, | AAAGGUGCCACCGAGUUC | SCN9A | | 90.9 |
| NO. 522 | +3A, +6U | | W897X | | |
| SEQ | −6U, +4A, | GAGGUUGCCACCGUAUGC | SCN9A | Rule 4 | 90.6 |
| NO. 523 | +6G | | W897X | | |
| SEQ | −6C, +4C | GAGGCUGCCACCGUCUAC | SCN9A | Rule 4 | 88.2 |
| NO. 524 | | | W897X | | |
| SEQ | −6C, −3U, | GAGGCUGUCACCGCGUAC | SCN9A | Rule 4 | 85.2 |
| NO. 525 | +3C | | W897X | | |
| SEQ | −6C, +6U, | GAGGCUGCCACCGUGUUA | SCN9A | Rule 4 | 84.4 |
| NO. 526 | +7A | | W897X | | |
| SEQ | −6C, −3A, | GAGGCUGACACCGUGUUC | SCN9A | Rule 4 | 83.3 |
| NO. 527 | +6U | | W897X | | |
| SEQ | −6U, −3A, | GAGGUUGACACCGUGUAU | SCN9A | Rule 4 | 82.3 |
| NO. 528 | +7U | | W897X | | |
| SEQ | −6C, +2U, | GAGGCUGCCACCUUGUGC | SCN9A | Rule 4 | 82.3 |
| NO. 529 | +6G | | W897X | | |
| SEQ | −6C, +6C, | GAGGCUGCCACCGUGUCA | SCN9A | Rule 4 | 82 |
| NO. 530 | +7A | | W897X | | |
| SEQ | −10C, −5C, | CAGGGCGCCACCGAGUAC | SCN9A | | 81.8 |
| NO. 531 | +3A | | W897X | | |
| SEQ | −10A, −6C, | AAGGCUGCCACCGUGUGC | SCN9A | Rule 4 | 81.8 |
| NO. 532 | +6G | | W897X | | |
| SEQ | +3A, +4A, | GAGGGUGCCACCGAAGAC | SCN9A | | 81.8 |
| NO. 533 | +5G | | W897X | | |
| SEQ | −6C, +5C, | GAGGCUGCCACCGUGCAA | SCN9A | Rule 4 | 81.8 |
| NO. 534 | +7A | | W897X | | |
| SEQ | −10C, +2C, | CAGGGUGCCACCCUGUAA | SCN9A | | 81.8 |
| NO. 535 | +7A | | W897X | | |
| SEQ | −8A, +4A, | GAAGGUGCCACCGUAUAU | SCN9A | | 81.8 |
| NO. 536 | +7U | | W897X | | |
| SEQ | +2C, +6C, | GAGGGUGCCACCCUGUCA | SCN9A | | 81.2 |
| NO. 537 | +7A | | W897X | | |
| SEQ | −9G, −8A, | GGAGGUGCCACCGUCUAG | SCN9A | | 81.2 |
| NO. 538 | +4C, +7G | | W897X | | |
| SEQ | −9U, −6U, | GUGGUUGCCACCGUGUAA | SCN9A | Rule 4 | 80.8 |
| NO. 539 | +7A | | W897X | | |
| SEQ | −6C, +6U | GAGGCUGCCACCGUGUUC | SCN9A | Rule 4 | 80.7 |
| NO. 540 | | | W897X | | |
| SEQ | −6C, +7U | GAGGCUGCCACCGUGUAU | SCN9A | Rule 4 | 80.4 |
| NO. 541 | | | W897X | | |
| SEQ | −10A, −6U, | AAGGUUGCCACCGUGUAU | SCN9A | Rule 4 | 80 |
| NO. 542 | +7U | | W897X | | |
| SEQ | −7A, +2A, | GAGAGUGCCACCAUGUAU | SCN9A | | 80 |
| NO. 543 | +7U | | W897X | | |
| SEQ | −9G, −6C, | GGGGCUGGCACCGUGUAC | SCN9A | Rule 4 | 80 |
| NO. 544 | −3G | | W897X | | |
| SEQ | −6C, −3G, | GAGGCUGGCACCGUGGAA | SCN9A | Rule 4 | 79.2 |
| NO. 545 | +5G, +7A | | W897X | | |
| SEQ | −4A, +2C, | GAGGGUACCACCCUGUUC | SCN9A | | 79.2 |
| NO. 546 | +6U | | W897X | | |
| SEQ | −6C, −3U, | GAGGCUGUCACCGAGUAC | SCN9A | Rule 4 | 79.1 |
| NO. 547 | +3A | | W897X | | |
| SEQ | −6C, +7A | GAGGCUGCCACCGUGUAA | SCN9A | Rule 4 | 79 |
| NO. 548 | | | W897X | | |
| SEQ | −10C, −6C, | CAGGCUGCCACCUUGUAC | SCN9A | Rule 4 | 78.6 |
| NO. 549 | +2U | | W897X | | |
| SEQ | −9G, +3A, | GGGGGUGCCACCGAGUGC | SCN9A | | 78.6 |
| NO. 550 | +6G | | W897X | | |
| SEQ | −10A, −6C, | AAGGCUGCCACCGUGUAA | SCN9A | Rule 4 | 78.4 |
| NO. 551 | +7A | | W897X | | |
| SEQ | −9G, −6C, | GGGGCUGCCACCCUGUAC | SCN9A | Rule 4 | 78.3 |
| NO. 552 | +2C | | W897X | | |
| SEQ | −6C, +2C, | GAGGCUGCCACCCUGUAU | SCN9A | Rule 4 | 77.8 |
| NO. 553 | +7U | | W897X | | |
| SEQ | −6C, +4U | GAGGCUGCCACCGUUUAC | SCN9A | Rule 4 | 77.8 |
| NO. 554 | | | W897X | | |
| SEQ | −10U, +3C, | UAGGGUGCCACCGCGUGC | SCN9A | | 77.8 |
| NO. 555 | +6G | | W897X | | |
| SEQ | −6C, −3G, | GAGGCUGGCACCGUGUAA | SCN9A | Rule 4 | 76.9 |
| NO. 556 | +7A | | W897X | | |
| SEQ | −9G, +3A, | GGGGGUGCCACCGAGUAU | SCN9A | | 76.9 |
| NO. 557 | +7U | | W897X | | |
| SEQ | −10U, −6C, | UAGGCUGCCACCGUGUAG | SCN9A | Rule 4 | 76.9 |
| NO. 558 | +7G | | W897X | | |
| SEQ | −6U, −3G, | GAGGUUGGCACCGAGUGC | SCN9A | Rule 4 | 76.5 |
| NO. 559 | +3A, +6G | | W897X | | |
| SEQ | −6C, −3U, | GAGGCUGUCACCGUGCAC | SCN9A | Rule 4 | 76.2 |
| NO. 560 | +5C | | W897X | | |
| SEQ | −6C, +7G | GAGGCUGCCACCGUGUAG | SCN9A | Rule 4 | 75.8 |
| NO. 561 | | | W897X | | |
| SEQ | +3A, +6C | GAGGGUGCCACCGAGUCC | SCN9A | | 75.8 |
| NO. 562 | | | W897X | | |
| SEQ | −9G, −8U, | GGUGGUGCCACCGUGUGC | SCN9A | | 75 |
| NO. 563 | +6G | | W897X | | |
| SEQ | −10U, −6C, | UAGGCUGCCACCGGGUAA | SCN9A | Rule 4 | 75 |
| NO. 564 | +3G, +7A | | W897X | | |
| SEQ | −5C, +1G, | GAGGGCGCCACGGUGGAC | SCN9A | | 75 |
| NO. 565 | +5G | | W897X | | |
| SEQ | −6U, +4C, | GAGGUUGCCACCGUCUUC | SCN9A | Rule 4 | 75 |
| NO. 566 | +6U | | W897X | | |
| SEQ | −6U, +4A | GAGGUUGCCACCGUAUAC | SCN9A | Rule 4 | 75 |
| NO. 567 | | | W897X | | |
| SEQ | −9G, +1G, | GGGGGUGCCACGGUGUAU | SCN9A | | 75 |
| NO. 568 | +7U | | W897X | | |
| SEQ | −8U, −4U | GAUGGUUCCACCGUGUAC | SCN9A | | 75 |
| NO. 569 | | | W897X | | |
| SEQ | −6C, −3U, | GAGGCUGUCACCCUGUAC | SCN9A | Rule 4 | 75 |
| NO. 570 | +2C | | W897X | | |
| SEQ | −6C, −3G, | GAGGCUGGCACCGUGUCC | SCN9A | Rule 4 | 75 |
| NO. 571 | +6C | | W897X | | |
| SEQ | −8A, +2A, | GAAGGUGCCACCAUUUAC | SCN9A | | 75 |
| NO. 572 | +4U | | W897X | | |
| SEQ | −6C, −3A, | GAGGCUGACACCGUGUUA | SCN9A | Rule 4 | 74.5 |
| NO. 573 | +6U, +7A | | W897X | | |
| SEQ | −6C, +3A | GAGGCUGCCACCGAGUAC | SCN9A | Rule 4 | 74.3 |
| NO. 574 | | | W897X | | |
| SEQ | −6U, +2U | GAGGUUGCCACCUUGUAC | SCN9A | Rule 4 | 74.2 |
| NO. 575 | | | W897X | | |
| SEQ | −6C, +4A | GAGGCUGCCACCGUAUAC | SCN9A | Rule 4 | 74.1 |
| NO. 576 | | | W897X | | |
| SEQ | −10A, −6U, | AAGGUUGCCACCGUGUUA | SCN9A | Rule 4 | 74.1 |
| NO. 577 | +6U, +7A | | W897X | | |
| SEQ | −6C, +5G | GAGGCUGCCACCGUGGAC | SCN9A | Rule 4 | 74 |
| NO. 578 | | | W897X | | |
| SEQ | −6C, +5A | GAGGCUGCCACCGUGAAC | SCN9A | Rule 4 | 73.8 |
| NO. 579 | | | W897X | | |
| SEQ | −6C, +3C, | GAGGCUGCCACCGCGUUC | SCN9A | Rule 4 | 73.8 |
| NO. 580 | +6U | | W897X | | |
| SEQ | −4A, +3C, | GAGGGUACCACCGCGUAU | SCN9A | | 73.7 |
| NO. 581 | +7U | | W897X | | |
| SEQ | −6U, +3A | GAGGUUGCCACCGAGUAC | SCN9A | Rule 4 | 73.4 |
| NO. 582 | | | W897X | | |
| SEQ | −6C, −3A, | GAGGCUGACACCGAGUAC | SCN9A | Rule 4 | 73.3 |
| NO. 583 | +3A | | W897X | | |
| SEQ | −10C, +4U, | CAGGGUGCCACCGUUUAA | SCN9A | | 73.3 |
| NO. 584 | +7A | | W897X | | |
| SEQ | −6U, +2A | GAGGUUGCCACCAUGUAC | SCN9A | Rule 4 | 73.3 |
| NO. 585 | | | W897X | | |
| SEQ | −6C, −3G, | GAGGCUGGCACCGUGCAC | SCN9A | Rule 4 | 73 |
| NO. 586 | +5C | | W897X | | |
| SEQ | −6C, +2C | GAGGCUGCCACCCUGUAC | SCN9A | Rule 4 | 72.9 |
| NO. 587 | | | W897X | | |
| SEQ | −9G, −8A, | GGAGGUGCCACCGAGUAC | SCN9A | | 72.7 |
| NO. 588 | +3A | | W897X | | |
| SEQ | −7U, −4U, | GAGUGUUCCACCGGGUAC | SCN9A | | 72.7 |
| NO. 589 | +3G | | W897X | | |
| SEQ | −6C | GAGGCUGCCACCGUGUAC | SCN9A | Rule 4 | 72.4 |
| NO. 590 | | | W897X | | |
| SEQ | −6C, +3C | GAGGCUGCCACCGCGUAC | SCN9A | Rule 4 | 71.8 |
| NO. 591 | | | W897X | | |
| SEQ | −10A, −6C, | AAGGCUGCCACCCUGUAC | SCN9A | Rule 4 | 71.7 |
| NO. 592 | +2C | | W897X | | |
| SEQ | −9U, −6C, | GUGGCUGCCACCGUGUAU | SCN9A | Rule 4 | 71.4 |
| NO. 593 | +7U | | W897X | | |
| SEQ | −10A, −9C, | ACGAUAGGAACCUGAGUC | HBB | | 80.9 |
| NO. 594 | −5A, +4A | | W38X | | |
| SEQ | +3C, +7G | CAGAUGGGAACCUCGGUG | HBB | | 79.5 |
| NO. 595 | | | W38X | | |
| SEQ | −10U, −5A, | UAGAUAGGAACCUGGAUC | HBB | | 79.4 |
| NO. 596 | +5A | | W38X | | |
| SEQ | −6C, +2G | CAGACGGGAACCGGGGUC | HBB | | 78.2 |
| NO. 597 | | | W38X | | |
| SEQ | −9G, +6A | CGGAUGGGAACCUGGGAC | HBB | | 77.7 |
| NO. 598 | | | W38X | | |
| SEQ | −8C, +4A, | CACAUGGGAACCUGAAUC | HBB | | 76.8 |
| NO. 599 | +5A | | W38X | | |
| SEQ | −5A, +6A, | CAGAUAGGAACCUGGGAA | HBB | | 76.5 |
| NO. 600 | +7A | | W38X | | |
| SEQ | −4C, +6C | CAGAUGCGAACCUGGGCC | HBB | | 76.3 |
| NO. 601 | | | W38X | | |
| SEQ | −8C, +3A, | CACAUGGGAACCUAGGUU | HBB | | 76.2 |
| NO. 602 | +7U | | W38X | | |
| SEQ | −10G, +5C | GAGAUGGGAACCUGGCUC | HBB | | 75.5 |
| NO. 603 | | | W38X | | |
| SEQ | −8C, +4A | CACAUGGGAACCUGAGUC | HBB | | 75.4 |
| NO. 604 | | | W38X | | |
| SEQ | −5A, +7U | CAGAUAGGAACCUGGGUU | HBB | | 74.9 |
| NO. 605 | | | W38X | | |
| SEQ | −5A, +5U | CAGAUAGGAACCUGGUUC | HBB | | 74.9 |
| NO. 606 | | | W38X | | |
| SEQ | −8C, +3A, | CACAUGGGAACCUAGGUA | HBB | | 74.7 |
| NO. 607 | +7A | | W38X | | |
| SEQ | −5A, +5C, | CAGAUAGGAACCUGGCUA | HBB | | 74.7 |
| NO. 608 | +7A | | W38X | | |
| SEQ | −8U, +4A | CAUAUGGGAACCUGAGUC | HBB | | 74.5 |
| NO. 609 | | | W38X | | |
| SEQ | +3A, +7U | CAGAUGGGAACCUAGGUU | HBB | | 74.4 |
| NO. 610 | | | W38X | | |
| SEQ | −9G, +2C, | CGGAUGGGAACCCGGGGA | HBB | | 74.3 |
| NO. 611 | +6G, +7A | | W38X | | |
| SEQ | −9C, −5A, | CCGAUAGGAACCCGGGUC | HBB | | 74.3 |
| NO. 612 | +2C | | W38X | | |
| SEQ | +3A, +7A | CAGAUGGGAACCUAGGUA | HBB | | 74.2 |
| NO. 613 | | | W38X | | |
| SEQ | −9G, −5A, | CGGAUAGGAACCUGGGCC | HBB | | 73.6 |
| NO. 614 | +6C | | W38X | | |
| SEQ | −5A, +3A | CAGAUAGGAACCUAGGUC | HBB | | 73.6 |
| NO. 615 | | | W38X | | |
| SEQ | −5A, +4A | CAGAUAGGAACCUGAGUC | HBB | | 73.5 |
| NO. 616 | | | W38X | | |
| SEQ | −5A, +5A | CAGAUAGGAACCUGGAUC | HBB | | 73.5 |
| NO. 617 | | | W38X | | |
| SEQ | −5A, −3U, | CAGAUAGUAACCUGGUUC | HBB | | 73.2 |
| NO. 618 | +5U | | W38X | | |
| SEQ | −5A, +6C | CAGAUAGGAACCUGGGCC | HBB | | 73.2 |
| NO. 619 | | | W38X | | |
| SEQ | −8C, +3A | CACAUGGGAACCUAGGUC | HBB | | 73 |
| NO. 620 | | | W38X | | |
| SEQ | −3A, +6C | CAGAUGGAAACCUGGGCC | HBB | | 72.9 |
| NO. 621 | | | W38X | | |
| SEQ | −8A, +3A | CAAAUGGGAACCUAGGUC | HBB | | 72.8 |
| NO. 622 | | | W38X | | |
| SEQ | −4U, +3C | CAGAUGUGAACCUCGGUC | HBB | | 72.8 |
| NO. 623 | | | W38X | | |
| SEQ | −8A, +2C, | CAAAUGGGAACCCGAGUC | HBB | | 72.7 |
| NO. 624 | +4A | | W38X | | |
| SEQ | +3C, +7U | CAGAUGGGAACCUCGGUU | HBB | | 72.7 |
| NO. 625 | | | W38X | | |
| SEQ | −7C, +2C, | CAGCUGGGAACCCGGGUA | HBB | | 72.5 |
| NO. 626 | +7A | | W38X | | |
| SEQ | −10G, +4A, | GAGAUGGGAACCUGAGUU | HBB | | 72.4 |
| NO. 627 | +7U | | W38X | | |
| SEQ | −5A, +3C | CAGAUAGGAACCUCGGUC | HBB | | 72.2 |
| NO. 628 | | | W38X | | |
| SEQ | +4A | CAGAUGGGAACCUGAGUC | HBB | | 72.2 |
| NO. 629 | | | W38X | | |
| SEQ | −4U, +5C | CAGAUGUGAACCUGGCUC | HBB | | 72.1 |
| NO. 630 | | | W38X | | |
| SEQ | +3A | CAGAUGGGAACCUAGGUC | HBB | | 72 |
| NO. 631 | | | W38X | | |
| SEQ | −10A, +3A, | AAGAUGGGAACCUAGGUG | HBB | | 71.9 |
| NO. 632 | +7G | | W38X | | |
| SEQ | +3A, +5A | CAGAUGGGAACCUAGAUC | HBB | | 71.9 |
| NO. 633 | | | W38X | | |
| SEQ | −5A, +2C | CAGAUAGGAACCCGGGUC | HBB | | 71.9 |
| NO. 634 | | | W38X | | |
| SEQ | −4A, +2U, | CAACCUAGACCAUCUCAU | KRAS | Rule 5 | 27.3 |
| NO. 635 | +4U | | G12D | | |
| SEQ | −3A, −1G, | CAACCUCAAGUACCGCAU | KRAS | | 27.3 |
| NO. 636 | 0U | | G12D | | |
| SEQ | −4U, +4A, | CAACCUUGACCACCAGAU | KRAS | | 25 |
| NO. 637 | +5G | | G12D | | |
| SEQ | −5C, +2U, | CAACCCCGACCAUCGCCU | KRAS | Rule 5 | 24.1 |
| NO. 638 | +6C | | G12D | | |
| SEQ | −10U, −4G, | UAACCUGGAGCACCGCAU | KRAS | | 23.1 |
| NO. 639 | −1G | | G12D | | |
| SEQ | −7U, +5G, | CAAUCUCGACCACCGGGU | KRAS | | 23.1 |
| NO. 640 | +6G | | G12D | | |
| SEQ | −4G, +5U, | CAACCUGGACCACCGUCU | KRAS | | 23.1 |
| NO. 641 | +6C | | G12D | | |
| SEQ | −6A, −3A, | CAACAUCAACCAUCGCUU | KRAS | Rule 5 | 20 |
| NO. 642 | +2U, +6U | | G12D | | |
| SEQ | −7G, −6A, | CAAGAUCGACCAUCGCAU | KRAS | Rule 5 | 20 |
| NO. 643 | +2U | | G12D | | |
| SEQ | +3G, +5G, | CAACCUCGACCACGGGGC | KRAS | | 19.4 |
| NO. 644 | +6G, +7C | | G12D | | |
| SEQ | −4A, +2U, | CAACCUAGACCAUCGCAG | KRAS | Rule 5 | 18.8 |
| NO. 645 | +7G | | G12D | | |
| SEQ | −1G, +2U | CAACCUCGAGCAUCGCAU | KRAS | Rule 5 | 18.5 |
| NO. 646 | | | G12D | | |
| SEQ | −7G, −1G, | CAAGCUCGAGCACCGCCU | KRAS | | 18.2 |
| NO. 647 | +6C | | G12D | | |
| SEQ | −10G, +6U | GAACCUCGACCACCGCUU | KRAS | | 18.2 |
| NO. 648 | | | G12D | | |
| SEQ | −10G, −5C, | GAACCCCGACCACCGGAU | KRAS | | 17.6 |
| NO. 649 | +5G | | G12D | | |
| SEQ | −9G, −8U, | CGUCCUCGACCACCGGAU | KRAS | | 17.2 |
| NO. 650 | +5G | | G12D | | |
| SEQ | −5C, +2U, | CAACCCCGACCAUCUCAU | KRAS | Rule 5 | 17.1 |
| NO. 651 | +4U | | G12D | | |
| SEQ | −5C, −3A | CAACCCCAACCACCGCAU | KRAS | | 16.1 |
| NO. 652 | | | G12D | | |
| SEQ | −9U, −6U, | CUACUUCGACCACCGCAC | KRAS | | 16 |
| NO. 653 | +7C | | G12D | | |
| SEQ | −8G, +5G | CAGCCUCGACCACCGGAU | KRAS | | 15.8 |
| NO. 654 | | | G12D | | |
| SEQ | −5C, +7A | CAACCCCGACCACCGCAA | KRAS | | 15.6 |
| NO. 655 | | | G12D | | |
| SEQ | −10U, −7U, | UAAUCUCGACCACCGCCU | KRAS | | 15.4 |
| NO. 656 | +6C | | G12D | | |
| SEQ | −6A, −5A, | CAACAACGACCACCGGAU | KRAS | | 15.4 |
| NO. 657 | +5G | | G12D | | |
| SEQ | −9C, +2U, | CCACCUCGACCAUCACAU | KRAS | Rule 5 | 13.9 |
| NO. 658 | +4A | | G12D | | |
| SEQ | −5C, +2U, | CAACCCCGACCAUCGCUA | KRAS | Rule 5 | 13.9 |
| NO. 659 | +6U, +7A | | G12D | | |
| SEQ | −6U, −5G, | CAACUGUGUCCAAGGCAU | KRAS | | 13.8 |
| NO. 660 | 4U−, −2U, | | G12D | | |
| | +2A, +3G | | | | |
| SEQ | −9U, −6A, | CUACAUCGACCACCUAAU | KRAS | | 13.3 |
| NO. 661 | +4U, +5A | | G12D | | |
| SEQ | −3A, −1G | CAACCUCAAGCACCGCAU | KRAS | | 13.3 |
| NO. 662 | | | G12D | | |
| SEQ | −10A, −4G, | AAACCUGGACCACCGCUU | KRAS | | 13.3 |
| NO. 663 | +6U | | G12D | | |
| SEQ | −9C, −4A, | CCACCUAGACCAUCGCAU | KRAS | Rule 5 | 12.9 |
| NO. 664 | +2U | | G12D | | |
| SEQ | −9C, −8C, | CCCCCACGACUACCGCAA | KRAS | | 12.5 |
| NO. 665 | −5A, 0U, | | G12D | | |
| | +7A | | | | |
| SEQ | −6U, +2U, | CAACUUCGACCAUGGCAU | KRAS | | 12.5 |
| NO. 666 | +3G | | G12D | | |
| SEQ | −9C, −5A, | CCACCACGACCAUCGCCU | KRAS | Rule 5 | 12.5 |
| NO. 667 | +2U, +6C | | G12D | | |
| SEQ | +6U, +7C | CAACCUCGACCACCGCUC | KRAS | | 12.5 |
| NO. 668 | | | G12D | | |
| SEQ | −5C, −1G, | CAACCCCGAGCACCGCAG | KRAS | | 12.5 |
| NO. 669 | +7G | | G12D | | |
| SEQ | −7G, +3G, | CAAGCUCGACCACGGAAU | KRAS | | 12.5 |
| NO. 670 | +5A | | G12D | | |
| SEQ | −8G, −1G, | CAGCCUCGAGCACAGCAU | KRAS | | 12.5 |
| NO. 671 | +3A | | G12D | | |
| SEQ | −10A, −3C, | AAACCUCCACCACAGCAU | KRAS | | 12.5 |
| NO. 672 | +3A | | G12D | | |
| SEQ | −10U, −8G, | UAGCCUCGACCAUCGCAU | KRAS | Rule 5 | 12.1 |
| NO. 673 | +2U | | G12D | | |
| SEQ | −9C, +2U, | CCACCUCGACCAUCGCCG | KRAS | Rule 5 | 12 |
| NO. 674 | +6C, +7G | | G12D | | |
| SEQ | −3A, +2U, | CAACCUCAACCAUGGCAU | KRAS | | 11.9 |
| NO. 675 | +3G | | G12D | | |
| SEQ | +5G, +6G | CAACCUCGACCACCGGGU | KRAS | | 11.9 |
| NO. 676 | | | G12D | | |
| SEQ | −5A, +5G, | CAACCACGACCACCGGGU | KRAS | | 11.8 |
| NO. 677 | +6G | | G12D | | |
| SEQ | −10U, −8U, | UAUACUCGACCACCGCAU | KRAS | | 11.8 |
| NO. 678 | −7A | | G12D | | |
| SEQ | −7A, −5A, | CAAACACCACCACCGCAU | KRAS | | 11.8 |
| NO. 679 | −3C | | G12D | | |
| SEQ | −9C, −6A, | CCACAGCGACCACCUCAU | KRAS | | 11.8 |
| NO. 680 | −5G, +4U | | G12D | | |
| SEQ | −1G, +4A | CAACCUCGAGCACCACAU | KRAS | | 11.8 |
| NO. 681 | | | G12D | | |
| SEQ | −9G, +2G, | CGACCUCGACCAGCGAAU | KRAS | | 11.8 |
| NO. 682 | +5A | | G12D | | |
| SEQ | −8G, +2U | CAGCCUCGACCAUCGCAU | KRAS | Rule 5 | 11.6 |
| NO. 683 | | | G12D | | |
| SEQ | −6A, +3U, | CAACAUCGACCACUGCGC | KRAS | | 11.5 |
| NO. 684 | +6G, +7C | | G12D | | |
| SEQ | −10A, −9G, | AGACCUCGACCAUCGUAU | KRAS | Rule 5 | 11.5 |
| NO. 685 | +2U, +5U | | G12D | | |
| SEQ | −3A, +2U, | CAACCUCAACCAUCACAU | KRAS | Rule 5 | 11.2 |
| NO. 686 | +4A | | G12D | | |
| SEQ | −4G, 0U, | CAACCUGGACUACCAGAU | KRAS | | 11.1 |
| NO. 687 | +4A, +5G | | G12D | | |
| SEQ | +4U, +5U | CAACCUCGACCACCUUAU | KRAS | | 10.8 |
| NO. 688 | | | G12D | | |
| SEQ | −9U, −8U, | CUUCCUCGACCACCAUAU | KRAS | | 10.8 |
| NO. 689 | +4A, +5U | | G12D | | |
| SEQ | −7U, −1G, | CAAUCUCGAGCACCGGAU | KRAS | | 10.8 |
| NO. 690 | +5G | | G12D | | |
| SEQ | −10G, +5U, | GAACCUCGACCACCGUAC | KRAS | | 10.8 |
| NO. 691 | +7C | | G12D | | |
| SEQ | −6A, +4A, | CAACAUCGACCACCAAAU | KRAS | | 10.8 |
| NO. 692 | +5A | | G12D | | |
| SEQ | −8C, +2U, | CACCCUCGACCAUCGAGU | KRAS | Rule 5 | 10.5 |
| NO. 693 | +5A, +6G | | G12D | | |
| SEQ | −4U, −1A, | CAACCUUGAACACCGCAA | KRAS | | 10.5 |
| NO. 694 | +7A | | G12D | | |
| SEQ | −8C, −6U, | UCCAUCUCGCCCACCCCA | KRAS | | 93.8 |
| NO. 695 | −1C, +5C | | G12S | | |
| SEQ | −6U, −1U, | UCAAUCUCGUCAACCGCA | KRAS | | 93.3 |
| NO. 696 | +1A | | G12S | | |
| SEQ | −10A, +2U, | ACAACCUCGACCUCCGGA | KRAS | Rule 6 | 76.2 |
| NO. 697 | +6G | | G12S | | |
| SEQ | −7G, −4G, | UCAGCCGCGACCACUGUA | KRAS | | 73.9 |
| NO. 698 | +4U, +6U | | G12S | | |
| SEQ | −5U, +2C, | UCAACUUCGACCCCCCCA | KRAS | Rule 6 | 72.7 |
| NO. 699 | +5C | | G12S | | |
| SEQ | +2C, +6G | UCAACCUCGACCCCCGGA | KRAS | Rule 6 | 72.3 |
| NO. 700 | | | G12S | | |
| SEQ | +2C, +6A | UCAACCUCGACCCCCGAA | KRAS | Rule 6 | 69.4 |
| NO. 701 | | | G12S | | |
| SEQ | +2C, +7G | UCAACCUCGACCCCCGCG | KRAS | Rule 6 | 69 |
| NO. 702 | | | G12S | | |
| SEQ | −5U, +4G | UCAACUUCGACCACGGCA | KRAS | | 68.8 |
| NO. 703 | | | G12S | | |
| SEQ | −10C, −2A, | CCAACCUCAACCCCCGCA | KRAS | Rule 6 | 68.3 |
| NO. 704 | +2C | | G12S | | |
| SEQ | +2U, +7U | UCAACCUCGACCUCCGCU | KRAS | Rule 6 | 68.2 |
| NO. 705 | | | G12S | | |
| SEQ | −10C, +2U, | CCAACCUCGACCUCCGGA | KRAS | Rule 6 | 67.1 |
| NO. 706 | +6G | | G12S | | |
| SEQ | −9U, −2A, | UUAACCUCAACCACCGCU | KRAS | | 66.7 |
| NO. 707 | +7U | | G12S | | |
| SEQ | +2U, +7C | UCAACCUCGACCUCCGCC | KRAS | Rule 6 | 64.7 |
| NO. 708 | | | G12S | | |
| SEQ | −3U, +2C | UCAACCUUGACCCCCGCA | KRAS | Rule 6 | 64 |
| NO. 709 | | | G12S | | |
| SEQ | −2A, +6G | UCAACCUCAACCACCGGA | KRAS | | 64 |
| NO. 710 | | | G12S | | |
| SEQ | −6U, +2U, | UCAAUCUCGACCUCCGGA | KRAS | Rule 6 | 63.6 |
| NO. 711 | +6G | | G12S | | |
| SEQ | −3U, +4A, | UCAACCUUGACCACAGGA | KRAS | | 62.8 |
| NO. 712 | +6G | | G12S | | |
| SEQ | −9U, +3G, | UUAACCUCGACCAGCGUA | KRAS | | 62.5 |
| NO. 713 | +6U | | G12S | | |
| SEQ | −2A, +2C | UCAACCUCAACCCCCGCA | KRAS | Rule 6 | 62.2 |
| NO. 714 | | | G12S | | |
| SEQ | −8U, −2A, | UCUACCUCAACCACAGCA | KRAS | | 61.5 |
| NO. 715 | +4A | | G12S | | |
| SEQ | +2U, +5A, | UCAACCUCGACCUCCACC | KRAS | Rule 6 | 61.3 |
| NO. 716 | +7C | | G12S | | |
| SEQ | −10C, −9A, | CAAACCUCGACCACCGCC | KRAS | | 61.1 |
| NO. 717 | +7C | | G12S | | |
| SEQ | −2A, +3U, | UCAACCUCAACCAUCGAA | KRAS | | 61.1 |
| NO. 718 | +6A | | G12S | | |
| SEQ | +2U, +6G | UCAACCUCGACCUCCGGA | KRAS | Rule 6 | 61 |
| NO. 719 | | | G12S | | |
| SEQ | +5A, +6A | UCAACCUCGACCACCAAA | KRAS | | 60.9 |
| NO. 720 | | | G12S | | |
| SEQ | −3U, +2U, | UCAACCUUGACCUCCGCG | KRAS | Rule 6 | 60.6 |
| NO. 721 | +7G | | G12S | | |
| SEQ | +2U, +4A | UCAACCUCGACCUCAGCA | KRAS | Rule 6 | 60.6 |
| NO. 722 | | | G12S | | |
| SEQ | −9U, −5U, | UUAACUUCGACCUCCGGA | KRAS | Rule 6 | 60.5 |
| NO. 723 | +2U, +6G | | G12S | | |
| SEQ | −8C, +4A, | UCCACCUCGACCACACCA | KRAS | | 60 |
| NO. 724 | +5C | | G12S | | |
| SEQ | −5G, +6U, | UCAACGUCGACCACCGUU | KRAS | | 60 |
| NO. 725 | +7U | | G12S | | |
| SEQ | −3U, +2U, | UCAACCUUGACCUCCGAA | KRAS | Rule 6 | 59.8 |
| NO. 726 | +6A | | G12S | | |
| SEQ | +2U, +6A | UCAACCUCGACCUCCGAA | KRAS | Rule 6 | 59.7 |
| NO. 727 | | | G12S | | |
| SEQ | +4G, +5U | UCAACCUCGACCACGUCA | KRAS | | 59.7 |
| NO. 728 | | | G12S | | |
| SEQ | +3A, +6G, | CCGACCUGUGCCUACGGG | MeCP2 | | 100 |
| NO. 729 | +7G | | R106Q | | |
| SEQ | −10U, +6G | UCGACCUGUGCCUUCGGA | MeCP2 | | 93.3 |
| NO. 730 | | | R106Q | | |
| SEQ | +4U, +6G | CCGACCUGUGCCUUUGGA | MeCP2 | | 89.5 |
| NO. 731 | | | R106Q | | |
| SEQ | −8U, +6U | CCUACCUGUGCCUUCGUA | MeCP2 | Rule 2 | 86.5 |
| NO. 732 | | | R106Q | | |
| SEQ | +4G, +6G | CCGACCUGUGCCUUGGGA | MeCP2 | | 85 |
| NO. 733 | | | R106Q | | |
| SEQ | −10G, −9A, | GAGAGCUGUGCCUGCGAA | MeCP2 | | 80 |
| NO. 734 | −6G, +3G | | R106Q | | |
| SEQ | −8U, +2C, | CCUACCUGUGCCCACGAA | MeCP2 | Rule 2 | 78.5 |
| NO. 735 | +3A | | R106Q | | |
| SEQ | −10G, +6G | GCGACCUGUGCCUUCGGA | MeCP2 | | 72.7 |
| NO. 736 | | | R106Q | | |
| SEQ | −10A, −9U, | AUGACCUGUGCAUUCGAA | MeCP2 | | 68.2 |
| NO. 737 | +1A | | R106Q | | |
| SEQ | −8C, −4C, | CCCACCCGUGCCUUCAAA | MeCP2 | Rule 2 | 66.7 |
| NO. 738 | +5A | | R106Q | | |
| SEQ | −8C, −3A | CCCACCUAUGCCUUCGAA | MeCP2 | Rule 2 | 64.8 |
| NO. 739 | | | R106Q | | |
| SEQ | −10A, −2A, | ACGACCUGAGCCUUAGAA | MeCP2 | | 61.5 |
| NO. 740 | +4A | | R106Q | | |
| SEQ | −6A, −4C, | CCGAACCGUGCCCUCGUA | MeCP2 | | 60 |
| NO. 741 | +2C, +6U | | R106Q | | |
| SEQ | −10G, −9A, | GAGACCUGUGCCUGCUAA | MeCP2 | | 47.8 |
| NO. 742 | +3G, +5U | | R106Q | | |
| SEQ | −8A, +3A, | CCAACCUGUGCCUAGGAA | MeCP2 | | 46.7 |
| NO. 743 | +4G | | R106Q | | |
| SEQ | −8C, +7G | CCCACCUGUGCCUUCGAG | MeCP2 | Rule 2 | 46.4 |
| NO. 744 | | | R106Q | | |
| SEQ | −8U, −4C, | CCUACCCGUGCCUUCGAC | MeCP2 | Rule 2 | 46.2 |
| NO. 745 | +7C | | R106Q | | |
| SEQ | −10G, −8A | GCAACCUGUGCCUUCGAA | MeCP2 | | 46.1 |
| NO. 746 | | | R106Q | | |
| SEQ | −4C, +3A, | CCGACCCGUGCCUACUAA | MeCP2 | | 45.5 |
| NO. 747 | +5U | | R106Q | | |
| SEQ | −8U, +2C | CCUACCUGUGCCCUCGAA | MeCP2 | Rule 2 | 44.4 |
| NO. 748 | | | R106Q | | |
| SEQ | −3C, +2C, | CCGACCUCUGCCCUCGAG | MeCP2 | | 44.4 |
| NO. 749 | +7G | | R106Q | | |
| SEQ | −5U, +6G | CCGACUUGUGCCUUCGGA | MeCP2 | | 44.3 |
| NO. 750 | | | R106Q | | |
| SEQ | −4A, +3A, | CCGACCAGUGCCUACUAA | MeCP2 | | 43.9 |
| NO. 751 | +5U | | R106Q | | |
| SEQ | −5A, −2A, | CCGACAUGAGCCCUCGAA | MeCP2 | | 43.8 |
| NO. 752 | +2C | | R106Q | | |
| SEQ | −8U, −4C, | CCUACCCGUGCCUUCGCA | MeCP2 | Rule 2 | 42.9 |
| NO. 753 | +6C | | R106Q | | |
| SEQ | −7A, −2U, | GACAUGUCUGCUCCUUAA | F5 R534Q | | 56.7 |
| NO. 754 | +7A | | | | |
| SEQ | −8G, +4C | GAGCUGUCCGCUCCCUAU | F5 R534Q | | 45.5 |
| NO. 755 | | | | | |
| SEQ | −10A, −6A, | AACCAGUCCGCUCCUGAU | F5 R534Q | | 44.4 |
| NO. 756 | +5G | | | | |
| SEQ | −5C, −3U, | GACCUCUUCGCUCCUUAC | F5 R534Q | | 41.7 |
| NO. 757 | +7C | | | | |
| SEQ | −10C, −8A, | CAAUUGUCCGAUCCUUAU | F5 R534Q | | 40.7 |
| NO. 758 | −7U, 0A | | | | |
| SEQ | −6C, +1C | GACCCGUCCGCCCCUUAU | F5 R534Q | | 40 |
| NO. 759 | | | | | |
| SEQ | −6A, +2U, | GACCAGUCCGCUUCUUAC | F5 R534Q | Rule 5 | 40 |
| NO. 760 | +7C | | | | |
| SEQ | −9U, +4G | GUCCUGUCCGCUCCGUAU | F5 R534Q | | 39.3 |
| NO. 761 | | | | | |
| SEQ | −6C, +2U | GACCCGUCCGCUUCUUAU | F5 R534Q | Rule 5 | 39.2 |
| NO. 762 | | | | | |
| SEQ | −9C, −5A, | GCCCUAUCCGCUCCUUAG | F5 R534Q | | 38.5 |
| NO. 763 | +7G | | | | |
| SEQ | +6G, +7G | GACCUGUCCGCUCCUUGG | F5 R534Q | | 38.5 |
| NO. 764 | | | | | |
| SEQ | +3U, +6G, | GACCUGUCCGCUCUUUGG | F5 R534Q | | 37.5 |
| NO. 765 | +7G | | | | |
| SEQ | −4G, +6G | GACCUGGCCGCUCCUUGU | F5 R534Q | | 35.7 |
| NO. 766 | | | | | |
| SEQ | −3U, +6G | GACCUGUUCGCUCCUUGU | F5 R534Q | | 34.8 |
| NO. 767 | | | | | |
| SEQ | −10C, +2U | CACCUGUCCGCUUCUUAU | F5 R534Q | Rule 5 | 34 |
| NO. 768 | | | | | |
| SEQ | +1C, +6G | GACCUGUCCGCCCCUUGU | F5 R534Q | | 33.3 |
| NO. 769 | | | | | |
| SEQ | −10C, −9G, | CGCCUGCCCGCCCCUUAU | F5 R534Q | | 33.3 |
| NO. 770 | −4C, +1C | | | | |
| SEQ | −10U, −6C, | UACCCGUCCGCUCGUUAU | F5 R534Q | | 33.3 |
| NO. 771 | +3G | | | | |
| SEQ | −9G, +2U | GGCCUGUCCGCUUCUUAU | F5 R534Q | Rule 5 | 33.3 |
| NO. 772 | | | | | |
| SEQ | +2G, +4C | GACCUGUCCGCUGCCUAU | F5 R534Q | | 33.3 |
| NO. 773 | | | | | |
| SEQ | +4G, +6U | GACCUGUCCGCUCCGUUU | F5 R534Q | | 33.3 |
| NO. 774 | | | | | |
| SEQ | +1C, +5G | GACCUGUCCGCCCCUGAU | F5 R534Q | | 32.1 |
| NO. 775 | | | | | |
| SEQ | −8G, +6C, | GAGCUGUCCGCUCCUUCG | F5 R534Q | | 31.6 |
| NO. 776 | +7G | | | | |
| SEQ | +1C, +6C | GACCUGUCCGCCCCUUCU | F5 R534Q | | 31.4 |
| NO. 777 | | | | | |
| SEQ | −9G, −4C, | GGCCUGCCCGCUCCGUAU | F5 R534Q | | 31.4 |
| NO. 778 | +4G | | | | |
| SEQ | −10U, +4G, | UACCUGUCCGCUCCGAAU | F5 R534Q | | 30.8 |
| NO. 779 | +5A | | | | |
| SEQ | −10C, −9U | CUCCUGUCCGCUCCUUAU | F5 R534Q | | 30.4 |
| NO. 780 | | | | | |
| SEQ | −8U, −7U, | GAUUCGUCCGCUCACUAU | F5 R534Q | | 30.4 |
| NO. 781 | −6C, +3A, | | | | |
| | +4C | | | | |
| SEQ | −4C, +1C | GACCUGCCCGCCCCUUAU | F5 R534Q | | 30.1 |
| NO. 782 | | | | | |
| SEQ | −6C, +3A, | GACCCGUCCGCUCACUAU | F5 R534Q | | 30 |
| NO. 783 | +4C | | | | |
| SEQ | +2U, +6C | GACCUGUCCGCUUCUUCU | F5 R534Q | Rule 5 | 29.8 |
| NO. 784 | | | | | |
| SEQ | −9G, +4A | GGCCUGUCCGCUCCAUAU | F5 R534Q | | 29.5 |
| NO. 785 | | | | | |
| SEQ | −6A, +4A, | GACCAGUCCGCUCCAUAC | F5 R534Q | | 29.4 |
| NO. 786 | +7C | | | | |
| SEQ | −9U, +2U | GUCCUGUCCGCUUCUUAU | F5 R534Q | Rule 5 | 29.2 |
| NO. 787 | | | | | |
| SEQ | −10C, −3U | CACCUGUUCGCUCCUUAU | F5 R534Q | | 29.2 |
| NO. 788 | | | | | |
| SEQ | −6G, −2U, | GACCGGUCUGGUCUUUAU | F5 R534Q | | 29.2 |
| NO. 789 | 0G, +3U | | | | |
| SEQ | −9G, −8A, | GGACUGUCCGCUCCUUAG | F5 R534Q | | 28.9 |
| NO. 790 | +7G | | | | |
| SEQ | +4A, +5A | GACCUGUCCGCUCCAAAU | F5 R534Q | | 28.6 |
| NO. 791 | | | | | |
| SEQ | +1C, +5C | GACCUGUCCGCCCCUCAU | F5 R534Q | | 28.6 |
| NO. 792 | | | | | |
| SEQ | −10C, +1C | CACCUGUCCGCCCCUUAU | F5 R534Q | | 28.6 |
| NO. 793 | | | | | |
| SEQ | −10A, −9U, | AUCCUGUCCGCCCUUUAA | F5 R534Q | | 28.6 |
| NO. 794 | +1C, +3U, | | | | |
| | +7A | | | | |
| SEQ | −10U, +1C, | UACCUGUCCGCCCCUUUU | F5 R534Q | | 28.6 |
| NO. 795 | +6U | | | | |
| SEQ | −6C, +7C | GACCCGUCCGCUCCUUAC | F5 R534Q | | 28.4 |
| NO. 796 | | | | | |
| SEQ | −1G, +3G | GACACACAGGCUCGAGCG | ABCA4 | | 42.9 |
| NO. 797 | | | G1961E | | |
| SEQ | −9G, −1G, | GGCACACAGGCUCAAGCC | ABCA4 | | 20.4 |
| NO. 798 | +7C | | G1961E | | |
| SEQ | −1U, +6A, | GACACACAGUCUCAAGAA | ABCA4 | | 19.1 |
| NO. 799 | +7A | | G1961E | | |
| SEQ | −1G, +4G, | GACACACAGGCUCAGACC | ABCA4 | | 18.2 |
| NO. 800 | +5A, +7C | | G1961E | | |
| SEQ | −9G, −1G, | GGCACACAGGCUCGAGCC | ABCA4 | | 17.4 |
| NO. 801 | +3G, +7C | | G1961E | | |
| SEQ | −9U, +4U, | GUCACACAGCCUCAUGAA | ABCA4 | | 15.8 |
| NO. 802 | +6A, +7A | | G1961E | | |
| SEQ | −6G, −2A, | GACAGACAACCUCAAGUG | ABCA4 | | 13.3 |
| NO. 803 | +6U | | G1961E | | |
| SEQ | −5G, −4G, | GACACGGACCCUCUAGGG | ABCA4 | | 13.3 |
| NO. 804 | −2C, +3U, | | G1961E | | |
| | +6G | | | | |
| SEQ | −1G, +3U, | GACACACAGGCUCUAUCG | ABCA4 | | 13.3 |
| NO. 805 | +5U | | G1961E | | |
| SEQ | −9G, −5U, | GGCACUCAGCCUCACGCU | ABCA4 | | 12.3 |
| NO. 806 | +4C, +7U | | G1961E | | |
| SEQ | −4G, −3C | GACACAGCGCCUCAAGCG | ABCA4 | | 12.1 |
| NO. 807 | | | G1961E | | |
| SEQ | −1G, +7U | GACACACAGGCUCAAGCU | ABCA4 | | 11.8 |
| NO. 808 | | | G1961E | | |
| SEQ | −1G, +5C, | GACACACAGGCUCAACCU | ABCA4 | | 11.7 |
| NO. 809 | +7U | | G1961E | | |
| SEQ | −7G, −2C, | GACGCACACCGUCAAGCG | ABCA4 | | 11.3 |
| NO. 810 | 0G | | G1961E | | |
| SEQ | −7G, −2A, | GACGCACAACCUUAAGGG | ABCA4 | Rule 5 | 11.1 |
| NO. 811 | +2U, +6G | | G1961E | | |
| SEQ | −9C, −1G, | GCCACACAGGCUCAACCG | ABCA4 | | 11.1 |
| NO. 812 | +5C | | G1961E | | |
| SEQ | −6G, −1G, | GACAGACAGGCUCGAGCG | ABCA4 | | 11.1 |
| NO. 813 | +3G | | G1961E | | |
| SEQ | −1G, +5C | GACACACAGGCUCAACCG | ABCA4 | | 11 |
| NO. 814 | | | G1961E | | |
| SEQ | −1G, +3U | GACACACAGGCUCUAGCG | ABCA4 | | 10.9 |
| NO. 815 | | | G1961E | | |
| SEQ | −9C, −1G, | GCCACACAGGCUCAAGAG | ABCA4 | | 10.6 |
| NO. 816 | +6A | | G1961E | | |
| SEQ | −10A, −8U, | AAUACACAGCCUCAAACG | ABCA4 | | 10.5 |
| NO. 817 | +5A | | G1961E | | |
| SEQ | −8G, −3U, | GAGACACUGCCUCAAACC | ABCA4 | | 10 |
| NO. 818 | +5A, +7C | | G1961E | | |
| SEQ | −10A, −9U, | AUCACACAGAUUCAAGGG | ABCA4 | | 10 |
| NO. 819 | −1A, 0U, | | G1961E | | |
| | +6G | | | | |
| SEQ | +1C, +4G, | GACACACAGCCCCAGGAG | ABCA4 | | 9.6 |
| NO. 820 | +6A | | G1961E | | |
| SEQ | +2A, +4C | GACACACAGCCUAACGCG | ABCA4 | | 9.5 |
| NO. 821 | | | G1961E | | |
| SEQ | −1G, +6U, | GACACACAGGCUCAAGUA | ABCA4 | | 9.3 |
| NO. 822 | +7A | | G1961E | | |
| SEQ | −6G, +1C | GACAGACAGCCCCAAGCG | ABCA4 | | 9.1 |
| NO. 823 | | | G1961E | | |
| SEQ | −3U, +2U, | GACACACUGCCUUUAGCG | ABCA4 | | 9.1 |
| NO. 824 | +3U | | G1961E | | |
| SEQ | −10U, −9G, | UGCACACAGGCUCAAGCU | ABCA4 | | 9.1 |
| NO. 825 | −1G, +7U | | G1961E | | |
| SEQ | −8G, +1C, | GAGACACAGCCCCAAGGC | ABCA4 | | 9.1 |
| NO. 826 | +6G, +7C | | G1961E | | |
| SEQ | −8G, 0A, | GAGACACAGCAUGAAGCG | ABCA4 | | 9.1 |
| NO. 827 | +2G | | G1961E | | |
| SEQ | −3G, −1G, | GACACACGGGCUCACGCG | ABCA4 | | 9.1 |
| NO. 828 | +4C | | G1961E | | |
| SEQ | −9G, −8U, | GGUACACAGCAUCAAGCG | ABCA4 | | 9.1 |
| NO. 829 | 0A | | G1961E | | |
| SEQ | −10A, −1G, | AACACACAGGCUCAACCG | ABCA4 | | 9 |
| NO. 830 | +5C | | G1961E | | |
| SEQ | −1G, +3C, | GACACACAGGCUCCUGCG | ABCA4 | | 9 |
| NO. 831 | +4U | | G1961E | | |
| SEQ | +2U, +6G | GACACACAGCCUUAAGGG | ABCA4 | Rule 5 | 8.9 |
| NO. 832 | | | G1961E | | |
| SEQ | −10C, −2A, | CACACACAACAUCAAUCG | ABCA4 | | 8.7 |
| NO. 833 | 0A, +5U | | G1961E | | |
| SEQ | −8U, −2A, | GAUACACAACCUCAGGCG | ABCA4 | | 8.7 |
| NO. 834 | +4G | | G1961E | | |
| SEQ | −10A, −9U, | AUCACACAGGCUCAAUCG | ABCA4 | | 8.6 |
| NO. 835 | −1G, +5U | | G1961E | | |
| SEQ | −4U, −1G, | GACACAUAGGCUCGAGCG | ABCA4 | | 8.5 |
| NO. 836 | +3G | | G1961E | | |
| SEQ | −8A, −2U, | GAAACACAUCCCCAAGCG | ABCA4 | | 8.3 |
| NO. 837 | +1C | | G1961E | | |
| SEQ | −4G, −3G, | GACACAGGGCCUCACGCG | ABCA4 | | 8.3 |
| NO. 838 | +4C | | G1961E | | |
| SEQ | −9C, −1G, | GCCACACAGGCUCAGGCG | ABCA4 | | 8.3 |
| NO. 839 | +4G | | G1961E | | |
| SEQ | −1G | GACACACAGGCUCAAGCG | ABCA4 | | 8.2 |
| NO. 840 | | | G1961E | | |
| SEQ | −7G, +5C, | GACGCACAGCCUCAACCU | ABCA4 | | 8.2 |
| NO. 841 | +7U | | G1961E | | |
| SEQ | −2A, +1C, | GACACACAACCCCACGCC | ABCA4 | | 8.2 |
| NO. 842 | +4C, +7C | | G1961E | | |
| SEQ | −9G, +1C, | GGCACACAGCCCCUAGCG | ABCA4 | | 8.1 |
| NO. 843 | +3U | | G1961E | | |
| SEQ | −3G, +3G | GACACACGGCCUCGAGCG | ABCA4 | | 8.1 |
| NO. 844 | | | G1961E | | |
| SEQ | −7C, −1U, | GACCCACAGUCUCUAGCG | ABCA4 | | 8 |
| NO. 845 | +3U | | G1961E | | |
| SEQ | −1G, +2U, | GACACACAGGCUUAAGCU | ABCA4 | Rule 5 | 7.8 |
| NO. 846 | +7U | | G1961E | | |
| SEQ | −5U, −1G | GACACUCAGGCUCAAGCG | ABCA4 | | 7.7 |
| NO. 847 | | | G1961E | | |
| SEQ | −10C, −3C, | CACACACCGCCUCAGGCG | ABCA4 | | 7.7 |
| NO. 848 | +4G | | G1961E | | |
| SEQ | −4A, +1C, | GACACAAAGCCCCAAGCU | ABCA4 | | 7.7 |
| NO. 849 | +7U | | G1961E | | |
| SEQ | −9C, −1A, | GCCACACAGACUCAAUCG | ABCA4 | | 7.7 |
| NO. 850 | +5U | | G1961E | | |
| SEQ | −4A, −1G, | GACACAAAGGUUCAAGCU | ABCA4 | | 7.6 |
| NO. 851 | 0U, +7U | | G1961E | | |
| SEQ | −10U, −1G | UACACACAGGCUCAAGCG | ABCA4 | | 7.5 |
| NO. 852 | | | G1961E | | |
| SEQ | −8U, +3G, | GAUACACAGCCUCGAGCC | ABCA4 | | 7.5 |
| NO. 853 | +7C | | G1961E | | |
| SEQ | −3G, −1G, | GACACACGGGCUCAAGCU | ABCA4 | | 7.4 |
| NO. 854 | +7U | | G1961E | | |
| SEQ | −1G, +4G | GACACACAGGCUCAGGCG | ABCA4 | | 7.4 |
| NO. 855 | | | G1961E | | |
| SEQ | −6U, −1G | GACAUACAGGCUCAAGCG | ABCA4 | | 7.3 |
| NO. 856 | | | G1961E | | |
| SEQ | −10U, −1G, | UACACACAGGCUCACGGG | ABCA4 | | 7.3 |
| NO. 857 | +4C, +6G | | G1961E | | |
| SEQ | −2C, 0A, | GACACACACCAUCUAGCU | ABCA4 | | 7.3 |
| NO. 858 | +3U, +7U | | G1961E | | |
| SEQ | −1G, +4C | GACACACAGGCUCACGCG | ABCA4 | | 7.3 |
| NO. 859 | | | G1961E | | |
| SEQ | −9U, −6G, | GUCAGACAGCCUCAAGAG | ABCA4 | | 7.1 |
| NO. 860 | +6A | | G1961E | | |
| SEQ | −10C, −7U | CACUCACAGCCUCAAGCG | ABCA4 | | 7.1 |
| NO. 861 | | | G1961E | | |
| SEQ | −10A, −2A, | AACACACAAAGUCAAGCG | ABCA4 | | 7.1 |
| NO. 862 | −1A, 0G | | G1961E | | |
| SEQ | −8G, −6U, | GAGAUUAAGCCUCAAGCG | ABCA4 | | 7.1 |
| NO. 863 | −5U, −4A | | G1961E | | |
| SEQ | 0A, +5A, | GACACACAGCAUCAAACA | ABCA4 | | 7.1 |
| NO. 864 | +7A | | G1961E | | |
| SEQ | −8U, −7U, | GAUUCCCAGCCCAAAGCG | ABCA4 | | 7.1 |
| NO. 865 | −5C, +1C, | | G1961E | | |
| | +2A | | | | |
| SEQ | −9C, −2C, | GCCACACACCGUCAAGCG | ABCA4 | | 7.1 |
| NO. 866 | 0G | | G1961E | | |
| SEQ | −10A, −2A, | AACACACAACCUCUAGCG | ABCA4 | | 7 |
| NO. 867 | +3U | | G1961E | | |
| SEQ | −2A, +6A, | GACACACAACCUCAAGAA | ABCA4 | | 7 |
| NO. 868 | +7A | | G1961E | | |
| SEQ | −5U, +5C, | GACACUCAGCCUCAACUG | ABCA4 | | 6.8 |
| NO. 869 | +6U | | G1961E | | |
| SEQ | −10U, +3G | UACACACAGCCUCGAGCG | ABCA4 | | 6.8 |
| NO. 870 | | | G1961E | | |
| SEQ | −9G, −1G, | GGCACACAGGCUCAAGGG | ABCA4 | | 6.8 |
| NO. 871 | +6G | | G1961E | | |
| SEQ | −5G, −1G, | GACACGCAGGCUCUAGCG | ABCA4 | | 6.7 |
| NO. 872 | +3U | | G1961E | | |
| SEQ | −7C, −1G, | GACCCACAGGCUCAACCG | ABCA4 | | 6.7 |
| NO. 873 | +5C | | G1961E | | |
| SEQ | −9U, +1A, | GUCACACAGCCACAUGCG | ABCA4 | | 6.7 |
| NO. 874 | +4U | | G1961E | | |
| SEQ | −7G, −4A, | GACGCAAAGCGUAAUGUG | ABCA4 | | 6.7 |
| NO. 875 | 0G, +2A, | | G1961E | | |
| | +4U, +6U | | | | |
| SEQ | +1C, +5C, | GACACACAGCCCCAACAG | ABCA4 | | 6.7 |
| NO. 876 | +6A | | G1961E | | |
| SEQ | +5U, +6A, | GACACACAGCCUCAAUAA | ABCA4 | | 6.7 |
| NO. 877 | +7A | | G1961E | | |
| SEQ | −9U, 0A | GUCACACAGCAUCAAGCG | ABCA4 | | 6.7 |
| NO. 878 | | | G1961E | | |
| SEQ | −1G, +5U | GACACACAGGCUCAAUCG | ABCA4 | | 6.6 |
| NO. 879 | | | G1961E | | |
| SEQ | −6A, +1C, | GACAAACAGCCCCAAGAG | ABCA4 | | 6.6 |
| NO. 880 | +6A | | G1961E | | |
| SEQ | −9G, −1G, | GGCACACAGGCUCAAGUG | ABCA4 | | 6.6 |
| NO. 881 | +6U | | G1961E | | |
| SEQ | −8A, −6U, | GAAAUACAGCCCCCAGCU | ABCA4 | | 6.6 |
| NO. 882 | +1C, +3C, | | G1961E | | |
| | +7U | | | | |
| SEQ | +4C, +6G | GACACACAGCCUCACGGG | ABCA4 | | 6.5 |
| NO. 883 | | | G1961E | | |
| SEQ | −6G, −1G | GACAGACAGGCUCAAGCG | ABCA4 | | 6.5 |
| NO. 884 | | | G1961E | | |
| SEQ | −9C, −5U, | GCCACUCAGCCCCAAGCU | ABCA4 | | 6.5 |
| NO. 885 | +1C, +7U | | G1961E | | |
| SEQ | −6A, −2A, | GACAAACAACCUCAACCG | ABCA4 | | 6.5 |
| NO. 886 | +5C | | G1961E | | |
| SEQ | −8U, −3C, | GAUACACCGCCUCAGGCG | ABCA4 | | 6.2 |
| NO. 887 | +4G | | G1961E | | |
| SEQ | −8A, −7G, | GAAGAGCAGCCUCAAGAG | ABCA4 | | 6.2 |
| NO. 888 | −6A, −5G, | | G1961E | | |
| | +6A | | | | |
| SEQ | 0A, +2U, | GACACACAGCAUUAGGCG | ABCA4 | Rule 5 | 6.2 |
| NO. 889 | +4G | | G1961E | | |
| SEQ | −9G, +6G | GGCACACAGCCUCAAGGG | ABCA4 | | 6.2 |
| NO. 890 | | | G1961E | | |
| SEQ | −1G, +7A | GACACACAGGCUCAAGCA | ABCA4 | | 6.2 |
| NO. 891 | | | G1961E | | |
| SEQ | −2C, 0G | GACACACACCGUCAAGCG | ABCA4 | | 6.1 |
| NO. 892 | | | G1961E | | |
| SEQ | −1G, +3G, | GACACACAGGCUCGAGAG | ABCA4 | | 6.1 |
| NO. 893 | +6A | | G1961E | | |
| SEQ | +3U, +7C | GACACACAGCCUCUAGCC | ABCA4 | | 6.1 |
| NO. 894 | | | G1961E | | |
| SEQ | −9C, −1G, | GCCACACAGGCUCUAGCG | ABCA4 | | 6.1 |
| NO. 895 | +3U | | G1961E | | |
| SEQ | −6U, +4G | GACAUACAGCCUCAGGCG | ABCA4 | | 6 |
| NO. 896 | | | G1961E | | |
| SEQ | −1G, 0U, | GACACACAGGUUCAAGCU | ABCA4 | | 6 |
| NO. 897 | +7U | | G1961E | | |
| SEQ | −7G, −3C, | GACGCACCACCUCAAGCU | ABCA4 | | 6 |
| NO. 898 | −2A, +7U | | G1961E | | |
| SEQ | −6G, −1G, | GACAGACAGGUUCAAGCG | ABCA4 | | 6 |
| NO. 899 | 0U | | G1961E | | |
| SEQ | −7U, −1A, | GACUCACAGAAUCAAACG | ABCA4 | | 5.9 |
| NO. 900 | 0A, +5A | | G1961E | | |
| SEQ | −10C, −1G, | CACACACAGGCUCAAGCA | ABCA4 | | 5.9 |
| NO. 901 | +7A | | G1961E | | |
| SEQ | −9G, −8A, | GGAACACAGCCUCAAGAA | ABCA4 | | 5.9 |
| NO. 902 | +6A, +7A | | G1961E | | |
| SEQ | −9G, −6A | GGCAAACAGCCUCAAGCG | ABCA4 | | 5.9 |
| NO. 903 | | | G1961E | | |
| SEQ | −10C, −8U, | CAUACACAGCCUCAAUCG | ABCA4 | | 5.9 |
| NO. 904 | +5U | | G1961E | | |
| SEQ | −6U, −2A, | GACAUACAACCCCAAGCG | ABCA4 | | 5.9 |
| NO. 905 | +1C | | G1961E | | |
| SEQ | −8U, −7G, | GAUGCACAGCCCCAUGCG | ABCA4 | | 5.9 |
| NO. 906 | +1C, +4U | | G1961E | | |
| SEQ | −8A, +2U, | GAAACACAGCCUUAAUCG | ABCA4 | Rule 5 | 5.9 |
| NO. 907 | +5U | | G1961E | | |
| SEQ | −4U, +4C, | GACACAUAGCCUCACUCG | ABCA4 | | 5.9 |
| NO. 908 | +5U | | G1961E | | |
| SEQ | −10U, −9G, | UGCACACAGGCUCAGACG | ABCA4 | | 5.9 |
| NO. 909 | −1G, +4G, | | G1961E | | |
| | +5A | | | | |
| SEQ | −7G, −3U, | GACGCACUGUCUCAAGCG | ABCA4 | | 5.9 |
| NO. 910 | −1U | | G1961E | | |
| SEQ | −9G, −8U, | GGUACACAGCCUCAAGCA | ABCA4 | | 5.8 |
| NO. 911 | +7A | | G1961E | | |
| SEQ | −6G, −2A | GACAGACAACCUCAAGCG | ABCA4 | | 5.8 |
| NO. 912 | | | G1961E | | |
| SEQ | −5C, 0U, | GACACCCAGCUUCAAGCU | ABCA4 | | 5.7 |
| NO. 913 | +7U | | G1961E | | |
| SEQ | −1G, +6U | GACACACAGGCUCAAGUG | ABCA4 | | 5.7 |
| NO. 914 | | | G1961E | | |
| SEQ | −10U, −7G, | UACGUACAGCCUCAAGCU | ABCA4 | | 5.6 |
| NO. 915 | −6U, +7U | | G1961E | | |
| SEQ | −10C, 0A, | CACACACAGCAUCAAGCU | ABCA4 | | 5.6 |
| NO. 916 | +7U | | G1961E | | |
| SEQ | −9G, −4U | GGCACAUAGCCUCAAGCG | ABCA4 | | 5.6 |
| NO. 917 | | | G1961E | | |
| SEQ | −2C, +4G, | GACACACACCCUCAGGGG | ABCA4 | | 5.6 |
| NO. 918 | +6G | | G1961E | | |
| SEQ | +3G, +4G, | GACACACAGCCUCGGGGG | ABCA4 | | 5.6 |
| NO. 919 | +6G | | G1961E | | |
| SEQ | −9U, −8U, | GUUACACAGCCUUGAGCG | ABCA4 | | 5.6 |
| NO. 920 | +2U, +3G | | G1961E | | |
| SEQ | +3G, +5U, | GACACACAGCCUCGAUCU | ABCA4 | | 5.6 |
| NO. 921 | +7U | | G1961E | | |
| SEQ | −9U, −2A, | GUCACACAACCUCAAGAU | ABCA4 | | 5.5 |
| NO. 922 | +6A, +7U | | G1961E | | |
| SEQ | −1G, +6A | GACACACAGGCUCAAGAG | ABCA4 | | 5.5 |
| NO. 923 | | | G1961E | | |
| SEQ | −8U, −1G | GAUACACAGGCUCAAGCG | ABCA4 | | 5.5 |
| NO. 924 | | | G1961E | | |
| SEQ | −8A, −1G, | GAAACACAGGCUCAACCG | ABCA4 | | 5.5 |
| NO. 925 | +5C | | G1961E | | |
| SEQ | −1U, +2A, | GACACACAGUCUAGAGCG | ABCA4 | | 5.4 |
| NO. 926 | +3G | | G1961E | | |
| SEQ | −10A, −7G, | AGAGGCGGUACGGACUUA | STAT3 | | 81.2 |
| NO. 927 | −5C, +7A | | Y705 | | |
| SEQ | −6A, +6C, | CGACAGGGUACGGACUCU | STAT3 | | 75 |
| NO. 928 | +7U | | Y705 | | |
| SEQ | −10G, +2U, | GGACGGGGUACGUACUAC | STAT3 | | 71.4 |
| NO. 929 | +6A | | Y705 | | |
| SEQ | −5U, +5C, | CGACGUGGUACGGACCGC | STAT3 | | 71 |
| NO. 930 | +6G | | Y705 | | |
| SEQ | −6U, −4C, | CGACUGCGUACGGACUAC | STAT3 | | 70 |
| NO. 931 | +6A | | Y705 | | |
| SEQ | −6C, −5A, | CGACCAGGUACGGCCUCA | STAT3 | | 70 |
| NO. 932 | +3C, +6C, | | Y705 | | |
| | +7A | | | | |
| SEQ | +2U, +7G | CGACGGGGUACGUACUUG | STAT3 | | 69.4 |
| NO. 933 | | | Y705 | | |
| SEQ | +3C, +7U | CGACGGGGUACGGCCUUU | STAT3 | | 69.1 |
| NO. 934 | | | Y705 | | |
| SEQ | −10U, −6C, | UGACCGAGUACGGACUUC | STAT3 | | 66.7 |
| NO. 935 | −4A | | Y705 | | |
| SEQ | −9U, −4C, | CUACGGCGUACGGACUCC | STAT3 | Rule 1 | 66.7 |
| NO. 936 | +6C | | Y705 | | |
| SEQ | −6C, −4A, | CGACCGAGUACGGUCUUC | STAT3 | | 66.7 |
| NO. 937 | +3U | | Y705 | | |
| SEQ | +2U, +3U, | CGACGGGGUACGUUCUUA | STAT3 | | 66.7 |
| NO. 938 | +7A | | Y705 | | |
| SEQ | −9U, +3C | CUACGGGGUACGGCCUUC | STAT3 | Rule 1 | 65.7 |
| NO. 939 | | | Y705 | | |
| SEQ | −9A, +3U | CAACGGGGUACGGUCUUC | STAT3 | | 65.4 |
| NO. 940 | | | Y705 | | |
| SEQ | +6A, +7U | CGACGGGGUACGGACUAU | STAT3 | | 65 |
| NO. 941 | | | Y705 | | |
| SEQ | −10A, −7G, | AGAGGGGGUACGGACUGC | STAT3 | | 65 |
| NO. 942 | +6G | | Y705 | | |
| SEQ | −6U, −5A, | CGACUAGGUACGGACUUG | STAT3 | | 64.7 |
| NO. 943 | +7G | | Y705 | | |
| SEQ | −6U, +2U, | CGACUGGGUACGUGCUUA | STAT3 | | 64.7 |
| NO. 944 | +3G, +7A | | Y705 | | |
| SEQ | −7A, +6A | CGAAGGGGUACGGACUAC | STAT3 | | 63.6 |
| NO. 945 | | | Y705 | | |
| SEQ | +6C, +7A | CGACGGGGUACGGACUCA | STAT3 | | 63.6 |
| NO. 946 | | | Y705 | | |
| SEQ | −9U, +3U | CUACGGGGUACGGUCUUC | STAT3 | Rule 1 | 63.3 |
| NO. 947 | | | Y705 | | |
| SEQ | +5G, +6A | CGACGGGGUACGGACGAC | STAT3 | | 63.2 |
| NO. 948 | | | Y705 | | |
| SEQ | −8G, −6U, | CGGCUGGGUACGGUUUUC | STAT3 | | 63.2 |
| NO. 949 | +3U, +4U | | Y705 | | |
| SEQ | +4G, +7U | CGACGGGGUACGGAGUUU | STAT3 | | 63.2 |
| NO. 950 | | | Y705 | | |
| SEQ | −8G, −7A, | CGGAGGGGUACGGAGUUC | STAT3 | | 62.5 |
| NO. 951 | +4G | | Y705 | | |
| SEQ | −7U, +6A, | CGAUGGGGUACGGACUAA | STAT3 | | 62.5 |
| NO. 952 | +7A | | Y705 | | |
| SEQ | −9A, −4U | CAACGGUGUACGGACUUC | STAT3 | | 62.5 |
| NO. 953 | | | Y705 | | |
| SEQ | −6A, +4A | CGACAGGGUACGGAAUUC | STAT3 | | 62.5 |
| NO. 954 | | | Y705 | | |
| SEQ | −7G, +3G, | CGAGGGGGUACGGGCGUC | STAT3 | | 62.5 |
| NO. 955 | +5G | | Y705 | | |
| SEQ | −8G, −6U, | CGGCUGGGUACGGUCUUA | STAT3 | | 62.5 |
| NO. 956 | +3U, +7A | | Y705 | | |
| SEQ | −10U, −6U, | UGACUGGUUACGGACUUC | STAT3 | | 61.5 |
| NO. 957 | −3U | | Y705 | | |
| SEQ | −6C, −5A, | CGACCAGGUACGGACUUG | STAT3 | | 61.1 |
| NO. 958 | +7G | | Y705 | | |
| SEQ | +2A, +6G, | CGACGGGGUACGAACUGA | STAT3 | | 60.9 |
| NO. 959 | +7A | | Y705 | | |
| SEQ | −6U, −5A, | CGACUAGGUACGGUCUUA | STAT3 | | 60.7 |
| NO. 960 | +3U, +7A | | Y705 | | |
| SEQ | −10G, −9C, | GCACUGGGUACGGACUGC | STAT3 | | 60.7 |
| NO. 961 | −6U, +6G | | Y705 | | |
| SEQ | −10U, +4G, | UGACGGGGUACGGAGUUA | STAT3 | | 60.5 |
| NO. 962 | +7A | | Y705 | | |
| SEQ | −7A, +7G | CGAAGGGGUACGGACUUG | STAT3 | | 60.2 |
| NO. 963 | | | Y705 | | |
| SEQ | −7U, −4C, | CGAUGGCGUACGGACUCC | STAT3 | | 60 |
| NO. 964 | +6C | | Y705 | | |
| SEQ | −9A, +3C, | CAACGGGGUACGGCCUAC | STAT3 | | 60 |
| NO. 965 | +6A | | Y705 | | |
| SEQ | −10U, +5A, | UGACGGGGUACGGACAAC | STAT3 | | 60 |
| NO. 966 | +6A | | Y705 | | |
| SEQ | −10G, −8G, | UUGCGUGGCUUACAUAGUUC | STAT1 | | 80.6 |
| NO. 967 | −5G, −3U | | Y701 | | |
| SEQ | −6C, +3U | UUCCUUCACCUACAUUGUUC | STAT1 | | 77.9 |
| NO. 968 | | | Y701 | | |
| SEQ | −6U, −4A, | UUCCUUUAACUACAUAGGUC | STAT1 | | 73.8 |
| NO. 969 | +5G | | Y701 | | |
| SEQ | −9U, −6C | UUCUUUCACCUACAUAGUUC | STAT1 | | 72.1 |
| NO. 970 | | | Y701 | | |
| SEQ | −7C, −6C, | UUCCUCCACCUACAUAGUUA | STAT1 | | 71 |
| NO. 971 | +7A | | Y701 | | |
| SEQ | −12G, +2G, | GUCCUUGACCUACAGAGGUC | STAT1 | | 68.2 |
| NO. 972 | +5G | | Y701 | | |
| SEQ | −12C, +2C, | CUCCUUGACCUACACAGAUC | STAT1 | | 66.7 |
| NO. 973 | +5A | | Y701 | | |
| SEQ | −6C, +3C | UUCCUUCACCUACAUCGUUC | STAT1 | | 65.9 |
| NO. 974 | | | Y701 | | |
| SEQ | −5U, −4A, | UUCCUUGUACUACAUAGUGC | STAT1 | | 64.3 |
| NO. 975 | +6G | | Y701 | | |
| SEQ | −9U, −8C, | UUCUCUGACCUACAUAGGCC | STAT1 | | 64.3 |
| NO. 976 | +5G, +6C | | Y701 | | |
| SEQ | −10U, +7G | UUUCUUGACCUACAUAGUUG | STAT1 | | 64.2 |
| NO. 977 | | | Y701 | | |
| SEQ | −12A, −9A | AUCAUUGACCUACAUAGUUC | STAT1 | | 63.6 |
| NO. 978 | | | Y701 | | |
| SEQ | −12C, −7A, | CUCCUAGACGUACAUAGUUC | STAT1 | | 63.6 |
| NO. 979 | −3G | | Y701 | | |
| SEQ | −11G, −7A, | UGCCUAGAUCUACACAGUUC | STAT1 | | 63.6 |
| NO. 980 | −4U, +2C | | Y701 | | |
| SEQ | −4G, −3U, | UUCCUUGAGUUACACAGUUC | STAT1 | | 63.6 |
| NO. 981 | +2C | | Y701 | | |
| SEQ | −6C, +5G, | UUCCUUCACCUACAUAGGCC | STAT1 | | 62.5 |
| NO. 982 | +6C | | Y701 | | |
| SEQ | −12G, −9U, | GUCUUUGACCUACAUUGUUC | STAT1 | | 62.5 |
| NO. 983 | +3U | | Y701 | | |
| SEQ | −6C, +7A | UUCCUUCACCUACAUAGUUA | STATI | | 62.5 |
| NO. 984 | | | Y701 | | |
| SEQ | −11G, −6C, | UGCCUUCACCUACAUAGUGC | STAT1 | | 60.9 |
| NO. 985 | +6G | | Y701 | | |
| SEQ | −12G, −10U, | GUUCUUGACCUACAUAGCUC | STAT1 | | 60.6 |
| NO. 986 | +5C | | Y701 | | |
| SEQ | +5G, +7U | UUCCUUGACCUACAUAGGUU | STAT1 | | 60.6 |
| NO. 987 | | | Y701 | | |
| SEQ | −6A, +5A, | UUCCUUAACCUACAUAGAAA | STAT1 | | 60 |
| NO. 988 | +6A, +7A | | Y701 | | |
| SEQ | −7C, −6U, | UUCCUCUACCUACAUAGCUC | STAT1 | | 60 |
| NO. 989 | +5C | | Y701 | | |
| SEQ | −6C, +3U, | UUCCUUCACCUACAUUGCUC | STAT1 | | 59.6 |
| NO. 990 | +5C | | Y701 | | |
| SEQ | −11A, −6C, | UACCUUCACCUACAUAGCUC | STAT1 | | 59.5 |
| NO. 991 | +5C | | Y701 | | |
| SEQ | −6C, +2G, | UUCCUUCACCUACAGAGUUA | STAT1 | | 59.3 |
| NO. 992 | +7A | | Y701 | | |
| SEQ | −11A, −6C, | UACCUUCACCUACAUAGUUA | STAT1 | | 59 |
| NO. 993 | +7A | | Y701 | | |
| SEQ | −12G, −4A, | GUCCUUGAACUACAUAGUUG | STAT1 | | 58.3 |
| NO. 994 | +7G | | Y701 | | |
| SEQ | −8C, +5A, | UUCCCUGACCUACAUAGAUU | STAT1 | | 58.1 |
| NO. 995 | +7U | | Y701 | | |
| SEQ | −12G, +3U | GUCCUUGACCUACAUUGUUC | STAT1 | | 58 |
| NO. 996 | | | Y701 | | |
| SEQ | +3U, +7A | UUCCUUGACCUACAUUGUUA | STAT1 | | 57.7 |
| NO. 997 | | | Y701 | | |
| SEQ | −6A, +3G, | UUCCUUAACCUACAUGGUCC | STAT1 | | 57.3 |
| NO. 998 | +6C | | Y701 | | |
| SEQ | −6A, −3U, | UUCCUUAACUUACAUAGUAC | STAT1 | | 57.1 |
| NO. 999 | +6A | | Y701 | | |
| SEQ | −11C, −7G, | UCCCUGUACCUACAUAGUUC | STAT1 | | 57.1 |
| NO. | −6U | | Y701 | | |
| 1000 | | | | | |
| SEQ | −12C, −10U, | CUUCUUGACCUACAUUGUUC | STAT1 | | 57.1 |
| NO. | +3U | | Y701 | | |
| 1001 | | | | | |
| SEQ | −12A, +3U | AUCCUUGACCUACAUUGUUC | STAT1 | | 57.1 |
| NO. | | | Y701 | | |
| 1002 | | | | | |
| SEQ | −11G, −6C, | UGCCUUCACGUACAUAGUUC | STAT1 | | 57.1 |
| NO. | −3G | | Y701 | | |
| 1003 | | | | | |
| SEQ | −12A, +2C | AUCCUUGACCUACACAGUUC | STAT1 | | 56.7 |
| NO. | | | Y701 | | |
| 1004 | | | | | |
| SEQ | −12A, −6C | AUCCUUCACCUACAUAGUUC | STAT1 | | 56.5 |
| NO. | | | Y701 | | |
| 1005 | | | | | |
| SEQ | −6G, +5G | CUACGUGGUUAACACAGGAG | IFNAR1 | Rule 8 | 89.3 |
| NO. | | | Y466 | | |
| 1006 | | | | | |
| SEQ | +2U, +5G | CUACGUAGUUAACAUAGGAG | IFNAR1 | Rule 5, | 88.3 |
| NO. | | | Y466 | Rule 8 | |
| 1007 | | | | | |
| SEQ | −12A, +5G | AUACGUAGUUAACACAGGAG | IFNAR1 | Rule 8 | 87.8 |
| NO. | | | Y466 | | |
| 1008 | | | | | |
| SEQ | −2C, +5G | CUACGUAGUUCACACAGGAG | IFNAR1 | Rule 8 | 85 |
| NO. | | | Y466 | | |
| 1009 | | | | | |
| SEQ | −7G, +5G | CUACGGAGUUAACACAGGAG | IFNAR1 | Rule 8 | 84.7 |
| NO. | | | Y466 | | |
| 1010 | | | | | |
| SEQ | +2A, +5G | CUACGUAGUUAACAAAGGAG | IFNAR1 | Rule 8 | 84.5 |
| NO. | | | Y466 | | |
| 1011 | | | | | |
| SEQ | −8C, +5G | CUACCUAGUUAACACAGGAG | IFNAR1 | Rule 2, | 83 |
| NO. | | | Y466 | Rule 8 | |
| 1012 | | | | | |
| SEQ | +2G, +5G | CUACGUAGUUAACAGAGGAG | IFNAR1 | Rule 8 | 81.3 |
| NO. | | | Y466 | | |
| 1013 | | | | | |
| SEQ | −8A, +5G | CUACAUAGUUAACACAGGAG | IFNAR1 | Rule 8 | 73.4 |
| NO. | | | Y466 | | |
| 1014 | | | | | |
| SEQ | −8C, +7U | CUACCUAGUUAACACAGAAU | IFNAR1 | Rule 2 | 71.7 |
| NO. | | | Y466 | | |
| 1015 | | | | | |
| SEQ | −6U, +5G | CUACGUUGUUAACACAGGAG | IFNAR1 | Rule 8 | 71.7 |
| NO. | | | Y466 | | |
| 1016 | | | | | |
| SEQ | −8U, +3G | CUACUUAGUUAACACGGAAG | IFNAR1 | Rule 2 | 71.6 |
| NO. | | | Y466 | | |
| 1017 | | | | | |
| SEQ | +1U, +5G | CUACGUAGUUAACUCAGGAG | IFNAR1 | Rule 8 | 71.4 |
| NO. | | | Y466 | | |
| 1018 | | | | | |
| SEQ | −5A, +5G | CUACGUAAUUAACACAGGAG | IFNAR1 | Rule 8 | 71.2 |
| NO. | | | Y466 | | |
| 1019 | | | | | |
| SEQ | +5G | CUACGUAGUUAACACAGGAG | IFNAR1 | Rule 8 | 71.1 |
| NO. | | | Y466 | | |
| 1020 | | | | | |
| SEQ | −11C, +7U | CCACGUAGUUAACACAGAAU | IFNAR1 | | 70.9 |
| NO. | | | Y466 | | |
| 1021 | | | | | |
| SEQ | +3C, +5G | CUACGUAGUUAACACCGGAG | IFNAR1 | Rule 8 | 70.5 |
| NO. | | | Y466 | | |
| 1022 | | | | | |
| SEQ | −11C, +7A | CCACGUAGUUAACACAGAAA | IFNAR1 | | 69.7 |
| NO. | | | Y466 | | |
| 1023 | | | | | |
| SEQ | −3G, +5G | CUACGUAGUGAACACAGGAG | IFNAR1 | Rule 8 | 68.5 |
| NO. | | | Y466 | | |
| 1024 | | | | | |
| SEQ | +3U, +7C | CUACGUAGUUAACACUGAAC | IFNAR1 | | 67.6 |
| NO. | | | Y466 | | |
| 1025 | | | | | |
| SEQ | −8C, +4C, | CUACCUAGUUAACACACGAG | IFNAR1 | Rule 2 | 67.6 |
| NO. | +5G | | Y466 | | |
| 1026 | | | | | |
| SEQ | −12G, −11C, | GCUCGUAGUUAACACUGAAG | IFNAR1 | | 66.8 |
| NO. | −10U, +3U | | Y466 | | |
| 1027 | | | | | |
| SEQ | −10G, −7A, | CUGCGAAGUUAACACAGAAU | IFNAR1 | | 66.5 |
| NO. | +7U | | Y466 | | |
| 1028 | | | | | |
| SEQ | −8C, +4A | CUACCUAGUUAACACAAAAG | IFNAR1 | Rule 2 | 66.2 |
| NO. | | | Y466 | | |
| 1029 | | | | | |
| SEQ | −9A, +1A, | AACACGGUGACAGCAUCU | CTNNB1 | | 91.7 |
| NO. | +3C, +4A, | | T41 | | |
| 1030 | +7U | | | | |
| SEQ | −10C, −5U, | CCCACUGUGAUGCUAUCG | CTNNB1 | | 90 |
| NO. | 0U, +2C, | | T41 | | |
| 1031 | +4A | | | | |
| SEQ | +1U, +3C, | ACCACGGUGACUGCGUUG | CTNNB1 | | 83.3 |
| NO. | +6U | | T41 | | |
| 1032 | | | | | |
| SEQ | −6A, +2C | ACCAAGGUGACGCUGUCG | CTNNB1 | | 81.2 |
| NO. | | | T41 | | |
| 1033 | | | | | |
| SEQ | −9A, −8A, | AAAACGGUGACGGUUUCG | CTNNB1 | | 70 |
| NO. | +4U | | T41 | | |
| 1034 | | | | | |
| SEQ | −10U, −6G, | UCCAGUGUGACAGUGUAG | CTNNB1 | | 66.7 |
| NO. | −5U, +1A, | | T41 | | |
| 1035 | +6A | | | | |
| SEQ | −8A, +3C, | ACAACGGUGACGGCGUCA | CTNNB1 | | 65 |
| NO. | +7A | | T41 | | |
| 1036 | | | | | |
| SEQ | +2A, +7C | ACCACGGUGACGAUGUCC | CTNNB1 | | 62.5 |
| NO. | | | T41 | | |
| 1037 | | | | | |
| SEQ | −10U, −9G, | UGCACGGUGACGGAGUCG | CTNNB1 | | 60.9 |
| NO. | +3A | | T41 | | |
| 1038 | | | | | |
| SEQ | −3C, +4U, | ACCACGGCGACGGUUUCC | CTNNB1 | | 57.1 |
| NO | +7C | | T41 | | |
| 1039 | | | | | |
| SEQ | −8G, −2C, | ACGACGGUCACGGAGUCC | CTNNB1 | | 57.1 |
| NO. | +3A, +7C | | T41 | | |
| 1040 | | | | | |
| SEQ | −10U, −8U, | UCUACGGUGACGGAGUCG | CTNNB1 | | 55.9 |
| NO. | +3A | | T41 | | |
| 1041 | | | | | |
| SEQ | −10G, +7C | GCCACGGUGACGGUGUCC | CTNNB1 | | 55.9 |
| NO. | | | T41 | | |
| 1042 | | | | | |
| SEQ | −9U, +7C | AUCACGGUGACGGUGUCC | CTNNB1 | | 55 |
| NO. | | | T41 | | |
| 1043 | | | | | |
| SEQ | −6U, +4C, | ACCAUGGUGACGGUCUCC | CTNNB1 | | 54.8 |
| NO. | +7C | | T41 | | |
| 1044 | | | | | |
| SEQ | −8U, +3C | ACUACGGUGACGGCGUCG | CTNNB1 | | 54.8 |
| NO. | | | T41 | | |
| 1045 | | | | | |
| SEQ | −10G, +3C, | GCCACGGUGACGGCGUCC | CTNNB1 | | 54.4 |
| NO. | +7C | | T41 | | |
| 1046 | | | | | |
| SEQ | +5A, +7U | ACCACGGUGACGGUGACU | CTNNB1 | | 53.5 |
| NO. | | | T41 | | |
| 1047 | | | | | |
| SEQ | −10U, +3C | UCCACGGUGACGGCGUCG | CTNNB1 | | 52.6 |
| NO. | | | T41 | | |
| 1048 | | | | | |
| SEQ | −10G, −6A, | GCCAAGGUGACGGCAUCG | CTNNB1 | | 52.4 |
| NO. | +3C, +4A | | T41 | | |
| 1049 | | | | | |
| SEQ | −6G, +5C, | ACCAGGGUGACGGUGCAG | CTNNB1 | | 52.4 |
| NO. | +6A | | T41 | | |
| 1050 | | | | | |
| SEQ | −6G, +4A, | ACCAGGGUGACGGUAUCC | CTNNB1 | | 52.2 |
| NO. | +7C | | T41 | | |
| 1051 | | | | | |
| SEQ | −10G, +3A | GCCACGGUGACGGAGUCG | CTNNB1 | | 52.2 |
| NO. | | | T41 | | |
| 1052 | | | | | |
| SEQ | −10C, +4A, | CCCACGGUGACGGUAUCU | CTNNB1 | | 52 |
| NO. | +7U | | T41 | | |
| 1053 | | | | | |
| SEQ | −8U, +7U | ACUACGGUGACGGUGUCU | CTNNB1 | | 51.6 |
| NO. | | | T41 | | |
| 1054 | | | | | |
| SEQ | −5U, +3C, | ACCACUGUGACGGCGUAG | CTNNB1 | | 51.6 |
| NO. | +6A | | T41 | | |
| 1055 | | | | | |
| SEQ | −8U, +4U, | ACUACGGUGACGGUUUAG | CTNNB1 | | 51.4 |
| NO. | +6A | | T41 | | |
| 1056 | | | | | |
| SEQ | −10G, +4A, | GCCACGGUGACGGUAUAG | CTNNB1 | | 51.2 |
| NO. | +6A | | T41 | | |
| 1057 | | | | | |
| SEQ | −10C, +3A | CCCACGGUGACGGAGUCG | CTNNB1 | | 51 |
| NO. | | | T41 | | |
| 1058 | | | | | |
| SEQ | −8U, +7C | ACUACGGUGACGGUGUCC | CTNNB1 | | 50.3 |
| NO. | | | T41 | | |
| 1059 | | | | | |
| SEQ | −6U, +3A | ACCAUGGUGACGGAGUCG | CTNNB1 | | 50 |
| NO. | | | T41 | | |
| 1060 | | | | | |
| SEQ | −8U, −7G, | ACUGCGGUGACGGCGUCC | CTNNB1 | | 50 |
| NO. | +3C, +7C | | T41 | | |
| 1061 | | | | | |
| SEQ | −9A, −6A | AACAAGGUGACGGUGUCG | CTNNB1 | | 50 |
| NO. | | | T41 | | |
| 1062 | | | | | |
| SEQ | −8U, +2A, | ACUACGGUGACGAAGUCG | CTNNB1 | | 50 |
| NO. | +3A | | T41 | | |
| 1063 | | | | | |
| SEQ | −10C, +3A, | CCCACGGUGACGGAGUGG | CTNNB1 | | 50 |
| NO. | +6G | | T41 | | |
| 1064 | | | | | |
| SEQ | −9G, +5G | AGCACGGUGACGGUGGCG | CTNNB1 | | 50 |
| NO. | | | T41 | | |
| 1065 | | | | | |
| SEQ | −10G, −8G, | GCGACGGUGACGGUGUAG | CTNNB1 | | 50 |
| NO. | +6A | | T41 | | |
| 1066 | | | | | |
| SEQ | +3C, +7U | ACCACGGUGACGGCGUCU | CTNNB1 | | 50 |
| NO. | | | T41 | | |
| 1067 | | | | | |
| SEQ | −6A, −5C, | ACCAACGUGACGAUGUCU | CTNNB1 | | 50 |
| NO. | +2A, +7U | | T41 | | |
| 1068 | | | | | |
| SEQ | −8G, −2A, | ACGACGGUAACGGCGUCG | CTNNB1 | | 50 |
| NO. | +3C | | T41 | | |
| 1069 | | | | | |
| SEQ | +5A, +7C | ACCACGGUGACGGUGACC | CTNNB1 | | 49.3 |
| NO. | | | T41 | | |
| 1070 | | | | | |
| SEQ | −6A, +3U | UUCCCUAAAAGUCGCUCCGG | TMEM173 | | 100 |
| NO. | | | N154 | | |
| 1071 | | | | | |
| SEQ | −4G, −1C, | UUCCCUUAGAGCCGCAACGG | TMEM173 | | 100 |
| NO. | +4A | | N154 | | |
| 1072 | | | | | |
| SEQ | −12G, −6A, | GUCCCUAAAAGUCGCACAGG | TMEM173 | | 100 |
| NO. | +5A | | N154 | | |
| 1073 | | | | | |
| SEQ | −12C, −8U, | CUCCUUUAAAGUCGUACCGG | TMEM173 | Rule 5 | 95 |
| NO. | +2U | | N154 | | |
| 1074 | | | | | |
| SEQ | −11C, +6U | UCCCCUUAAAGUCGCACCUG | TMEM173 | | 92.3 |
| NO. | | | N154 | | |
| 1075 | | | | | |
| SEQ | −5C, −4G | UUCCCUUCGAGUCGCACCGG | TMEM173 | | 92.3 |
| NO. | | | N154 | | |
| 1076 | | | | | |
| SEQ | −12C, −10A, | CUACCUUAUAGUCGCACCGG | TMEM173 | | 92.3 |
| NO. | −4U | | N154 | | |
| 1077 | | | | | |
| SEQ | −4G, +6U | UUCCCUUAGAGUCGCACCUG | TMEM173 | | 90.9 |
| NO. | | | N154 | | |
| 1078 | | | | | |
| SEQ | +2U, +5G, | UUCCCUUAAAGUCGUACGCU | TMEM173 | Rule 5 | 90 |
| NO. | +6C, +7U | | N154 | | |
| 1079 | | | | | |
| SEQ | −12A, −8U, | AUCCUUUAAAGUCGCACCUG | TMEM173 | | 83.9 |
| NO. | +6U | | N154 | | |
| 1080 | | | | | |
| SEQ | −6G, +3G | UUCCCUGAAAGUCGCGCCGG | TMEM173 | | 83.3 |
| NO. | | | N154 | | |
| 1081 | | | | | |
| SEQ | −8G, +7C | UUCCGUUAAAGUCGCACCGC | TMEM173 | | 83.3 |
| NO. | | | N154 | | |
| 1082 | | | | | |
| SEQ | −9A, +3G, | UUCACUUAAAGUCGCGCCCG | TMEM173 | | 82.8 |
| NO. | +6C | | N154 | | |
| 1083 | | | | | |
| SEQ | −12C, +3C | CUCCCUUAAAGUCGCCCCGG | TMEM173 | | 81.7 |
| NO. | | | N154 | | |
| 1084 | | | | | |
| SEQ | −7A, +7C | UUCCCAUAAAGUCGCACCGC | TMEM173 | | 80 |
| NO. | | | N154 | | |
| 1085 | | | | | |
| SEQ | −10A, +7U | UUACCUUAAAGUCGCACCGU | TMEM173 | | 80 |
| NO. | | | N154 | | |
| 1086 | | | | | |
| SEQ | −9G, 0U, | UUCGCUUAAAGUUGUACCUG | TMEM173 | Rule 5 | 78.6 |
| NO. | +2U, +6U | | N154 | | |
| 1087 | | | | | |
| SEQ | −6A, −1A, | UUCCCUAAAAGACGCACGGG | TMEM173 | | 78.3 |
| NO. | +5G | | N154 | | |
| 1088 | | | | | |
| SEQ | −12A, −8U | AUCCUUUAAAGUCGCACCGG | TMEM173 | | 74.1 |
| NO. | | | N154 | | |
| 1089 | | | | | |
| SEQ | −6G, +5U, | UUCCCUGAAAGUCGCACUAG | TMEM173 | | 72.7 |
| NO. | +6A | | N154 | | |
| 1090 | | | | | |
| SEQ | −11A, −7A, | UACCCAUAUAGUCGCUCAGG | TMEM173 | | 72.7 |
| NO. | −4U, +3U, | | N154 | | |
| 1091 | +5A | | | | |
| SEQ | −9U, +2G | UUCUCUUAAAGUCGGACCGG | TMEM173 | | 70.6 |
| NO. | | | N154 | | |
| 1092 | | | | | |
| SEQ | −6A, −4U, | UUCCCUAAUGGUCGCAAUGG | TMEM173 | | 70 |
| NO. | 3−G, +4A, | | N154 | | |
| 1093 | +5U | | | | |
| SEQ | −12A, +2U, | AUCCCUUAAAGUCGUACCAG | TMEM173 | Rule 5 | 70 |
| NO. | +6A | | N154 | | |
| 1094 | | | | | |
| SEQ | −10G, −6G | UUGCCUGAAAGUCGCACCGG | TMEM173 | | 69.2 |
| NO. | | | N154 | | |
| 1095 | | | | | |
| SEQ | −9G, +2U, | UUCGCUUAAAGUCGUACGGG | TMEM173 | Rule 5 | 66.7 |
| NO. | +5G | | N154 | | |
| 1096 | | | | | |
| SEQ | +3G, +4A, | UUCCCUUAAAGUCGCGACAG | TMEM173 | | 66.7 |
| NO. | +6A | | N154 | | |
| 1097 | | | | | |
| SEQ | −2C, +7C | UUCCCUUAAACUCGCACCGC | TMEM173 | | 66.7 |
| NO. | | | N154 | | |
| 1098 | | | | | |
| SEQ | −7A, +4G, | UUCCCAUAAAGUCGCAGGGG | TMEM173 | | 65.2 |
| NO. | +5G | | N154 | | |
| 1099 | | | | | |
| SEQ | −12G, −6A | GUCCCUAAAAGUCGCACCGG | TMEM173 | | 63.4 |
| NO. | | | N154 | | |
| 1100 | | | | | |
| SEQ | −12G, +6A | GUCCCUUAAAGUCGCACCAG | TMEM173 | | 61.8 |
| NO. | | | N154 | | |
| 1101 | | | | | |
| SEQ | −10U, −3C | UUUCCUUAACGUCGCACCGG | TMEM173 | | 61.5 |
| NO. | | | N154 | | |
| 1102 | | | | | |
| SEQ | −12G, −11A, | GACUCUUAAAAUCGCAACGG | TMEM173 | | 61.5 |
| NO. | −9U, −2A, | | N154 | | |
| 1103 | +4A | | | | |
| SEQ | −11A, −10U | UAUCCUUAAAGUCGCACCGG | TMEM173 | | 59.5 |
| NO. | | | N154 | | |
| 1104 | | | | | |
| SEQ | −12A, +2G, | AUCCCUUAAAGUCGGGCCGG | TMEM173 | | 59.1 |
| NO. | +3G | | N154 | | |
| 1105 | | | | | |
| SEQ | −7C, +6C, | UUCCCCUAAAGUCGCACCCA | TMEM173 | | 59.1 |
| NO. | +7A | | N154 | | |
| 1106 | | | | | |
| SEQ | +2U, +5A | UUCCCUUAAAGUCGUACAGG | TMEM173 | Rule 5 | 58.9 |
| NO. | | | N154 | | |
| 1107 | | | | | |
| SEQ | −8G, +3U | UUCCGUUAAAGUCGCUCCGG | TMEM173 | | 58.3 |
| NO. | | | N154 | | |
| 1108 | | | | | |
| SEQ | −8G, −6A, | UUCCGUAAAAGUCGCACCUG | TMEM173 | | 57.9 |
| NO. | +6U | | N154 | | |
| 1109 | | | | | |
| SEQ | +5G, +7U | UUCCCUUAAAGUCGCACGGU | TMEM173 | | 57.4 |
| NO. | | | N154 | | |
| 1110 | | | | | |
| SEQ | −8A, −6G, | UUCCAUGAAAGUCGCACGGG | TMEM173 | | 56.6 |
| NO. | +5G | | N154 | | |
| 1111 | | | | | |
| SEQ | −8G, −6A, | UUCCGUAAAAGUCGUACCAG | TMEM173 | Rule 5 | 56.2 |
| NO. | +2U, +6A | | N154 | | |
| 1112 | | | | | |
| SEQ | −7C, −6C, | UUCCCCCAAAGUCGCGCCGC | TMEM173 | | 55.6 |
| NO. | +3G, +7C | | N154 | | |
| 1113 | | | | | |
| SEQ | −9G, −7G, | UUCGCGUAAAGACGCACCGG | TMEM173 | | 54.8 |
| NO. | −1A | | N154 | | |
| 1114 | | | | | |
| SEQ | −4U, −2A | UUCCCUUAUAAUCGCACCGG | TMEM173 | | 54.5 |
| NO. | | | N154 | | |
| 1115 | | | | | |
| SEQ | −8U, −7C, | UUCCUCUAAAGUUGCACCUG | TMEM173 | | 53.9 |
| NO. | 0U, +6U | | N154 | | |
| 1116 | | | | | |
| SEQ | −6C, +4A | UUCCCUCAAAGUCGCAACGG | TMEM173 | | 53.9 |
| NO. | | | N154 | | |
| 1117 | | | | | |
| SEQ | −12A, −11G, | AGCCCUAAAAGUCGCACGGG | TMEM173 | | 53.3 |
| NO. | −6A, +5G | | N154 | | |
| 1118 | | | | | |
| SEQ | +3G, +5G | UUCCCUUAAAGUCGCGCGGG | TMEM173 | | 52.9 |
| NO. | | | N154 | | |
| 1119 | | | | | |
| SEQ | +2U, +5G | UUCCCUUAAAGUCGUACGGG | TMEM173 | Rule 5 | 52.8 |
| NO. | | | N154 | | |
| 1120 | | | | | |
| SEQ | −9A, +5G | UUCACUUAAAGUCGCACGGG | TMEM173 | | 52.6 |
| NO. | | | N154 | | |
| 1121 | | | | | |
| SEQ | +2G, +6U | UUCCCUUAAAGUCGGACCUG | TMEM173 | | 52 |
| NO. | | | N154 | | |
| 1122 | | | | | |
| SEQ | −2A, +6A | UUCCCUUAAAAUCGCACCAG | TMEM173 | | 52 |
| NO. | | | N154 | | |
| 1123 | | | | | |
| SEQ | +2U, +3G | UUCCCUUAAAGUCGUGCCGG | TMEM173 | | 51.6 |
| NO. | | | N154 | | |
| 1124 | | | | | |
| SEQ | +2U, +6A | UUCCCUUAAAGUCGUACCAG | TMEM173 | Rule 5 | 51.6 |
| NO. | | | N154 | | |
| 1125 | | | | | |
| SEQ | −12G, +3U | GUCCCUUAAAGUCGCUCCGG | TMEM173 | | 51 |
| NO. | | | N154 | | |
| 1126 | | | | | |
| SEQ | −11A, −5U | UACCCUUUAAGUCGCACCGG | TMEM173 | | 50 |
| NO. | | | N154 | | |
| 1127 | | | | | |
| SEQ | −8U, −7C, | UUCCUCUAAAGUCGUACAGG | TMEM173 | Rule 5 | 50 |
| NO. | +2U, +5A | | N154 | | |
| 1128 | | | | | |
| SEQ | −10U, −7G, | UUUCCGUAAAGUCGCAGGAG | TMEM173 | | 50 |
| NO. | +4G, +5G, | | N154 | | |
| 1129 | +6A | | | | |
| SEQ | −5U, 0U, | UUCCCUUUAAGUUGCACUGG | TMEM173 | | 50 |
| NO. | +5U | | N154 | | |
| 1130 | | | | | |
| SEQ | −6A, +6A | UUCCCUAAAAGUCGCACCAG | TMEM173 | | 50 |
| NO. | | | N154 | | |
| 1131 | | | | | |
| SEQ | −12A, −11A, | AACCCUUAAAAUCGCACCGG | TMEM173 | | 50 |
| NO. | −2A | | N154 | | |
| 1132 | | | | | |
| SEQ | −7G, +2U, | UUCCCGUAAAGUCGUACCGC | TMEM173 | Rule 5 | 50 |
| NO. | +7C | | N154 | | |
| 1133 | | | | | |
| SEQ | −10U, −9A, | UUUACUUAAAGUCGGACCGG | TMEM173 | | 50 |
| NO. | +2G | | N154 | | |
| 1134 | | | | | |
| SEQ | −8A, −6U, | GGACUUCCUACCGAAACU | TMEM173 | | 94.3 |
| NO. | +2G, +3A | | R284 | | |
| 1135 | | | | | |
| SEQ | −7A, −5A, | GGCACAACUACCCGACCU | TMEM173 | | 90.1 |
| NO. | −4A, +5C | | R284 | | |
| 1136 | | | | | |
| SEQ | +5G, +6A | GGCCCUCCUACCCGAGAU | TMEM173 | | 80.6 |
| NO. | | | R284 | | |
| 1137 | | | | | |
| SEQ | −6G, −5G, | GGCCGGCCUACCCCAACU | TMEM173 | | 79 |
| NO. | +3C | | R284 | | |
| 1138 | | | | | |
| SEQ | −5C, +6G | GGCCCCCCUACCCGAAGU | TMEM173 | | 78 |
| NO. | | | R284 | | |
| 1139 | | | | | |
| SEQ | +3C, +6G | GGCCCUCCUACCCCAAGU | TMEM173 | | 72 |
| NO. | | | R284 | | |
| 1140 | | | | | |
| SEQ | −8U, −5C, | GGUCCCCCUACCCGAACC | TMEM173 | | 71 |
| NO. | +7C | | R284 | | |
| 1141 | | | | | |
| SEQ | −5C, +3A | GGCCCCCCUACCCAAACU | TMEM173 | | 70.5 |
| NO. | | | R284 | | |
| 1142 | | | | | |
| SEQ | +2U, +3C, | GGCCCUCCUACCUCAAGU | TMEM173 | | 69.9 |
| NO. | +6G | | R284 | | |
| 1143 | | | | | |
| SEQ | −8G, −7A, | GGGACUCCUACCCGAAGU | TMEM173 | | 69.7 |
| NO. | +6G | | R284 | | |
| 1144 | | | | | |
| SEQ | −6U, +3C, | GGCCUUCCUACCCCAAAU | TMEM173 | | 69.4 |
| NO. | +6A | | R284 | | |
| 1145 | | | | | |
| SEQ | +2U, +3A, | GGCCCUCCUACCUAAACA | TMEM173 | | 68.4 |
| NO. | +7A | | R284 | | |
| 1146 | | | | | |
| SEQ | −5C, +3A, | GGCCCCCCUACCCAAAUU | TMEM173 | | 66.7 |
| NO. | +6U | | R284 | | |
| 1147 | | | | | |
| SEQ | −5C, +2U, | GGCCCCCCUACCUGAACC | TMEM173 | Rule 5 | 66.1 |
| NO. | +7C | | R284 | | |
| 1148 | | | | | |
| SEQ | −4G, +6U | GGCCCUGCUACCCGAAUU | TMEM173 | | 65.6 |
| NO. | | | R284 | | |
| 1149 | | | | | |
| SEQ | −7A, +3C, | GGCACUCCUACCCCAAGU | TMEM173 | | 64.8 |
| NO. | +6G | | R284 | | |
| 1150 | | | | | |
| SEQ | −10A, −7A, | AGCACUCCUACCCCAAGU | TMEM173 | | 64.2 |
| NO. | +3C, +6G | | R284 | | |
| 1151 | | | | | |
| SEQ | −3A, +4G, | GGCCCUCAUACCCGGAAU | TMEM173 | | 63.6 |
| NO. | +6A | | R284 | | |
| 1152 | | | | | |
| SEQ | −3U, +3C, | GGCCCUCUUACCCCCACU | TMEM173 | | 63.2 |
| NO. | +4C | | R284 | | |
| 1153 | | | | | |
| SEQ | +2U, +3C, | GGCCCUCCUACCUCAGCU | TMEM173 | | 62.9 |
| NO. | +5G | | R284 | | |
| 1154 | | | | | |
| SEQ | −10A, +3A, | AGCCCUCCUACCCAAAGU | TMEM173 | | 62.5 |
| NO. | +6G | | R284 | | |
| 1155 | | | | | |
| SEQ | +2U, +6G | GGCCCUCCUACCUGAAGU | TMEM173 | Rule 5 | 62.4 |
| NO. | | | R284 | | |
| 1156 | | | | | |
| SEQ | +4G, +5G | GGCCCUCCUACCCGGGCU | TMEM173 | | 62.3 |
| NO. | | | R284 | | |
| 1157 | | | | | |
| SEQ | −10C, +5C, | CGCCCUCCUACCCGACUU | TMEM173 | | 62.1 |
| NO. | +6U | | R284 | | |
| 1158 | | | | | |
| SEQ | −7G, +3C | GGCGCUCCUACCCCAACU | TMEM173 | | 61.5 |
| NO. | | | R284 | | |
| 1159 | | | | | |
| SEQ | −6A, −5A, | GGCCAACCUACCCCAACU | TMEM173 | | 61.2 |
| NO. | +3C | | R284 | | |
| 1160 | | | | | |
| SEQ | −10A, +3C | AGCCCUCCUACCCCAACU | TMEM173 | | 61.2 |
| NO. | | | R284 | | |
| 1161 | | | | | |
| SEQ | −8G, +3C | GGGCCUCCUACCCCAACU | TMEM173 | | 61.1 |
| NO. | | | R284 | | |
| 1162 | | | | | |
| SEQ | −8G, −7A, | GGGAAUCCUACCCGAACG | TMEM173 | | 61.1 |
| NO. | −6A, +7G | | R284 | | |
| 1163 | | | | | |
| SEQ | +4C, +5G, | GGCCCUCCUACCCGCGUU | TMEM173 | | 61.1 |
| NO. | +6U | | R284 | | |
| 1164 | | | | | |
| SEQ | −4U, −3U, | GGCCCUUUUACCCGAAGU | TMEM173 | | 60.6 |
| NO. | +6G | | R284 | | |
| 1165 | | | | | |

### REFERENCES

1. Bass, B. L., and Weintraub H. (1987). A developmentally regulated activity that unwinds RNA duplexes. Cell. 48:607-13.
2. Merkle T., Merz S., Reautschnig P., Blaha A., Li Q., Vogel P., Wettengel J., Li J. B. and Stafforst T. (2019). Precise RNA editing by recruiting endogenous ADARs with antisense oligonucleotides. Nature Biotechnology, 37:133-138.
3. Rebagliati, M. R.; Melton D. A. (1987) Antisense RNA injections in fertilized frog eggs reveal an RNA duplex unwinding activity. Cell. 48:599-605.Zinshteyn, B., and Nishikura, K. (2009). Adenosine-to-inosine RNA editing. Rev. Syst. Biol. Med. 1 (2): 202-209.
4. Tomaselli, S., Locatelli, F., Gallo, A. (2014). The RNA editing enzymes ADARs: mechanism of action and human disease. Cell and Tissue Research. 356:527-532.
5. Wulff, B.-E., and Nishikura, K. (2010). Substitutional A-to-IRNA editing. Interdiscip. Rev. RNA. 1 (1): 90-101.
6. Zinshteyn B., Nishikura K. (2009). Adenosine-to-inosine RNA editing. WIREs Systems Biology and Medicine. 1 (2): 202-209.

1. An oligonucleotide for adenosine to inosine (A-to-I) editing of a target RNA sequence, wherein the oligonucleotide comprises a general core sequence:
3′-N₋₁₀ N₋₉ N₋₈ N₋₇ N₋₆ N₋₅ N₋₄ N₋₃ N₋₂ N₋₁ N₀ N₊₁ N₊₂ N₊₃ N₊₄ N₊₅ N₊₆ N₊₇ N₊₈ N₊₉-5′;
wherein (3′-N₋₁ N₀ N₊₁-5′) is a central base triplet (CBT) with the central nucleotide (N₀) directly opposite the target adenosine (A) to be edited, when the oligonucleotide is hybridized to the target RNA sequence, and
wherein the target RNA sequence has a core sequence of:
5′-n₋₁₀ n₋₉ n₋₈ n₋₇ n₋₆ n₋₅ n₋₄ n₋₃ n₋₂ n₋₁ A n₊₁ n₊₂ n₊₃ n₊₄ n₊₅ n₊₆ n₊₇ n₊₈ n₊₉-3′;
and wherein
(i) there is a mismatch at N₀, when the oligonucleotide is hybridized to the target RNA sequence;
(ii) there is one or more nucleotide mismatches outside of the CBT that is/are not complementary to the corresponding position in the target RNA sequence; and
(iii) the one or more nucleotide mismatch(es) outside of the CBT is/are as defined by:
a) a third generalizable rule (Rule 3), wherein the oligonucleotide contains a C or U at N₋₇, and wherein the target sequence contains a C residue at n₋₇, flanked by a 5′ G or C and a 3′ A or U;
b) a first generalizable rule (Rule 1), wherein the oligonucleotide contains a C or U at N₋₉, and wherein the target sequence contains a C residue at n₋₉, flanked by a 5′ C or G and 3′ A or U;
c) a second generalizable rule (Rule 2), wherein the oligonucleotide contains a Cor U at N₋₈, and wherein the target sequence contains a C at n₋₈, flanked by a 5′ C or G and 3′ A or U;
d) a fourth generalizable rule (Rule 4), wherein the oligonucleotide contains a Cor U at N₋₆, and wherein the target sequence contains a C residue at n₋₆, flanked by a 5′ G or C and a 3′ A or U;
e) a fifth generalizable rule (Rule 5), wherein the oligonucleotide contains a U at N₊₂, and wherein the target sequence contains a G residue at n₊₂;
f) a sixth generalizable rule (Rule 6), wherein the oligonucleotide contains a C or U at N₊₂, and wherein the target sequence contains a U at n₊₂;
g) a seventh generalizable rule (Rule 7), wherein the oligonucleotide contains an A at N₊₄, and wherein the target sequence contains a C residue at n₊₄, flanked by a 5′ G and 3′ C; and/or
h) an eighth generalizable rule (Rule 8), wherein the oligonucleotide contains a G at N₊₅, and wherein the target sequence contains a U residue at n₊₅, flanked by a 5′ C and 3′ U.

2. The oligonucleotide of claim 1, wherein
i) under section a), the target sequence is SERPINA1 E342K;
ii) under section b), the target sequence is SNCA A53T or STAT3 Y705;
iii) under section c), the target sequence is ALDH2 E504K, MeCP2 R106Q, or IFNAR1 Y466;
iv) under section d), the target sequence is SCN9A W897X;
v) under section e), the target sequence is SERPINA1 E342K, KRAS G12D, F5 R534Q, ABCA4 G1961E, IFNAR1 Y466, TMEM173 N154, or TMEM173 R284;
vi) under section f), the target sequence is GFER R194H, TP53 R175H, or KRAS G12S;
vii) under section g), the target sequence is TP53 R175H; or
viii) under section h), the target sequence is IFNAR1 Y466.

3. The oligonucleotide of claim 1 or 2, wherein the oligonucleotide comprises two nucleotide mismatches outside of the CBT that are not complementary to the corresponding position in the target nucleic sequence when the oligonucleotides is hybridized to the target nucleic acid sequence, wherein
a) a first mismatch is defined according to Rule 3 and a second mismatch is defined according to Rule 5, optionally wherein the target sequence is SERPINA1 E342K;
b) a first mismatch is defined according to Rule 6 and a second mismatch is defined according to Rule 7, optionally wherein the target sequence is TP53 R175H;
c) a first mismatch is defined according to Rule 5 and a second mismatch is defined according to Rule 8 optionally wherein the target sequence is IFNAR1 Y466; or
d) a first mismatch is defined according to Rule 2 and a second mismatch is defined according to Rule 8, optionally wherein the target sequence is IFNAR1 Y466.

4. An oligonucleotide of claim 1, wherein the oligonucleotide comprises one of the following 3′-to-5′ core sequence:
a) 3′-N₋₈ N₋₇ N₋₆ N₋₅ N₋₄ N₋₃ N₋₂ N₋₁ C-5′, wherein
C is the cytosine opposite the target adenosine in the target nucleic acid sequence; wherein
N₋₈ is C or G, forming a Watson-Crick base-pair with G or C in the target sequence;
N₋₇ is C or U, forming a mismatch with C in the target sequence; and
N₋₆ is A or U, forming a Watson-Crick base-pair with U or A in the target sequence;
b) 3′-N₋₁₀ N₋₉ N₋₈ N₋₇ N₋₆ N₋₅ N₋₄ N₋₃ N₋₂ N₋₁ C-5′, wherein
C is the cytosine opposite the target adenosine in the target nucleic acid sequence; wherein
N₋₁₀ is C or G, forming a Watson-Crick base-pair with G or C in the target sequence;
N₋₉ is C or U, forming a mismatch with C in the target sequence; and
N₋₈ is A or U, forming a Watson-Crick base-pair with U or A in the target sequence;
c) 3′-N₋₉ N₋₈ N₋₇ N₋₆ N₋₅ N₋₄ N₋₃ N₋₂ N₋₁ C-5′, wherein
C is the cytosine opposite the target adenosine in the target nucleic acid sequence; wherein
N₋₉ is C or G, forming a Watson-Crick base-pair with G or C in the target sequence;
N₋₈ is C or U, forming a mismatch with C in the target sequence; and
N₋₇ is A or U, forming a Watson-Crick base-pair with U or A in the target sequence;
d) 3′-N₋₇ N₋₆ N₋₅ N₋₄ N₋₃ N₋₂ N₋₁ C-5′, wherein
C is the cytosine opposite the target adenosine in the target nucleic acid sequence; wherein
N₋₇ is C or G, forming a Watson-Crick base-pair with G or C in the target sequence;
N₋₆ is C or U, forming a mismatch with C in the target sequence; and
N₋₅ is A or U, forming a Watson-Crick base-pair with U or A in the target sequence;
c) 3′-C N₊₁ N₊₂ N₊₃-5′, wherein
C is the cytosine opposite the target adenosine in the target nucleic acid sequence; wherein
N₊₂ is U, forming a wobble base-pair with G in the target sequence, and N₊₁ and N₊₃ are complementary to the corresponding positions in the target sequence;
f) 3′-C N₊₁ N₊₂ N₊₃-5′, wherein
C is the cytosine opposite the target adenosine in the target nucleic acid sequence; wherein
N₊₂ is C or U, forming a mismatch with U in the target sequence, and N₊₁ and N₊₃ are complementary to the corresponding positions in the target sequence;
g) 3′-C N₊₁ N₊₂ N₊₃ N₊₄ N45-5′, wherein
C is the cytosine opposite the target adenosine in the target nucleic acid sequence; wherein
N₊₃ is C, forming a Watson-Crick base-pair with G in the target sequence,
N₊₄ is A, forming a mismatch with C in the target sequence, and
N₊₅ is G, forming a Watson-Crick base-pair with C in the target sequence; or
h) 3′-C N₊₁ N₊₂ N₊₃ N₊₄ N₊₅ N₊₆-5′, wherein
C is the cytosine opposite the target adenosine in the target nucleic acid sequence; wherein
N₊₄ is G, forming a Watson-Crick base-pair with C in the target sequence,
N₊₅ is G, forming a wobble base-pair with U in the target sequence, and
N₊₆ is A, forming a Watson-Crick base-pair with U in the target sequence.

5. The oligonucleotide of any one of claims 1-4, wherein there is a mismatch at position N₋₁ and/or at position N₊₁, when the oligonucleotide is hybridized to the target RNA sequence.

6. The oligonucleotide of claim 5, wherein the oligonucleotide contains a G at N₋₁, and wherein there is a mismatch at N₋₁, when the oligonucleotide is hybridized to the target RNA sequence, preferably wherein the mismatch is a G/A.

7. The oligonucleotide of claim 6, wherein the target RNA sequence is SNCA A53T, ABCA4 G1961E, and KRAS G12D.

8. An oligonucleotide for adenosine to inosine (A-to-I editing) of a target RNA sequence,
wherein the oligonucleotide comprises one of the sequences selected from
a) SEQ ID NOs: 1 to 926; or
b) SEQ ID NOs: 927 to 1165; or
a sequence with at least 80% sequence identity to any of said sequences.

9. The oligonucleotide of claim 8, wherein the oligonucleotide has at least 85%, at least 90%, at least 95%, at least 99%, or 100% sequence identity to any of SEQ ID NOs: 223 to 291; SEQ ID NOs: 1-222; SEQ ID NOs: 292 to 339; SEQ ID NOs: 340 to 399; SEQ ID NOs: 400 to 518; SEQ ID NOs: 519 to 593; SEQ ID NOs: 594 to 634; SEQ ID NOs: 635 to 694; SEQ ID NOs: 695 to 728; SEQ ID NOs: 729 to 753; SEQ ID NOs: 754 to 796; SEQ ID NOs: 797 to 926; SEQ ID NOs: 927-966; SEQ ID NOs: 967 to 1005; SEQ ID NOs: 1006 to 1029; SEQ ID NOs: 1030 to 1070; SEQ ID NOs: 1071 to 1134; and SEQ ID NOs: 1135 to 1165.

10. The oligonucleotide of claim 8 or 9, wherein the intended target RNA sequence codes for one of the following proteins selected from a list consisting of:
a) SERPINA1 carrying the E342K mutation, and wherein the oligonucleotide comprises one of the sequences selected from a group consisting of SEQ ID NOs: 223 to 291;
b) SNCA carrying the A53T mutation, and wherein the oligonucleotide comprises one of the sequences selected from a group consisting of SEQ ID NOs: 1 to 222;
c) GFER carrying the R194H mutation, and wherein the oligonucleotide comprises one of the sequences selected from a group consisting of SEQ ID NOs: 292 to 339;
d) ALDH2 carrying the E504K mutation, and wherein the oligonucleotide comprises one of the sequences selected from a group consisting of SEQ ID NOs: 340 to 399;
e) TP53 carrying the oncogenic R175H mutation, and wherein the oligonucleotide comprises one of the sequences selected from a group consisting of SEQ ID NOs: 400 to 518;
f) SCN9A carrying the W897X mutation, and wherein the oligonucleotide comprises one of the sequences selected from a group consisting of SEQ ID NOs: 519 to 593;
g) HBB carrying the W38X mutation, and wherein the oligonucleotide comprises one of the sequences selected from a group consisting of SEQ ID NOs: 594 to 634;
h) KRAS carrying the oncogenic G12D mutation, and wherein the oligonucleotide comprises one of the sequences selected from a group consisting of SEQ ID NOs: 635 to 694;
i) KRAS carrying the oncogenic G12S mutation, and wherein the oligonucleotide comprises one of the sequences selected from a group consisting of SEQ ID NOs: 695 to 728;
j) MeCP2 carrying the R106Q mutation, and wherein the oligonucleotide comprises one of the sequences selected from a group consisting of SEQ ID NOs: 729 to 753;
k) F5 carrying the R534Q mutation, and wherein the oligonucleotide comprises one of the sequences selected from a group consisting of SEQ ID NOs: 754 to 796; and
l) ABCA4 carrying the G1961E mutation, and wherein the oligonucleotide comprises one of the sequences selected from a group consisting of SEQ ID NOs: 797 to 926.

11. The oligonucleotide of any one of claims 1-10, wherein the oligonucleotide is partially complementary to SEQ ID NO: 1169 and wherein the oligonucleotide comprises one of the following sequences selected from SEQ ID NOs: 242, 243, 248, 252-275, 277-279, 281-285, and 287-291.

12. The oligonucleotide of any one of claims 1-10, wherein the oligonucleotide is partially complementary to SEQ ID NO: 1168, and comprises one of the following sequences selected from: SEQ ID NOs: 2, 4, 6, 9-14, 16-18, 20, 22, 23, 24, 26, 27, 30, 35-37, 39, 42, 43, 45, 47-50, 52-56, 58, 59, 61, 63, 66, 68, 74, 78, 82, 83, 86, 87, 90, 93, 97, 98, 101, 102, 105, 106, 112, 115, 116, 118, 122, 126-128, 137-140, 143, 146, 147, 154, 155, 159, 161-164, 169, 170, 174, 182, 185, 189, 190, 198-200, 203, 208, and 209.

13. The oligonucleotide of any one of claims 1-10, wherein the oligonucleotide is partially complementary to SEQ ID NO: 1170 and comprises one of the following sequences selected from: SEQ ID NOs: 292, 298, 300, 305, 311-313, 319, 325, 327-330, 332, 333, 335, and 338.

14. The oligonucleotide of any one of claims 1-10, wherein the oligonucleotide is partially complementary to SEQ ID NO: 1171 and comprises one of the following sequences selected from: SEQ ID NOs: 347, 350, 358, 360, 361, 363, 364, 366, 368, 369, 371, 375, and 381.

15. The oligonucleotide of any one of claims 1-10, wherein the oligonucleotide is partially complementary to SEQ ID NO: 1172 and comprises one of the following sequences selected from:
a) SEQ ID NOs: 402, 407, 415-417, 421, 425, 428, 431, 432, 440, 443, 447, 449, 450, 453, 467, 468, 476, 480, 482, 489, 490, 492, 495, 496, 497, 499, 502, 503, 505, and 513; or
b) SEQ ID NOs: 405, 408, 409, 412-414, 418-420, 422-424, 426-427, 429, 430, 433-437, 439, 441, 442, 444-446, 448, 451, 452, 454-466, 469-472, 474, 478, 479, 481, 483-486, 493, 498, 500, 504, 506, 508, 509, 511, 512, and 514-518.

16. The oligonucleotide of any one of claims 1-10, wherein the oligonucleotide is partially complementary to SEQ ID NO: 1173 and comprises one of the following sequences selected from: SEQ ID NOs: 521, 523-530, 532, 534, 539-542, 544, 545, 547-549, 551-554, 556, 558-561, 564, 566, 567, 570, 571, 573-580, 582, 583, 585-587, and 590-593.

17. The oligonucleotide of any one of claims 1-10, wherein the oligonucleotide is partially complementary to SEQ ID NO: 1174 and comprises one of the following sequences selected from: SEQ ID NOs: 594-633.

18. The oligonucleotide of any one of claims 1-10, wherein the oligonucleotide is partially complementary to SEQ ID NO: 1175 and comprises one of the following sequences selected from: SEQ ID NOs: 635, 638, 642, 643, 645, 646, 651, 658, 659, 664, 667, 673, 674, 683, 685, 686, and 693.

19. The oligonucleotide of any one of claims 1-10, wherein the oligonucleotide is partially complementary to SEQ ID NO: 1176 and comprises one of the following sequences selected from: SEQ ID NOs: 697, 699-702, 704-706, 708, 709, 711, 714, 716, 719, 721-723, 726, and 727.

20. The oligonucleotide of any one of claims 1-10, wherein the oligonucleotide is partially complementary to SEQ ID NO: 1177 and comprises one of the following sequences selected from: SEQ ID NOs: 732, 735, 738, 739, 744, 745, 748, and 753.

21. The oligonucleotide of any one of claims 1-10, wherein the oligonucleotide is partially complementary to SEQ ID NO: 1178 and comprises one of the following sequences selected from: SEQ ID NOs: 760, 762, 768, 772, 784, and 787.

22. The oligonucleotide of any one of claims 1-10, wherein the oligonucleotide is partially complementary to SEQ ID NO: 1179 and comprises one of the following sequences selected from: SEQ ID NOs: 811, 832, 846, 889, and 907.

23. The oligonucleotide of claim 8 or 9, wherein the target RNA sequence codes for one of the following proteins selected from a list consisting of:
a) STAT3 containing the Y705 phosphorylation site, and wherein the oligonucleotide comprises one of the sequences selected from a group consisting of SEQ ID NOs: 927-966;
b) STAT1 containing the Y701phosphorylation site, and wherein the oligonucleotide comprises one of the sequences selected from a group consisting of SEQ ID NO: 967-1005;
c) IFNAR1 containing the Y466 phosphorylation site, and wherein the oligonucleotide comprises one of the sequences selected from a group consisting of SEQ ID NOs: 1006-1029;
d) CTNNB1 containing the T41 phosphorylation site, and wherein the oligonucleotide comprises one of the sequences selected from a group consisting of SEQ ID NOs: 1030-1070;
e) TMEM173 containing the N154 residue, and wherein the oligonucleotide comprises one of the sequences selected from a group consisting of SEQ ID NOs: 1071-1134; and
f) TMEM173 containing the R284 residue, and wherein the oligonucleotide comprises one of the sequences selected from a group consisting of SEQ ID NOs: 1135-1165.

24. The oligonucleotide of claim 23, wherein the oligonucleotide is partially complementary to SEQ ID NO: 1180 and comprises one of the following sequences selected from: SEQ ID NOs: 936, 939, and 947.

25. The oligonucleotide of claim23, wherein the oligonucleotide is partially complementary to SEQ ID NO: 1181 and comprises one of the following sequences selected from: SEQ ID NOs: 967-1005.

26. The oligonucleotide of claim23, wherein the oligonucleotide is partially complementary to SEQ ID NO: 1182 and comprises one of the following sequences selected from: SEQ ID NOs: 1006-1020, 1022, 1024, 1026, and 1029.

27. The oligonucleotide of claim23, wherein the oligonucleotide is partially complementary to SEQ ID NO: 1183 and comprises one of the following sequences selected from: SEQ ID NOs: 1030-1070.

28. The oligonucleotide of claim23, wherein the oligonucleotide is partially complementary to SEQ ID NO: 1184 and comprises one of the following sequences selected from: SEQ ID NOs: 1074, 1079, 1087, 1094, 1096, 1107, 1112, 1120, 1125, 1128, and 1133.

29. The oligonucleotide of claim23, wherein the oligonucleotide is partially complementary to SEQ ID NO: 1185 and comprises one of the following sequences selected from: SEQ ID NOs: 1148 and 1156.

30. The oligonucleotide of any one of claim 1-29, wherein the oligonucleotide has an editing efficacy of at least 20%, at least 30%, at least 40%, at least 50%, at least 60%, or at least 70%, wherein the percentage is defined as % of A converted to I at the target site.

31. The oligonucleotide of any one of claim 1-30, wherein the oligonucleotide has an editing efficacy of between 20% and 100%.

32. The oligonucleotide of any one of claim 1-31, wherein the oligonucleotide has an editing enhancement that is at least 1.1-fold higher than that of a control, optionally wherein the editing enhancement is 1.1-3 fold higher.

33. The oligonucleotide of any one of claims 30-32, wherein the control is an oligonucleotide fully complementary to the target sequence except for a mismatch at No opposite the target A.

34. The oligonucleotide of any one of claims 1-33, wherein the oligonucleotide has a length of 15-101nt, preferably wherein the oligonucleotide has a length of 18-45 nt.

35. The oligonucleotide of any one of claims 1-34, wherein the oligonucleotide comprises between 4-50 nucleotides on either the 3′ or 5′ side of the target adenosine to be edited.

36. The oligonucleotide of any one of claims 1-35, wherein the oligonucleotide is asymmetric, optionally wherein there are at least 6 nucleotides (nt) at the 5′ terminus and at least 10 nt at the 3′ terminus.

37. The oligonucleotide of any one of claims 1-36, wherein the oligonucleotide is chemically modified, optionally wherein the oligonucleotide comprises one or more chemical modifications.

38. The oligonucleotide of claim 37, wherein the oligonucleotide comprises a modification at the 2′-position of the sugar residue.

39. The oligonucleotide of claim 37 or 38,
wherein 20-70% of nucleotides are 2′-F-modified, preferably wherein 35-65% of nucleotides are 2′-F-modified; and/or
wherein 20-60% of nucleotides are 2′-O-methyl(2′-OMe)-modified, preferably wherein 25-55% of nucleotides are 2′-OMe-modified.

40. The oligonucleotide of any one of claims 37-39, wherein at least 10% of nucleotides are 2′-F-modified and at least 10% of nucleotides are 2′-O-alkyl-modified, wherein no more than 6 consecutive nucleotides have the same 2′-modification.

41. The oligonucleotide of any one of claims 37-40, wherein
(i) no more than 4, 5, or 6 consecutive nucleotides are 2′-F-modified; and/or
(ii) no more than 4, 5, or 6 consecutive nucleotides are 2′-O-alkyl-modified.

42. The oligonucleotide of any one of claims 37-41, wherein the internucleoside linkage modification content is at least 30%, optionally wherein the internucleoside linkage modification content is between 30-90%.

43. The oligonucleotide of any one of claims 37-42, wherein the internucleoside linkage modification is selected from the group consisting of phosphorothioatc (PS), 3′-methylenephosphonate, 5′-methylenephosphonate, 3′-phosphoroamidate, 2′-5′phosphodiester, and phosphoryl guanidine (PN), preferably wherein the internucleoside linkage is PS.

44. The oligonucleotide of any one of claims 37-43, wherein the chemically modified oligonucleotide does not comprise a stereopure phosphorothioate (PS) linkage modification.

45. The oligonucleotide of any one of claims 37-44, wherein at least two of the three nucleotides of the CBT are chemically modified at the 2′ position of the sugar moiety, are deoxyribonucleosides, or a combination thereof.

46. The modified antisense oligonucleotide according to any one of claims 37-45, wherein
(i) N₊₁ is 2′-F, 2′-FANA (2′-deoxy-2′-fluoro-D-arabinonucleic acid), DNA, or 2′-O-methyl; and/or
(ii) N₀ is 2′-FANA or DNA; and/or
(iii)N₋₁ is 2′-FANA, DNA, or 2′-O-methyl.

47. The oligonucleotide of any one of claims 37-46, wherein the N₋₂ nucleotide carries a 2′-O-alkyl-modification; and wherein the N₋₃ nucleotide carries a 2′-fluoro (2′-F)-modification.

48. The oligonucleotide of any one of claims 37-47, wherein the oligonucleotide does not contain a stem loop secondary structure.

49. The oligonucleotide according to any one of claims 37-48, wherein the oligonucleotide is conjugated to a moiety that enhances cellular uptake of the oligonucleotide.

50. A composition comprising the oligonucleotide according to any one of claims 1-49.

51. A method for site-directed A-to-I editing of a target RNA, comprising providing to a cell or subject an oligonucleotide according to any one of claims 1-49 or a composition according to claim 50.

52. A method for creating a de-novo A-to-G beneficial protein variant, wherein the beneficial protein variant is derived from a wild-type protein selected from the group consisting of:
a) STAT3 containing the Y705 phosphorylation site;
b) STAT1 containing the Y701phosphorylation site;
c) IFNAR1 containing the Y466 phosphorylation site;
d) CTNNB1 containing the T41 phosphorylation site; and
e) TMEM173 containing the N154 or R284 residue;
wherein the method comprises contacting a cell with an oligonucleotide of any one of claims 1-6, 8, 9 or any one of claims 20, 21, 23-49.

53. An oligonucleotide of any one of claims 1-49 or a composition of claim 50 for use in therapy.

54. An oligonucleotide for use of any one of claims 1-22 and 30-49, or a composition of claim 50 for use in a method of treating or preventing a disease or condition associated with a point mutation in a subject.

55. The oligonucleotide or the composition for use of claim 54, wherein the disease or condition is selected from the group consisting of: a (1) AT (AAT) deficiency, Parkinson's disease, Myopathy, ALDH2 deficiency, Cancer, Congenital insensitivity to pain, Beta-Thalassemia, Thrombophilia, Rett Syndrome and Stargardt disease.

56. An oligonucleotide of any one of claims 1-22 and 30-49 or composition of claim 50 for use in a method of treating or preventing a disease or condition associated with a point mutation in a protein, wherein the point mutation is selected from the group consisting of:
a) SERPINA1 carrying the E342K mutation;
b) SNCA carrying the A53T mutation;
c) GFER carrying the R194H mutation;
d) ALDH2 carrying the E504K mutation;
e) TP53 carrying the oncogenic R175H mutation;
f) SCN9A carrying the W897X mutation;
g) HBB carrying the W38X mutation;
h) KRAS carrying the oncogenic G12D mutation;
i) KRAS carrying the oncogenic G12S mutation;
j) MeCP2 carrying the R106Q mutation;
k) F5 carrying the R534Q mutation; and
l) ABCA4 carrying the G1961E mutation.

57. An oligonucleotide for use of any one of claims 1-6, 8, 9 and 20, 21, 23-49 or a composition of claim 50 for use in a method of treating or preventing a disease or condition associated with wild-type proteins in a subject.

58. An oligonucleotide or composition of claim 57 for use in a method of treating or preventing a disease or condition associated with a wild-type protein, and wherein the wild-type protein is selected from the group consisting of:
a) STAT3 containing the Y705 phosphorylation site;
b) STAT1 containing the Y701phosphorylation site;
c) IFNAR1 containing the Y466 phosphorylation site;
d) CTNNB1 containing the T41 phosphorylation site; and
e) TMEM173 containing the N154 or R284 residue.

59. A method of treating or preventing a disease or condition associated with a point mutation or a wild-type protein in a subject, wherein the method comprises administering to the subject a therapeutically effective amount of an oligonucleotide of any one of claims 1-49, or a composition according to claim 50.

60. The method of claim 59, wherein the disease or condition is a (1) AT deficiency or an associated pathology, the target transcript comprises SERPINA1 carrying the E342K mutation, and the guide sequence comprises a sequence, or variant thereof, selected from any of SEQ ID NOs: 223-291.

61. The method of claim 59, wherein the disease or condition is Parkinson's Disease, the target transcript comprises the SNCA A53T mutation, and the guide sequence comprises a sequence, or variant thereof, selected from any of SEQ ID NOs: 1-222.

62. The method of claim 59, wherein the disease or condition is associated with the GFER R194H mutation, the target transcript comprises GFER carrying the R194H mutation, and the guide sequence comprises a sequence, or variant thereof, selected from any of SEQ ID NOs: 292-339.

63. The method of claim 59, wherein the disease or condition is deficiency in aldehyde metabolism, the target transcript comprises ALDH2 carrying the E504K mutation, and the guide sequence comprises a sequence, or variant thereof, selected from any of SEQ ID NOs: 340-399.

64. The method of claim 59, wherein the disease or condition is cancer or risk of cancer, the target transcript comprises TP53 carrying the R175H mutation, and the guide sequence comprises a sequence, or variant thereof, selected from any of SEQ ID NOs: 400-518.

65. The method of claim 59, wherein the disease or condition is associated with defects in the alpha subunit of a sodium channel NaV1.7, the target transcript comprises SCN9A carrying the W897X mutation, and the guide sequence comprises a sequence, or variant thereof, selected from any of SEQ ID NOs: 519-593.

66. The method of claim 59, wherein the disease or condition is beta thalassemia or a condition associated with hemoglobin subunit beta defects, the target transcript comprises HBB carrying the W38X mutation, and the guide sequence comprises a sequence, or variant thereof, selected from any of SEQ ID NOs: 594-634.

67. The method of claim 59, wherein the disease or condition is cancer or risk of cancer, the target transcript comprises KRAS carrying the G12D mutation, and the guide sequence comprises a sequence, or variant thereof, selected from any of SEQ ID NOs: 635-694.

68. The method of claim 59, wherein the disease or condition is cancer or risk of cancer, the target transcript comprises KRAS carrying the G12S mutation, and the guide sequence comprises a sequence, or variant thereof, selected from any of SEQ ID NOs: 695-728.

69. The method of claim 59, wherein the disease or condition is Rett Syndrome, the target transcript comprises McCP2 carrying the R106Q mutation, and the guide sequence comprises a sequence, or variant thereof, selected from any of SEQ ID NOs: 729-753.

70. The method of claim 59, wherein the disease or condition is associated with Factor V defects, the target transcript comprises F5 carrying the R534Q mutation, and the guide sequence comprises a sequence, or variant thereof, selected from any of SEQ ID NOs: 754-796.

71. The method of claim 59, wherein the disease or condition is retinal disease, a maculopathy, or Stargardt disease, the target transcript comprises ABCA4 carrying the G1961E mutation, and the guide sequence comprises a sequence, or variant thereof, selected from any of SEQ ID NOs: 797-926.

72. The method of claim 59, wherein the method comprises creating a beneficial protein variant and the target sequence comprises a sequence coding for the wild-type protein.

73. The method of claim 72, wherein the target transcript comprises wild-type STAT3, the guide sequence comprises a sequence, or variant thereof, selected from any of SEQ ID NOs: 927-966, and the beneficial protein variant comprises STAT3 Y705C.

74. The method of claim 72, wherein the target transcript comprises wild-type STAT1, the guide sequence comprises a sequence, or variant thereof, selected from any of SEQ ID NOs: 967-1005, and the beneficial protein variant comprises STAT1 Y701C.

75. The method of claim 72, wherein the target transcript comprises wild-type IFNAR1, the guide sequence comprises a sequence, or variant thereof, selected from any of SEQ ID NOs: 1006-1029, and the beneficial protein variant comprises IFNAR1 Y466C.

76. The method of claim 72, wherein the target transcript comprises wild-type CTNNB1, the guide sequence comprises a sequence, or variant thereof, selected from any of SEQ ID NOs: 1030-1070, and the beneficial protein variant comprises CTNNB1 T41A.

77. The method of claim 72, wherein the target transcript comprises wild-type TMEM173, the guide sequence comprises a sequence, or variant thereof, selected from any of SEQ ID NOs: 1071-1134, and the beneficial protein variant comprises TMEM173 N154S.

78. The method of claim 72, wherein the target transcript comprises wild-type TMEM173, the guide sequence comprises a sequence, or variant thereof, selected from any of SEQ ID NOs: 1135-1165, and the beneficial protein variant comprises TMEM173 R284G.


SEQUENCE LISTING (1318 sequences)

SEQ ID NO: 1 - RNA, 18 nt, Synthetic construct
  source 1..18: mol_type = other RNA
cgtgccacagcgctgtta

SEQ ID NO: 2 - RNA, 18 nt, Synthetic construct
  source 1..18: mol_type = other RNA
cctacaactgcgttgtcc

SEQ ID NO: 3 - RNA, 18 nt, Synthetic construct
  source 1..18: mol_type = other RNA
cgtaccagagcgttctga

SEQ ID NO: 4 - RNA, 20 nt, Synthetic construct
  source 1..20: mol_type = other RNA
cacctaccacagcgttgtca

SEQ ID NO: 5 - RNA, 20 nt, Synthetic construct
  source 1..20: mol_type = other RNA
cacataccacgccgttctca

SEQ ID NO: 6 - RNA, 20 nt, Synthetic construct
  source 1..20: mol_type = other RNA
cacctaccacagcgttgtcg

SEQ ID NO: 7 - RNA, 20 nt, Synthetic construct
  source 1..20: mol_type = other RNA
cacgtaccacagcgttgtat

SEQ ID NO: 8 - RNA, 18 nt, Synthetic construct
  source 1..18: mol_type = other RNA
cataccacagcgttttcg

SEQ ID NO: 9 - RNA, 20 nt, Synthetic construct
  source 1..20: mol_type = other RNA
cacttaccacagcgttgtca

SEQ ID NO: 10 - RNA, 18 nt, Synthetic construct
  source 1..18: mol_type = other RNA
cctaccacagcgttgtcc

SEQ ID NO: 11 - RNA, 20 nt, Synthetic construct
  source 1..20: mol_type = other RNA
cacctaccacagcgttttca

SEQ ID NO: 12 - RNA, 18 nt, Synthetic construct
  source 1..18: mol_type = other RNA
cttaccacagcgtagtca

SEQ ID NO: 13 - RNA, 18 nt, Synthetic construct
  source 1..18: mol_type = other RNA
cctaccacagcgttatct

SEQ ID NO: 14 - RNA, 18 nt, Synthetic construct
  source 1..18: mol_type = other RNA
cctaccacagcgctgtgg

SEQ ID NO: 15 - RNA, 20 nt, Synthetic construct
  source 1..20: mol_type = other RNA
cacgtaccacaccgtagcca

SEQ ID NO: 16 - RNA, 20 nt, Synthetic construct
  source 1..20: mol_type = other RNA
cacctaccacaacgttgtca

SEQ ID NO: 17 - RNA, 18 nt, Synthetic construct
  source 1..18: mol_type = other RNA
cctaccacagcgttgtag

SEQ ID NO: 18 - RNA, 18 nt, Synthetic construct
  source 1..18: mol_type = other RNA
cctaccacagcgttgtaa

SEQ ID NO: 19 - RNA, 18 nt, Synthetic construct
  source 1..18: mol_type = other RNA
cgtaccacagcgtagtgg

SEQ ID NO: 20 - RNA, 18 nt, Synthetic construct
  source 1..18: mol_type = other RNA
cctaccacagcgttgtga

SEQ ID NO: 21 - RNA, 18 nt, Synthetic construct
  source 1..18: mol_type = other RNA
cataccacagcgttctcg

SEQ ID NO: 22 - RNA, 18 nt, Synthetic construct
  source 1..18: mol_type = other RNA
cttaccacagcgttgtga

SEQ ID NO: 23 - RNA, 20 nt, Synthetic construct
  source 1..20: mol_type = other RNA
cacttaccacagcgctgtca

SEQ ID NO: 24 - RNA, 18 nt, Synthetic construct
  source 1..18: mol_type = other RNA
cttaccacagcgtagtca

SEQ ID NO: 25 - RNA, 18 nt, Synthetic construct
  source 1..18: mol_type = other RNA
cctaccacagcgttatct

SEQ ID NO: 26 - RNA, 18 nt, Synthetic construct
  source 1..18: mol_type = other RNA
cctaccacagcgctgtgg

SEQ ID NO: 27 - RNA, 20 nt, Synthetic construct
  source 1..20: mol_type = other RNA
cacgtaccacaccgtagcca

SEQ ID NO: 28 - RNA, 20 nt, Synthetic construct
  source 1..20: mol_type = other RNA
cacctaccacaacgttgtca

SEQ ID NO: 29 - RNA, 18 nt, Synthetic construct
  source 1..18: mol_type = other RNA
cctaccacagcgttgtag

SEQ ID NO: 30 - RNA, 18 nt, Synthetic construct
  source 1..18: mol_type = other RNA
cctaccacagcgttgtaa

SEQ ID NO: 31 - RNA, 18 nt, Synthetic construct
  source 1..18: mol_type = other RNA
cgtaccacagcgtagtgg

SEQ ID NO: 32 - RNA, 18 nt, Synthetic construct
  source 1..18: mol_type = other RNA
cctaccacagcgttgtga

SEQ ID NO: 33 - RNA, 18 nt, Synthetic construct
  source 1..18: mol_type = other RNA
cataccacagcgttctcg

SEQ ID NO: 34 - RNA, 18 nt, Synthetic construct
  source 1..18: mol_type = other RNA
cttaccacagcgttgtga

SEQ ID NO: 35 - RNA, 20 nt, Synthetic construct
  source 1..20: mol_type = other RNA
cacttaccacagcgctgtca

SEQ ID NO: 36 - RNA, 18 nt, Synthetic construct
  source 1..18: mol_type = other RNA
cctaccacagcgttctga

SEQ ID NO: 37 - RNA, 20 nt, Synthetic construct
  source 1..20: mol_type = other RNA
cacctaccacaccgttatct

SEQ ID NO: 38 - RNA, 20 nt, Synthetic construct
  source 1..20: mol_type = other RNA
cacgtaccacaccgttgtat

SEQ ID NO: 39 - RNA, 18 nt, Synthetic construct
  source 1..18: mol_type = other RNA
cttaccacagcgttgtaa

SEQ ID NO: 40 - RNA, 18 nt, Synthetic construct
  source 1..18: mol_type = other RNA
attaccacagcgttgtaa

SEQ ID NO: 41 - RNA, 18 nt, Synthetic construct
  source 1..18: mol_type = other RNA
tgcaccacagcgttgtga

SEQ ID NO: 42 - RNA, 20 nt, Synthetic construct
  source 1..20: mol_type = other RNA
aacctaccacaccgttgtga

SEQ ID NO: 43 - RNA, 18 nt, Synthetic construct
  source 1..18: mol_type = other RNA
cttaccacagcgttatct

SEQ ID NO: 44 - RNA, 18 nt, Synthetic construct
  source 1..18: mol_type = other RNA
cgtaccacagcgttttcc

SEQ ID NO: 45 - RNA, 18 nt, Synthetic construct
  source 1..18: mol_type = other RNA
cctaccacagcggtgtaa

SEQ ID NO: 46 - RNA, 18 nt, Synthetic construct
  source 1..18: mol_type = other RNA
cgtaccacagcgtcgtcg

SEQ ID NO: 47 - RNA, 18 nt, Synthetic construct
  source 1..18: mol_type = other RNA
cctaccacagcgttatca

SEQ ID NO: 48 - RNA, 18 nt, Synthetic construct
  source 1..18: mol_type = other RNA
cctaccatagcgttgtct

SEQ ID NO: 49 - RNA, 18 nt, Synthetic construct
  source 1..18: mol_type = other RNA
cttaccacagcgttgtcg

SEQ ID NO: 50 - RNA, 20 nt, Synthetic construct
  source 1..20: mol_type = other RNA
cacctacctcaccgttgtca

SEQ ID NO: 51 - RNA, 20 nt, Synthetic construct
  source 1..20: mol_type = other RNA
cacgtaccacagcgttgtcc

SEQ ID NO: 52 - RNA, 20 nt, Synthetic construct
  source 1..20: mol_type = other RNA
cacctaccacaccgctgtga

SEQ ID NO: 53 - RNA, 18 nt, Synthetic construct
  source 1..18: mol_type = other RNA
cttaccacagcgttacct

SEQ ID NO: 54 - RNA, 18 nt, Synthetic construct
  source 1..18: mol_type = other RNA
cttaccacagcgttgtcc

SEQ ID NO: 55 - RNA, 20 nt, Synthetic construct
  source 1..20: mol_type = other RNA
tacctaccacaacgttgtca

SEQ ID NO: 56 - RNA, 20 nt, Synthetic construct
  source 1..20: mol_type = other RNA
cacctaccacaccgttgtga

SEQ ID NO: 57 - RNA, 18 nt, Synthetic construct
  source 1..18: mol_type = other RNA
tgtaccacagcgtcgtca

SEQ ID NO: 58 - RNA, 18 nt, Synthetic construct
  source 1..18: mol_type = other RNA
cctaccacagcgttaaca

SEQ ID NO: 59 - RNA, 18 nt, Synthetic construct
  source 1..18: mol_type = other RNA
cctaccacagcgtcgcca

SEQ ID NO: 60 - RNA, 18 nt, Synthetic construct
  source 1..18: mol_type = other RNA
cgtaccacagcgtcgtcc

SEQ ID NO: 61 - RNA, 18 nt, Synthetic construct
  source 1..18: mol_type = other RNA
cttaccacagcgttgcaa

SEQ ID NO: 62 - RNA, 18 nt, Synthetic construct
  source 1..18: mol_type = other RNA
ggtaccacagcgttgtcc

SEQ ID NO: 63 - RNA, 18 nt, Synthetic construct
  source 1..18: mol_type = other RNA
cctaccacagcgtcgttt

SEQ ID NO: 64 - RNA, 18 nt, Synthetic construct
  source 1..18: mol_type = other RNA
gctaccacagcgtagtcg

SEQ ID NO: 65 - RNA, 18 nt, Synthetic construct
  source 1..18: mol_type = other RNA
tgtaccacagcgttgaaa

SEQ ID NO: 66 - RNA, 20 nt, Synthetic construct
  source 1..20: mol_type = other RNA
cacctaccataccgttgtcc

SEQ ID NO: 67 - RNA, 18 nt, Synthetic construct
  source 1..18: mol_type = other RNA
gctaccacagcgttgtcc

SEQ ID NO: 68 - RNA, 20 nt, Synthetic construct
  source 1..20: mol_type = other RNA
cacctaccacaccgtagtca

SEQ ID NO: 69 - RNA, 18 nt, Synthetic construct
  source 1..18: mol_type = other RNA
cgtaccacagcgtatgca

SEQ ID NO: 70 - RNA, 18 nt, Synthetic construct
  source 1..18: mol_type = other RNA
cgtcccactgcgtcggca

SEQ ID NO: 71 - RNA, 18 nt, Synthetic construct
  source 1..18: mol_type = other RNA
cataccacagcgttgtaa

SEQ ID NO: 72 - RNA, 20 nt, Synthetic construct
  source 1..20: mol_type = other RNA
cccgtcccacaacgttgaca

SEQ ID NO: 73 - RNA, 18 nt, Synthetic construct
  source 1..18: mol_type = other RNA
cggacaacagcgttgtaa

SEQ ID NO: 74 - RNA, 18 nt, Synthetic construct
  source 1..18: mol_type = other RNA
cctaccacagcgtagtca

SEQ ID NO: 75 - RNA, 18 nt, Synthetic construct
  source 1..18: mol_type = other RNA
cataccacagcgtagtca

SEQ ID NO: 76 - RNA, 18 nt, Synthetic construct
  source 1..18: mol_type = other RNA
cgtaccacaacgttgtca

SEQ ID NO: 77 - RNA, 18 nt, Synthetic construct
  source 1..18: mol_type = other RNA
tgtaccacagcgttgtga

SEQ ID NO: 78 - RNA, 20 nt, Synthetic construct
  source 1..20: mol_type = other RNA
cacttaccacaccgtcgtaa

SEQ ID NO: 79 - RNA, 20 nt, Synthetic construct
  source 1..20: mol_type = other RNA
cacgtaccacagcgttgtct

SEQ ID NO: 80 - RNA, 18 nt, Synthetic construct
  source 1..18: mol_type = other RNA
cataccacagcgttgtct

SEQ ID NO: 81 - RNA, 18 nt, Synthetic construct
  source 1..18: mol_type = other RNA
cgtaccacagcgttgtaa

SEQ ID NO: 82 - RNA, 18 nt, Synthetic construct
  source 1..18: mol_type = other RNA
cttaccccagcgttttca

SEQ ID NO: 83 - RNA, 18 nt, Synthetic construct
  source 1..18: mol_type = other RNA
cctaccacagcgttctca

SEQ ID NO: 84 - RNA, 18 nt, Synthetic construct
  source 1..18: mol_type = other RNA
actaccacagcgttgtga

SEQ ID NO: 85 - RNA, 18 nt, Synthetic construct
  source 1..18: mol_type = other RNA
cataccacggcgttgtct

SEQ ID NO: 86 - RNA, 20 nt, Synthetic construct
  source 1..20: mol_type = other RNA
cacttaccacaccgtagtca

SEQ ID NO: 87 - RNA, 20 nt, Synthetic construct
  source 1..20: mol_type = other RNA
cacctaccacaccgttttcg

SEQ ID NO: 88 - RNA, 18 nt, Synthetic construct
  source 1..18: mol_type = other RNA
gataccacagcgttttca

SEQ ID NO: 89 - RNA, 18 nt, Synthetic construct
  source 1..18: mol_type = other RNA
cgtaccacagcgtaggca

SEQ ID NO: 90 - RNA, 20 nt, Synthetic construct
  source 1..20: mol_type = other RNA
cacttaccacaacgttgtca

SEQ ID NO: 91 - RNA, 20 nt, Synthetic construct
  source 1..20: mol_type = other RNA
cacataccatagcgttgtca

SEQ ID NO: 92 - RNA, 20 nt, Synthetic construct
  source 1..20: mol_type = other RNA
cacgtaccacagcgttggca

SEQ ID NO: 93 - RNA, 18 nt, Synthetic construct
  source 1..18: mol_type = other RNA
cttaccacagcgtcgtcg

SEQ ID NO: 94 - RNA, 18 nt, Synthetic construct
  source 1..18: mol_type = other RNA
cgtaccacagcgttgtag

SEQ ID NO: 95 - RNA, 18 nt, Synthetic construct
  source 1..18: mol_type = other RNA
tgtaccacagcgttggcc

SEQ ID NO: 96 - RNA, 20 nt, Synthetic construct
  source 1..20: mol_type = other RNA
cacgtaccacaccgtggtcg

SEQ ID NO: 97 - RNA, 20 nt, Synthetic construct
  source 1..20: mol_type = other RNA
cacctaccacaccgttgtca

SEQ ID NO: 98 - RNA, 20 nt, Synthetic construct
  source 1..20: mol_type = other RNA
cacctaccacaccgtcgtca

SEQ ID NO: 99 - RNA, 18 nt, Synthetic construct
  source 1..18: mol_type = other RNA
cgtaccacagcgtagtcc

SEQ ID NO: 100 - RNA, 18 nt, Synthetic construct
  source 1..18: mol_type = other RNA
cgcaccacagcgttgtat

SEQ ID NO: 101 - RNA, 18 nt, Synthetic construct
  source 1..18: mol_type = other RNA
cctaccacagcgttctta

SEQ ID NO: 102 - RNA, 18 nt, Synthetic construct
  source 1..18: mol_type = other RNA
cctaccacagcgttgcaa

SEQ ID NO: 103 - RNA, 18 nt, Synthetic construct
  source 1..18: mol_type = other RNA
tgtaccacagcgttgtaa

SEQ ID NO: 104 - RNA, 18 nt, Synthetic construct
  source 1..18: mol_type = other RNA
actaccacagcgttatcc

SEQ ID NO: 105 - RNA, 20 nt, Synthetic construct
  source 1..20: mol_type = other RNA
cacctaccacaccgtggtca

SEQ ID NO: 106 - RNA, 20 nt, Synthetic construct
  source 1..20: mol_type = other RNA
tacctaccacaccgtagtca

SEQ ID NO: 107 - RNA, 20 nt, Synthetic construct
  source 1..20: mol_type = other RNA
cacgtaccacagcgtcgtca

SEQ ID NO: 108 - RNA, 20 nt, Synthetic construct
  source 1..20: mol_type = other RNA
catgtaccacaccgtagtca

SEQ ID NO: 109 - RNA, 18 nt, Synthetic construct
  source 1..18: mol_type = other RNA
ggtaccacagcgttgcct

SEQ ID NO: 110 - RNA, 18 nt, Synthetic construct
  source 1..18: mol_type = other RNA
cgtaccatagcgttgtgg

SEQ ID NO: 111 - RNA, 20 nt, Synthetic construct
  source 1..20: mol_type = other RNA
cacgtaccacagcgttgtga

SEQ ID NO: 112 - RNA, 18 nt, Synthetic construct
  source 1..18: mol_type = other RNA
cctaccacggcgttgtcg

SEQ ID NO: 113 - RNA, 18 nt, Synthetic construct
  source 1..18: mol_type = other RNA
attaccacagcgttgaca

SEQ ID NO: 114 - RNA, 18 nt, Synthetic construct
  source 1..18: mol_type = other RNA
cgtaccatagcgttctct

SEQ ID NO: 115 - RNA, 18 nt, Synthetic construct
  source 1..18: mol_type = other RNA
cttactacagcgttgtga

SEQ ID NO: 116 - RNA, 18 nt, Synthetic construct
  source 1..18: mol_type = other RNA
cctaccacagcgtactca

SEQ ID NO: 117 - RNA, 18 nt, Synthetic construct
  source 1..18: mol_type = other RNA
tgtaccacagcgttgtcc

SEQ ID NO: 118 - RNA, 20 nt, Synthetic construct
  source 1..20: mol_type = other RNA
cacctaccacactgttgtca

SEQ ID NO: 119 - RNA, 20 nt, Synthetic construct
  source 1..20: mol_type = other RNA
cacgtaccccagcgttgtca

SEQ ID NO: 120 - RNA, 18 nt, Synthetic construct
  source 1..18: mol_type = other RNA
cataccacagcgttgtga

SEQ ID NO: 121 - RNA, 18 nt, Synthetic construct
  source 1..18: mol_type = other RNA
gttaccacagcgttgtga

SEQ ID NO: 122 - RNA, 18 nt, Synthetic construct
  source 1..18: mol_type = other RNA
cctaccacagcgttggca

SEQ ID NO: 123 - RNA, 18 nt, Synthetic construct
  source 1..18: mol_type = other RNA
ggtaccacagcgttgtga

SEQ ID NO: 124 - RNA, 18 nt, Synthetic construct
  source 1..18: mol_type = other RNA
cgtaccacagcgttgtcg

SEQ ID NO: 125 - RNA, 18 nt, Synthetic construct
  source 1..18: mol_type = other RNA
cgtactacagcgtagtga

SEQ ID NO: 126 - RNA, 20 nt, Synthetic construct
  source 1..20: mol_type = other RNA
aacataccacaccgttgtga

SEQ ID NO: 127 - RNA, 20 nt, Synthetic construct
  source 1..20: mol_type = other RNA
cacttaccacaccggtgtca

SEQ ID NO: 128 - RNA, 18 nt, Synthetic construct
  source 1..18: mol_type = other RNA
cttaccacagcgttgtgg

SEQ ID NO: 129 - RNA, 18 nt, Synthetic construct
  source 1..18: mol_type = other RNA
gttaccacagcgttgtcc

SEQ ID NO: 130 - RNA, 18 nt, Synthetic construct
  source 1..18: mol_type = other RNA
ggtaccacagcgttgaaa

SEQ ID NO: 131 - RNA, 18 nt, Synthetic construct
  source 1..18: mol_type = other RNA
cgtaccacagcgttttct

SEQ ID NO: 132 - RNA, 18 nt, Synthetic construct
  source 1..18: mol_type = other RNA
cgtaccacagcgtagtct

SEQ ID NO: 133 - RNA, 18 nt, Synthetic construct
  source 1..18: mol_type = other RNA
cgtaccatagcgttgtcg

SEQ ID NO: 134 - RNA, 20 nt, Synthetic construct
  source 1..20: mol_type = other RNA
cgcgtaccacagcgttgtca

SEQ ID NO: 135 - RNA, 18 nt, Synthetic construct
  source 1..18: mol_type = other RNA
cataccacagcgttgtcg

SEQ ID NO: 136 - RNA, 20 nt, Synthetic construct
  source 1..20: mol_type = other RNA
cccgtaccacagcgttgtca

SEQ ID NO: 137 - RNA, 18 nt, Synthetic construct
  source 1..18: mol_type = other RNA
cttaccacagcgtagtct

SEQ ID NO: 138 - RNA, 18 nt, Synthetic construct
  source 1..18: mol_type = other RNA
cttaccacagcgttgtct

SEQ ID NO: 139 - RNA, 18 nt, Synthetic construct
  source 1..18: mol_type = other RNA
cttaccatagcgttgtaa

SEQ ID NO: 140 - RNA, 20 nt, Synthetic construct
  source 1..20: mol_type = other RNA
cacttaccacaacgttctca

SEQ ID NO: 141 - RNA, 18 nt, Synthetic construct
  source 1..18: mol_type = other RNA
cgtaccacagcgtcgttg

SEQ ID NO: 142 - RNA, 18 nt, Synthetic construct
  source 1..18: mol_type = other RNA
cgtaccacagcgtcgtct

SEQ ID NO: 143 - RNA, 20 nt, Synthetic construct
  source 1..20: mol_type = other RNA
cacctaccacaccggtgtca

SEQ ID NO: 144 - RNA, 20 nt, Synthetic construct
  source 1..20: mol_type = other RNA
tatgtaccacaccgttgtca

SEQ ID NO: 145 - RNA, 20 nt, Synthetic construct
  source 1..20: mol_type = other RNA
cacgtaccacgccgttgtcc

SEQ ID NO: 146 - RNA, 18 nt, Synthetic construct
  source 1..18: mol_type = other RNA
cctaccacagcgttgtta

SEQ ID NO: 147 - RNA, 18 nt, Synthetic construct
  source 1..18: mol_type = other RNA
cttaccacagcgttgcga

SEQ ID NO: 148 - RNA, 18 nt, Synthetic construct
  source 1..18: mol_type = other RNA
cataccacagcgttgtgc

SEQ ID NO: 149 - RNA, 18 nt, Synthetic construct
  source 1..18: mol_type = other RNA
cggacggcagcgttgtca

SEQ ID NO: 150 - RNA, 18 nt, Synthetic construct
  source 1..18: mol_type = other RNA
cataccacagcgtcgtcc

SEQ ID NO: 151 - RNA, 18 nt, Synthetic construct
  source 1..18: mol_type = other RNA
cgtaccacagcgttaaaa

SEQ ID NO: 152 - RNA, 18 nt, Synthetic construct
  source 1..18: mol_type = other RNA
cgtaccacggcgttgtct

SEQ ID NO: 153 - RNA, 18 nt, Synthetic construct
  source 1..18: mol_type = other RNA
cgtactacagcgttatcg

SEQ ID NO: 154 - RNA, 18 nt, Synthetic construct
  source 1..18: mol_type = other RNA
cttaccacagcgttatta

SEQ ID NO: 155 - RNA, 18 nt, Synthetic construct
  source 1..18: mol_type = other RNA
cttactacagagttgtaa

SEQ ID NO: 156 - RNA, 18 nt, Synthetic construct
  source 1..18: mol_type = other RNA
ggtaccacagcgttgtgg

SEQ ID NO: 157 - RNA, 18 nt, Synthetic construct
  source 1..18: mol_type = other RNA
cataccacagcgttgtta

SEQ ID NO: 158 - RNA, 18 nt, Synthetic construct
  source 1..18: mol_type = other RNA
tataccacagcgttatca

SEQ ID NO: 159 - RNA, 18 nt, Synthetic construct
  source 1..18: mol_type = other RNA
cttaccatagcgtagtaa

SEQ ID NO: 160 - RNA, 18 nt, Synthetic construct
  source 1..18: mol_type = other RNA
cgtaccacagcgtcgttc

SEQ ID NO: 161 - RNA, 20 nt, Synthetic construct
  source 1..20: mol_type = other RNA
cacttaccacgccgttgtaa

SEQ ID NO: 162 - RNA, 20 nt, Synthetic construct
  source 1..20: mol_type = other RNA
cacttaccacaccgttctca

SEQ ID NO: 163 - RNA, 20 nt, Synthetic construct
  source 1..20: mol_type = other RNA
cacttaccacaccgttttca

SEQ ID NO: 164 - RNA, 18 nt, Synthetic construct
  source 1..18: mol_type = other RNA
cctaccacagcgttgcca

SEQ ID NO: 165 - RNA, 18 nt, Synthetic construct
  source 1..18: mol_type = other RNA
cgtaccacagcgttgttt

SEQ ID NO: 166 - RNA, 18 nt, Synthetic construct
  source 1..18: mol_type = other RNA
cgtaccacagcgtcgtga

SEQ ID NO: 167 - RNA, 18 nt, Synthetic construct
  source 1..18: mol_type = other RNA
cgtaccacagcgtagtga

SEQ ID NO: 168 - RNA, 20 nt, Synthetic construct
  source 1..20: mol_type = other RNA
cccgtaccacagcgttctca

SEQ ID NO: 169 - RNA, 20 nt, Synthetic construct
  source 1..20: mol_type = other RNA
cacttaccacaccgtcgtca

SEQ ID NO: 170 - RNA, 20 nt, Synthetic construct
  source 1..20: mol_type = other RNA
cacttaccacaccgtcgtcg

SEQ ID NO: 171 - RNA, 20 nt, Synthetic construct
  source 1..20: mol_type = other RNA
catgtaccacagcgttgtca

SEQ ID NO: 172 - RNA, 18 nt, Synthetic construct
  source 1..18: mol_type = other RNA
cgtaccacagcgttatct

SEQ ID NO: 173 - RNA, 18 nt, Synthetic construct
  source 1..18: mol_type = other RNA
cgtaccacagcgttgtgg

SEQ ID NO: 174 - RNA, 20 nt, Synthetic construct
  source 1..20: mol_type = other RNA
cacttaccacaccgtcatta

SEQ ID NO: 175 - RNA, 18 nt, Synthetic construct
  source 1..18: mol_type = other RNA
cataccacagcgtggtcc

SEQ ID NO: 176 - RNA, 20 nt, Synthetic construct
  source 1..20: mol_type = other RNA
cacgtaccacagcgtagtca

SEQ ID NO: 177 - RNA, 18 nt, Synthetic construct
  source 1..18: mol_type = other RNA
cgtaccacagcgttctcg

SEQ ID NO: 178 - RNA, 18 nt, Synthetic construct
  source 1..18: mol_type = other RNA
cgtaccatagcgttttaa

SEQ ID NO: 179 - RNA, 20 nt, Synthetic construct
  source 1..20: mol_type = other RNA
cacgtaccacagcgctgtaa

SEQ ID NO: 180 - RNA, 18 nt, Synthetic construct
  source 1..18: mol_type = other RNA
ggtaccacagcgtagtca

SEQ ID NO: 181 - RNA, 18 nt, Synthetic construct
  source 1..18: mol_type = other RNA
cgtaccacagcgttttcg

SEQ ID NO: 182 - RNA, 18 nt, Synthetic construct
  source 1..18: mol_type = other RNA
cttaccacagcgttgtta

SEQ ID NO: 183 - RNA, 18 nt, Synthetic construct
  source 1..18: mol_type = other RNA
cgtaccacggcgttgtga

SEQ ID NO: 184 - RNA, 18 nt, Synthetic construct
  source 1..18: mol_type = other RNA
cgtaccacagcgttttaa

SEQ ID NO: 185 - RNA, 18 nt, Synthetic construct
  source 1..18: mol_type = other RNA
cttaccacagcgtagtcg

SEQ ID NO: 186 - RNA, 18 nt, Synthetic construct
  source 1..18: mol_type = other RNA
cataccatagcgttgtct

SEQ ID NO: 187 - RNA, 18 nt, Synthetic construct
  source 1..18: mol_type = other RNA
cataccacagcgttgacc

SEQ ID NO: 188 - RNA, 18 nt, Synthetic construct
  source 1..18: mol_type = other RNA
cgtaccacagcgtttata

SEQ ID NO: 189 - RNA, 20 nt, Synthetic construct
  source 1..20: mol_type = other RNA
cacataccacaccgttgtaa

SEQ ID NO: 190 - RNA, 18 nt, Synthetic construct
  source 1..18: mol_type = other RNA
cttaccacagcgttgaca

SEQ ID NO: 191 - RNA, 18 nt, Synthetic construct
  source 1..18: mol_type = other RNA
gctaccacagcgttgtca

SEQ ID NO: 192 - RNA, 18 nt, Synthetic construct
  source 1..18: mol_type = other RNA
cgtaccacagcgttatga

SEQ ID NO: 193 - RNA, 18 nt, Synthetic construct
  source 1..18: mol_type = other RNA
cgtaccacggcgttctca

SEQ ID NO: 194 - RNA, 18 nt, Synthetic construct
  source 1..18: mol_type = other RNA
ggtaccacagcgttgtct

SEQ ID NO: 195 - RNA, 18 nt, Synthetic construct
  source 1..18: mol_type = other RNA
tttaccacagcgttctca

SEQ ID NO: 196 - RNA, 18 nt, Synthetic construct
  source 1..18: mol_type = other RNA
tgtaccacagcgttgtag

SEQ ID NO: 197 - RNA, 18 nt, Synthetic construct
  source 1..18: mol_type = other RNA
cgtaccacagcgctgtct

SEQ ID NO: 198 - RNA, 20 nt, Synthetic construct
  source 1..20: mol_type = other RNA
cacttaccacaccgttgtca

SEQ ID NO: 199 - RNA, 20 nt, Synthetic construct
  source 1..20: mol_type = other RNA
cacataccacaccgttgtga

SEQ ID NO: 200 - RNA, 20 nt, Synthetic construct
  source 1..20: mol_type = other RNA
cacctaccacaccgttgcca

SEQ ID NO: 201 - RNA, 18 nt, Synthetic construct
  source 1..18: mol_type = other RNA
actaccatagcgttgtca

SEQ ID NO: 202 - RNA, 18 nt, Synthetic construct
  source 1..18: mol_type = other RNA
tgtatcactgcgctgtca

SEQ ID NO: 203 - RNA, 18 nt, Synthetic construct
  source 1..18: mol_type = other RNA
cttaccacagcgtcatca

SEQ ID NO: 204 - RNA, 18 nt, Synthetic construct
  source 1..18: mol_type = other RNA
cgtaccacagcgtagtaa

SEQ ID NO: 205 - RNA, 18 nt, Synthetic construct
  source 1..18: mol_type = other RNA
tgtaccacagcgttgtcg

SEQ ID NO: 206 - RNA, 18 nt, Synthetic construct
  source 1..18: mol_type = other RNA
tgtaccacagcgctgtaa

SEQ ID NO: 207 - RNA, 18 nt, Synthetic construct
  source 1..18: mol_type = other RNA
cgtaccatagcgtcgtca

SEQ ID NO: 208 - RNA, 20 nt, Synthetic construct
  source 1..20: mol_type = other RNA
cacataccacaccgttgtct

SEQ ID NO: 209 - RNA, 18 nt, Synthetic construct
  source 1..18: mol_type = other RNA
cctaccacagcgttatag

SEQ ID NO: 210 - RNA, 18 nt, Synthetic construct
  source 1..18: mol_type = other RNA
cgtaccacagcgtcatta

SEQ ID NO: 211 - RNA, 18 nt, Synthetic construct
  source 1..18: mol_type = other RNA
cgtaccacagcgttgaaa

SEQ ID NO: 212 - RNA, 18 nt, Synthetic construct
  source 1..18: mol_type = other RNA
agtaccacagcgttgtcg

SEQ ID NO: 213 - RNA, 18 nt, Synthetic construct
  source 1..18: mol_type = other RNA
cgtaccacagcgttctta

SEQ ID NO: 214 - RNA, 18 nt, Synthetic construct
  source 1..18: mol_type = other RNA
tctaccacagcgttgcca

SEQ ID NO: 215 - RNA, 18 nt, Synthetic construct
  source 1..18: mol_type = other RNA
tgtaccacagcgtagtca

SEQ ID NO: 216 - RNA, 18 nt, Synthetic construct
  source 1..18: mol_type = other RNA
cgaaccacagcgttctct

SEQ ID NO: 217 - RNA, 18 nt, Synthetic construct
  source 1..18: mol_type = other RNA
agtaccacagcgtcatca

SEQ ID NO: 218 - RNA, 20 nt, Synthetic construct
  source 1..20: mol_type = other RNA
caagtaccacaccgttgtgt

SEQ ID NO: 219 - RNA, 18 nt, Synthetic construct
  source 1..18: mol_type = other RNA
cgtaccatagcgtagtcc

SEQ ID NO: 220 - RNA, 18 nt, Synthetic construct
  source 1..18: mol_type = other RNA
actaccacagcgttgtca

SEQ ID NO: 221 - RNA, 18 nt, Synthetic construct
  source 1..18: mol_type = other RNA
tgtaccacagcgttctca

SEQ ID NO: 222 - RNA, 18 nt, Synthetic construct
  source 1..18: mol_type = other RNA
cgtaccacagcgtggtag

SEQ ID NO: 223 - RNA, 18 nt, Synthetic construct
  source 1..18: mol_type = other RNA
ttggtagctgctcttacg

SEQ ID NO: 224 - RNA, 18 nt, Synthetic construct
  source 1..18: mol_type = other RNA
ctggtctcttttctttcc

SEQ ID NO: 225 - RNA, 18 nt, Synthetic construct
  source 1..18: mol_type = other RNA
ccggcagctgcattttct

SEQ ID NO: 226 - RNA, 18 nt, Synthetic construct
  source 1..18: mol_type = other RNA
ttggtagcagctcttgcc

SEQ ID NO: 227 - RNA, 18 nt, Synthetic construct
  source 1..18: mol_type = other RNA
ctgatagatgttcactcc

SEQ ID NO: 228 - RNA, 18 nt, Synthetic construct
  source 1..18: mol_type = other RNA
atggtagctgcactttgc

SEQ ID NO: 229 - RNA, 18 nt, Synthetic construct
  source 1..18: mol_type = other RNA
ctgggagcagctctttcg

SEQ ID NO: 230 - RNA, 18 nt, Synthetic construct
  source 1..18: mol_type = other RNA
taggtagctgcactttcc

SEQ ID NO: 231 - RNA, 18 nt, Synthetic construct
  source 1..18: mol_type = other RNA
ctggtagttgcgcttcac

SEQ ID NO: 232 - RNA, 18 nt, Synthetic construct
  source 1..18: mol_type = other RNA
agggtagctgctctttta

SEQ ID NO: 233 - RNA, 18 nt, Synthetic construct
  source 1..18: mol_type = other RNA
ctggtagcttcgcctttc

SEQ ID NO: 234 - RNA, 18 nt, Synthetic construct
  source 1..18: mol_type = other RNA
agcgtagctgctctatcc

SEQ ID NO: 235 - RNA, 18 nt, Synthetic construct
  source 1..18: mol_type = other RNA
ctagtagcagctcttacg

SEQ ID NO: 236 - RNA, 18 nt, Synthetic construct
  source 1..18: mol_type = other RNA
gtggtagcggctcttact

SEQ ID NO: 237 - RNA, 18 nt, Synthetic construct
  source 1..18: mol_type = other RNA
gtgggagttactcttgcc

SEQ ID NO: 238 - RNA, 18 nt, Synthetic construct
  source 1..18: mol_type = other RNA
cgcgtagctgctcggacc

SEQ ID NO: 239 - RNA, 18 nt, Synthetic construct
  source 1..18: mol_type = other RNA
ctgatagctgctgcttcc

SEQ ID NO: 240 - RNA, 18 nt, Synthetic construct
  source 1..18: mol_type = other RNA
ctggaagctgcttatacc

SEQ ID NO: 241 - RNA, 18 nt, Synthetic construct
  source 1..18: mol_type = other RNA
ctggtagccgctctatac

SEQ ID NO: 242 - RNA, 18 nt, Synthetic construct
  source 1..18: mol_type = other RNA
ctgttagcgtctctttgt

SEQ ID NO: 243 - RNA, 18 nt, Synthetic construct
  source 1..18: mol_type = other RNA
ctggtagctgcttttgca

SEQ ID NO: 244 - RNA, 18 nt, Synthetic construct
  source 1..18: mol_type = other RNA
ctggtaggcgctcgttac

SEQ ID NO: 245 - RNA, 18 nt, Synthetic construct
  source 1..18: mol_type = other RNA
ttggtggcagctcttacc

SEQ ID NO: 246 - RNA, 18 nt, Synthetic construct
  source 1..18: mol_type = other RNA
atgggaactgctcgttcg

SEQ ID NO: 247 - RNA, 18 nt, Synthetic construct
  source 1..18: mol_type = other RNA
cttgaagctgctgattac

SEQ ID NO: 248 - RNA, 18 nt, Synthetic construct
  source 1..18: mol_type = other RNA
ctgctagctgctttttcc

SEQ ID NO: 249 - RNA, 18 nt, Synthetic construct
  source 1..18: mol_type = other RNA
tttgtagctgctctttcg

SEQ ID NO: 250 - RNA, 18 nt, Synthetic construct
  source 1..18: mol_type = other RNA
ctggttggtgggccttcc

SEQ ID NO: 251 - RNA, 18 nt, Synthetic construct
  source 1..18: mol_type = other RNA
cttggagctgctccatcc

SEQ ID NO: 252 - RNA, 18 nt, Synthetic construct
  source 1..18: mol_type = other RNA
ctgttagctgctctctct

SEQ ID NO: 253 - RNA, 18 nt, Synthetic construct
  source 1..18: mol_type = other RNA
ctgctagctgctttttca

SEQ ID NO: 254 - RNA, 18 nt, Synthetic construct
  source 1..18: mol_type = other RNA
ctgttagctgctctttac

SEQ ID NO: 255 - RNA, 18 nt, Synthetic construct
  source 1..18: mol_type = other RNA
ctgctagctgctccttcc

SEQ ID NO: 256 - RNA, 18 nt, Synthetic construct
  source 1..18: mol_type = other RNA
ctgctagttgctctttcc

SEQ ID NO: 257 - RNA, 18 nt, Synthetic construct
  source 1..18: mol_type = other RNA
ctgttagctgctttttcc

SEQ ID NO: 258 - RNA, 18 nt, Synthetic construct
  source 1..18: mol_type = other RNA
ctgttagctgctccttcc

SEQ ID NO: 259 - RNA, 18 nt, Synthetic construct
  source 1..18: mol_type = other RNA
ctgctagctgccctttcc

SEQ ID NO: 260 - RNA, 18 nt, Synthetic construct
  source 1..18: mol_type = other RNA
ctgctagctgctctgtct

SEQ ID NO: 261 - RNA, 18 nt, Synthetic construct
  source 1..18: mol_type = other RNA
ctgctagctgccctatcc

SEQ ID NO: 262 - RNA, 18 nt, Synthetic construct
  source 1..18: mol_type = other RNA
ctgctagctgctctctct

SEQ ID NO: 263 - RNA, 18 nt, Synthetic construct
  source 1..18: mol_type = other RNA
atggtagctgctttttcc

SEQ ID NO: 264 - RNA, 18 nt, Synthetic construct
  source 1..18: mol_type = other RNA
ctgctagcagctctttct

SEQ ID NO: 265 - RNA, 18 nt, Synthetic construct
  source 1..18: mol_type = other RNA
ctgctagctgctcattcc

SEQ ID NO: 266 - RNA, 18 nt, Synthetic construct
  source 1..18: mol_type = other RNA
gtgctagctgctttttcc

SEQ ID NO: 267 - RNA, 18 nt, Synthetic construct
  source 1..18: mol_type = other RNA
ctgttagctgctttttac

SEQ ID NO: 268 - RNA, 18 nt, Synthetic construct
  source 1..18: mol_type = other RNA
ctgttagctgctctctcc

SEQ ID NO: 269 - RNA, 18 nt, Synthetic construct
  source 1..18: mol_type = other RNA
ctgctagctgctctctcc

SEQ ID NO: 270 - RNA, 18 nt, Synthetic construct
  source 1..18: mol_type = other RNA
ctgctagctgctctttcc

SEQ ID NO: 271 - RNA, 18 nt, Synthetic construct
  source 1..18: mol_type = other RNA
ctgctagctgctctttct

SEQ ID NO: 272 - RNA, 18 nt, Synthetic construct
  source 1..18: mol_type = other RNA
ctgctagcagctctttcc

SEQ ID NO: 273 - RNA, 18 nt, Synthetic construct
  source 1..18: mol_type = other RNA
ctgctagctgctctttac

SEQ ID NO: 274 - RNA, 18 nt, Synthetic construct
  source 1..18: mol_type = other RNA
ttgctagctgctctctcc

SEQ ID NO: 275 - RNA, 18 nt, Synthetic construct
  source 1..18: mol_type = other RNA
ctgctagctgctctgtcc

SEQ ID NO: 276 - RNA, 18 nt, Synthetic construct
  source 1..18: mol_type = other RNA
ctggtacctgctcctccc

SEQ ID NO: 277 - RNA, 18 nt, Synthetic construct
  source 1..18: mol_type = other RNA
ctgttagctgctccctcc

SEQ ID NO: 278 - RNA, 18 nt, Synthetic construct
  source 1..18: mol_type = other RNA
ctgttagctgctctttct

SEQ ID NO: 279 - RNA, 18 nt, Synthetic construct
  source 1..18: mol_type = other RNA
ctgctagcagctctctcc

SEQ ID NO: 280 - RNA, 18 nt, Synthetic construct
  source 1..18: mol_type = other RNA
ttggtaggtgctcatacc

SEQ ID NO: 281 - RNA, 18 nt, Synthetic construct
  source 1..18: mol_type = other RNA
ctgctagttgccctttcc

SEQ ID NO: 282 - RNA, 18 nt, Synthetic construct
  source 1..18: mol_type = other RNA
atgctagctgctctttct

SEQ ID NO: 283 - RNA, 18 nt, Synthetic construct
  source 1..18: mol_type = other RNA
ctgctagctgctctctcg

SEQ ID NO: 284 - RNA, 18 nt, Synthetic construct
  source 1..18: mol_type = other RNA
ctgctagctgctctgacc

SEQ ID NO: 285 - RNA, 18 nt, Synthetic construct
  source 1..18: mol_type = other RNA
gtgctagctgctccttcc

SEQ ID NO: 286 - RNA, 18 nt, Synthetic construct
  source 1..18: mol_type = other RNA
caggtagctgctctattc

SEQ ID NO: 287 - RNA, 18 nt, Synthetic construct
  source 1..18: mol_type = other RNA
ctgctagctgctcttcgc

SEQ ID NO: 288 - RNA, 18 nt, Synthetic construct
  source 1..18: mol_type = other RNA
ctgctagctgctctttca

SEQ ID NO: 289 - RNA, 18 nt, Synthetic construct
  source 1..18: mol_type = other RNA
ctgctagctgctctatcc

SEQ ID NO: 290 - RNA, 18 nt, Synthetic construct
  source 1..18: mol_type = other RNA
ctgctagctgctcttatc

SEQ ID NO: 291 - RNA, 18 nt, Synthetic construct
  source 1..18: mol_type = other RNA
ctgttagctgctctatca

SEQ ID NO: 292 - RNA, 18 nt, Synthetic construct
  source 1..18: mol_type = other RNA
cacctactcgtgccagcc

SEQ ID NO: 293 - RNA, 18 nt, Synthetic construct
  source 1..18: mol_type = other RNA
cctctagtcgcgaccgct

SEQ ID NO: 294 - RNA, 18 nt, Synthetic construct
  source 1..18: mol_type = other RNA
cacccactcgcgaccccg

SEQ ID NO: 295 - RNA, 18 nt, Synthetic construct
  source 1..18: mol_type = other RNA
cacctagtcgcgaccgag

SEQ ID NO: 296 - RNA, 18 nt, Synthetic construct
  source 1..18: mol_type = other RNA
cacctacacgcgaccggg

SEQ ID NO: 297 - RNA, 18 nt, Synthetic construct
  source 1..18: mol_type = other RNA
cacctagtcgcgaacttg

SEQ ID NO: 298 - RNA, 18 nt, Synthetic construct
  source 1..18: mol_type = other RNA
catctactcgcgcccgcg

SEQ ID NO: 299 - RNA, 18 nt, Synthetic construct
  source 1..18: mol_type = other RNA
cacctacacgcgaccacc

SEQ ID NO: 300 - RNA, 18 nt, Synthetic construct
  source 1..18: mol_type = other RNA
cacctacacgcgtcagcg

SEQ ID NO: 301 - RNA, 18 nt, Synthetic construct
  source 1..18: mol_type = other RNA
ctcctactcgcgaccccg

SEQ ID NO: 302 - RNA, 18 nt, Synthetic construct
  source 1..18: mol_type = other RNA
gagctactcgcgaccgca

SEQ ID NO: 303 - RNA, 18 nt, Synthetic construct
  source 1..18: mol_type = other RNA
cagctactcgcgaccacg

SEQ ID NO: 304 - RNA, 18 nt, Synthetic construct
  source 1..18: mol_type = other RNA
cgactactcgcgagcgcg

SEQ ID NO: 305 - RNA, 18 nt, Synthetic construct
  source 1..18: mol_type = other RNA
cacctactcgcgtccgag

SEQ ID NO: 306 - RNA, 18 nt, Synthetic construct
  source 1..18: mol_type = other RNA
cacctactcgcgacagct

SEQ ID NO: 307 - RNA, 18 nt, Synthetic construct
  source 1..18: mol_type = other RNA
cacctacccgcgaccggg

SEQ ID NO: 308 - RNA, 18 nt, Synthetic construct
  source 1..18: mol_type = other RNA
accctactcgcgacggct

SEQ ID NO: 309 - RNA, 18 nt, Synthetic construct
  source 1..18: mol_type = other RNA
gacctactcgcgaccgag

SEQ ID NO: 310 - RNA, 18 nt, Synthetic construct
  source 1..18: mol_type = other RNA
tacatactcgcgaccgcg

SEQ ID NO: 311 - RNA, 18 nt, Synthetic construct
  source 1..18: mol_type = other RNA
cccctactcgcgcccgag

SEQ ID NO: 312 - RNA, 18 nt, Synthetic construct
  source 1..18: mol_type = other RNA
cgtctactcgcgtccgcg

SEQ ID NO: 313 - RNA, 18 nt, Synthetic construct
  source 1..18: mol_type = other RNA
tacccactcgcgcccgcg

SEQ ID NO: 314 - RNA, 18 nt, Synthetic construct
  source 1..18: mol_type = other RNA
cacctagtcgcgacggct

SEQ ID NO: 315 - RNA, 18 nt, Synthetic construct
  source 1..18: mol_type = other RNA
tacctactcgcgaccgag

SEQ ID NO: 316 - RNA, 18 nt, Synthetic construct
  source 1..18: mol_type = other RNA
tacctactcgcgacggag

SEQ ID NO: 317 - RNA, 18 nt, Synthetic construct
  source 1..18: mol_type = other RNA
ccactacacgcgaccgcg

SEQ ID NO: 318 - RNA, 18 nt, Synthetic construct
  source 1..18: mol_type = other RNA
cacccactcgcgaccgct

SEQ ID NO: 319 - RNA, 18 nt, Synthetic construct
  source 1..18: mol_type = other RNA
cacccactcgcgcccgcg

SEQ ID NO: 320 - RNA, 18 nt, Synthetic construct
  source 1..18: mol_type = other RNA
agcctactcgcgaccgag

SEQ ID NO: 321 - RNA, 18 nt, Synthetic construct
  source 1..18: mol_type = other RNA
cccctactcgcgacagcg

SEQ ID NO: 322 - RNA, 18 nt, Synthetic construct
  source 1..18: mol_type = other RNA
gacctactcgcgaccacg

SEQ ID NO: 323 - RNA, 18 nt, Synthetic construct
  source 1..18: mol_type = other RNA
cacatactcgcgacatcg

SEQ ID NO: 324 - RNA, 18 nt, Synthetic construct
  source 1..18: mol_type = other RNA
gacttactcgcgaccgcg

SEQ ID NO: 325 - RNA, 18 nt, Synthetic construct
  source 1..18: mol_type = other RNA
cacctagtcgcgtccgag

SEQ ID NO: 326 - RNA, 18 nt, Synthetic construct
  source 1..18: mol_type = other RNA
cacctactcgcgactggg

SEQ ID NO: 327 - RNA, 18 nt, Synthetic construct
  source 1..18: mol_type = other RNA
cacctactcgcgcccggg

SEQ ID NO: 328 - RNA, 18 nt, Synthetic construct
  source 1..18: mol_type = other RNA
cacctactcgcgcccgcg

SEQ ID NO: 329 - RNA, 18 nt, Synthetic construct
  source 1..18: mol_type = other RNA
cacctactcgcgcccgag

SEQ ID NO: 330 - RNA, 18 nt, Synthetic construct
  source 1..18: mol_type = other RNA
cacctactcgcgcccgct

SEQ ID NO: 331 - RNA, 18 nt, Synthetic construct
  source 1..18: mol_type = other RNA
gacatactcgcgaccgca

SEQ ID NO: 332 - RNA, 18 nt, Synthetic construct
  source 1..18: mol_type = other RNA
cacttactcgcgtccgcg

SEQ ID NO: 333 - RNA, 18 nt, Synthetic construct
  source 1..18: mol_type = other RNA
cgcatactcgcgcccggg

SEQ ID NO: 334 - RNA, 18 nt, Synthetic construct
  source 1..18: mol_type = other RNA
cgcatactcgcgaccgcg

SEQ ID NO: 335 - RNA, 18 nt, Synthetic construct
  source 1..18: mol_type = other RNA
caccaactcgcgcctgcg

SEQ ID NO: 336 - RNA, 18 nt, Synthetic construct
  source 1..18: mol_type = other RNA
cacctagtcgcgacctcg

SEQ ID NO: 337 - RNA, 18 nt, Synthetic construct
  source 1..18: mol_type = other RNA
aaactactcgcgaccgca

SEQ ID NO: 338 - RNA, 18 nt, Synthetic construct
  source 1..18: mol_type = other RNA
catctactcgcgcccgcc

SEQ ID NO: 339 - RNA, 18 nt, Synthetic construct
  source 1..18: mol_type = other RNA
cacctactcgcgaccgat

SEQ ID NO: 340 - RNA, 20 nt, Synthetic construct
  source 1..20: mol_type = other RNA
gtccgtatgtggcttcacta

SEQ ID NO: 341 - RNA, 20 nt, Synthetic construct
  source 1..20: mol_type = other RNA
gtccgtatgtgacttcgatt

SEQ ID NO: 342 - RNA, 20 nt, Synthetic construct
  source 1..20: mol_type = other RNA
ggccgcctgtgacttcactt

SEQ ID NO: 343 - RNA, 20 nt, Synthetic construct
  source 1..20: mol_type = other RNA
gtccctatgtggcttcactt

SEQ ID NO: 344 - RNA, 20 nt, Synthetic construct
  source 1..20: mol_type = other RNA
gaccgtatgtgacttctctt

SEQ ID NO: 345 - RNA, 20 nt, Synthetic construct
  source 1..20: mol_type = other RNA
gaccgtatgtgacctcactc

SEQ ID NO: 346 - RNA, 20 nt, Synthetic construct
  source 1..20: mol_type = other RNA
gtctggatgtgacttcactt

SEQ ID NO: 347 - RNA, 20 nt, Synthetic construct
  source 1..20: mol_type = other RNA
ttccttatgtgacttcactt

SEQ ID NO: 348 - RNA, 20 nt, Synthetic construct
  source 1..20: mol_type = other RNA
gtccatatgtgacttcaccg

SEQ ID NO: 349 - RNA, 20 nt, Synthetic construct
  source 1..20: mol_type = other RNA
gtccgtatgtaacttctctt

SEQ ID NO: 350 - RNA, 20 nt, Synthetic construct
  source 1..20: mol_type = other RNA
gtccttatgtgacttcactg

SEQ ID NO: 351 - RNA, 20 nt, Synthetic construct
  source 1..20: mol_type = other RNA
gtccgtatgcgacttccttg

SEQ ID NO: 352 - RNA, 20 nt, Synthetic construct
  source 1..20: mol_type = other RNA
gtccatatgtgacttcacgt

SEQ ID NO: 353 - RNA, 20 nt, Synthetic construct
  source 1..20: mol_type = other RNA
gtccgtatgcgacttcgcta

SEQ ID NO: 354 - RNA, 20 nt, Synthetic construct
  source 1..20: mol_type = other RNA
gtccgtatgtgacttcacac

SEQ ID NO: 355 - RNA, 20 nt, Synthetic construct
  source 1..20: mol_type = other RNA
gtccgtttgcgacttcactt

SEQ ID NO: 356 - RNA, 20 nt, Synthetic construct
  source 1..20: mol_type = other RNA
gtctgaatgtgacttcactt

SEQ ID NO: 357 - RNA, 20 nt, Synthetic construct
  source 1..20: mol_type = other RNA
gtccgtatgtggctgcactt

SEQ ID NO: 358 - RNA, 20 nt, Synthetic construct
  source 1..20: mol_type = other RNA
gtccctatgtgacttgactt

SEQ ID NO: 359 - RNA, 20 nt, Synthetic construct
  source 1..20: mol_type = other RNA
gtctctatgtgacttcactt

SEQ ID NO: 360 - RNA, 20 nt, Synthetic construct
  source 1..20: mol_type = other RNA
gtccttatgtgacttaactt

SEQ ID NO: 361 - RNA, 20 nt, Synthetic construct
  source 1..20: mol_type = other RNA
gtccctatgtgacttcattt

SEQ ID NO: 362 - RNA, 20 nt, Synthetic construct
  source 1..20: mol_type = other RNA
ttgcgtatgtgacttcactt

SEQ ID NO: 363 - RNA, 20 nt, Synthetic construct
  source 1..20: mol_type = other RNA
gtccttatctgacttcgctt

SEQ ID NO: 364 - RNA, 20 nt, Synthetic construct
  source 1..20: mol_type = other RNA
gtccctatgtgacttcacta

SEQ ID NO: 365 - RNA, 20 nt, Synthetic construct
  source 1..20: mol_type = other RNA
gtccgtatgtggcttcattt

SEQ ID NO: 366 - RNA, 20 nt, Synthetic construct
  source 1..20: mol_type = other RNA
gtccttatgtgacttcacgt

SEQ ID NO: 367 - RNA, 20 nt, Synthetic construct
  source 1..20: mol_type = other RNA
gtccgttagtgacttcaggc

SEQ ID NO: 368 - RNA, 20 nt, Synthetic construct
  source 1..20: mol_type = other RNA
gtccttatgtgactccactc

SEQ ID NO: 369 - RNA, 20 nt, Synthetic construct
  source 1..20: mol_type = other RNA
atccctatgtgacttcactt

SEQ ID NO: 370 - RNA, 20 nt, Synthetic construct
  source 1..20: mol_type = other RNA
atcagtatgtgacttcactt

SEQ ID NO: 371 - RNA, 20 nt, Synthetic construct
  source 1..20: mol_type = other RNA
atccctatgtgacttcagtt

SEQ ID NO: 372 - RNA, 20 nt, Synthetic construct
  source 1..20: mol_type = other RNA
gtccgtatgagactccactt

SEQ ID NO: 373 - RNA, 20 nt, Synthetic construct
  source 1..20: mol_type = other RNA
atcagtatgtgacttcaatt

SEQ ID NO: 374 - RNA, 20 nt, Synthetic construct
  source 1..20: mol_type = other RNA
gtctatatgtgacttcacgt

SEQ ID NO: 375 - RNA, 20 nt, Synthetic construct
  source 1..20: mol_type = other RNA
gtccctatgtgacttcactc

SEQ ID NO: 376 - RNA, 20 nt, Synthetic construct
  source 1..20: mol_type = other RNA
gcccgaatgtgacttcactt

SEQ ID NO: 377 - RNA, 20 nt, Synthetic construct
  source 1..20: mol_type = other RNA
gcctgtatgtaacttcactt

SEQ ID NO: 378 - RNA, 20 nt, Synthetic construct
  source 1..20: mol_type = other RNA
ggccgtatgcgacttcactt

SEQ ID NO: 379 - RNA, 20 nt, Synthetic construct
  source 1..20: mol_type = other RNA
ttcagtatgtgacttcactt

SEQ ID NO: 380 - RNA, 20 nt, Synthetic construct
  source 1..20: mol_type = other RNA
gtccgcatgtaacttcactc

SEQ ID NO: 381 - RNA, 20 nt, Synthetic construct
  source 1..20: mol_type = other RNA
gtccctatgtgacttccctt

SEQ ID NO: 382 - RNA, 20 nt, Synthetic construct
  source 1..20: mol_type = other RNA
gtccgtatgtgacttcacca

SEQ ID NO: 383 - RNA, 20 nt, Synthetic construct
  source 1..20: mol_type = other RNA
gtccgaatgtgactacactt

SEQ ID NO: 384 - RNA, 20 nt, Synthetic construct
  source 1..20: mol_type = other RNA
ggccatatgtgacttcactt

SEQ ID NO: 385 - RNA, 20 nt, Synthetic construct
  source 1..20: mol_type = other RNA
ctcagtatgtgacttcactt

SEQ ID NO: 386 - RNA, 20 nt, Synthetic construct
  source 1..20: mol_type = other RNA
gtcctgatgtgacttcactt

SEQ ID NO: 387 - RNA, 20 nt, Synthetic construct
  source 1..20: mol_type = other RNA
gtccatatgtgacttccctt

SEQ ID NO: 388 - RNA, 20 nt, Synthetic construct
  source 1..20: mol_type = other RNA
ggccgtatgtaacttccctt

SEQ ID NO: 389 - RNA, 20 nt, Synthetic construct
  source 1..20: mol_type = other RNA
gtctgtatgcgacttcactt

SEQ ID NO: 390 - RNA, 20 nt, Synthetic construct
  source 1..20: mol_type = other RNA
ctccgtaagtgacttcactt

SEQ ID NO: 391 - RNA, 20 nt, Synthetic construct
  source 1..20: mol_type = other RNA
gtccatatgtgacttcactc

SEQ ID NO: 392 - RNA, 20 nt, Synthetic construct
  source 1..20: mol_type = other RNA
gtccgtttgtgacttcacgt

SEQ ID NO: 393 - RNA, 20 nt, Synthetic construct
  source 1..20: mol_type = other RNA
gctcgtctgtgacttgactt

SEQ ID NO: 394 - RNA, 20 nt, Synthetic construct
  source 1..20: mol_type = other RNA
cctcgtatgtgacttcactt

SEQ ID NO: 395 - RNA, 20 nt, Synthetic construct
  source 1..20: mol_type = other RNA
gtccgtctgcgacttcactt

SEQ ID NO: 396 - RNA, 20 nt, Synthetic construct
  source 1..20: mol_type = other RNA
ctctgtatgtgacttcactt

SEQ ID NO: 397 - RNA, 20 nt, Synthetic construct
  source 1..20: mol_type = other RNA
gtccgtctgtgactttactt

SEQ ID NO: 398 - RNA, 20 nt, Synthetic construct
  source 1..20: mol_type = other RNA
gtctgtatgtgacttgacct

SEQ ID NO: 399 - RNA, 20 nt, Synthetic construct
  source 1..20: mol_type = other RNA
atccgtatgtgacttcccgt

SEQ ID NO: 400 - RNA, 18 nt, Synthetic construct
  source 1..18: mol_type = other RNA
caacagtccgcgataggg

SEQ ID NO: 401 - RNA, 18 nt, Synthetic construct
  source 1..18: mol_type = other RNA
cattactccgcggcgagc

SEQ ID NO: 402 - RNA, 18 nt, Synthetic construct
  source 1..18: mol_type = other RNA
cgacactccgcgtcagga

SEQ ID NO: 403 - RNA, 18 nt, Synthetic construct
  source 1..18: mol_type = other RNA
caacactccgttaaaggc

SEQ ID NO: 404 - RNA, 18 nt, Synthetic construct
  source 1..18: mol_type = other RNA
ctatactccgtgagaggg

SEQ ID NO: 405 - RNA, 18 nt, Synthetic construct
  source 1..18: mol_type = other RNA
cacgtaaccgcgacaggg

SEQ ID NO: 406 - RNA, 18 nt, Synthetic construct
  source 1..18: mol_type = other RNA
cgacggtccgcgacgggg

SEQ ID NO: 407 - RNA, 18 nt, Synthetic construct
  source 1..18: mol_type = other RNA
caacactccgcgccaggg

SEQ ID NO: 408 - RNA, 18 nt, Synthetic construct
  source 1..18: mol_type = other RNA
acacactccgcgacaggg

SEQ ID NO: 409 - RNA, 18 nt, Synthetic construct
  source 1..18: mol_type = other RNA
caacactccgcgacagag

SEQ ID NO: 410 - RNA, 18 nt, Synthetic construct
  source 1..18: mol_type = other RNA
caaccctccgcgagagcg

SEQ ID NO: 411 - RNA, 18 nt, Synthetic construct
  source 1..18: mol_type = other RNA
caacactcaagcacaaga

SEQ ID NO: 412 - RNA, 18 nt, Synthetic construct
  source 1..18: mol_type = other RNA
caacactccgcgacagat

SEQ ID NO: 413 - RNA, 18 nt, Synthetic construct
  source 1..18: mol_type = other RNA
caacactccgcgacaggt

SEQ ID NO: 414 - RNA, 18 nt, Synthetic construct
  source 1..18: mol_type = other RNA
caacaccccgcgacaggc

SEQ ID NO: 415 - RNA, 18 nt, Synthetic construct
  source 1..18: mol_type = other RNA
caacactccgcgtcaggt

SEQ ID NO: 416 - RNA, 18 nt, Synthetic construct
  source 1..18: mol_type = other RNA
caacactccgcgccagag

SEQ ID NO: 417 - RNA, 18 nt, Synthetic construct
  source 1..18: mol_type = other RNA
cagcactccgcgtcaggg

SEQ ID NO: 418 - RNA, 18 nt, Synthetic construct
  source 1..18: mol_type = other RNA
caacatcccgcgacagcg

SEQ ID NO: 419 - RNA, 18 nt, Synthetic construct
  source 1..18: mol_type = other RNA
caacaccccgcgacagag

SEQ ID NO: 420 - RNA, 18 nt, Synthetic construct
  source 1..18: mol_type = other RNA
caacactccgcggcagcg

SEQ ID NO: 421 - RNA, 18 nt, Synthetic construct
  source 1..18: mol_type = other RNA
caacactccgcgccaggc

SEQ ID NO: 422 - RNA, 18 nt, Synthetic construct
  source 1..18: mol_type = other RNA
caacactccgcggcagta

SEQ ID NO: 423 - RNA, 18 nt, Synthetic construct
  source 1..18: mol_type = other RNA
cgacactccgcgacagaa

SEQ ID NO: 424 - RNA, 18 nt, Synthetic construct
  source 1..18: mol_type = other RNA
caacactccgcgacagtt

SEQ ID NO: 425 - RNA, 18 nt, Synthetic construct
  source 1..18: mol_type = other RNA
cgacactccgcgccaggg

SEQ ID NO: 426 - RNA, 18 nt, Synthetic construct
  source 1..18: mol_type = other RNA
caacactccgcgacagtg

SEQ ID NO: 427 - RNA, 18 nt, Synthetic construct
  source 1..18: mol_type = other RNA
caacactccgcgacaggg

SEQ ID NO: 428 - RNA, 18 nt, Synthetic construct
  source 1..18: mol_type = other RNA
taacactccgcgccagag

SEQ ID NO: 429 - RNA, 18 nt, Synthetic construct
  source 1..18: mol_type = other RNA
caagactccgcgacaggc

SEQ ID NO: 430 - RNA, 18 nt, Synthetic construct
  source 1..18: mol_type = other RNA
caacacttcgcgacagtg

SEQ ID NO: 431 - RNA, 18 nt, Synthetic construct
  source 1..18: mol_type = other RNA
caacactccgcgccagtg

SEQ ID NO: 432 - RNA, 18 nt, Synthetic construct
  source 1..18: mol_type = other RNA
taacaccccgcgccaggg

SEQ ID NO: 433 - RNA, 18 nt, Synthetic construct
  source 1..18: mol_type = other RNA
caagactccgcgacagag

SEQ ID NO: 434 - RNA, 18 nt, Synthetic construct
  source 1..18: mol_type = other RNA
caacactccgcggcagag

SEQ ID NO: 435 - RNA, 18 nt, Synthetic construct
  source 1..18: mol_type = other RNA
caacactccgcgacagaa

SEQ ID NO: 436 - RNA, 18 nt, Synthetic construct
  source 1..18: mol_type = other RNA
caaccatccgcgacagcg

SEQ ID NO: 437 - RNA, 18 nt, Synthetic construct
  source 1..18: mol_type = other RNA
taacactccgcgacaggt

SEQ ID NO: 438 - RNA, 18 nt, Synthetic construct
  source 1..18: mol_type = other RNA
caacactccgcgacgggg

SEQ ID NO: 439 - RNA, 18 nt, Synthetic construct
  source 1..18: mol_type = other RNA
caacactccgcgacaggc

SEQ ID NO: 440 - RNA, 18 nt, Synthetic construct
  source 1..18: mol_type = other RNA
caacactccgcgccaggt

SEQ ID NO: 441 - RNA, 18 nt, Synthetic construct
  source 1..18: mol_type = other RNA
caacagtccgcgacagta

SEQ ID NO: 442 - RNA, 18 nt, Synthetic construct
  source 1..18: mol_type = other RNA
caacactccgcgacagcg

SEQ ID NO: 443 - RNA, 18 nt, Synthetic construct
  source 1..18: mol_type = other RNA
caacactccgcgtcagag

SEQ ID NO: 444 - RNA, 18 nt, Synthetic construct
  source 1..18: mol_type = other RNA
caacactccgcgacagcc

SEQ ID NO: 445 - RNA, 18 nt, Synthetic construct
  source 1..18: mol_type = other RNA
taacaccccgcgacagca

SEQ ID NO: 446 - RNA, 18 nt, Synthetic construct
  source 1..18: mol_type = other RNA
caagaccccgcgacaggg

SEQ ID NO: 447 - RNA, 18 nt, Synthetic construct
  source 1..18: mol_type = other RNA
taacactccgcgccaggg

SEQ ID NO: 448 - RNA, 18 nt, Synthetic construct
  source 1..18: mol_type = other RNA
taacactccgcgacagtg

SEQ ID NO: 449 - RNA, 18 nt, Synthetic construct
  source 1..18: mol_type = other RNA
caacactccgcgccagcg

SEQ ID NO: 450 - RNA, 18 nt, Synthetic construct
  source 1..18: mol_type = other RNA
gaacactccgcgccaggg

SEQ ID NO: 451 - RNA, 18 nt, Synthetic construct
  source 1..18: mol_type = other RNA
caacactccgcgacagct

SEQ ID NO: 452 - RNA, 18 nt, Synthetic construct
  source 1..18: mol_type = other RNA
caacaccccgcgacagcg

SEQ ID NO: 453 - RNA, 18 nt, Synthetic construct
  source 1..18: mol_type = other RNA
caagactccgcgccaggg

SEQ ID NO: 454 - RNA, 18 nt, Synthetic construct
  source 1..18: mol_type = other RNA
caacactccgcggcagtg

SEQ ID NO: 455 - RNA, 18 nt, Synthetic construct
  source 1..18: mol_type = other RNA
caataccccgcgacagag

SEQ ID NO: 456 - RNA, 18 nt, Synthetic construct
  source 1..18: mol_type = other RNA
caacaccccgcgacaggt

SEQ ID NO: 457 - RNA, 18 nt, Synthetic construct
  source 1..18: mol_type = other RNA
caagactccgcgacaggg

SEQ ID NO: 458 - RNA, 18 nt, Synthetic construct
  source 1..18: mol_type = other RNA
caacactccgcgacagga

SEQ ID NO: 459 - RNA, 18 nt, Synthetic construct
  source 1..18: mol_type = other RNA
caacatcccgcgacagag

SEQ ID NO: 460 - RNA, 18 nt, Synthetic construct
  source 1..18: mol_type = other RNA
caactcccagcgacaggg

SEQ ID NO: 461 - RNA, 18 nt, Synthetic construct
  source 1..18: mol_type = other RNA
taacaccccgcgacagag

SEQ ID NO: 462 - RNA, 18 nt, Synthetic construct
  source 1..18: mol_type = other RNA
caacattccgcggcaggt

SEQ ID NO: 463 - RNA, 18 nt, Synthetic construct
  source 1..18: mol_type = other RNA
taacactccgcgacagcg

SEQ ID NO: 464 - RNA, 18 nt, Synthetic construct
  source 1..18: mol_type = other RNA
caagactccgcggcaggg

SEQ ID NO: 465 - RNA, 18 nt, Synthetic construct
  source 1..18: mol_type = other RNA
caagactccgcgacagtg

SEQ ID NO: 466 - RNA, 18 nt, Synthetic construct
  source 1..18: mol_type = other RNA
caatactccgcgacaggg

SEQ ID NO: 467 - RNA, 18 nt, Synthetic construct
  source 1..18: mol_type = other RNA
caacaccccgcgccaggg

SEQ ID NO: 468 - RNA, 18 nt, Synthetic construct
  source 1..18: mol_type = other RNA
cgacagtccgcgccaggc

SEQ ID NO: 469 - RNA, 18 nt, Synthetic construct
  source 1..18: mol_type = other RNA
cggcactccgcgacaggt

SEQ ID NO: 470 - RNA, 18 nt, Synthetic construct
  source 1..18: mol_type = other RNA
ccacactccgcgacagag

SEQ ID NO: 471 - RNA, 18 nt, Synthetic construct
  source 1..18: mol_type = other RNA
caacaccccgcgacaggg

SEQ ID NO: 472 - RNA, 18 nt, Synthetic construct
  source 1..18: mol_type = other RNA
caacactccgcggcaggt

SEQ ID NO: 473 - RNA, 18 nt, Synthetic construct
  source 1..18: mol_type = other RNA
caatactccgcgacaagg

SEQ ID NO: 474 - RNA, 18 nt, Synthetic construct
  source 1..18: mol_type = other RNA
caacactccgcgacagta

SEQ ID NO: 475 - RNA, 18 nt, Synthetic construct
  source 1..18: mol_type = other RNA
caacaccccgcgacaaga

SEQ ID NO: 476 - RNA, 18 nt, Synthetic construct
  source 1..18: mol_type = other RNA
caacactccgcgtcgagt

SEQ ID NO: 477 - RNA, 18 nt, Synthetic construct
  source 1..18: mol_type = other RNA
caagactccgcgacaagg

SEQ ID NO: 478 - RNA, 18 nt, Synthetic construct
  source 1..18: mol_type = other RNA
caacgctccgcgacagtg

SEQ ID NO: 479 - RNA, 18 nt, Synthetic construct
  source 1..18: mol_type = other RNA
caaccatccgcgacaggg

SEQ ID NO: 480 - RNA, 18 nt, Synthetic construct
  source 1..18: mol_type = other RNA
caacactccgcgtcaggg

SEQ ID NO: 481 - RNA, 18 nt, Synthetic construct
  source 1..18: mol_type = other RNA
ccacactccgcggcaggg

SEQ ID NO: 482 - RNA, 18 nt, Synthetic construct
  source 1..18: mol_type = other RNA
caacactccgcgccagga

SEQ ID NO: 483 - RNA, 18 nt, Synthetic construct
  source 1..18: mol_type = other RNA
caacacttcgcgacaggg

SEQ ID NO: 484 - RNA, 18 nt, Synthetic construct
  source 1..18: mol_type = other RNA
aatcacaccgcgacaggg

SEQ ID NO: 485 - RNA, 18 nt, Synthetic construct
  source 1..18: mol_type = other RNA
aaacaatccgcgacagga

SEQ ID NO: 486 - RNA, 18 nt, Synthetic construct
  source 1..18: mol_type = other RNA
ccacaccccgcgacaggg

SEQ ID NO: 487 - RNA, 18 nt, Synthetic construct
  source 1..18: mol_type = other RNA
aaacacaccgcgccaggg

SEQ ID NO: 488 - RNA, 18 nt, Synthetic construct
  source 1..18: mol_type = other RNA
caacactccgcgacaaag

SEQ ID NO: 489 - RNA, 18 nt, Synthetic construct
  source 1..18: mol_type = other RNA
cgacactccgcgtcaggc

SEQ ID NO: 490 - RNA, 18 nt, Synthetic construct
  source 1..18: mol_type = other RNA
caacactccgcgccagat

SEQ ID NO: 491 - RNA, 18 nt, Synthetic construct
  source 1..18: mol_type = other RNA
caacactccgcgacaagg

SEQ ID NO: 492 - RNA, 18 nt, Synthetic construct
  source 1..18: mol_type = other RNA
ccacactccgcgccaggg

SEQ ID NO: 493 - RNA, 18 nt, Synthetic construct
  source 1..18: mol_type = other RNA
caacactccgcggcaggg

SEQ ID NO: 494 - RNA, 18 nt, Synthetic construct
  source 1..18: mol_type = other RNA
caacactccgcgacatgt

SEQ ID NO: 495 - RNA, 18 nt, Synthetic construct
  source 1..18: mol_type = other RNA
caacactccgcgccaagg

SEQ ID NO: 496 - RNA, 18 nt, Synthetic construct
  source 1..18: mol_type = other RNA
ttacactccgcgccagga

SEQ ID NO: 497 - RNA, 18 nt, Synthetic construct
  source 1..18: mol_type = other RNA
taacactccgcgccaagg

SEQ ID NO: 498 - RNA, 18 nt, Synthetic construct
  source 1..18: mol_type = other RNA
taacactccgcgacaggg

SEQ ID NO: 499 - RNA, 18 nt, Synthetic construct
  source 1..18: mol_type = other RNA
aaacactccgcgccaggg

SEQ ID NO: 500 - RNA, 18 nt, Synthetic construct
  source 1..18: mol_type = other RNA
cctcactccgcgacaggg

SEQ ID NO: 501 - RNA, 18 nt, Synthetic construct
  source 1..18: mol_type = other RNA
caacactccgcgacatgg

SEQ ID NO: 502 - RNA, 18 nt, Synthetic construct
  source 1..18: mol_type = other RNA
caatactccgcgtcaggg

SEQ ID NO: 503 - RNA, 18 nt, Synthetic construct
  source 1..18: mol_type = other RNA
caacactccgcgtcagcg

SEQ ID NO: 504 - RNA, 18 nt, Synthetic construct
  source 1..18: mol_type = other RNA
caacagtccgcgacagag

SEQ ID NO: 505 - RNA, 18 nt, Synthetic construct
  source 1..18: mol_type = other RNA
cagcattccgcgccaggg

SEQ ID NO: 506 - RNA, 18 nt, Synthetic construct
  source 1..18: mol_type = other RNA
caacacttcgcgacagag

SEQ ID NO: 507 - RNA, 18 nt, Synthetic construct
  source 1..18: mol_type = other RNA
caacagtccgcgacatgg

SEQ ID NO: 508 - RNA, 18 nt, Synthetic construct
  source 1..18: mol_type = other RNA
caacaatccgcgacagcc

SEQ ID NO: 509 - RNA, 18 nt, Synthetic construct
  source 1..18: mol_type = other RNA
ccacactccgcgacaggg

SEQ ID NO: 510 - RNA, 18 nt, Synthetic construct
  source 1..18: mol_type = other RNA
caacactccgcgacaaga

SEQ ID NO: 511 - RNA, 18 nt, Synthetic construct
  source 1..18: mol_type = other RNA
caacacaccgcgacagcg

SEQ ID NO: 512 - RNA, 18 nt, Synthetic construct
  source 1..18: mol_type = other RNA
gaacactccgcgacagga

SEQ ID NO: 513 - RNA, 18 nt, Synthetic construct
  source 1..18: mol_type = other RNA
caacactccgcgccgagg

SEQ ID NO: 514 - RNA, 18 nt, Synthetic construct
  source 1..18: mol_type = other RNA
caacacttcgcgacaggt

SEQ ID NO: 515 - RNA, 18 nt, Synthetic construct
  source 1..18: mol_type = other RNA
ccacattccgcgacaggg

SEQ ID NO: 516 - RNA, 18 nt, Synthetic construct
  source 1..18: mol_type = other RNA
aaacactccgcgacaggg

SEQ ID NO: 517 - RNA, 18 nt, Synthetic construct
  source 1..18: mol_type = other RNA
gcacactccgcgacaggt

SEQ ID NO: 518 - RNA, 18 nt, Synthetic construct
  source 1..18: mol_type = other RNA
cgacacaccgcgacaggg

SEQ ID NO: 519 - RNA, 18 nt, Synthetic construct
  source 1..18: mol_type = other RNA
gagggtgacgccatgtac

SEQ ID NO: 520 - RNA, 18 nt, Synthetic construct
  source 1..18: mol_type = other RNA
aagggtcccaccgcgtac

SEQ ID NO: 521 - RNA, 18 nt, Synthetic construct
  source 1..18: mol_type = other RNA
ggggctgccaccgtgtgc

SEQ ID NO: 522 - RNA, 18 nt, Synthetic construct
  source 1..18: mol_type = other RNA
aaaggtgccaccgagttc

SEQ ID NO: 523 - RNA, 18 nt, Synthetic construct
  source 1..18: mol_type = other RNA
gaggttgccaccgtatgc

SEQ ID NO: 524 - RNA, 18 nt, Synthetic construct
  source 1..18: mol_type = other RNA
gaggctgccaccgtctac

SEQ ID NO: 525 - RNA, 18 nt, Synthetic construct
  source 1..18: mol_type = other RNA
gaggctgtcaccgcgtac

SEQ ID NO: 526 - RNA, 18 nt, Synthetic construct
  source 1..18: mol_type = other RNA
gaggctgccaccgtgtta

SEQ ID NO: 527 - RNA, 18 nt, Synthetic construct
  source 1..18: mol_type = other RNA
gaggctgacaccgtgttc

SEQ ID NO: 528 - RNA, 18 nt, Synthetic construct
  source 1..18: mol_type = other RNA
gaggttgacaccgtgtat

SEQ ID NO: 529 - RNA, 18 nt, Synthetic construct
  source 1..18: mol_type = other RNA
gaggctgccaccttgtgc

SEQ ID NO: 530 - RNA, 18 nt, Synthetic construct
  source 1..18: mol_type = other RNA
gaggctgccaccgtgtca

SEQ ID NO: 531 - RNA, 18 nt, Synthetic construct
  source 1..18: mol_type = other RNA
cagggcgccaccgagtac

SEQ ID NO: 532 - RNA, 18 nt, Synthetic construct
  source 1..18: mol_type = other RNA
aaggctgccaccgtgtgc

SEQ ID NO: 533 - RNA, 18 nt, Synthetic construct
  source 1..18: mol_type = other RNA
gagggtgccaccgaagac

SEQ ID NO: 534 - RNA, 18 nt, Synthetic construct
  source 1..18: mol_type = other RNA
gaggctgccaccgtgcaa

SEQ ID NO: 535 - RNA, 18 nt, Synthetic construct
  source 1..18: mol_type = other RNA
cagggtgccaccctgtaa

SEQ ID NO: 536 - RNA, 18 nt, Synthetic construct
  source 1..18: mol_type = other RNA
gaaggtgccaccgtatat

SEQ ID NO: 537 - RNA, 18 nt, Synthetic construct
  source 1..18: mol_type = other RNA
gagggtgccaccctgtca

SEQ ID NO: 538 - RNA, 18 nt, Synthetic construct
  source 1..18: mol_type = other RNA
ggaggtgccaccgtctag

SEQ ID NO: 539 - RNA, 18 nt, Synthetic construct
  source 1..18: mol_type = other RNA
gtggttgccaccgtgtaa

SEQ ID NO: 540 - RNA, 18 nt, Synthetic construct
  source 1..18: mol_type = other RNA
gaggctgccaccgtgttc

SEQ ID NO: 541 - RNA, 18 nt, Synthetic construct
  source 1..18: mol_type = other RNA
gaggctgccaccgtgtat

SEQ ID NO: 542 - RNA, 18 nt, Synthetic construct
  source 1..18: mol_type = other RNA
aaggttgccaccgtgtat

SEQ ID NO: 543 - RNA, 18 nt, Synthetic construct
  source 1..18: mol_type = other RNA
gagagtgccaccatgtat

SEQ ID NO: 544 - RNA, 18 nt, Synthetic construct
  source 1..18: mol_type = other RNA
ggggctggcaccgtgtac

SEQ ID NO: 545 - RNA, 18 nt, Synthetic construct
  source 1..18: mol_type = other RNA
gaggctggcaccgtggaa

SEQ ID NO: 546 - RNA, 18 nt, Synthetic construct
  source 1..18: mol_type = other RNA
gagggtaccaccctgttc

SEQ ID NO: 547 - RNA, 18 nt, Synthetic construct
  source 1..18: mol_type = other RNA
gaggctgtcaccgagtac

SEQ ID NO: 548 - RNA, 18 nt, Synthetic construct
  source 1..18: mol_type = other RNA
gaggctgccaccgtgtaa

SEQ ID NO: 549 - RNA, 18 nt, Synthetic construct
  source 1..18: mol_type = other RNA
caggctgccaccttgtac

SEQ ID NO: 550 - RNA, 18 nt, Synthetic construct
  source 1..18: mol_type = other RNA
gggggtgccaccgagtgc

SEQ ID NO: 551 - RNA, 18 nt, Synthetic construct
  source 1..18: mol_type = other RNA
aaggctgccaccgtgtaa

SEQ ID NO: 552 - RNA, 18 nt, Synthetic construct
  source 1..18: mol_type = other RNA
ggggctgccaccctgtac

SEQ ID NO: 553 - RNA, 18 nt, Synthetic construct
  source 1..18: mol_type = other RNA
gaggctgccaccctgtat

SEQ ID NO: 554 - RNA, 18 nt, Synthetic construct
  source 1..18: mol_type = other RNA
gaggctgccaccgtttac

SEQ ID NO: 555 - RNA, 18 nt, Synthetic construct
  source 1..18: mol_type = other RNA
tagggtgccaccgcgtgc

SEQ ID NO: 556 - RNA, 18 nt, Synthetic construct
  source 1..18: mol_type = other RNA
gaggctggcaccgtgtaa

SEQ ID NO: 557 - RNA, 18 nt, Synthetic construct
  source 1..18: mol_type = other RNA
gggggtgccaccgagtat

SEQ ID NO: 558 - RNA, 18 nt, Synthetic construct
  source 1..18: mol_type = other RNA
taggctgccaccgtgtag

SEQ ID NO: 559 - RNA, 18 nt, Synthetic construct
  source 1..18: mol_type = other RNA
gaggttggcaccgagtgc

SEQ ID NO: 560 - RNA, 18 nt, Synthetic construct
  source 1..18: mol_type = other RNA
gaggctgtcaccgtgcac

SEQ ID NO: 561 - RNA, 18 nt, Synthetic construct
  source 1..18: mol_type = other RNA
gaggctgccaccgtgtag

SEQ ID NO: 562 - RNA, 18 nt, Synthetic construct
  source 1..18: mol_type = other RNA
gagggtgccaccgagtcc

SEQ ID NO: 563 - RNA, 18 nt, Synthetic construct
  source 1..18: mol_type = other RNA
ggtggtgccaccgtgtgc

SEQ ID NO: 564 - RNA, 18 nt, Synthetic construct
  source 1..18: mol_type = other RNA
taggctgccaccgggtaa

SEQ ID NO: 565 - RNA, 18 nt, Synthetic construct
  source 1..18: mol_type = other RNA
gagggcgccacggtggac

SEQ ID NO: 566 - RNA, 18 nt, Synthetic construct
  source 1..18: mol_type = other RNA
gaggttgccaccgtcttc

SEQ ID NO: 567 - RNA, 18 nt, Synthetic construct
  source 1..18: mol_type = other RNA
gaggttgccaccgtatac

SEQ ID NO: 568 - RNA, 18 nt, Synthetic construct
  source 1..18: mol_type = other RNA
gggggtgccacggtgtat

SEQ ID NO: 569 - RNA, 18 nt, Synthetic construct
  source 1..18: mol_type = other RNA
gatggttccaccgtgtac

SEQ ID NO: 570 - RNA, 18 nt, Synthetic construct
  source 1..18: mol_type = other RNA
gaggctgtcaccctgtac

SEQ ID NO: 571 - RNA, 18 nt, Synthetic construct
  source 1..18: mol_type = other RNA
gaggctggcaccgtgtcc

SEQ ID NO: 572 - RNA, 18 nt, Synthetic construct
  source 1..18: mol_type = other RNA
gaaggtgccaccatttac

SEQ ID NO: 573 - RNA, 18 nt, Synthetic construct
  source 1..18: mol_type = other RNA
gaggctgacaccgtgtta

SEQ ID NO: 574 - RNA, 18 nt, Synthetic construct
  source 1..18: mol_type = other RNA
gaggctgccaccgagtac

SEQ ID NO: 575 - RNA, 18 nt, Synthetic construct
  source 1..18: mol_type = other RNA
gaggttgccaccttgtac

SEQ ID NO: 576 - RNA, 18 nt, Synthetic construct
  source 1..18: mol_type = other RNA
gaggctgccaccgtatac

SEQ ID NO: 577 - RNA, 18 nt, Synthetic construct
  source 1..18: mol_type = other RNA
aaggttgccaccgtgtta

SEQ ID NO: 578 - RNA, 18 nt, Synthetic construct
  source 1..18: mol_type = other RNA
gaggctgccaccgtggac

SEQ ID NO: 579 - RNA, 18 nt, Synthetic construct
  source 1..18: mol_type = other RNA
gaggctgccaccgtgaac

SEQ ID NO: 580 - RNA, 18 nt, Synthetic construct
  source 1..18: mol_type = other RNA
gaggctgccaccgcgttc

SEQ ID NO: 581 - RNA, 18 nt, Synthetic construct
  source 1..18: mol_type = other RNA
gagggtaccaccgcgtat

SEQ ID NO: 582 - RNA, 18 nt, Synthetic construct
  source 1..18: mol_type = other RNA
gaggttgccaccgagtac

SEQ ID NO: 583 - RNA, 18 nt, Synthetic construct
  source 1..18: mol_type = other RNA
gaggctgacaccgagtac

SEQ ID NO: 584 - RNA, 18 nt, Synthetic construct
  source 1..18: mol_type = other RNA
cagggtgccaccgtttaa

SEQ ID NO: 585 - RNA, 18 nt, Synthetic construct
  source 1..18: mol_type = other RNA
gaggttgccaccatgtac

SEQ ID NO: 586 - RNA, 18 nt, Synthetic construct
  source 1..18: mol_type = other RNA
gaggctggcaccgtgcac

SEQ ID NO: 587 - RNA, 18 nt, Synthetic construct
  source 1..18: mol_type = other RNA
gaggctgccaccctgtac

SEQ ID NO: 588 - RNA, 18 nt, Synthetic construct
  source 1..18: mol_type = other RNA
ggaggtgccaccgagtac

SEQ ID NO: 589 - RNA, 18 nt, Synthetic construct
  source 1..18: mol_type = other RNA
gagtgttccaccgggtac

SEQ ID NO: 590 - RNA, 18 nt, Synthetic construct
  source 1..18: mol_type = other RNA
gaggctgccaccgtgtac

SEQ ID NO: 591 - RNA, 18 nt, Synthetic construct
  source 1..18: mol_type = other RNA
gaggctgccaccgcgtac

SEQ ID NO: 592 - RNA, 18 nt, Synthetic construct
  source 1..18: mol_type = other RNA
aaggctgccaccctgtac

SEQ ID NO: 593 - RNA, 18 nt, Synthetic construct
  source 1..18: mol_type = other RNA
gtggctgccaccgtgtat

SEQ ID NO: 594 - RNA, 18 nt, Synthetic construct
  source 1..18: mol_type = other RNA
acgataggaacctgagtc

SEQ ID NO: 595 - RNA, 18 nt, Synthetic construct
  source 1..18: mol_type = other RNA
cagatgggaacctcggtg

SEQ ID NO: 596 - RNA, 18 nt, Synthetic construct
  source 1..18: mol_type = other RNA
tagataggaacctggatc

SEQ ID NO: 597 - RNA, 18 nt, Synthetic construct
  source 1..18: mol_type = other RNA
cagacgggaaccggggtc

SEQ ID NO: 598 - RNA, 18 nt, Synthetic construct
  source 1..18: mol_type = other RNA
cggatgggaacctgggac

SEQ ID NO: 599 - RNA, 18 nt, Synthetic construct
  source 1..18: mol_type = other RNA
cacatgggaacctgaatc

SEQ ID NO: 600 - RNA, 18 nt, Synthetic construct
  source 1..18: mol_type = other RNA
cagataggaacctgggaa

SEQ ID NO: 601 - RNA, 18 nt, Synthetic construct
  source 1..18: mol_type = other RNA
cagatgcgaacctgggcc

SEQ ID NO: 602 - RNA, 18 nt, Synthetic construct
  source 1..18: mol_type = other RNA
cacatgggaacctaggtt

SEQ ID NO: 603 - RNA, 18 nt, Synthetic construct
  source 1..18: mol_type = other RNA
gagatgggaacctggctc

SEQ ID NO: 604 - RNA, 18 nt, Synthetic construct
  source 1..18: mol_type = other RNA
cacatgggaacctgagtc

SEQ ID NO: 605 - RNA, 18 nt, Synthetic construct
  source 1..18: mol_type = other RNA
cagataggaacctgggtt

SEQ ID NO: 606 - RNA, 18 nt, Synthetic construct
  source 1..18: mol_type = other RNA
cagataggaacctggttc

SEQ ID NO: 607 - RNA, 18 nt, Synthetic construct
  source 1..18: mol_type = other RNA
cacatgggaacctaggta

SEQ ID NO: 608 - RNA, 18 nt, Synthetic construct
  source 1..18: mol_type = other RNA
cagataggaacctggcta

SEQ ID NO: 609 - RNA, 18 nt, Synthetic construct
  source 1..18: mol_type = other RNA
catatgggaacctgagtc

SEQ ID NO: 610 - RNA, 18 nt, Synthetic construct
  source 1..18: mol_type = other RNA
cagatgggaacctaggtt

SEQ ID NO: 611 - RNA, 18 nt, Synthetic construct
  source 1..18: mol_type = other RNA
cggatgggaacccgggga

SEQ ID NO: 612 - RNA, 18 nt, Synthetic construct
  source 1..18: mol_type = other RNA
ccgataggaacccgggtc

SEQ ID NO: 613 - RNA, 18 nt, Synthetic construct
  source 1..18: mol_type = other RNA
cagatgggaacctaggta

SEQ ID NO: 614 - RNA, 18 nt, Synthetic construct
  source 1..18: mol_type = other RNA
cggataggaacctgggcc

SEQ ID NO: 615 - RNA, 18 nt, Synthetic construct
  source 1..18: mol_type = other RNA
cagataggaacctaggtc

SEQ ID NO: 616 - RNA, 18 nt, Synthetic construct
  source 1..18: mol_type = other RNA
cagataggaacctgagtc

SEQ ID NO: 617 - RNA, 18 nt, Synthetic construct
  source 1..18: mol_type = other RNA
cagataggaacctggatc

SEQ ID NO: 618 - RNA, 18 nt, Synthetic construct
  source 1..18: mol_type = other RNA
cagatagtaacctggttc

SEQ ID NO: 619 - RNA, 18 nt, Synthetic construct
  source 1..18: mol_type = other RNA
cagataggaacctgggcc

SEQ ID NO: 620 - RNA, 18 nt, Synthetic construct
  source 1..18: mol_type = other RNA
cacatgggaacctaggtc

SEQ ID NO: 621 - RNA, 18 nt, Synthetic construct
  source 1..18: mol_type = other RNA
cagatggaaacctgggcc

SEQ ID NO: 622 - RNA, 18 nt, Synthetic construct
  source 1..18: mol_type = other RNA
caaatgggaacctaggtc

SEQ ID NO: 623 - RNA, 18 nt, Synthetic construct
  source 1..18: mol_type = other RNA
cagatgtgaacctcggtc

SEQ ID NO: 624 - RNA, 18 nt, Synthetic construct
  source 1..18: mol_type = other RNA
caaatgggaacccgagtc

SEQ ID NO: 625 - RNA, 18 nt, Synthetic construct
  source 1..18: mol_type = other RNA
cagatgggaacctcggtt

SEQ ID NO: 626 - RNA, 18 nt, Synthetic construct
  source 1..18: mol_type = other RNA
cagctgggaacccgggta

SEQ ID NO: 627 - RNA, 18 nt, Synthetic construct
  source 1..18: mol_type = other RNA
gagatgggaacctgagtt

SEQ ID NO: 628 - RNA, 18 nt, Synthetic construct
  source 1..18: mol_type = other RNA
cagataggaacctcggtc

SEQ ID NO: 629 - RNA, 18 nt, Synthetic construct
  source 1..18: mol_type = other RNA
cagatgggaacctgagtc

SEQ ID NO: 630 - RNA, 18 nt, Synthetic construct
  source 1..18: mol_type = other RNA
cagatgtgaacctggctc

SEQ ID NO: 631 - RNA, 18 nt, Synthetic construct
  source 1..18: mol_type = other RNA
cagatgggaacctaggtc

SEQ ID NO: 632 - RNA, 18 nt, Synthetic construct
  source 1..18: mol_type = other RNA
aagatgggaacctaggtg

SEQ ID NO: 633 - RNA, 18 nt, Synthetic construct
  source 1..18: mol_type = other RNA
cagatgggaacctagatc

SEQ ID NO: 634 - RNA, 18 nt, Synthetic construct
  source 1..18: mol_type = other RNA
cagataggaacccgggtc

SEQ ID NO: 635 - RNA, 18 nt, Synthetic construct
  source 1..18: mol_type = other RNA
caacctagaccatctcat

SEQ ID NO: 636 - RNA, 18 nt, Synthetic construct
  source 1..18: mol_type = other RNA
caacctcaagtaccgcat

SEQ ID NO: 637 - RNA, 18 nt, Synthetic construct
  source 1..18: mol_type = other RNA
caaccttgaccaccagat

SEQ ID NO: 638 - RNA, 18 nt, Synthetic construct
  source 1..18: mol_type = other RNA
caaccccgaccatcgcct

SEQ ID NO: 639 - RNA, 18 nt, Synthetic construct
  source 1..18: mol_type = other RNA
taacctggagcaccgcat

SEQ ID NO: 640 - RNA, 18 nt, Synthetic construct
  source 1..18: mol_type = other RNA
caatctcgaccaccgggt

SEQ ID NO: 641 - RNA, 18 nt, Synthetic construct
  source 1..18: mol_type = other RNA
caacctggaccaccgtct

SEQ ID NO: 642 - RNA, 18 nt, Synthetic construct
  source 1..18: mol_type = other RNA
caacatcaaccatcgctt

SEQ ID NO: 643 - RNA, 18 nt, Synthetic construct
  source 1..18: mol_type = other RNA
caagatcgaccatcgcat

SEQ ID NO: 644 - RNA, 18 nt, Synthetic construct
  source 1..18: mol_type = other RNA
caacctcgaccacggggc

SEQ ID NO: 645 - RNA, 18 nt, Synthetic construct
  source 1..18: mol_type = other RNA
caacctagaccatcgcag

SEQ ID NO: 646 - RNA, 18 nt, Synthetic construct
  source 1..18: mol_type = other RNA
caacctcgagcatcgcat

SEQ ID NO: 647 - RNA, 18 nt, Synthetic construct
  source 1..18: mol_type = other RNA
caagctcgagcaccgcct

SEQ ID NO: 648 - RNA, 18 nt, Synthetic construct
  source 1..18: mol_type = other RNA
gaacctcgaccaccgctt

SEQ ID NO: 649 - RNA, 18 nt, Synthetic construct
  source 1..18: mol_type = other RNA
gaaccccgaccaccggat

SEQ ID NO: 650 - RNA, 18 nt, Synthetic construct
  source 1..18: mol_type = other RNA
cgtcctcgaccaccggat

SEQ ID NO: 651 - RNA, 18 nt, Synthetic construct
  source 1..18: mol_type = other RNA
caaccccgaccatctcat

SEQ ID NO: 652 - RNA, 18 nt, Synthetic construct
  source 1..18: mol_type = other RNA
caaccccaaccaccgcat

SEQ ID NO: 653 - RNA, 18 nt, Synthetic construct
  source 1..18: mol_type = other RNA
ctacttcgaccaccgcac

SEQ ID NO: 654 - RNA, 18 nt, Synthetic construct
  source 1..18: mol_type = other RNA
cagcctcgaccaccggat

SEQ ID NO: 655 - RNA, 18 nt, Synthetic construct
  source 1..18: mol_type = other RNA
caaccccgaccaccgcaa

SEQ ID NO: 656 - RNA, 18 nt, Synthetic construct
  source 1..18: mol_type = other RNA
taatctcgaccaccgcct

SEQ ID NO: 657 - RNA, 18 nt, Synthetic construct
  source 1..18: mol_type = other RNA
caacaacgaccaccggat

SEQ ID NO: 658 - RNA, 18 nt, Synthetic construct
  source 1..18: mol_type = other RNA
ccacctcgaccatcacat

SEQ ID NO: 659 - RNA, 18 nt, Synthetic construct
  source 1..18: mol_type = other RNA
caaccccgaccatcgcta

SEQ ID NO: 660 - RNA, 18 nt, Synthetic construct
  source 1..18: mol_type = other RNA
caactgtgtccaaggcat

SEQ ID NO: 661 - RNA, 18 nt, Synthetic construct
  source 1..18: mol_type = other RNA
ctacatcgaccacctaat

SEQ ID NO: 662 - RNA, 18 nt, Synthetic construct
  source 1..18: mol_type = other RNA
caacctcaagcaccgcat

SEQ ID NO: 663 - RNA, 18 nt, Synthetic construct
  source 1..18: mol_type = other RNA
aaacctggaccaccgctt

SEQ ID NO: 664 - RNA, 18 nt, Synthetic construct
  source 1..18: mol_type = other RNA
ccacctagaccatcgcat

SEQ ID NO: 665 - RNA, 18 nt, Synthetic construct
  source 1..18: mol_type = other RNA
cccccacgactaccgcaa

SEQ ID NO: 666 - RNA, 18 nt, Synthetic construct
  source 1..18: mol_type = other RNA
caacttcgaccatggcat

SEQ ID NO: 667 - RNA, 18 nt, Synthetic construct
  source 1..18: mol_type = other RNA
ccaccacgaccatcgcct

SEQ ID NO: 668 - RNA, 18 nt, Synthetic construct
  source 1..18: mol_type = other RNA
caacctcgaccaccgctc

SEQ ID NO: 669 - RNA, 18 nt, Synthetic construct
  source 1..18: mol_type = other RNA
caaccccgagcaccgcag

SEQ ID NO: 670 - RNA, 18 nt, Synthetic construct
  source 1..18: mol_type = other RNA
caagctcgaccacggaat

SEQ ID NO: 671 - RNA, 18 nt, Synthetic construct
  source 1..18: mol_type = other RNA
cagcctcgagcacagcat

SEQ ID NO: 672 - RNA, 18 nt, Synthetic construct
  source 1..18: mol_type = other RNA
aaacctccaccacagcat

SEQ ID NO: 673 - RNA, 18 nt, Synthetic construct
  source 1..18: mol_type = other RNA
tagcctcgaccatcgcat

SEQ ID NO: 674 - RNA, 18 nt, Synthetic construct
  source 1..18: mol_type = other RNA
ccacctcgaccatcgccg

SEQ ID NO: 675 - RNA, 18 nt, Synthetic construct
  source 1..18: mol_type = other RNA
caacctcaaccatggcat

SEQ ID NO: 676 - RNA, 18 nt, Synthetic construct
  source 1..18: mol_type = other RNA
caacctcgaccaccgggt

SEQ ID NO: 677 - RNA, 18 nt, Synthetic construct
  source 1..18: mol_type = other RNA
caaccacgaccaccgggt

SEQ ID NO: 678 - RNA, 18 nt, Synthetic construct
  source 1..18: mol_type = other RNA
tatactcgaccaccgcat

SEQ ID NO: 679 - RNA, 18 nt, Synthetic construct
  source 1..18: mol_type = other RNA
caaacaccaccaccgcat

SEQ ID NO: 680 - RNA, 18 nt, Synthetic construct
  source 1..18: mol_type = other RNA
ccacagcgaccacctcat

SEQ ID NO: 681 - RNA, 18 nt, Synthetic construct
  source 1..18: mol_type = other RNA
caacctcgagcaccacat

SEQ ID NO: 682 - RNA, 18 nt, Synthetic construct
  source 1..18: mol_type = other RNA
cgacctcgaccagcgaat

SEQ ID NO: 683 - RNA, 18 nt, Synthetic construct
  source 1..18: mol_type = other RNA
cagcctcgaccatcgcat

SEQ ID NO: 684 - RNA, 18 nt, Synthetic construct
  source 1..18: mol_type = other RNA
caacatcgaccactgcgc

SEQ ID NO: 685 - RNA, 18 nt, Synthetic construct
  source 1..18: mol_type = other RNA
agacctcgaccatcgtat

SEQ ID NO: 686 - RNA, 18 nt, Synthetic construct
  source 1..18: mol_type = other RNA
caacctcaaccatcacat

SEQ ID NO: 687 - RNA, 18 nt, Synthetic construct
  source 1..18: mol_type = other RNA
caacctggactaccagat

SEQ ID NO: 688 - RNA, 18 nt, Synthetic construct
  source 1..18: mol_type = other RNA
caacctcgaccaccttat

SEQ ID NO: 689 - RNA, 18 nt, Synthetic construct
  source 1..18: mol_type = other RNA
cttcctcgaccaccatat

SEQ ID NO: 690 - RNA, 18 nt, Synthetic construct
  source 1..18: mol_type = other RNA
caatctcgagcaccggat

SEQ ID NO: 691 - RNA, 18 nt, Synthetic construct
  source 1..18: mol_type = other RNA
gaacctcgaccaccgtac

SEQ ID NO: 692 - RNA, 18 nt, Synthetic construct
  source 1..18: mol_type = other RNA
caacatcgaccaccaaat

SEQ ID NO: 693 - RNA, 18 nt, Synthetic construct
  source 1..18: mol_type = other RNA
caccctcgaccatcgagt

SEQ ID NO: 694 - RNA, 18 nt, Synthetic construct
  source 1..18: mol_type = other RNA
caaccttgaacaccgcaa

SEQ ID NO: 695 - RNA, 18 nt, Synthetic construct
  source 1..18: mol_type = other RNA
tccatctcgcccacccca

SEQ ID NO: 696 - RNA, 18 nt, Synthetic construct
  source 1..18: mol_type = other RNA
tcaatctcgtcaaccgca

SEQ ID NO: 697 - RNA, 18 nt, Synthetic construct
  source 1..18: mol_type = other RNA
acaacctcgacctccgga

SEQ ID NO: 698 - RNA, 18 nt, Synthetic construct
  source 1..18: mol_type = other RNA
tcagccgcgaccactgta

SEQ ID NO: 699 - RNA, 18 nt, Synthetic construct
  source 1..18: mol_type = other RNA
tcaacttcgaccccccca

SEQ ID NO: 700 - RNA, 18 nt, Synthetic construct
  source 1..18: mol_type = other RNA
tcaacctcgacccccgga

SEQ ID NO: 701 - RNA, 18 nt, Synthetic construct
  source 1..18: mol_type = other RNA
tcaacctcgacccccgaa

SEQ ID NO: 702 - RNA, 18 nt, Synthetic construct
  source 1..18: mol_type = other RNA
tcaacctcgacccccgcg

SEQ ID NO: 703 - RNA, 18 nt, Synthetic construct
  source 1..18: mol_type = other RNA
tcaacttcgaccacggca

SEQ ID NO: 704 - RNA, 18 nt, Synthetic construct
  source 1..18: mol_type = other RNA
ccaacctcaacccccgca

SEQ ID NO: 705 - RNA, 18 nt, Synthetic construct
  source 1..18: mol_type = other RNA
tcaacctcgacctccgct

SEQ ID NO: 706 - RNA, 18 nt, Synthetic construct
  source 1..18: mol_type = other RNA
ccaacctcgacctccgga

SEQ ID NO: 707 - RNA, 18 nt, Synthetic construct
  source 1..18: mol_type = other RNA
ttaacctcaaccaccgct

SEQ ID NO: 708 - RNA, 18 nt, Synthetic construct
  source 1..18: mol_type = other RNA
tcaacctcgacctccgcc

SEQ ID NO: 709 - RNA, 18 nt, Synthetic construct
  source 1..18: mol_type = other RNA
tcaaccttgacccccgca

SEQ ID NO: 710 - RNA, 18 nt, Synthetic construct
  source 1..18: mol_type = other RNA
tcaacctcaaccaccgga

SEQ ID NO: 711 - RNA, 18 nt, Synthetic construct
  source 1..18: mol_type = other RNA
tcaatctcgacctccgga

SEQ ID NO: 712 - RNA, 18 nt, Synthetic construct
  source 1..18: mol_type = other RNA
tcaaccttgaccacagga

SEQ ID NO: 713 - RNA, 18 nt, Synthetic construct
  source 1..18: mol_type = other RNA
ttaacctcgaccagcgta

SEQ ID NO: 714 - RNA, 18 nt, Synthetic construct
  source 1..18: mol_type = other RNA
tcaacctcaacccccgca

SEQ ID NO: 715 - RNA, 18 nt, Synthetic construct
  source 1..18: mol_type = other RNA
tctacctcaaccacagca

SEQ ID NO: 716 - RNA, 18 nt, Synthetic construct
  source 1..18: mol_type = other RNA
tcaacctcgacctccacc

SEQ ID NO: 717 - RNA, 18 nt, Synthetic construct
  source 1..18: mol_type = other RNA
caaacctcgaccaccgcc

SEQ ID NO: 718 - RNA, 18 nt, Synthetic construct
  source 1..18: mol_type = other RNA
tcaacctcaaccatcgaa

SEQ ID NO: 719 - RNA, 18 nt, Synthetic construct
  source 1..18: mol_type = other RNA
tcaacctcgacctccgga

SEQ ID NO: 720 - RNA, 18 nt, Synthetic construct
  source 1..18: mol_type = other RNA
tcaacctcgaccaccaaa

SEQ ID NO: 721 - RNA, 18 nt, Synthetic construct
  source 1..18: mol_type = other RNA
tcaaccttgacctccgcg

SEQ ID NO: 722 - RNA, 18 nt, Synthetic construct
  source 1..18: mol_type = other RNA
tcaacctcgacctcagca

SEQ ID NO: 723 - RNA, 18 nt, Synthetic construct
  source 1..18: mol_type = other RNA
ttaacttcgacctccgga

SEQ ID NO: 724 - RNA, 18 nt, Synthetic construct
  source 1..18: mol_type = other RNA
tccacctcgaccacacca

SEQ ID NO: 725 - RNA, 18 nt, Synthetic construct
  source 1..18: mol_type = other RNA
tcaacgtcgaccaccgtt

SEQ ID NO: 726 - RNA, 18 nt, Synthetic construct
  source 1..18: mol_type = other RNA
tcaaccttgacctccgaa

SEQ ID NO: 727 - RNA, 18 nt, Synthetic construct
  source 1..18: mol_type = other RNA
tcaacctcgacctccgaa

SEQ ID NO: 728 - RNA, 18 nt, Synthetic construct
  source 1..18: mol_type = other RNA
tcaacctcgaccacgtca

SEQ ID NO: 729 - RNA, 18 nt, Synthetic construct
  source 1..18: mol_type = other RNA
ccgacctgtgcctacggg

SEQ ID NO: 730 - RNA, 18 nt, Synthetic construct
  source 1..18: mol_type = other RNA
tcgacctgtgccttcgga

SEQ ID NO: 731 - RNA, 18 nt, Synthetic construct
  source 1..18: mol_type = other RNA
ccgacctgtgcctttgga

SEQ ID NO: 732 - RNA, 18 nt, Synthetic construct
  source 1..18: mol_type = other RNA
cctacctgtgccttcgta

SEQ ID NO: 733 - RNA, 18 nt, Synthetic construct
  source 1..18: mol_type = other RNA
ccgacctgtgccttggga

SEQ ID NO: 734 - RNA, 18 nt, Synthetic construct
  source 1..18: mol_type = other RNA
gagagctgtgcctgcgaa

SEQ ID NO: 735 - RNA, 18 nt, Synthetic construct
  source 1..18: mol_type = other RNA
cctacctgtgcccacgaa

SEQ ID NO: 736 - RNA, 18 nt, Synthetic construct
  source 1..18: mol_type = other RNA
gcgacctgtgccttcgga

SEQ ID NO: 737 - RNA, 18 nt, Synthetic construct
  source 1..18: mol_type = other RNA
atgacctgtgcattcgaa

SEQ ID NO: 738 - RNA, 18 nt, Synthetic construct
  source 1..18: mol_type = other RNA
cccacccgtgccttcaaa

SEQ ID NO: 739 - RNA, 18 nt, Synthetic construct
  source 1..18: mol_type = other RNA
cccacctatgccttcgaa

SEQ ID NO: 740 - RNA, 18 nt, Synthetic construct
  source 1..18: mol_type = other RNA
acgacctgagccttagaa

SEQ ID NO: 741 - RNA, 18 nt, Synthetic construct
  source 1..18: mol_type = other RNA
ccgaaccgtgccctcgta

SEQ ID NO: 742 - RNA, 18 nt, Synthetic construct
  source 1..18: mol_type = other RNA
gagacctgtgcctgctaa

SEQ ID NO: 743 - RNA, 18 nt, Synthetic construct
  source 1..18: mol_type = other RNA
ccaacctgtgcctaggaa

SEQ ID NO: 744 - RNA, 18 nt, Synthetic construct
  source 1..18: mol_type = other RNA
cccacctgtgccttcgag

SEQ ID NO: 745 - RNA, 18 nt, Synthetic construct
  source 1..18: mol_type = other RNA
cctacccgtgccttcgac

SEQ ID NO: 746 - RNA, 18 nt, Synthetic construct
  source 1..18: mol_type = other RNA
gcaacctgtgccttcgaa

SEQ ID NO: 747 - RNA, 18 nt, Synthetic construct
  source 1..18: mol_type = other RNA
ccgacccgtgcctactaa

SEQ ID NO: 748 - RNA, 18 nt, Synthetic construct
  source 1..18: mol_type = other RNA
cctacctgtgccctcgaa

SEQ ID NO: 749 - RNA, 18 nt, Synthetic construct
  source 1..18: mol_type = other RNA
ccgacctctgccctcgag

SEQ ID NO: 750 - RNA, 18 nt, Synthetic construct
  source 1..18: mol_type = other RNA
ccgacttgtgccttcgga

SEQ ID NO: 751 - RNA, 18 nt, Synthetic construct
  source 1..18: mol_type = other RNA
ccgaccagtgcctactaa

SEQ ID NO: 752 - RNA, 18 nt, Synthetic construct
  source 1..18: mol_type = other RNA
ccgacatgagccctcgaa

SEQ ID NO: 753 - RNA, 18 nt, Synthetic construct
  source 1..18: mol_type = other RNA
cctacccgtgccttcgca

SEQ ID NO: 754 - RNA, 18 nt, Synthetic construct
  source 1..18: mol_type = other RNA
gacatgtctgctccttaa

SEQ ID NO: 755 - RNA, 18 nt, Synthetic construct
  source 1..18: mol_type = other RNA
gagctgtccgctccctat

SEQ ID NO: 756 - RNA, 18 nt, Synthetic construct
  source 1..18: mol_type = other RNA
aaccagtccgctcctgat

SEQ ID NO: 757 - RNA, 18 nt, Synthetic construct
  source 1..18: mol_type = other RNA
gacctcttcgctccttac

SEQ ID NO: 758 - RNA, 18 nt, Synthetic construct
  source 1..18: mol_type = other RNA
caattgtccgatccttat

SEQ ID NO: 759 - RNA, 18 nt, Synthetic construct
  source 1..18: mol_type = other RNA
gacccgtccgccccttat

SEQ ID NO: 760 - RNA, 18 nt, Synthetic construct
  source 1..18: mol_type = other RNA
gaccagtccgcttcttac

SEQ ID NO: 761 - RNA, 18 nt, Synthetic construct
  source 1..18: mol_type = other RNA
gtcctgtccgctccgtat

SEQ ID NO: 762 - RNA, 18 nt, Synthetic construct
  source 1..18: mol_type = other RNA
gacccgtccgcttcttat

SEQ ID NO: 763 - RNA, 18 nt, Synthetic construct
  source 1..18: mol_type = other RNA
gccctatccgctccttag

SEQ ID NO: 764 - RNA, 18 nt, Synthetic construct
  source 1..18: mol_type = other RNA
gacctgtccgctccttgg

SEQ ID NO: 765 - RNA, 18 nt, Synthetic construct
  source 1..18: mol_type = other RNA
gacctgtccgctctttgg

SEQ ID NO: 766 - RNA, 18 nt, Synthetic construct
  source 1..18: mol_type = other RNA
gacctggccgctccttgt

SEQ ID NO: 767 - RNA, 18 nt, Synthetic construct
  source 1..18: mol_type = other RNA
gacctgttcgctccttgt

SEQ ID NO: 768 - RNA, 18 nt, Synthetic construct
  source 1..18: mol_type = other RNA
cacctgtccgcttcttat

SEQ ID NO: 769 - RNA, 18 nt, Synthetic construct
  source 1..18: mol_type = other RNA
gacctgtccgccccttgt

SEQ ID NO: 770 - RNA, 18 nt, Synthetic construct
  source 1..18: mol_type = other RNA
cgcctgcccgccccttat

SEQ ID NO: 771 - RNA, 18 nt, Synthetic construct
  source 1..18: mol_type = other RNA
tacccgtccgctcgttat

SEQ ID NO: 772 - RNA, 18 nt, Synthetic construct
  source 1..18: mol_type = other RNA
ggcctgtccgcttcttat

SEQ ID NO: 773 - RNA, 18 nt, Synthetic construct
  source 1..18: mol_type = other RNA
gacctgtccgctgcctat

SEQ ID NO: 774 - RNA, 18 nt, Synthetic construct
  source 1..18: mol_type = other RNA
gacctgtccgctccgttt

SEQ ID NO: 775 - RNA, 18 nt, Synthetic construct
  source 1..18: mol_type = other RNA
gacctgtccgcccctgat

SEQ ID NO: 776 - RNA, 18 nt, Synthetic construct
  source 1..18: mol_type = other RNA
gagctgtccgctccttcg

SEQ ID NO: 777 - RNA, 18 nt, Synthetic construct
  source 1..18: mol_type = other RNA
gacctgtccgccccttct

SEQ ID NO: 778 - RNA, 18 nt, Synthetic construct
  source 1..18: mol_type = other RNA
ggcctgcccgctccgtat

SEQ ID NO: 779 - RNA, 18 nt, Synthetic construct
  source 1..18: mol_type = other RNA
tacctgtccgctccgaat

SEQ ID NO: 780 - RNA, 18 nt, Synthetic construct
  source 1..18: mol_type = other RNA
ctcctgtccgctccttat

SEQ ID NO: 781 - RNA, 18 nt, Synthetic construct
  source 1..18: mol_type = other RNA
gattcgtccgctcactat

SEQ ID NO: 782 - RNA, 18 nt, Synthetic construct
  source 1..18: mol_type = other RNA
gacctgcccgccccttat

SEQ ID NO: 783 - RNA, 18 nt, Synthetic construct
  source 1..18: mol_type = other RNA
gacccgtccgctcactat

SEQ ID NO: 784 - RNA, 18 nt, Synthetic construct
  source 1..18: mol_type = other RNA
gacctgtccgcttcttct

SEQ ID NO: 785 - RNA, 18 nt, Synthetic construct
  source 1..18: mol_type = other RNA
ggcctgtccgctccatat

SEQ ID NO: 786 - RNA, 18 nt, Synthetic construct
  source 1..18: mol_type = other RNA
gaccagtccgctccatac

SEQ ID NO: 787 - RNA, 18 nt, Synthetic construct
  source 1..18: mol_type = other RNA
gtcctgtccgcttcttat

SEQ ID NO: 788 - RNA, 18 nt, Synthetic construct
  source 1..18: mol_type = other RNA
cacctgttcgctccttat

SEQ ID NO: 789 - RNA, 18 nt, Synthetic construct
  source 1..18: mol_type = other RNA
gaccggtctggtctttat

SEQ ID NO: 790 - RNA, 18 nt, Synthetic construct
  source 1..18: mol_type = other RNA
ggactgtccgctccttag

SEQ ID NO: 791 - RNA, 18 nt, Synthetic construct
  source 1..18: mol_type = other RNA
gacctgtccgctccaaat

SEQ ID NO: 792 - RNA, 18 nt, Synthetic construct
  source 1..18: mol_type = other RNA
gacctgtccgcccctcat

SEQ ID NO: 793 - RNA, 18 nt, Synthetic construct
  source 1..18: mol_type = other RNA
cacctgtccgccccttat

SEQ ID NO: 794 - RNA, 18 nt, Synthetic construct
  source 1..18: mol_type = other RNA
atcctgtccgccctttaa

SEQ ID NO: 795 - RNA, 18 nt, Synthetic construct
  source 1..18: mol_type = other RNA
tacctgtccgcccctttt

SEQ ID NO: 796 - RNA, 18 nt, Synthetic construct
  source 1..18: mol_type = other RNA
gacccgtccgctccttac

SEQ ID NO: 797 - RNA, 18 nt, Synthetic construct
  source 1..18: mol_type = other RNA
gacacacaggctcgagcg

SEQ ID NO: 798 - RNA, 18 nt, Synthetic construct
  source 1..18: mol_type = other RNA
ggcacacaggctcaagcc

SEQ ID NO: 799 - RNA, 18 nt, Synthetic construct
  source 1..18: mol_type = other RNA
gacacacagtctcaagaa

SEQ ID NO: 800 - RNA, 18 nt, Synthetic construct
  source 1..18: mol_type = other RNA
gacacacaggctcagacc

SEQ ID NO: 801 - RNA, 18 nt, Synthetic construct
  source 1..18: mol_type = other RNA
ggcacacaggctcgagcc

SEQ ID NO: 802 - RNA, 18 nt, Synthetic construct
  source 1..18: mol_type = other RNA
gtcacacagcctcatgaa

SEQ ID NO: 803 - RNA, 18 nt, Synthetic construct
  source 1..18: mol_type = other RNA
gacagacaacctcaagtg

SEQ ID NO: 804 - RNA, 18 nt, Synthetic construct
  source 1..18: mol_type = other RNA
gacacggaccctctaggg

SEQ ID NO: 805 - RNA, 18 nt, Synthetic construct
  source 1..18: mol_type = other RNA
gacacacaggctctatcg

SEQ ID NO: 806 - RNA, 18 nt, Synthetic construct
  source 1..18: mol_type = other RNA
ggcactcagcctcacgct

SEQ ID NO: 807 - RNA, 18 nt, Synthetic construct
  source 1..18: mol_type = other RNA
gacacagcgcctcaagcg

SEQ ID NO: 808 - RNA, 18 nt, Synthetic construct
  source 1..18: mol_type = other RNA
gacacacaggctcaagct

SEQ ID NO: 809 - RNA, 18 nt, Synthetic construct
  source 1..18: mol_type = other RNA
gacacacaggctcaacct

SEQ ID NO: 810 - RNA, 18 nt, Synthetic construct
  source 1..18: mol_type = other RNA
gacgcacaccgtcaagcg

SEQ ID NO: 811 - RNA, 18 nt, Synthetic construct
  source 1..18: mol_type = other RNA
gacgcacaaccttaaggg

SEQ ID NO: 812 - RNA, 18 nt, Synthetic construct
  source 1..18: mol_type = other RNA
gccacacaggctcaaccg

SEQ ID NO: 813 - RNA, 18 nt, Synthetic construct
  source 1..18: mol_type = other RNA
gacagacaggctcgagcg

SEQ ID NO: 814 - RNA, 18 nt, Synthetic construct
  source 1..18: mol_type = other RNA
gacacacaggctcaaccg

SEQ ID NO: 815 - RNA, 18 nt, Synthetic construct
  source 1..18: mol_type = other RNA
gacacacaggctctagcg

SEQ ID NO: 816 - RNA, 18 nt, Synthetic construct
  source 1..18: mol_type = other RNA
gccacacaggctcaagag

SEQ ID NO: 817 - RNA, 18 nt, Synthetic construct
  source 1..18: mol_type = other RNA
aatacacagcctcaaacg

SEQ ID NO: 818 - RNA, 18 nt, Synthetic construct
  source 1..18: mol_type = other RNA
gagacactgcctcaaacc

SEQ ID NO: 819 - RNA, 18 nt, Synthetic construct
  source 1..18: mol_type = other RNA
atcacacagattcaaggg

SEQ ID NO: 820 - RNA, 18 nt, Synthetic construct
  source 1..18: mol_type = other RNA
gacacacagccccaggag

SEQ ID NO: 821 - RNA, 18 nt, Synthetic construct
  source 1..18: mol_type = other RNA
gacacacagcctaacgcg

SEQ ID NO: 822 - RNA, 18 nt, Synthetic construct
  source 1..18: mol_type = other RNA
gacacacaggctcaagta

SEQ ID NO: 823 - RNA, 18 nt, Synthetic construct
  source 1..18: mol_type = other RNA
gacagacagccccaagcg

SEQ ID NO: 824 - RNA, 18 nt, Synthetic construct
  source 1..18: mol_type = other RNA
gacacactgcctttagcg

SEQ ID NO: 825 - RNA, 18 nt, Synthetic construct
  source 1..18: mol_type = other RNA
tgcacacaggctcaagct

SEQ ID NO: 826 - RNA, 18 nt, Synthetic construct
  source 1..18: mol_type = other RNA
gagacacagccccaaggc

SEQ ID NO: 827 - RNA, 18 nt, Synthetic construct
  source 1..18: mol_type = other RNA
gagacacagcatgaagcg

SEQ ID NO: 828 - RNA, 18 nt, Synthetic construct
  source 1..18: mol_type = other RNA
gacacacgggctcacgcg

SEQ ID NO: 829 - RNA, 18 nt, Synthetic construct
  source 1..18: mol_type = other RNA
ggtacacagcatcaagcg

SEQ ID NO: 830 - RNA, 18 nt, Synthetic construct
  source 1..18: mol_type = other RNA
aacacacaggctcaaccg

SEQ ID NO: 831 - RNA, 18 nt, Synthetic construct
  source 1..18: mol_type = other RNA
gacacacaggctcctgcg

SEQ ID NO: 832 - RNA, 18 nt, Synthetic construct
  source 1..18: mol_type = other RNA
gacacacagccttaaggg

SEQ ID NO: 833 - RNA, 18 nt, Synthetic construct
  source 1..18: mol_type = other RNA
cacacacaacatcaatcg

SEQ ID NO: 834 - RNA, 18 nt, Synthetic construct
  source 1..18: mol_type = other RNA
gatacacaacctcaggcg

SEQ ID NO: 835 - RNA, 18 nt, Synthetic construct
  source 1..18: mol_type = other RNA
atcacacaggctcaatcg

SEQ ID NO: 836 - RNA, 18 nt, Synthetic construct
  source 1..18: mol_type = other RNA
gacacataggctcgagcg

SEQ ID NO: 837 - RNA, 18 nt, Synthetic construct
  source 1..18: mol_type = other RNA
gaaacacatccccaagcg

SEQ ID NO: 838 - RNA, 18 nt, Synthetic construct
  source 1..18: mol_type = other RNA
gacacagggcctcacgcg

SEQ ID NO: 839 - RNA, 18 nt, Synthetic construct
  source 1..18: mol_type = other RNA
gccacacaggctcaggcg

SEQ ID NO: 840 - RNA, 18 nt, Synthetic construct
  source 1..18: mol_type = other RNA
gacacacaggctcaagcg

SEQ ID NO: 841 - RNA, 18 nt, Synthetic construct
  source 1..18: mol_type = other RNA
gacgcacagcctcaacct

SEQ ID NO: 842 - RNA, 18 nt, Synthetic construct
  source 1..18: mol_type = other RNA
gacacacaaccccacgcc

SEQ ID NO: 843 - RNA, 18 nt, Synthetic construct
  source 1..18: mol_type = other RNA
ggcacacagcccctagcg

SEQ ID NO: 844 - RNA, 18 nt, Synthetic construct
  source 1..18: mol_type = other RNA
gacacacggcctcgagcg

SEQ ID NO: 845 - RNA, 18 nt, Synthetic construct
  source 1..18: mol_type = other RNA
gacccacagtctctagcg

SEQ ID NO: 846 - RNA, 18 nt, Synthetic construct
  source 1..18: mol_type = other RNA
gacacacaggcttaagct

SEQ ID NO: 847 - RNA, 18 nt, Synthetic construct
  source 1..18: mol_type = other RNA
gacactcaggctcaagcg

SEQ ID NO: 848 - RNA, 18 nt, Synthetic construct
  source 1..18: mol_type = other RNA
cacacaccgcctcaggcg

SEQ ID NO: 849 - RNA, 18 nt, Synthetic construct
  source 1..18: mol_type = other RNA
gacacaaagccccaagct

SEQ ID NO: 850 - RNA, 18 nt, Synthetic construct
  source 1..18: mol_type = other RNA
gccacacagactcaatcg

SEQ ID NO: 851 - RNA, 18 nt, Synthetic construct
  source 1..18: mol_type = other RNA
gacacaaaggttcaagct

SEQ ID NO: 852 - RNA, 18 nt, Synthetic construct
  source 1..18: mol_type = other RNA
tacacacaggctcaagcg

SEQ ID NO: 853 - RNA, 18 nt, Synthetic construct
  source 1..18: mol_type = other RNA
gatacacagcctcgagcc

SEQ ID NO: 854 - RNA, 18 nt, Synthetic construct
  source 1..18: mol_type = other RNA
gacacacgggctcaagct

SEQ ID NO: 855 - RNA, 18 nt, Synthetic construct
  source 1..18: mol_type = other RNA
gacacacaggctcaggcg

SEQ ID NO: 856 - RNA, 18 nt, Synthetic construct
  source 1..18: mol_type = other RNA
gacatacaggctcaagcg

SEQ ID NO: 857 - RNA, 18 nt, Synthetic construct
  source 1..18: mol_type = other RNA
tacacacaggctcacggg

SEQ ID NO: 858 - RNA, 18 nt, Synthetic construct
  source 1..18: mol_type = other RNA
gacacacaccatctagct

SEQ ID NO: 859 - RNA, 18 nt, Synthetic construct
  source 1..18: mol_type = other RNA
gacacacaggctcacgcg

SEQ ID NO: 860 - RNA, 18 nt, Synthetic construct
  source 1..18: mol_type = other RNA
gtcagacagcctcaagag

SEQ ID NO: 861 - RNA, 18 nt, Synthetic construct
  source 1..18: mol_type = other RNA
cactcacagcctcaagcg

SEQ ID NO: 862 - RNA, 18 nt, Synthetic construct
  source 1..18: mol_type = other RNA
aacacacaaagtcaagcg

SEQ ID NO: 863 - RNA, 18 nt, Synthetic construct
  source 1..18: mol_type = other RNA
gagattaagcctcaagcg

SEQ ID NO: 864 - RNA, 18 nt, Synthetic construct
  source 1..18: mol_type = other RNA
gacacacagcatcaaaca

SEQ ID NO: 865 - RNA, 18 nt, Synthetic construct
  source 1..18: mol_type = other RNA
gattcccagcccaaagcg

SEQ ID NO: 866 - RNA, 18 nt, Synthetic construct
  source 1..18: mol_type = other RNA
gccacacaccgtcaagcg

SEQ ID NO: 867 - RNA, 18 nt, Synthetic construct
  source 1..18: mol_type = other RNA
aacacacaacctctagcg

SEQ ID NO: 868 - RNA, 18 nt, Synthetic construct
  source 1..18: mol_type = other RNA
gacacacaacctcaagaa

SEQ ID NO: 869 - RNA, 18 nt, Synthetic construct
  source 1..18: mol_type = other RNA
gacactcagcctcaactg

SEQ ID NO: 870 - RNA, 18 nt, Synthetic construct
  source 1..18: mol_type = other RNA
tacacacagcctcgagcg

SEQ ID NO: 871 - RNA, 18 nt, Synthetic construct
  source 1..18: mol_type = other RNA
ggcacacaggctcaaggg

SEQ ID NO: 872 - RNA, 18 nt, Synthetic construct
  source 1..18: mol_type = other RNA
gacacgcaggctctagcg

SEQ ID NO: 873 - RNA, 18 nt, Synthetic construct
  source 1..18: mol_type = other RNA
gacccacaggctcaaccg

SEQ ID NO: 874 - RNA, 18 nt, Synthetic construct
  source 1..18: mol_type = other RNA
gtcacacagccacatgcg

SEQ ID NO: 875 - RNA, 18 nt, Synthetic construct
  source 1..18: mol_type = other RNA
gacgcaaagcgtaatgtg

SEQ ID NO: 876 - RNA, 18 nt, Synthetic construct
  source 1..18: mol_type = other RNA
gacacacagccccaacag

SEQ ID NO: 877 - RNA, 18 nt, Synthetic construct
  source 1..18: mol_type = other RNA
gacacacagcctcaataa

SEQ ID NO: 878 - RNA, 18 nt, Synthetic construct
  source 1..18: mol_type = other RNA
gtcacacagcatcaagcg

SEQ ID NO: 879 - RNA, 18 nt, Synthetic construct
  source 1..18: mol_type = other RNA
gacacacaggctcaatcg

SEQ ID NO: 880 - RNA, 18 nt, Synthetic construct
  source 1..18: mol_type = other RNA
gacaaacagccccaagag

SEQ ID NO: 881 - RNA, 18 nt, Synthetic construct
  source 1..18: mol_type = other RNA
ggcacacaggctcaagtg

SEQ ID NO: 882 - RNA, 18 nt, Synthetic construct
  source 1..18: mol_type = other RNA
gaaatacagcccccagct

SEQ ID NO: 883 - RNA, 18 nt, Synthetic construct
  source 1..18: mol_type = other RNA
gacacacagcctcacggg

SEQ ID NO: 884 - RNA, 18 nt, Synthetic construct
  source 1..18: mol_type = other RNA
gacagacaggctcaagcg

SEQ ID NO: 885 - RNA, 18 nt, Synthetic construct
  source 1..18: mol_type = other RNA
gccactcagccccaagct

SEQ ID NO: 886 - RNA, 18 nt, Synthetic construct
  source 1..18: mol_type = other RNA
gacaaacaacctcaaccg

SEQ ID NO: 887 - RNA, 18 nt, Synthetic construct
  source 1..18: mol_type = other RNA
gatacaccgcctcaggcg

SEQ ID NO: 888 - RNA, 18 nt, Synthetic construct
  source 1..18: mol_type = other RNA
gaagagcagcctcaagag

SEQ ID NO: 889 - RNA, 18 nt, Synthetic construct
  source 1..18: mol_type = other RNA
gacacacagcattaggcg

SEQ ID NO: 890 - RNA, 18 nt, Synthetic construct
  source 1..18: mol_type = other RNA
ggcacacagcctcaaggg

SEQ ID NO: 891 - RNA, 18 nt, Synthetic construct
  source 1..18: mol_type = other RNA
gacacacaggctcaagca

SEQ ID NO: 892 - RNA, 18 nt, Synthetic construct
  source 1..18: mol_type = other RNA
gacacacaccgtcaagcg

SEQ ID NO: 893 - RNA, 18 nt, Synthetic construct
  source 1..18: mol_type = other RNA
gacacacaggctcgagag

SEQ ID NO: 894 - RNA, 18 nt, Synthetic construct
  source 1..18: mol_type = other RNA
gacacacagcctctagcc

SEQ ID NO: 895 - RNA, 18 nt, Synthetic construct
  source 1..18: mol_type = other RNA
gccacacaggctctagcg

SEQ ID NO: 896 - RNA, 18 nt, Synthetic construct
  source 1..18: mol_type = other RNA
gacatacagcctcaggcg

SEQ ID NO: 897 - RNA, 18 nt, Synthetic construct
  source 1..18: mol_type = other RNA
gacacacaggttcaagct

SEQ ID NO: 898 - RNA, 18 nt, Synthetic construct
  source 1..18: mol_type = other RNA
gacgcaccacctcaagct

SEQ ID NO: 899 - RNA, 18 nt, Synthetic construct
  source 1..18: mol_type = other RNA
gacagacaggttcaagcg

SEQ ID NO: 900 - RNA, 18 nt, Synthetic construct
  source 1..18: mol_type = other RNA
gactcacagaatcaaacg

SEQ ID NO: 901 - RNA, 18 nt, Synthetic construct
  source 1..18: mol_type = other RNA
cacacacaggctcaagca

SEQ ID NO: 902 - RNA, 18 nt, Synthetic construct
  source 1..18: mol_type = other RNA
ggaacacagcctcaagaa

SEQ ID NO: 903 - RNA, 18 nt, Synthetic construct
  source 1..18: mol_type = other RNA
ggcaaacagcctcaagcg

SEQ ID NO: 904 - RNA, 18 nt, Synthetic construct
  source 1..18: mol_type = other RNA
catacacagcctcaatcg

SEQ ID NO: 905 - RNA, 18 nt, Synthetic construct
  source 1..18: mol_type = other RNA
gacatacaaccccaagcg

SEQ ID NO: 906 - RNA, 18 nt, Synthetic construct
  source 1..18: mol_type = other RNA
gatgcacagccccatgcg

SEQ ID NO: 907 - RNA, 18 nt, Synthetic construct
  source 1..18: mol_type = other RNA
gaaacacagccttaatcg

SEQ ID NO: 908 - RNA, 18 nt, Synthetic construct
  source 1..18: mol_type = other RNA
gacacatagcctcactcg

SEQ ID NO: 909 - RNA, 18 nt, Synthetic construct
  source 1..18: mol_type = other RNA
tgcacacaggctcagacg

SEQ ID NO: 910 - RNA, 18 nt, Synthetic construct
  source 1..18: mol_type = other RNA
gacgcactgtctcaagcg

SEQ ID NO: 911 - RNA, 18 nt, Synthetic construct
  source 1..18: mol_type = other RNA
ggtacacagcctcaagca

SEQ ID NO: 912 - RNA, 18 nt, Synthetic construct
  source 1..18: mol_type = other RNA
gacagacaacctcaagcg

SEQ ID NO: 913 - RNA, 18 nt, Synthetic construct
  source 1..18: mol_type = other RNA
gacacccagcttcaagct

SEQ ID NO: 914 - RNA, 18 nt, Synthetic construct
  source 1..18: mol_type = other RNA
gacacacaggctcaagtg

SEQ ID NO: 915 - RNA, 18 nt, Synthetic construct
  source 1..18: mol_type = other RNA
tacgtacagcctcaagct

SEQ ID NO: 916 - RNA, 18 nt, Synthetic construct
  source 1..18: mol_type = other RNA
cacacacagcatcaagct

SEQ ID NO: 917 - RNA, 18 nt, Synthetic construct
  source 1..18: mol_type = other RNA
ggcacatagcctcaagcg

SEQ ID NO: 918 - RNA, 18 nt, Synthetic construct
  source 1..18: mol_type = other RNA
gacacacaccctcagggg

SEQ ID NO: 919 - RNA, 18 nt, Synthetic construct
  source 1..18: mol_type = other RNA
gacacacagcctcggggg

SEQ ID NO: 920 - RNA, 18 nt, Synthetic construct
  source 1..18: mol_type = other RNA
gttacacagccttgagcg

SEQ ID NO: 921 - RNA, 18 nt, Synthetic construct
  source 1..18: mol_type = other RNA
gacacacagcctcgatct

SEQ ID NO: 922 - RNA, 18 nt, Synthetic construct
  source 1..18: mol_type = other RNA
gtcacacaacctcaagat

SEQ ID NO: 923 - RNA, 18 nt, Synthetic construct
  source 1..18: mol_type = other RNA
gacacacaggctcaagag

SEQ ID NO: 924 - RNA, 18 nt, Synthetic construct
  source 1..18: mol_type = other RNA
gatacacaggctcaagcg

SEQ ID NO: 925 - RNA, 18 nt, Synthetic construct
  source 1..18: mol_type = other RNA
gaaacacaggctcaaccg

SEQ ID NO: 926 - RNA, 18 nt, Synthetic construct
  source 1..18: mol_type = other RNA
gacacacagtctagagcg

SEQ ID NO: 927 - RNA, 18 nt, Synthetic construct
  source 1..18: mol_type = other RNA
agaggcggtacggactta

SEQ ID NO: 928 - RNA, 18 nt, Synthetic construct
  source 1..18: mol_type = other RNA
cgacagggtacggactct

SEQ ID NO: 929 - RNA, 18 nt, Synthetic construct
  source 1..18: mol_type = other RNA
ggacggggtacgtactac

SEQ ID NO: 930 - RNA, 18 nt, Synthetic construct
  source 1..18: mol_type = other RNA
cgacgtggtacggaccgc

SEQ ID NO: 931 - RNA, 18 nt, Synthetic construct
  source 1..18: mol_type = other RNA
cgactgcgtacggactac

SEQ ID NO: 932 - RNA, 18 nt, Synthetic construct
  source 1..18: mol_type = other RNA
cgaccaggtacggcctca

SEQ ID NO: 933 - RNA, 18 nt, Synthetic construct
  source 1..18: mol_type = other RNA
cgacggggtacgtacttg

SEQ ID NO: 934 - RNA, 18 nt, Synthetic construct
  source 1..18: mol_type = other RNA
cgacggggtacggccttt

SEQ ID NO: 935 - RNA, 18 nt, Synthetic construct
  source 1..18: mol_type = other RNA
tgaccgagtacggacttc

SEQ ID NO: 936 - RNA, 18 nt, Synthetic construct
  source 1..18: mol_type = other RNA
ctacggcgtacggactcc

SEQ ID NO: 937 - RNA, 18 nt, Synthetic construct
  source 1..18: mol_type = other RNA
cgaccgagtacggtcttc

SEQ ID NO: 938 - RNA, 18 nt, Synthetic construct
  source 1..18: mol_type = other RNA
cgacggggtacgttctta

SEQ ID NO: 939 - RNA, 18 nt, Synthetic construct
  source 1..18: mol_type = other RNA
ctacggggtacggccttc

SEQ ID NO: 940 - RNA, 18 nt, Synthetic construct
  source 1..18: mol_type = other RNA
caacggggtacggtcttc

SEQ ID NO: 941 - RNA, 18 nt, Synthetic construct
  source 1..18: mol_type = other RNA
cgacggggtacggactat

SEQ ID NO: 942 - RNA, 18 nt, Synthetic construct
  source 1..18: mol_type = other RNA
agagggggtacggactgc

SEQ ID NO: 943 - RNA, 18 nt, Synthetic construct
  source 1..18: mol_type = other RNA
cgactaggtacggacttg

SEQ ID NO: 944 - RNA, 18 nt, Synthetic construct
  source 1..18: mol_type = other RNA
cgactgggtacgtgctta

SEQ ID NO: 945 - RNA, 18 nt, Synthetic construct
  source 1..18: mol_type = other RNA
cgaaggggtacggactac

SEQ ID NO: 946 - RNA, 18 nt, Synthetic construct
  source 1..18: mol_type = other RNA
cgacggggtacggactca

SEQ ID NO: 947 - RNA, 18 nt, Synthetic construct
  source 1..18: mol_type = other RNA
ctacggggtacggtcttc

SEQ ID NO: 948 - RNA, 18 nt, Synthetic construct
  source 1..18: mol_type = other RNA
cgacggggtacggacgac

SEQ ID NO: 949 - RNA, 18 nt, Synthetic construct
  source 1..18: mol_type = other RNA
cggctgggtacggttttc

SEQ ID NO: 950 - RNA, 18 nt, Synthetic construct
  source 1..18: mol_type = other RNA
cgacggggtacggagttt

SEQ ID NO: 951 - RNA, 18 nt, Synthetic construct
  source 1..18: mol_type = other RNA
cggaggggtacggagttc

SEQ ID NO: 952 - RNA, 18 nt, Synthetic construct
  source 1..18: mol_type = other RNA
cgatggggtacggactaa

SEQ ID NO: 953 - RNA, 18 nt, Synthetic construct
  source 1..18: mol_type = other RNA
caacggtgtacggacttc

SEQ ID NO: 954 - RNA, 18 nt, Synthetic construct
  source 1..18: mol_type = other RNA
cgacagggtacggaattc

SEQ ID NO: 955 - RNA, 18 nt, Synthetic construct
  source 1..18: mol_type = other RNA
cgagggggtacgggcgtc

SEQ ID NO: 956 - RNA, 18 nt, Synthetic construct
  source 1..18: mol_type = other RNA
cggctgggtacggtctta

SEQ ID NO: 957 - RNA, 18 nt, Synthetic construct
  source 1..18: mol_type = other RNA
tgactggttacggacttc

SEQ ID NO: 958 - RNA, 18 nt, Synthetic construct
  source 1..18: mol_type = other RNA
cgaccaggtacggacttg

SEQ ID NO: 959 - RNA, 18 nt, Synthetic construct
  source 1..18: mol_type = other RNA
cgacggggtacgaactga

SEQ ID NO: 960 - RNA, 18 nt, Synthetic construct
  source 1..18: mol_type = other RNA
cgactaggtacggtctta

SEQ ID NO: 961 - RNA, 18 nt, Synthetic construct
  source 1..18: mol_type = other RNA
gcactgggtacggactgc

SEQ ID NO: 962 - RNA, 18 nt, Synthetic construct
  source 1..18: mol_type = other RNA
tgacggggtacggagtta

SEQ ID NO: 963 - RNA, 18 nt, Synthetic construct
  source 1..18: mol_type = other RNA
cgaaggggtacggacttg

SEQ ID NO: 964 - RNA, 18 nt, Synthetic construct
  source 1..18: mol_type = other RNA
cgatggcgtacggactcc

SEQ ID NO: 965 - RNA, 18 nt, Synthetic construct
  source 1..18: mol_type = other RNA
caacggggtacggcctac

SEQ ID NO: 966 - RNA, 18 nt, Synthetic construct
  source 1..18: mol_type = other RNA
tgacggggtacggacaac

SEQ ID NO: 967 - RNA, 20 nt, Synthetic construct
  source 1..20: mol_type = other RNA
ttgcgtggcttacatagttc

SEQ ID NO: 968 - RNA, 20 nt, Synthetic construct
  source 1..20: mol_type = other RNA
ttccttcacctacattgttc

SEQ ID NO: 969 - RNA, 20 nt, Synthetic construct
  source 1..20: mol_type = other RNA
ttcctttaactacataggtc

SEQ ID NO: 970 - RNA, 20 nt, Synthetic construct
  source 1..20: mol_type = other RNA
ttctttcacctacatagttc

SEQ ID NO: 971 - RNA, 20 nt, Synthetic construct
  source 1..20: mol_type = other RNA
ttcctccacctacatagtta

SEQ ID NO: 972 - RNA, 20 nt, Synthetic construct
  source 1..20: mol_type = other RNA
gtccttgacctacagaggtc

SEQ ID NO: 973 - RNA, 20 nt, Synthetic construct
  source 1..20: mol_type = other RNA
ctccttgacctacacagatc

SEQ ID NO: 974 - RNA, 20 nt, Synthetic construct
  source 1..20: mol_type = other RNA
ttccttcacctacatcgttc

SEQ ID NO: 975 - RNA, 20 nt, Synthetic construct
  source 1..20: mol_type = other RNA
ttccttgtactacatagtgc

SEQ ID NO: 976 - RNA, 20 nt, Synthetic construct
  source 1..20: mol_type = other RNA
ttctctgacctacataggcc

SEQ ID NO: 977 - RNA, 20 nt, Synthetic construct
  source 1..20: mol_type = other RNA
tttcttgacctacatagttg

SEQ ID NO: 978 - RNA, 20 nt, Synthetic construct
  source 1..20: mol_type = other RNA
atcattgacctacatagttc

SEQ ID NO: 979 - RNA, 20 nt, Synthetic construct
  source 1..20: mol_type = other RNA
ctcctagacgtacatagttc

SEQ ID NO: 980 - RNA, 20 nt, Synthetic construct
  source 1..20: mol_type = other RNA
tgcctagatctacacagttc

SEQ ID NO: 981 - RNA, 20 nt, Synthetic construct
  source 1..20: mol_type = other RNA
ttccttgagttacacagttc

SEQ ID NO: 982 - RNA, 20 nt, Synthetic construct
  source 1..20: mol_type = other RNA
ttccttcacctacataggcc

SEQ ID NO: 983 - RNA, 20 nt, Synthetic construct
  source 1..20: mol_type = other RNA
gtctttgacctacattgttc

SEQ ID NO: 984 - RNA, 20 nt, Synthetic construct
  source 1..20: mol_type = other RNA
ttccttcacctacatagtta

SEQ ID NO: 985 - RNA, 20 nt, Synthetic construct
  source 1..20: mol_type = other RNA
tgccttcacctacatagtgc

SEQ ID NO: 986 - RNA, 20 nt, Synthetic construct
  source 1..20: mol_type = other RNA
gttcttgacctacatagctc

SEQ ID NO: 987 - RNA, 20 nt, Synthetic construct
  source 1..20: mol_type = other RNA
ttccttgacctacataggtt

SEQ ID NO: 988 - RNA, 20 nt, Synthetic construct
  source 1..20: mol_type = other RNA
ttccttaacctacatagaaa

SEQ ID NO: 989 - RNA, 20 nt, Synthetic construct
  source 1..20: mol_type = other RNA
ttcctctacctacatagctc

SEQ ID NO: 990 - RNA, 20 nt, Synthetic construct
  source 1..20: mol_type = other RNA
ttccttcacctacattgctc

SEQ ID NO: 991 - RNA, 20 nt, Synthetic construct
  source 1..20: mol_type = other RNA
taccttcacctacatagctc

SEQ ID NO: 992 - RNA, 20 nt, Synthetic construct
  source 1..20: mol_type = other RNA
ttccttcacctacagagtta

SEQ ID NO: 993 - RNA, 20 nt, Synthetic construct
  source 1..20: mol_type = other RNA
taccttcacctacatagtta

SEQ ID NO: 994 - RNA, 20 nt, Synthetic construct
  source 1..20: mol_type = other RNA
gtccttgaactacatagttg

SEQ ID NO: 995 - RNA, 20 nt, Synthetic construct
  source 1..20: mol_type = other RNA
ttccctgacctacatagatt

SEQ ID NO: 996 - RNA, 20 nt, Synthetic construct
  source 1..20: mol_type = other RNA
gtccttgacctacattgttc

SEQ ID NO: 997 - RNA, 20 nt, Synthetic construct
  source 1..20: mol_type = other RNA
ttccttgacctacattgtta

SEQ ID NO: 998 - RNA, 20 nt, Synthetic construct
  source 1..20: mol_type = other RNA
ttccttaacctacatggtcc

SEQ ID NO: 999 - RNA, 20 nt, Synthetic construct
  source 1..20: mol_type = other RNA
ttccttaacttacatagtac

SEQ ID NO: 1000 - RNA, 20 nt, Synthetic construct
  source 1..20: mol_type = other RNA
tccctgtacctacatagttc

SEQ ID NO: 1001 - RNA, 20 nt, Synthetic construct
  source 1..20: mol_type = other RNA
cttcttgacctacattgttc

SEQ ID NO: 1002 - RNA, 20 nt, Synthetic construct
  source 1..20: mol_type = other RNA
atccttgacctacattgttc

SEQ ID NO: 1003 - RNA, 20 nt, Synthetic construct
  source 1..20: mol_type = other RNA
tgccttcacgtacatagttc

SEQ ID NO: 1004 - RNA, 20 nt, Synthetic construct
  source 1..20: mol_type = other RNA
atccttgacctacacagttc

SEQ ID NO: 1005 - RNA, 20 nt, Synthetic construct
  source 1..20: mol_type = other RNA
atccttcacctacatagttc

SEQ ID NO: 1006 - RNA, 20 nt, Synthetic construct
  source 1..20: mol_type = other RNA
ctacgtggttaacacaggag

SEQ ID NO: 1007 - RNA, 20 nt, Synthetic construct
  source 1..20: mol_type = other RNA
ctacgtagttaacataggag

SEQ ID NO: 1008 - RNA, 20 nt, Synthetic construct
  source 1..20: mol_type = other RNA
atacgtagttaacacaggag

SEQ ID NO: 1009 - RNA, 20 nt, Synthetic construct
  source 1..20: mol_type = other RNA
ctacgtagttcacacaggag

SEQ ID NO: 1010 - RNA, 20 nt, Synthetic construct
  source 1..20: mol_type = other RNA
ctacggagttaacacaggag

SEQ ID NO: 1011 - RNA, 20 nt, Synthetic construct
  source 1..20: mol_type = other RNA
ctacgtagttaacaaaggag

SEQ ID NO: 1012 - RNA, 20 nt, Synthetic construct
  source 1..20: mol_type = other RNA
ctacctagttaacacaggag

SEQ ID NO: 1013 - RNA, 20 nt, Synthetic construct
  source 1..20: mol_type = other RNA
ctacgtagttaacagaggag

SEQ ID NO: 1014 - RNA, 20 nt, Synthetic construct
  source 1..20: mol_type = other RNA
ctacatagttaacacaggag

SEQ ID NO: 1015 - RNA, 20 nt, Synthetic construct
  source 1..20: mol_type = other RNA
ctacctagttaacacagaat

SEQ ID NO: 1016 - RNA, 20 nt, Synthetic construct
  source 1..20: mol_type = other RNA
ctacgttgttaacacaggag

SEQ ID NO: 1017 - RNA, 20 nt, Synthetic construct
  source 1..20: mol_type = other RNA
ctacttagttaacacggaag

SEQ ID NO: 1018 - RNA, 20 nt, Synthetic construct
  source 1..20: mol_type = other RNA
ctacgtagttaactcaggag

SEQ ID NO: 1019 - RNA, 20 nt, Synthetic construct
  source 1..20: mol_type = other RNA
ctacgtaattaacacaggag

SEQ ID NO: 1020 - RNA, 20 nt, Synthetic construct
  source 1..20: mol_type = other RNA
ctacgtagttaacacaggag

SEQ ID NO: 1021 - RNA, 20 nt, Synthetic construct
  source 1..20: mol_type = other RNA
ccacgtagttaacacagaat

SEQ ID NO: 1022 - RNA, 20 nt, Synthetic construct
  source 1..20: mol_type = other RNA
ctacgtagttaacaccggag

SEQ ID NO: 1023 - RNA, 20 nt, Synthetic construct
  source 1..20: mol_type = other RNA
ccacgtagttaacacagaaa

SEQ ID NO: 1024 - RNA, 20 nt, Synthetic construct
  source 1..20: mol_type = other RNA
ctacgtagtgaacacaggag

SEQ ID NO: 1025 - RNA, 20 nt, Synthetic construct
  source 1..20: mol_type = other RNA
ctacgtagttaacactgaac

SEQ ID NO: 1026 - RNA, 20 nt, Synthetic construct
  source 1..20: mol_type = other RNA
ctacctagttaacacacgag

SEQ ID NO: 1027 - RNA, 20 nt, Synthetic construct
  source 1..20: mol_type = other RNA
gctcgtagttaacactgaag

SEQ ID NO: 1028 - RNA, 20 nt, Synthetic construct
  source 1..20: mol_type = other RNA
ctgcgaagttaacacagaat

SEQ ID NO: 1029 - RNA, 20 nt, Synthetic construct
  source 1..20: mol_type = other RNA
ctacctagttaacacaaaag

SEQ ID NO: 1030 - RNA, 18 nt, Synthetic construct
  source 1..18: mol_type = other RNA
aacacggtgacagcatct

SEQ ID NO: 1031 - RNA, 18 nt, Synthetic construct
  source 1..18: mol_type = other RNA
cccactgtgatgctatcg

SEQ ID NO: 1032 - RNA, 18 nt, Synthetic construct
  source 1..18: mol_type = other RNA
accacggtgactgcgttg

SEQ ID NO: 1033 - RNA, 18 nt, Synthetic construct
  source 1..18: mol_type = other RNA
accaaggtgacgctgtcg

SEQ ID NO: 1034 - RNA, 18 nt, Synthetic construct
  source 1..18: mol_type = other RNA
aaaacggtgacggtttcg

SEQ ID NO: 1035 - RNA, 18 nt, Synthetic construct
  source 1..18: mol_type = other RNA
tccagtgtgacagtgtag

SEQ ID NO: 1036 - RNA, 18 nt, Synthetic construct
  source 1..18: mol_type = other RNA
acaacggtgacggcgtca

SEQ ID NO: 1037 - RNA, 18 nt, Synthetic construct
  source 1..18: mol_type = other RNA
accacggtgacgatgtcc

SEQ ID NO: 1038 - RNA, 18 nt, Synthetic construct
  source 1..18: mol_type = other RNA
tgcacggtgacggagtcg

SEQ ID NO: 1039 - RNA, 18 nt, Synthetic construct
  source 1..18: mol_type = other RNA
accacggcgacggtttcc

SEQ ID NO: 1040 - RNA, 18 nt, Synthetic construct
  source 1..18: mol_type = other RNA
acgacggtcacggagtcc

SEQ ID NO: 1041 - RNA, 18 nt, Synthetic construct
  source 1..18: mol_type = other RNA
tctacggtgacggagtcg

SEQ ID NO: 1042 - RNA, 18 nt, Synthetic construct
  source 1..18: mol_type = other RNA
gccacggtgacggtgtcc

SEQ ID NO: 1043 - RNA, 18 nt, Synthetic construct
  source 1..18: mol_type = other RNA
atcacggtgacggtgtcc

SEQ ID NO: 1044 - RNA, 18 nt, Synthetic construct
  source 1..18: mol_type = other RNA
accatggtgacggtctcc

SEQ ID NO: 1045 - RNA, 18 nt, Synthetic construct
  source 1..18: mol_type = other RNA
actacggtgacggcgtcg

SEQ ID NO: 1046 - RNA, 18 nt, Synthetic construct
  source 1..18: mol_type = other RNA
gccacggtgacggcgtcc

SEQ ID NO: 1047 - RNA, 18 nt, Synthetic construct
  source 1..18: mol_type = other RNA
accacggtgacggtgact

SEQ ID NO: 1048 - RNA, 18 nt, Synthetic construct
  source 1..18: mol_type = other RNA
tccacggtgacggcgtcg

SEQ ID NO: 1049 - RNA, 18 nt, Synthetic construct
  source 1..18: mol_type = other RNA
gccaaggtgacggcatcg

SEQ ID NO: 1050 - RNA, 18 nt, Synthetic construct
  source 1..18: mol_type = other RNA
accagggtgacggtgcag

SEQ ID NO: 1051 - RNA, 18 nt, Synthetic construct
  source 1..18: mol_type = other RNA
accagggtgacggtatcc

SEQ ID NO: 1052 - RNA, 18 nt, Synthetic construct
  source 1..18: mol_type = other RNA
gccacggtgacggagtcg

SEQ ID NO: 1053 - RNA, 18 nt, Synthetic construct
  source 1..18: mol_type = other RNA
cccacggtgacggtatct

SEQ ID NO: 1054 - RNA, 18 nt, Synthetic construct
  source 1..18: mol_type = other RNA
actacggtgacggtgtct

SEQ ID NO: 1055 - RNA, 18 nt, Synthetic construct
  source 1..18: mol_type = other RNA
accactgtgacggcgtag

SEQ ID NO: 1056 - RNA, 18 nt, Synthetic construct
  source 1..18: mol_type = other RNA
actacggtgacggtttag

SEQ ID NO: 1057 - RNA, 18 nt, Synthetic construct
  source 1..18: mol_type = other RNA
gccacggtgacggtatag

SEQ ID NO: 1058 - RNA, 18 nt, Synthetic construct
  source 1..18: mol_type = other RNA
cccacggtgacggagtcg

SEQ ID NO: 1059 - RNA, 18 nt, Synthetic construct
  source 1..18: mol_type = other RNA
actacggtgacggtgtcc

SEQ ID NO: 1060 - RNA, 18 nt, Synthetic construct
  source 1..18: mol_type = other RNA
accatggtgacggagtcg

SEQ ID NO: 1061 - RNA, 18 nt, Synthetic construct
  source 1..18: mol_type = other RNA
actgcggtgacggcgtcc

SEQ ID NO: 1062 - RNA, 18 nt, Synthetic construct
  source 1..18: mol_type = other RNA
aacaaggtgacggtgtcg

SEQ ID NO: 1063 - RNA, 18 nt, Synthetic construct
  source 1..18: mol_type = other RNA
actacggtgacgaagtcg

SEQ ID NO: 1064 - RNA, 18 nt, Synthetic construct
  source 1..18: mol_type = other RNA
cccacggtgacggagtgg

SEQ ID NO: 1065 - RNA, 18 nt, Synthetic construct
  source 1..18: mol_type = other RNA
agcacggtgacggtggcg

SEQ ID NO: 1066 - RNA, 18 nt, Synthetic construct
  source 1..18: mol_type = other RNA
gcgacggtgacggtgtag

SEQ ID NO: 1067 - RNA, 18 nt, Synthetic construct
  source 1..18: mol_type = other RNA
accacggtgacggcgtct

SEQ ID NO: 1068 - RNA, 18 nt, Synthetic construct
  source 1..18: mol_type = other RNA
accaacgtgacgatgtct

SEQ ID NO: 1069 - RNA, 18 nt, Synthetic construct
  source 1..18: mol_type = other RNA
acgacggtaacggcgtcg

SEQ ID NO: 1070 - RNA, 18 nt, Synthetic construct
  source 1..18: mol_type = other RNA
accacggtgacggtgacc

SEQ ID NO: 1071 - RNA, 20 nt, Synthetic construct
  source 1..20: mol_type = other RNA
ttccctaaaagtcgctccgg

SEQ ID NO: 1072 - RNA, 20 nt, Synthetic construct
  source 1..20: mol_type = other RNA
ttcccttagagccgcaacgg

SEQ ID NO: 1073 - RNA, 20 nt, Synthetic construct
  source 1..20: mol_type = other RNA
gtccctaaaagtcgcacagg

SEQ ID NO: 1074 - RNA, 20 nt, Synthetic construct
  source 1..20: mol_type = other RNA
ctcctttaaagtcgtaccgg

SEQ ID NO: 1075 - RNA, 20 nt, Synthetic construct
  source 1..20: mol_type = other RNA
tccccttaaagtcgcacctg

SEQ ID NO: 1076 - RNA, 20 nt, Synthetic construct
  source 1..20: mol_type = other RNA
ttcccttcgagtcgcaccgg

SEQ ID NO: 1077 - RNA, 20 nt, Synthetic construct
  source 1..20: mol_type = other RNA
ctaccttatagtcgcaccgg

SEQ ID NO: 1078 - RNA, 20 nt, Synthetic construct
  source 1..20: mol_type = other RNA
ttcccttagagtcgcacctg

SEQ ID NO: 1079 - RNA, 20 nt, Synthetic construct
  source 1..20: mol_type = other RNA
ttcccttaaagtcgtacgct

SEQ ID NO: 1080 - RNA, 20 nt, Synthetic construct
  source 1..20: mol_type = other RNA
atcctttaaagtcgcacctg

SEQ ID NO: 1081 - RNA, 20 nt, Synthetic construct
  source 1..20: mol_type = other RNA
ttccctgaaagtcgcgccgg

SEQ ID NO: 1082 - RNA, 20 nt, Synthetic construct
  source 1..20: mol_type = other RNA
ttccgttaaagtcgcaccgc

SEQ ID NO: 1083 - RNA, 20 nt, Synthetic construct
  source 1..20: mol_type = other RNA
ttcacttaaagtcgcgcccg

SEQ ID NO: 1084 - RNA, 20 nt, Synthetic construct
  source 1..20: mol_type = other RNA
ctcccttaaagtcgccccgg

SEQ ID NO: 1085 - RNA, 20 nt, Synthetic construct
  source 1..20: mol_type = other RNA
ttcccataaagtcgcaccgc

SEQ ID NO: 1086 - RNA, 20 nt, Synthetic construct
  source 1..20: mol_type = other RNA
ttaccttaaagtcgcaccgt

SEQ ID NO: 1087 - RNA, 20 nt, Synthetic construct
  source 1..20: mol_type = other RNA
ttcgcttaaagttgtacctg

SEQ ID NO: 1088 - RNA, 20 nt, Synthetic construct
  source 1..20: mol_type = other RNA
ttccctaaaagacgcacggg

SEQ ID NO: 1089 - RNA, 20 nt, Synthetic construct
  source 1..20: mol_type = other RNA
atcctttaaagtcgcaccgg

SEQ ID NO: 1090 - RNA, 20 nt, Synthetic construct
  source 1..20: mol_type = other RNA
ttccctgaaagtcgcactag

SEQ ID NO: 1091 - RNA, 20 nt, Synthetic construct
  source 1..20: mol_type = other RNA
tacccatatagtcgctcagg

SEQ ID NO: 1092 - RNA, 20 nt, Synthetic construct
  source 1..20: mol_type = other RNA
ttctcttaaagtcggaccgg

SEQ ID NO: 1093 - RNA, 20 nt, Synthetic construct
  source 1..20: mol_type = other RNA
ttccctaatggtcgcaatgg

SEQ ID NO: 1094 - RNA, 20 nt, Synthetic construct
  source 1..20: mol_type = other RNA
atcccttaaagtcgtaccag

SEQ ID NO: 1095 - RNA, 20 nt, Synthetic construct
  source 1..20: mol_type = other RNA
ttgcctgaaagtcgcaccgg

SEQ ID NO: 1096 - RNA, 20 nt, Synthetic construct
  source 1..20: mol_type = other RNA
ttcgcttaaagtcgtacggg

SEQ ID NO: 1097 - RNA, 20 nt, Synthetic construct
  source 1..20: mol_type = other RNA
ttcccttaaagtcgcgacag

SEQ ID NO: 1098 - RNA, 20 nt, Synthetic construct
  source 1..20: mol_type = other RNA
ttcccttaaactcgcaccgc

SEQ ID NO: 1099 - RNA, 20 nt, Synthetic construct
  source 1..20: mol_type = other RNA
ttcccataaagtcgcagggg

SEQ ID NO: 1100 - RNA, 20 nt, Synthetic construct
  source 1..20: mol_type = other RNA
gtccctaaaagtcgcaccgg

SEQ ID NO: 1101 - RNA, 20 nt, Synthetic construct
  source 1..20: mol_type = other RNA
gtcccttaaagtcgcaccag

SEQ ID NO: 1102 - RNA, 20 nt, Synthetic construct
  source 1..20: mol_type = other RNA
tttccttaacgtcgcaccgg

SEQ ID NO: 1103 - RNA, 20 nt, Synthetic construct
  source 1..20: mol_type = other RNA
gactcttaaaatcgcaacgg

SEQ ID NO: 1104 - RNA, 20 nt, Synthetic construct
  source 1..20: mol_type = other RNA
tatccttaaagtcgcaccgg

SEQ ID NO: 1105 - RNA, 20 nt, Synthetic construct
  source 1..20: mol_type = other RNA
atcccttaaagtcgggccgg

SEQ ID NO: 1106 - RNA, 20 nt, Synthetic construct
  source 1..20: mol_type = other RNA
ttcccctaaagtcgcaccca

SEQ ID NO: 1107 - RNA, 20 nt, Synthetic construct
  source 1..20: mol_type = other RNA
ttcccttaaagtcgtacagg

SEQ ID NO: 1108 - RNA, 20 nt, Synthetic construct
  source 1..20: mol_type = other RNA
ttccgttaaagtcgctccgg

SEQ ID NO: 1109 - RNA, 20 nt, Synthetic construct
  source 1..20: mol_type = other RNA
ttccgtaaaagtcgcacctg

SEQ ID NO: 1110 - RNA, 20 nt, Synthetic construct
  source 1..20: mol_type = other RNA
ttcccttaaagtcgcacggt

SEQ ID NO: 1111 - RNA, 20 nt, Synthetic construct
  source 1..20: mol_type = other RNA
ttccatgaaagtcgcacggg

SEQ ID NO: 1112 - RNA, 20 nt, Synthetic construct
  source 1..20: mol_type = other RNA
ttccgtaaaagtcgtaccag

SEQ ID NO: 1113 - RNA, 20 nt, Synthetic construct
  source 1..20: mol_type = other RNA
ttcccccaaagtcgcgccgc

SEQ ID NO: 1114 - RNA, 20 nt, Synthetic construct
  source 1..20: mol_type = other RNA
ttcgcgtaaagacgcaccgg

SEQ ID NO: 1115 - RNA, 20 nt, Synthetic construct
  source 1..20: mol_type = other RNA
ttcccttataatcgcaccgg

SEQ ID NO: 1116 - RNA, 20 nt, Synthetic construct
  source 1..20: mol_type = other RNA
ttcctctaaagttgcacctg

SEQ ID NO: 1117 - RNA, 20 nt, Synthetic construct
  source 1..20: mol_type = other RNA
ttccctcaaagtcgcaacgg

SEQ ID NO: 1118 - RNA, 20 nt, Synthetic construct
  source 1..20: mol_type = other RNA
agccctaaaagtcgcacggg

SEQ ID NO: 1119 - RNA, 20 nt, Synthetic construct
  source 1..20: mol_type = other RNA
ttcccttaaagtcgcgcggg

SEQ ID NO: 1120 - RNA, 20 nt, Synthetic construct
  source 1..20: mol_type = other RNA
ttcccttaaagtcgtacggg

SEQ ID NO: 1121 - RNA, 20 nt, Synthetic construct
  source 1..20: mol_type = other RNA
ttcacttaaagtcgcacggg

SEQ ID NO: 1122 - RNA, 20 nt, Synthetic construct
  source 1..20: mol_type = other RNA
ttcccttaaagtcggacctg

SEQ ID NO: 1123 - RNA, 20 nt, Synthetic construct
  source 1..20: mol_type = other RNA
ttcccttaaaatcgcaccag

SEQ ID NO: 1124 - RNA, 20 nt, Synthetic construct
  source 1..20: mol_type = other RNA
ttcccttaaagtcgtgccgg

SEQ ID NO: 1125 - RNA, 20 nt, Synthetic construct
  source 1..20: mol_type = other RNA
ttcccttaaagtcgtaccag

SEQ ID NO: 1126 - RNA, 20 nt, Synthetic construct
  source 1..20: mol_type = other RNA
gtcccttaaagtcgctccgg

SEQ ID NO: 1127 - RNA, 20 nt, Synthetic construct
  source 1..20: mol_type = other RNA
taccctttaagtcgcaccgg

SEQ ID NO: 1128 - RNA, 20 nt, Synthetic construct
  source 1..20: mol_type = other RNA
ttcctctaaagtcgtacagg

SEQ ID NO: 1129 - RNA, 20 nt, Synthetic construct
  source 1..20: mol_type = other RNA
tttccgtaaagtcgcaggag

SEQ ID NO: 1130 - RNA, 20 nt, Synthetic construct
  source 1..20: mol_type = other RNA
ttccctttaagttgcactgg

SEQ ID NO: 1131 - RNA, 20 nt, Synthetic construct
  source 1..20: mol_type = other RNA
ttccctaaaagtcgcaccag

SEQ ID NO: 1132 - RNA, 20 nt, Synthetic construct
  source 1..20: mol_type = other RNA
aacccttaaaatcgcaccgg

SEQ ID NO: 1133 - RNA, 20 nt, Synthetic construct
  source 1..20: mol_type = other RNA
ttcccgtaaagtcgtaccgc

SEQ ID NO: 1134 - RNA, 20 nt, Synthetic construct
  source 1..20: mol_type = other RNA
tttacttaaagtcggaccgg

SEQ ID NO: 1135 - RNA, 18 nt, Synthetic construct
  source 1..18: mol_type = other RNA
ggacttcctaccgaaact

SEQ ID NO: 1136 - RNA, 18 nt, Synthetic construct
  source 1..18: mol_type = other RNA
ggcacaactacccgacct

SEQ ID NO: 1137 - RNA, 18 nt, Synthetic construct
  source 1..18: mol_type = other RNA
ggccctcctacccgagat

SEQ ID NO: 1138 - RNA, 18 nt, Synthetic construct
  source 1..18: mol_type = other RNA
ggccggcctaccccaact

SEQ ID NO: 1139 - RNA, 18 nt, Synthetic construct
  source 1..18: mol_type = other RNA
ggcccccctacccgaagt

SEQ ID NO: 1140 - RNA, 18 nt, Synthetic construct
  source 1..18: mol_type = other RNA
ggccctcctaccccaagt

SEQ ID NO: 1141 - RNA, 18 nt, Synthetic construct
  source 1..18: mol_type = other RNA
ggtccccctacccgaacc

SEQ ID NO: 1142 - RNA, 18 nt, Synthetic construct
  source 1..18: mol_type = other RNA
ggcccccctacccaaact

SEQ ID NO: 1143 - RNA, 18 nt, Synthetic construct
  source 1..18: mol_type = other RNA
ggccctcctacctcaagt

SEQ ID NO: 1144 - RNA, 18 nt, Synthetic construct
  source 1..18: mol_type = other RNA
gggactcctacccgaagt

SEQ ID NO: 1145 - RNA, 18 nt, Synthetic construct
  source 1..18: mol_type = other RNA
ggccttcctaccccaaat

SEQ ID NO: 1146 - RNA, 18 nt, Synthetic construct
  source 1..18: mol_type = other RNA
ggccctcctacctaaaca

SEQ ID NO: 1147 - RNA, 18 nt, Synthetic construct
  source 1..18: mol_type = other RNA
ggcccccctacccaaatt

SEQ ID NO: 1148 - RNA, 18 nt, Synthetic construct
  source 1..18: mol_type = other RNA
ggcccccctacctgaacc

SEQ ID NO: 1149 - RNA, 18 nt, Synthetic construct
  source 1..18: mol_type = other RNA
ggccctgctacccgaatt

SEQ ID NO: 1150 - RNA, 18 nt, Synthetic construct
  source 1..18: mol_type = other RNA
ggcactcctaccccaagt

SEQ ID NO: 1151 - RNA, 18 nt, Synthetic construct
  source 1..18: mol_type = other RNA
agcactcctaccccaagt

SEQ ID NO: 1152 - RNA, 18 nt, Synthetic construct
  source 1..18: mol_type = other RNA
ggccctcatacccggaat

SEQ ID NO: 1153 - RNA, 18 nt, Synthetic construct
  source 1..18: mol_type = other RNA
ggccctcttacccccact

SEQ ID NO: 1154 - RNA, 18 nt, Synthetic construct
  source 1..18: mol_type = other RNA
ggccctcctacctcagct

SEQ ID NO: 1155 - RNA, 18 nt, Synthetic construct
  source 1..18: mol_type = other RNA
agccctcctacccaaagt

SEQ ID NO: 1156 - RNA, 18 nt, Synthetic construct
  source 1..18: mol_type = other RNA
ggccctcctacctgaagt

SEQ ID NO: 1157 - RNA, 18 nt, Synthetic construct
  source 1..18: mol_type = other RNA
ggccctcctacccgggct

SEQ ID NO: 1158 - RNA, 18 nt, Synthetic construct
  source 1..18: mol_type = other RNA
cgccctcctacccgactt

SEQ ID NO: 1159 - RNA, 18 nt, Synthetic construct
  source 1..18: mol_type = other RNA
ggcgctcctaccccaact

SEQ ID NO: 1160 - RNA, 18 nt, Synthetic construct
  source 1..18: mol_type = other RNA
ggccaacctaccccaact

SEQ ID NO: 1161 - RNA, 18 nt, Synthetic construct
  source 1..18: mol_type = other RNA
agccctcctaccccaact

SEQ ID NO: 1162 - RNA, 18 nt, Synthetic construct
  source 1..18: mol_type = other RNA
gggcctcctaccccaact

SEQ ID NO: 1163 - RNA, 18 nt, Synthetic construct
  source 1..18: mol_type = other RNA
gggaatcctacccgaacg

SEQ ID NO: 1164 - RNA, 18 nt, Synthetic construct
  source 1..18: mol_type = other RNA
ggccctcctacccgcgtt

SEQ ID NO: 1165 - RNA, 18 nt, Synthetic construct
  source 1..18: mol_type = other RNA
ggcccttttacccgaagt

SEQ ID NO: 1166 - RNA, 55 nt, Synthetic construct
  source 1..55: mol_type = other RNA
ggtgtcgagaagaggagaacaatatgccaaatgttgttctcgtctcctcgacacc

SEQ ID NO: 1167 - skipped

SEQ ID NO: 1168 - RNA, 101 nt, Synthetic construct
  source 1..101: mol_type = other RNA
gtgttctctatgtaggctccaaaaccaaggagggagtggtgcatggtgtgacaacagtggctgagaagaccaaagagcaagtgacaaatgttggaggagca

SEQ ID NO: 1169 - RNA, 101 nt, Synthetic construct
  source 1..101: mol_type = other RNA
cacccctgaagctctccaaggccgtgcataaggctgtgctgaccatcgacaagaaagggactgaagctgctggggccatgtttttagaggccatacccatg

SEQ ID NO: 1170 - RNA, 101 nt, Synthetic construct
  source 1..101: mol_type = other RNA
gaaccgcaagctgggcaagcctgacttcgactgctcaaaagtggatgagcactggcgcgacggctggaaggatggctcctgtgactagagggtggtcagcc

SEQ ID NO: 1171 - RNA, 101 nt, Synthetic construct
  source 1..101: mol_type = other RNA
tgtcggggagtggccgggagttgggcgagtacgggctgcaggcatacactaaagtgaaaactgtcacagtcaaagtgcctcagaagaactcataagaatca

SEQ ID NO: 1172 - RNA, 101 nt, Synthetic construct
  source 1..101: mol_type = other RNA
cgccatggccatctacaagcagtcacagcacatgacggaggttgtgaggcactgcccccaccatgagcgctgctcagatagcgatggtctggcccctcctc

SEQ ID NO: 1173 - RNA, 101 nt, Synthetic construct
  source 1..101: mol_type = other RNA
ctacaaagaatgtgtctgcaagatcaatgatgactgtacgctcccacggtagcacatgaacgacttcttccactccttcctgattgtgttccgcgtgctgt

SEQ ID NO: 1174 - RNA, 101 nt, Synthetic construct
  source 1..101: mol_type = other RNA
ggatgaagttggtggtgaggccctgggcaggctgctggtggtctacccttagacccagaggttctttgagtcctttggggatctgtccactcctgatgctg

SEQ ID NO: 1175 - RNA, 101 nt, Synthetic construct
  source 1..101: mol_type = other RNA
gagaggcctgctgaaaatgactgaatataaacttgtggtagttggagctgatggcgtaggcaagagtgccttgacgatacagctaattcagaatcattttg

SEQ ID NO: 1176 - RNA, 101 nt, Synthetic construct
  source 1..101: mol_type = other RNA
agagaggcctgctgaaaatgactgaatataaacttgtggtagttggagctagtggcgtaggcaagagtgccttgacgatacagctaattcagaatcatttt

SEQ ID NO: 1177 - RNA, 101 nt, Synthetic construct
  source 1..101: mol_type = other RNA
tgaccggggacccatgtatgatgaccccaccctgcctgaaggctggacacagaagcttaagcaaaggaaatctggccgctctgctgggaagtatgatgtgt

SEQ ID NO: 1178 - RNA, 101 nt, Synthetic construct
  source 1..101: mol_type = other RNA
tgggctaataggactacttctaatctgtaagagcagatccctggacaggcaaggaatacagagggcagcagacatcgaacagcaggctgtgtttgctgtgt

SEQ ID NO: 1179 - RNA, 101 nt, Synthetic construct
  source 1..101: mol_type = other RNA
caagatttatccaggcacctccagcccagcagtggacaggctgtgtgtcgaagttcgccctggagagtgctttggcctcctgggagtgaatggtgccggca

SEQ ID NO: 1180 - RNA, 101 nt, Synthetic construct
  source 1..101: mol_type = other RNA
gccagagagccaggagcatcctgaagctgacccaggtagcgctgccccatacctgaagaccaagtttatctgtgtgacaccaacgacctgcagcaatacca

SEQ ID NO: 1181 - RNA, 101 nt, Synthetic construct
  source 1..101: mol_type = other RNA
aaaggaagcaccagagccaatggaacttgatggccctaaaggaactggatatatcaagactgagttgatttctgtgtctgaagttcacccttctagacttc

SEQ ID NO: 1182 - RNA, 101 nt, Synthetic construct
  source 1..101: mol_type = other RNA
tctcccgtttgtcatttatgctgcgaaagtcttcttgagatgcatcaattatgtcttctttccatcacttaaaccttcttccagtatagatgagtatttct

SEQ ID NO: 1183 - RNA, 101 nt, Synthetic construct
  source 1..101: mol_type = other RNA
actggcagcaacagtcttacctggactctggaatccattctggtgccactaccacagctccttctctgagtggtaaaggcaatcctgaggaagaggatgtg

SEQ ID NO: 1184 - RNA, 101 nt, Synthetic construct
  source 1..101: mol_type = other RNA
gggcctggccccagctgagatctctgcagtgtgtgaaaaagggaatttcaacgtggcccatgggctggcatggtcatattacatcggatatctgcggctga

SEQ ID NO: 1185 - RNA, 101 nt, Synthetic construct
  source 1..101: mol_type = other RNA
ctttgtttgccatgtcacaatacagtcaagctggctttagccgggaggataggcttgagcaggccaaactcttctgccggacacttgaggacatcctggca

SEQ ID NO: 1186 - RNA, 101 nt, Synthetic construct
  source 1..101: mol_type = other RNA
cacaagagatacatccgaggttttggttcctccctcaccacgtaccacaccgttgtcaccgactcttctggtttctcgttcactgtttacaacctcctcgt

SEQ ID NO: 1187 - RNA, 101 nt, Synthetic construct
  source 1..101: mol_type = other RNA
cacaagagatacatccgaggttttggttcctccctcaccacctaccacaccgttgtcaccgactcttctggtttctcgttcactgtttacaacctcctcgt

SEQ ID NO: 1188 - RNA, 101 nt, Synthetic construct
  source 1..101: mol_type = other RNA
cacaagagatacatccgaggttttggttcctccctcaccacttaccacaccgttgtcaccgactcttctggtttctcgttcactgtttacaacctcctcgt

SEQ ID NO: 1189 - RNA, 33 nt, Synthetic construct
  source 1..33: mol_type = other RNA
cacctaccacaccgttgtcaccgactcttctgg

SEQ ID NO: 1190 - RNA, 33 nt, Synthetic construct
  source 1..33: mol_type = other RNA
cacttaccacaccgttgtcaccgactcttctgg

SEQ ID NO: 1191 - RNA, 101 nt, Synthetic construct
  source 1..101: mol_type = other RNA
cacaagagatacatccgaggttttggttcctccctcaccacctaccacagcgttgtcaccgactcttctggtttctcgttcactgtttacaacctcctcgt

SEQ ID NO: 1192 - RNA, 101 nt, Synthetic construct
  source 1..101: mol_type = other RNA
cacaagagatacatccgaggttttggttcctccctcaccacttaccacagcgttgtcaccgactcttctggtttctcgttcactgtttacaacctcctcgt

SEQ ID NO: 1193 - RNA, 33 nt, Synthetic construct
  source 1..33: mol_type = other RNA
cacctaccacagcgttgtcaccgactcttctgg

SEQ ID NO: 1194 - RNA, 33 nt, Synthetic construct
  source 1..33: mol_type = other RNA
cacttaccacagcgttgtcaccgactcttctgg

SEQ ID NO: 1195 - RNA, 75 nt, Synthetic construct
  source 1..75: mol_type = other RNA
cacctaccacaccgttgtcaggtgtcgagaagaggagaacaatatgccaaatgttgttctcgtctcctcgacacc

SEQ ID NO: 1196 - RNA, 75 nt, Synthetic construct
  source 1..75: mol_type = other RNA
cacttaccacaccgttgtcaggtgtcgagaagaggagaacaatatgccaaatgttgttctcgtctcctcgacacc

SEQ ID NO: 1197 - RNA, 101 nt, Synthetic construct
  source 1..101: mol_type = other RNA
ccacagacctcgccctgtatttcaagaagaggacgatgttccaaaagatcatggatcagtctaagacatatgagagtgaacaggagtggacacagtacatg

SEQ ID NO: 1198 - RNA, 101 nt, Synthetic construct
  source 1..101: mol_type = other RNA
ggtgtctggagcgggacataaagttcttctcctgctacaaggttttctagcacctagtcagattctgtatactctcacttgtcctcacctgtgtcatgtac

SEQ ID NO: 1199 - RNA, 100 nt, Synthetic construct
  source 1..100: mol_type = other RNA
ggtgtctggagcgggacataaagttcttctcctgctacaagcttttctagcacctagtcagattctgtatactctcacttgtcctcacctgtgtcatgta

SEQ ID NO: 1200 - RNA, 100 nt, Synthetic construct
  source 1..100: mol_type = other RNA
ggtgtctggagcgggacataaagttcttctcctgctacaagtttttctagcacctagtcagattctgtatactctcacttgtcctcacctgtgtcatgta

SEQ ID NO: 1201 - RNA, 33 nt, Synthetic construct
  source 1..33: mol_type = other RNA
aagcttttctagcacctagtcagattctgtata

SEQ ID NO: 1202 - RNA, 33 nt, Synthetic construct
  source 1..33: mol_type = other RNA
aagtttttctagcacctagtcagattctgtata

SEQ ID NO: 1203 - RNA, 100 nt, Synthetic construct
  source 1..100: mol_type = other RNA
ggtgtctggagcgggacataaagttcttctcctgctacaagcttttctagcatctagtcagattctgtatactctcacttgtcctcacctgtgtcatgta

SEQ ID NO: 1204 - RNA, 100 nt, Synthetic construct
  source 1..100: mol_type = other RNA
ggtgtctggagcgggacataaagttcttctcctgctacaagtttttctagcatctagtcagattctgtatactctcacttgtcctcacctgtgtcatgta

SEQ ID NO: 1205 - RNA, 33 nt, Synthetic construct
  source 1..33: mol_type = other RNA
aagcttttctagcatctagtcagattctgtata

SEQ ID NO: 1206 - RNA, 33 nt, Synthetic construct
  source 1..33: mol_type = other RNA
aagtttttctagcatctagtcagattctgtata

SEQ ID NO: 1207 - RNA, 75 nt, Synthetic construct
  source 1..75: mol_type = other RNA
aagcttttctagcatctagtggtgtcgagaagaggagaacaatatgccaaatgttgttctcgtctcctcgacacc

SEQ ID NO: 1208 - RNA, 75 nt, Synthetic construct
  source 1..75: mol_type = other RNA
aagtttttctagcatctagtggtgtcgagaagaggagaacaatatgccaaatgttgttctcgtctcctcgacacc

SEQ ID NO: 1209 - RNA, 101 nt, Synthetic construct
  source 1..101: mol_type = other RNA
acagcccctcaccggccctcaacccgctcatgcccgacgtccgtatgtgacttcacttttgacagtgtcagtttcacggagtcttcttgagtattcttagt

SEQ ID NO: 1210 - RNA, 101 nt, Synthetic construct
  source 1..101: mol_type = other RNA
acagcccctcaccggccctcaacccgctcatgcccgacgtccctatgtgacttcacttttgacagtgtcagtttcacggagtcttcttgagtattcttagt

SEQ ID NO: 1211 - RNA, 33 nt, Synthetic construct
  source 1..33: mol_type = other RNA
gtccctatgtgacttcacttttgacagtgtcag

SEQ ID NO: 1212 - RNA, 75 nt, Synthetic construct
  source 1..75: mol_type = other RNA
gtccctatgtgacttcacttggtgtcgagaagaggagaacaatatgccaaatgttgttctcgtctcctcgacacc

SEQ ID NO: 1213 - RNA, 101 nt, Synthetic construct
  source 1..101: mol_type = other RNA
actggcccctgggtacatactactggggtgggacggacttccgacctgtgccttcgaattcgtttcctttagaccggcgagacgacccttcatactacaca

SEQ ID NO: 1214 - RNA, 101 nt, Synthetic construct
  source 1..101: mol_type = other RNA
actggcccctgggtacatactactggggtgggacggacttcccacctgtgccttcgaattcgtttcctttagaccggcgagacgacccttcatactacaca

SEQ ID NO: 1215 - RNA, 101 nt, Synthetic construct
  source 1..101: mol_type = other RNA
actggcccctgggtacatactactggggtgggacggacttcctacctgtgccttcgaattcgtttcctttagaccggcgagacgacccttcatactacaca

SEQ ID NO: 1216 - RNA, 33 nt, Synthetic construct
  source 1..33: mol_type = other RNA
ttcccacctgtgccttcgaattcgtttccttta

SEQ ID NO: 1217 - RNA, 33 nt, Synthetic construct
  source 1..33: mol_type = other RNA
ttcctacctgtgccttcgaattcgtttccttta

SEQ ID NO: 1218 - RNA, 75 nt, Synthetic construct
  source 1..75: mol_type = other RNA
ttcccacctgtgccttcgaaggtgtcgagaagaggagaacaatatgccaaatgttgttctcgtctcctcgacacc

SEQ ID NO: 1219 - RNA, 75 nt, Synthetic construct
  source 1..75: mol_type = other RNA
ttcctacctgtgccttcgaaggtgtcgagaagaggagaacaatatgccaaatgttgttctcgtctcctcgacacc

SEQ ID NO: 1220 - RNA, 101 nt, Synthetic construct
  source 1..101: mol_type = other RNA
agagggcaaacagtaaatacgacgctttcagaagaactctacgtagttaacacagaagaaaggtagtgaatttggaagaaggtcatatctactcataaaga

SEQ ID NO: 1221 - RNA, 101 nt, Synthetic construct
  source 1..101: mol_type = other RNA
agagggcaaacagtaaatacgacgctttcagaagaactctacctagttaacacagaagaaaggtagtgaatttggaagaaggtcatatctactcataaaga

SEQ ID NO: 1222 - RNA, 101 nt, Synthetic construct
  source 1..101: mol_type = other RNA
agagggcaaacagtaaatacgacgctttcagaagaactctacttagttaacacagaagaaaggtagtgaatttggaagaaggtcatatctactcataaaga

SEQ ID NO: 1223 - RNA, 33 nt, Synthetic construct
  source 1..33: mol_type = other RNA
ctacctagttaacacagaagaaaggtagtgaat

SEQ ID NO: 1224 - RNA, 33 nt, Synthetic construct
  source 1..33: mol_type = other RNA
ctacttagttaacacagaagaaaggtagtgaat

SEQ ID NO: 1225 - RNA, 101 nt, Synthetic construct
  source 1..101: mol_type = other RNA
agagggcaaacagtaaatacgacgctttcagaagaactctacctagttaacatagaagaaaggtagtgaatttggaagaaggtcatatctactcataaaga

SEQ ID NO: 1226 - RNA, 101 nt, Synthetic construct
  source 1..101: mol_type = other RNA
agagggcaaacagtaaatacgacgctttcagaagaactctacttagttaacatagaagaaaggtagtgaatttggaagaaggtcatatctactcataaaga

SEQ ID NO: 1227 - RNA, 33 nt, Synthetic construct
  source 1..33: mol_type = other RNA
ctacctagttaacatagaagaaaggtagtgaat

SEQ ID NO: 1228 - RNA, 33 nt, Synthetic construct
  source 1..33: mol_type = other RNA
ctacttagttaacatagaagaaaggtagtgaat

SEQ ID NO: 1229 - RNA, 75 nt, Synthetic construct
  source 1..75: mol_type = other RNA
ctacctagttaacacagaagggtgtcgagaagaggagaacaatatgccaaatgttgttctcgtctcctcgacacc

SEQ ID NO: 1230 - RNA, 75 nt, Synthetic construct
  source 1..75: mol_type = other RNA
ctacttagttaacacagaagggtgtcgagaagaggagaacaatatgccaaatgttgttctcgtctcctcgacacc

SEQ ID NO: 1231 - RNA, 101 nt, Synthetic construct
  source 1..101: mol_type = other RNA
gtggggacttcgagaggttccggcacgtattccgacacgactggtagctgctctttccctgacttcgacgaccccggtacaaaaatctccggtatgggtac

SEQ ID NO: 1232 - RNA, 101 nt, Synthetic construct
  source 1..101: mol_type = other RNA
gtggggacttcgagaggttccggcacgtattccgacacgactgctagctgctctttccctgacttcgacgaccccggtacaaaaatctccggtatgggtac

SEQ ID NO: 1233 - RNA, 101 nt, Synthetic construct
  source 1..101: mol_type = other RNA
gtggggacttcgagaggttccggcacgtattccgacacgactgttagctgctctttccctgacttcgacgaccccggtacaaaaatctccggtatgggtac

SEQ ID NO: 1234 - RNA, 33 nt, Synthetic construct
  source 1..33: mol_type = other RNA
gactgctagctgctctttccctgacttcgacga

SEQ ID NO: 1235 - RNA, 33 nt, Synthetic construct
  source 1..33: mol_type = other RNA
gactgttagctgctctttccctgacttcgacga

SEQ ID NO: 1236 - RNA, 101 nt, Synthetic construct
  source 1..101: mol_type = other RNA
gtggggacttcgagaggttccggcacgtattccgacacgactgctagctgctttttccctgacttcgacgaccccggtacaaaaatctccggtatgggtac

SEQ ID NO: 1237 - RNA, 101 nt, Synthetic construct
  source 1..101: mol_type = other RNA
gtggggacttcgagaggttccggcacgtattccgacacgactgttagctgctttttccctgacttcgacgaccccggtacaaaaatctccggtatgggtac

SEQ ID NO: 1238 - RNA, 33 nt, Synthetic construct
  source 1..33: mol_type = other RNA
gactgctagctgctttttccctgacttcgacga

SEQ ID NO: 1239 - RNA, 33 nt, Synthetic construct
  source 1..33: mol_type = other RNA
gactgttagctgctttttccctgacttcgacga

SEQ ID NO: 1240 - RNA, 75 nt, Synthetic construct
  source 1..75: mol_type = other RNA
gactgctagctgctctttccggtgtcgagaagaggagaacaatatgccaaatgttgttctcgtctcctcgacacc

SEQ ID NO: 1241 - RNA, 75 nt, Synthetic construct
  source 1..75: mol_type = other RNA
gactgttagctgctctttccggtgtcgagaagaggagaacaatatgccaaatgttgttctcgtctcctcgacacc

SEQ ID NO: 1242 - RNA, 101 nt, Synthetic construct
  source 1..101: mol_type = other RNA
gatgtttcttacacagacgttctagttactactgacatgcgagggtgccaccgtgtacttgctgaagaaggtgaggaaggactaacacaaggcgcacgaca

SEQ ID NO: 1243 - RNA, 101 nt, Synthetic construct
  source 1..101: mol_type = other RNA
gatgtttcttacacagacgttctagttactactgacatgcgaggctgccaccgtgtacttgctgaagaaggtgaggaaggactaacacaaggcgcacgaca

SEQ ID NO: 1244 - RNA, 101 nt, Synthetic construct
  source 1..101: mol_type = other RNA
gatgtttcttacacagacgttctagttactactgacatgcgaggttgccaccgtgtacttgctgaagaaggtgaggaaggactaacacaaggcgcacgaca

SEQ ID NO: 1245 - RNA, 33 nt, Synthetic construct
  source 1..33: mol_type = other RNA
gcgaggctgccaccgtgtacttgctgaagaagg

SEQ ID NO: 1246 - RNA, 33 nt, Synthetic construct
  source 1..33: mol_type = other RNA
gcgaggttgccaccgtgtacttgctgaagaagg

SEQ ID NO: 1247 - RNA, 75 nt, Synthetic construct
  source 1..75: mol_type = other RNA
gcgaggctgccaccgtgtacggtgtcgagaagaggagaacaatatgccaaatgttgttctcgtctcctcgacacc

SEQ ID NO: 1248 - RNA, 75 nt, Synthetic construct
  source 1..75: mol_type = other RNA
gcgaggttgccaccgtgtacggtgtcgagaagaggagaacaatatgccaaatgttgttctcgtctcctcgacacc

SEQ ID NO: 1249 - RNA, 101 nt, Synthetic construct
  source 1..101: mol_type = other RNA
gtggggacttcgagaggttccggcacgtattccgacacgactggtagctgctttttccctgacttcgacgaccccggtacaaaaatctccggtatgggtac

SEQ ID NO: 1250 - RNA, 33 nt, Synthetic construct
  source 1..33: mol_type = other RNA
gactggtagctgctttttccctgacttcgacga

SEQ ID NO: 1251 - RNA, 101 nt, Synthetic construct
  source 1..101: mol_type = other RNA
ggtgtctggagcgggacataaagttcttctcctgctacaaggttttctagcatctagtcagattctgtatactctcacttgtcctcacctgtgtcatgtac

SEQ ID NO: 1252 - RNA, 33 nt, Synthetic construct
  source 1..33: mol_type = other RNA
aaggttttctagcatctagtcagattctgtata

SEQ ID NO: 1253 - RNA, 101 nt, Synthetic construct
  source 1..101: mol_type = other RNA
ctctccggacgacttttactgacttatatttgaacaccatcaacctcgaccaccgcatccgttctcacggaactgctatgtcgattaagtcttagtaaaac

SEQ ID NO: 1254 - RNA, 101 nt, Synthetic construct
  source 1..101: mol_type = other RNA
ctctccggacgacttttactgacttatatttgaacaccatcaacctcgaccatcgcatccgttctcacggaactgctatgtcgattaagtcttagtaaaac

SEQ ID NO: 1255 - RNA, 33 nt, Synthetic construct
  source 1..33: mol_type = other RNA
atcaacctcgaccatcgcatccgttctcacgga

SEQ ID NO: 1256 - RNA, 101 nt, Synthetic construct
  source 1..101: mol_type = other RNA
acccgattatcctgatgaagattagacattctcgtctagggacctgtccgctccttatgtctcccgtcgtctgtagcttgtcgtccgacacaaacgacaca

SEQ ID NO: 1257 - RNA, 101 nt, Synthetic construct
  source 1..101: mol_type = other RNA
acccgattatcctgatgaagattagacattctcgtctagggacctgtccgcttcttatgtctcccgtcgtctgtagcttgtcgtccgacacaaacgacaca

SEQ ID NO: 1258 - RNA, 33 nt, Synthetic construct
  source 1..33: mol_type = other RNA
gggacctgtccgcttcttatgtctcccgtcgtc

SEQ ID NO: 1259 - RNA, 101 nt, Synthetic construct
  source 1..101: mol_type = other RNA
gttctaaataggtccgtggaggtcgggtcgtcacctgtccgacacacagcctcaagcgggacctctcacgaaaccggaggaccctcacttaccacggccgt

SEQ ID NO: 1260 - RNA, 101 nt, Synthetic construct
  source 1..101: mol_type = other RNA
gttctaaataggtccgtggaggtcgggtcgtcacctgtccgacacacagccttaagcgggacctctcacgaaaccggaggaccctcacttaccacggccgt

SEQ ID NO: 1261 - RNA, 33 nt, Synthetic construct
  source 1..33: mol_type = other RNA
ccgacacacagccttaagcgggacctctcacga

SEQ ID NO: 1262 - RNA, 101 nt, Synthetic construct
  source 1..101: mol_type = other RNA
agagggcaaacagtaaatacgacgctttcagaagaactctacgtagttaacatagaagaaaggtagtgaatttggaagaaggtcatatctactcataaaga

SEQ ID NO: 1263 - RNA, 33 nt, Synthetic construct
  source 1..33: mol_type = other RNA
ctacgtagttaacatagaagaaaggtagtgaat

SEQ ID NO: 1264 - RNA, 101 nt, Synthetic construct
  source 1..101: mol_type = other RNA
gaaacaaacggtacagtgttatgtcagttcgaccgaaatcggccctcctacccgaactcgtccggtttgagaagacggcctgtgaactcctgtaggaccgt

SEQ ID NO: 1265 - RNA, 101 nt, Synthetic construct
  source 1..101: mol_type = other RNA
gaaacaaacggtacagtgttatgtcagttcgaccgaaatcggccctcctacctgaactcgtccggtttgagaagacggcctgtgaactcctgtaggaccgt

SEQ ID NO: 1266 - RNA, 33 nt, Synthetic construct
  source 1..33: mol_type = other RNA
tcggccctcctacctgaactcgtccggtttgag

SEQ ID NO: 1267 - RNA, 101 nt, Synthetic construct
  source 1..101: mol_type = other RNA
gttctaaataggtccgtggaggtcgggtcgtcacctgtccgacacacaggcttaagcgggacctctcacgaaaccggaggaccctcacttaccacggccgt

SEQ ID NO: 1268 - RNA, 33 nt, Synthetic construct
  source 1..33: mol_type = other RNA
ccgacacacaggcttaagcgggacctctcacga

SEQ ID NO: 1269 - RNA, 75 nt, Synthetic construct
  source 1..75: mol_type = other RNA
gactggtagctgctctttccggtgtcgagaagaggagaacaatatgccaaatgttgttctcgtctcctcgacacc

SEQ ID NO: 1270 - RNA, 101 nt, Synthetic construct
  source 1..101: mol_type = other RNA
cttggcgttcgacccgttcggactgaagctgacgagttttcacctactcgcgaccgcgctgccgaccttcctaccgaggacactgatctcccaccagtcgg

SEQ ID NO: 1271 - RNA, 101 nt, Synthetic construct
  source 1..101: mol_type = other RNA
cttggcgttcgacccgttcggactgaagctgacgagttttcacctactcgcgcccgcgctgccgaccttcctaccgaggacactgatctcccaccagtcgg

SEQ ID NO: 1272 - RNA, 101 nt, Synthetic construct
  source 1..101: mol_type = other RNA
cttggcgttcgacccgttcggactgaagctgacgagttttcacctactcgcgtccgcgctgccgaccttcctaccgaggacactgatctcccaccagtcgg

SEQ ID NO: 1273 - RNA, 33 nt, Synthetic construct
  source 1..33: mol_type = other RNA
ttcacctactcgcgcccgcgctgccgaccttcc

SEQ ID NO: 1274 - RNA, 33 nt, Synthetic construct
  source 1..33: mol_type = other RNA
ttcacctactcgcgtccgcgctgccgaccttcc

SEQ ID NO: 1275 - RNA, 101 nt, Synthetic construct
  source 1..101: mol_type = other RNA
tctctccggacgacttttactgacttatatttgaacaccatcaacctcgaccaccgcatccgttctcacggaactgctatgtcgattaagtcttagtaaaa

SEQ ID NO: 1276 - RNA, 101 nt, Synthetic construct
  source 1..101: mol_type = other RNA
tctctccggacgacttttactgacttatatttgaacaccatcaacctcgacccccgcatccgttctcacggaactgctatgtcgattaagtcttagtaaaa

SEQ ID NO: 1277 - RNA, 101 nt, Synthetic construct
  source 1..101: mol_type = other RNA
tctctccggacgacttttactgacttatatttgaacaccatcaacctcgacctccgcatccgttctcacggaactgctatgtcgattaagtcttagtaaaa

SEQ ID NO: 1278 - RNA, 33 nt, Synthetic construct
  source 1..33: mol_type = other RNA
catcaacctcgacccccgcatccgttctcacgg

SEQ ID NO: 1279 - RNA, 33 nt, Synthetic construct
  source 1..33: mol_type = other RNA
catcaacctcgacctccgcatccgttctcacgg

SEQ ID NO: 1280 - RNA, 101 nt, Synthetic construct
  source 1..101: mol_type = other RNA
gcggtaccggtagatgttcgtcagtgtcgtgtactgcctccaacactccgcgacgggggtggtactcgcgacgagtctatcgctaccagaccggggaggag

SEQ ID NO: 1281 - RNA, 101 nt, Synthetic construct
  source 1..101: mol_type = other RNA
gcggtaccggtagatgttcgtcagtgtcgtgtactgcctccaacactccgcgccgggggtggtactcgcgacgagtctatcgctaccagaccggggaggag

SEQ ID NO: 1282 - RNA, 101 nt, Synthetic construct
  source 1..101: mol_type = other RNA
gcggtaccggtagatgttcgtcagtgtcgtgtactgcctccaacactccgcgtcgggggtggtactcgcgacgagtctatcgctaccagaccggggaggag

SEQ ID NO: 1283 - RNA, 33 nt, Synthetic construct
  source 1..33: mol_type = other RNA
tccaacactccgcgccgggggtggtactcgcga

SEQ ID NO: 1284 - RNA, 33 nt, Synthetic construct
  source 1..33: mol_type = other RNA
tccaacactccgcgtcgggggtggtactcgcga

SEQ ID NO: 1285 - RNA, 101 nt, Synthetic construct
  source 1..101: mol_type = other RNA
aaagggaacgattccatcactatccatctttactttgatgatatttgatgaatgccacaacactagtaaacaacacccgtacaatatgatcatgtttaatt

SEQ ID NO: 1286 - RNA, 101 nt, Synthetic construct
  source 1..101: mol_type = other RNA
tttcccttgctaaggtagtgataggtagaaatgaaactactataaactacctacggtgttgtgatcatttgttgtgggcatgttatactagtacaaattaa

SEQ ID NO: 1287 - RNA, 101 nt, Synthetic construct
  source 1..101: mol_type = other RNA
tttcccttgctaaggtagtgataggtagaaatgaaactactataaactacctccggtgttgtgatcatttgttgtgggcatgttatactagtacaaattaa

SEQ ID NO: 1288 - RNA, 101 nt, Synthetic construct
  source 1..101: mol_type = other RNA
tttcccttgctaaggtagtgataggtagaaatgaaactactataaactaccttcggtgttgtgatcatttgttgtgggcatgttatactagtacaaattaa

SEQ ID NO: 1289 - RNA, 33 nt, Synthetic construct
  source 1..33: mol_type = other RNA
actataaactacctccggtgttgtgatcatttg

SEQ ID NO: 1290 - RNA, 33 nt, Synthetic construct
  source 1..33: mol_type = other RNA
actataaactaccttcggtgttgtgatcatttg

SEQ ID NO: 1291 - RNA, 101 nt, Synthetic construct
  source 1..101: mol_type = other RNA
agaagatgctggtgttcaattgtcagacttttccctcattatcattgatgaatgtcatcacaccaacaaagaagcagtgtataataacatcatgaggcatt

SEQ ID NO: 1292 - RNA, 101 nt, Synthetic construct
  source 1..101: mol_type = other RNA
tcttctacgaccacaagttaacagtctgaaaagggagtaatagtaactacctacagtagtgtggttgtttcttcgtcacatattattgtagtactccgtaa

SEQ ID NO: 1293 - RNA, 101 nt, Synthetic construct
  source 1..101: mol_type = other RNA
tcttctacgaccacaagttaacagtctgaaaagggagtaatagtaactacctccagtagtgtggttgtttcttcgtcacatattattgtagtactccgtaa

SEQ ID NO: 1294 - RNA, 101 nt, Synthetic construct
  source 1..101: mol_type = other RNA
tcttctacgaccacaagttaacagtctgaaaagggagtaatagtaactaccttcagtagtgtggttgtttcttcgtcacatattattgtagtactccgtaa

SEQ ID NO: 1295 - RNA, 33 nt, Synthetic construct
  source 1..33: mol_type = other RNA
aatagtaactacctccagtagtgtggttgtttc

SEQ ID NO: 1296 - RNA, 33 nt, Synthetic construct
  source 1..33: mol_type = other RNA
aatagtaactaccttcagtagtgtggttgtttc

SEQ ID NO: 1297 - RNA, 101 nt, Synthetic construct
  source 1..101: mol_type = other RNA
aaatgagtccaatgtcagattttggaactcaaccctatgaacaatgggccattcaaatggaaaaaaaagctgcaaaagaaggaaatcgcaaagaacgtgtt

SEQ ID NO: 1298 - RNA, 101 nt, Synthetic construct
  source 1..101: mol_type = other RNA
tttactcaggttacagtctaaaaccttgagttgggatacttgttacccggcaagtttaccttttttttcgacgttttcttcctttagcgtttcttgcacaa

SEQ ID NO: 1299 - RNA, 101 nt, Synthetic construct
  source 1..101: mol_type = other RNA
tttactcaggttacagtctaaaaccttgagttgggatacttgttacccggcacgtttaccttttttttcgacgttttcttcctttagcgtttcttgcacaa

SEQ ID NO: 1300 - RNA, 101 nt, Synthetic construct
  source 1..101: mol_type = other RNA
tttactcaggttacagtctaaaaccttgagttgggatacttgttacccggcatgtttaccttttttttcgacgttttcttcctttagcgtttcttgcacaa

SEQ ID NO: 1301 - RNA, 32 nt, Synthetic construct
  source 1..32: mol_type = other RNA
cttgttacccggcacgtttaccttttttttcg

SEQ ID NO: 1302 - RNA, 32 nt, Synthetic construct
  source 1..32: mol_type = other RNA
cttgttacccggcatgtttaccttttttttcg

SEQ ID NO: 1303 - RNA, 101 nt, Synthetic construct
  source 1..101: mol_type = other RNA
gcggtaccggtagatgttcgtcagtgtcgtgtactgcctccaacactccgcgccaggggtggtactcgcgacgagtctatcgctaccagaccggggaggag

SEQ ID NO: 1304 - RNA, 101 nt, Synthetic construct
  source 1..101: mol_type = other RNA
gcggtaccggtagatgttcgtcagtgtcgtgtactgcctccaacactccgcgtcaggggtggtactcgcgacgagtctatcgctaccagaccggggaggag

SEQ ID NO: 1305 - RNA, 33 nt, Synthetic construct
  source 1..33: mol_type = other RNA
tccaacactccgcgccaggggtggtactcgcga

SEQ ID NO: 1306 - RNA, 33 nt, Synthetic construct
  source 1..33: mol_type = other RNA
tccaacactccgcgtcaggggtggtactcgcga

SEQ ID NO: 1307 - RNA, 75 nt, Synthetic construct
  source 1..75: mol_type = other RNA
tccaacactccgcgccggggggtgtcgagaagaggagaacaatatgccaaatgttgttctcgtctcctcgacacc

SEQ ID NO: 1308 - RNA, 75 nt, Synthetic construct
  source 1..75: mol_type = other RNA
tccaacactccgcgtcggggggtgtcgagaagaggagaacaatatgccaaatgttgttctcgtctcctcgacacc

SEQ ID NO: 1309 - RNA, 101 nt, Synthetic construct
  source 1..101: mol_type = other RNA
gcggtaccggtagatgttcgtcagtgtcgtgtactgcctccaacactccgcgacaggggtggtactcgcgacgagtctatcgctaccagaccggggaggag

SEQ ID NO: 1310 - RNA, 33 nt, Synthetic construct
  source 1..33: mol_type = other RNA
tccaacactccgcgacaggggtggtactcgcga

SEQ ID NO: 1311 - RNA, 101 nt, Synthetic construct
  source 1..101: mol_type = other RNA
tttcccttgctaaggtagtgataggtagaaatgaaactactataaactacctacagtgttgtgatcatttgttgtgggcatgttatactagtacaaattaa

SEQ ID NO: 1312 - RNA, 33 nt, Synthetic construct
  source 1..33: mol_type = other RNA
actataaactacctacagtgttgtgatcatttg

SEQ ID NO: 1313 - RNA, 75 nt, Synthetic construct
  source 1..75: mol_type = other RNA
tccaacactccgcgacagggggtgtcgagaagaggagaacaatatgccaaatgttgttctcgtctcctcgacacc

SEQ ID NO: 1314 - RNA, 101 nt, Synthetic construct
  source 1..101: mol_type = other RNA
agagggcaaacagtaaatacgacgctttcagaagaactctacgtagttaacacaggagaaaggtagtgaatttggaagaaggtcatatctactcataaaga

SEQ ID NO: 1315 - RNA, 33 nt, Synthetic construct
  source 1..33: mol_type = other RNA
ctacgtagttaacacaggagaaaggtagtgaat

SEQ ID NO: 1316 - RNA, 101 nt, Synthetic construct
  source 1..101: mol_type = other RNA
agagggcaaacagtaaatacgacgctttcagaagaactctacgtagttaacataggagaaaggtagtgaatttggaagaaggtcatatctactcataaaga

SEQ ID NO: 1317 - RNA, 33 nt, Synthetic construct
  source 1..33: mol_type = other RNA
ctacgtagttaacataggagaaaggtagtgaat

SEQ ID NO: 1318 - RNA, 75 nt, Synthetic construct
  source 1..75: mol_type = other RNA
ctacgtagttaacacaggagggtgtcgagaagaggagaacaatatgccaaatgttgttctcgtctcctcgacacc